(19) United States
(12) Patent Application Publication
(10) Pub. No.: US 2024/0207422 A1
(43) Pub. Date: Jun. 27, 2024

(54) NERVE TARGETING PEPTIDE CONJUGATES AND METHODS OF USE

(71) Applicant: Alume Biosciences, Inc., San Diego, CA (US)

(72) Inventors: Michael Whitney, San Diego, CA (US); Quyen Nguyen, San Diego, CA (US)

(21) Appl. No.: 18/531,565

(22) Filed: Dec. 6, 2023

Related U.S. Application Data
(60) Provisional application No. 63/386,470, filed on Dec. 7, 2022.

(51) Int. Cl. A61K 47/64; A61K 41/00; A61K 49/00

(52) U.S. Cl. CPC A61K 47/64; A61K 41/0057; A61K 49/0056

(57) ABSTRACT

Nerve targeting peptides, including those comprising the amino acid sequence of any one of SEQ ID NOS:1-557, Formulas (I)-(XLIV), and Subformulas (Ia)-(XLIVa); conjugates comprising such nerve targeting peptides and a cargo molecule; compositions comprising such peptides and conjugates; and their use in methods of fluorescent guided surgery and other applications.

### CROSS REFERENCE TO RELATED APPLICATION

The present application claims benefit of U.S. Provisional Application No. 63/386,470 filed on Dec. 7, 2022 and is incorporated by reference in its entirety.

### REFERENCE TO AN ELECTRONIC SEQUENCE LISTING

The contents of the electronic sequence listing (409_SeqListing.xml; Size: 504,874 bytes; and Date of Creation: Dec. 5, 2023) is herein incorporated by reference in its entirety.

### FIELD

The present invention relates to nerve targeting peptides and conjugates thereof and their use in intraoperative nerve visualization and other targeted surgical and medical applications.

### BACKGROUND

Preserving human neurons and human nerves is an important goal of surgical procedures; for example, accidental transection of nerves can lead to significant morbidity, such as chronic pain and loss of function. Nerves are typically identified by their elongated whitish appearance and relationship to nearby structures or by electrophysiological studies. However, such nerve identification can be difficult. Nerve structures can be obscured or distorted in complex surgical settings, such as those involving trauma, tumors, inflammation, or infection. Nerve visibility can be further restricted in minimally invasive procedures, such an endoscopy, which rely on small incisions with limited access. Indeed, inadvertent nerve damage is a leading complication associated with many surgeries using both open and minimal access procedures.

These observations highlight the need for methods and tools to identify neurons or nerves reliably and efficiently. Electromyography (EMG) offers one approach to neuron or nerve identification prior to direct exposure during surgery or confirmation of neuron or nerve identity in instances of uncertainty. A disadvantage of this technique, however, is the lack of visual feedback to the operating surgeon. Even if a nerve has been identified in one location, either through accidental or purposeful stimulation, there is no visual guidance to the operating surgeon as to how far away from the stimulation site the nerve lies or the direction of travel the nerve takes away from the stimulation site. Furthermore, EMG only traces motor pathways, not sensory fibers. EMG can fail if neuron or nerve conduction or neuromuscular transmission is temporarily blocked anywhere distal to the recording site. Such blockade easily occurs due to neuron or nerve compression, trauma, local anesthetics, or neuromuscular blockers.

Other approaches include neuron or nerve labeling, which primarily depends on retrograde or anterograde tracing of individually identified axonal tracts via the use of fluorescent dyes. However, methods of labeling neurons or nerves by locally applied fluorescent tracers can be limited. First, depending on where the dye has been injected, this technique can label only one neuron or nerve fiber tract at a time. Second, this technique may result in only limited labeling of fluorescent dyes along the axonal tracts because retrograde axonal tracers typically accumulate in the neural cell body. Third, retrograde transport is relatively slow (on the order of millimeters per day) and therefore takes a long time to label human neuron or nerves, which are often longer than a meter, such as in the case of the sciatic neuron or nerve and its arborizations. Fourth, the application of fluorescent dyes to innervation targets, such as direct intramuscular injections to label a motor neuron or nerves, is typically messy with a variable amount of the tracer dye remaining at the injection site. As dissection of neurons or nerves depends on accurate visualization of adjacent structures prior to encountering them, a surgical site that is contaminated with fluorescent dyes would not be desirable. Finally, the direct injection of the fluorescent dye itself may be damaging to the target organs or neuron or nerve of interest, either by mechanical damage or by the very high local concentration of dye and vehicle at the injection site.

Accordingly, there is a need in the art to identify peptides capable of selective binding to human nerves and neurons, to facilitate surgical procedures and human nerve protection. The present disclosure provides peptide sequences that selectively bind to human nerves or neurons, as well as methods of using those peptides, for example in conjunction with selected cargo molecules to allow fluorescent visualization of nerves during surgery, or to target drugs and other agents to nerves.

### BRIEF SUMMARY

In one aspect, the present disclosure provides a nerve targeting peptide conjugate comprising: a peptide consisting or comprising the amino acid sequence of any one of SEQ ID NOS: 1-557; and a cargo molecule. In one aspect, the present disclosure provides a nerve targeting peptide conjugate comprising: a peptide consisting or comprising the amino acid sequence of any one of Formulas I-XLIV, as described herein, including Subformulas Ia-XLIVa; and a cargo molecule.

In some embodiments, the cargo molecule comprises a drug, fluorescent moiety, or photosensitizing agent.

In some embodiments, the cargo molecule is joined to the N-terminus or C-terminus of the peptide.

In some embodiments, the cargo molecule is joined to the peptide via a linker.

In some embodiments, the linker is a straight or branched-chain carbon linker, heterocyclic carbon linker, amino acid linker, lipophilic residue, peptide linker, peptide nucleic acid linker, hydrazone linker, SPDB disulfide, sulfo-SPDB, maleimidomethyl cyclohexane-1-carboxylate (MCC), aminohexanoic acid linker, polyether linker, or polyethylene glycol linker.

In some embodiments, the cargo molecule comprises a fluorescent moiety.

In some embodiments, the fluorescent moiety is a fluorescent protein, a fluorescent peptide, a fluorophore, or any combination thereof.

In some embodiments, the fluorescent moiety is selected from the group consisting of: a xanthene; a bimane; a coumarin; an aromatic amines; a benzofuran; a fluorescent cyanine; a carbazole; a dicyanomethylene pyrane; polymethine; oxabenzanthrane; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives thereof.

In some embodiments, the fluorescent moiety is selected from the group consisting of: 5-carboxyfluorescein; fluorescein-5-isothiocyanate; 6-carboxyfluorescein; tetramethylrhodamine-6-isothiocyanate; 5-carboxytetramethylrhodamine; 5-carboxy rhodol derivatives; tetramethyl and tetraethyl rhodamine; diphenyldimethyl and diphenyldiethyl rhodamine; dinaphthyl rhodamine; rhodamine 101 sulfonyl chloride; Cy3, Cy3B, Cy3.5, Cy5, Cy5.5, Cy 7, indocyanine green, IR800CW, cyan fluorescent protein (CFP), EGFP, 6-FAM, FAM, fluorescein, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carboxyfluorescein quaternary ammonium, carboxyfluorescein phosphonate, carboxyfluorescein GABA, carboxyfluorescein-cys-Cy5,5′(6′)-carboxyfluorescein, fluorescein glutathione, or any combination thereof.

In some embodiments, the cargo molecule comprises a photosensitizing agent.

In some embodiments, the photosensitizing agent is selected from the group consisting of: a porphyrin, chlorin, and dye.

In some embodiments, the photosensitizing agent is selected from the group consisting of: porphyrin, protoporfin IX, purlytin, verteporfin, HPPH, temoporfin, methylene blue, photofrin, protofrin, hematoporphyrin, Talaporfin, benzopophyrin derivative monoacid, 5-aminileuvolinic acid, Lutetium texaphyrin, metallophthalocyanine, metallo-naphthocyaninesulfobenzo-porphyrazine, metallo-naphthalocyanines, zinc tetrasulfophthalocyanine, bacteriochlorins, metallochlorins, chlorine derivative, Tetra(m-hydroxyphenyl)chlorin (mTHPC), pheophorbide, dibromofluorescein (DBF), IR700DX, naphthalocyanine, and porphyrin derivatives.

In some embodiments, the cargo molecule comprises a drug.

In some embodiments, the drug is selected from the group consisting of: an antihistamine, a GABA receptor modulator, a neurotransmitter reuptake inhibitor, a local anesthetic, an anticholinergic, a sodium channel blocker, a calcium channel blocker, a thyrotropin-releasing hormone, a γ-secretase inhibitor, an AMPA receptor agonist or antagonist, an NMDA receptor agonist or antagonist, an mGlu receptor agonist or antagonist, a growth factor, an antiemetic agent, a corticosteroid; a nonsteroidal anti-inflammatory drug (NSAID); an anti-epileptic agent; a neurotropic agent; a cytotoxic agent; an antioxidant, an iron chelator, a mitochondrial modulator, a sirtuin modulator, a nitric oxide (NO) and/or nitric oxide synthase (NOS) modulator, a potassium channel agonist or antagonist, a purigenic receptor agonist or antagonist, or any combination thereof.

In some embodiments, the drug is selected from the group consisting of: benzocaine; carticaine; cinchocaine; cyclomethycaine; lidocaine; prilocaine; propxycaine; proparacaine; tetracaine; tocainide; and trimecaine; methotrexate; cyclophosphamide; thalidomide; paclitaxel; pemetrexed; pentostatin; pipobroman; pixantrone; plicamycin; platonin; procarbazine; raltitrexed; rebeccamycin; rubitecan; SN-38; salinosporamide A; satraplatin; streptozotocin; swainsonine; tariquidar; taxane; tegafur-uracil; temozolomide; testolactone; thioTEPA; tioguanine; topotecan; trabectedin; tretinoin; triplatin tetranitrate; tris(2-chloroethyl)amine; troxacitabine; uracil mustard; valrubicin; vinblastine; vincristine; vinorelbine; vorinostat; zosuquidar; carbamazepine; oxcarbazepine; phenytein; valproic acid; sodium valproate; cinnarizine; flunarizine; nimodipine; brain-derived neurotrophic factor (BDNF); ciliary neurotrophic factor (CNTF); glial cell-line derived neurotrophic factor (GDNF); neurotrophin-3; neurotrophin-4; fibroblast growth factor (FGF) receptor; insulin-like growth factor (IGF); prednisone; prednisolone; dexamethasone; gabapentin; or any combination thereof.

In another aspect, the present disclosure provides a pharmaceutical composition comprising a nerve targeting peptide conjugate described herein and a pharmaceutically acceptable carrier.

In yet another aspect, the present disclosure provides a method of delivering a cargo molecule to a neuron or nerve comprising contacting the neuron or nerve with a nerve targeting peptide conjugate of the present disclosure.

In yet another aspect, the present disclosure provides a method of identifying a neuron or nerve comprising contacting the neuron or nerve with a nerve targeting peptide conjugate of the present disclosure.

In yet another aspect, the present disclosure provides a method of delivering a drug to a neuron or nerve comprising contacting the neuron or nerve with a nerve targeting peptide conjugate of the present disclosure.

In another aspect, the present disclosure provides a method of delivering a photosensitizing agent to a neuron or nerve comprising contacting the neuron or nerve with a nerve targeting peptide conjugate of the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B are Venn diagrams depicting: (FIG. 1A) the overlap of peptide sequence subsets identified from phage display screens directed to two independent human muscle samples (muscle 2 and muscle 3) and three independent human nerve samples (nerve2, nerve3, and plated_nerve2); and (FIG. 1B) the source of the representative sample of nerve-specific peptides, namely those comprising peptides unique to each nerve subset (highlighted in gray), peptides in common between two nerve subsets (highlighted in yellow), and peptides found in all three nerve subsets (highlighted in green).

FIGS. 2A-2B are graphical depictions of the most prevalent amino acids at each position of the representative sample of nerve-binding peptides identified in Example 1, based on (FIG. 2A) unweighted or (FIG. 2B) weighted probabilities.

FIG. 3 shows the alignment of peptide sequences comprising cluster 1, based on Clustal Omega analysis of the representative sample of nerve-binding peptides. The ID number of each peptide is indicated, and the “majority rule consensus” residues at each position are highlighted to identify patterns reflected in corresponding Formula I and Subformula Ia.

FIG. 4 shows the alignment of peptide sequences comprising cluster 2, based on Clustal Omega analysis of the representative sample of nerve-binding peptides. The corresponding ID number is indicated for each peptide, and the “majority rule consensus” residues at each position are highlighted to identify patterns reflected in corresponding Formula II and Subformula IIa.

FIG. 5 shows the alignment of peptide sequences comprising cluster 3, based on Clustal Omega analysis of the representative sample of nerve-binding peptides. The corresponding ID number is indicated for each peptide, and the “majority rule consensus” residues at each position are highlighted to identify patterns reflected in corresponding Formula III and Subformula IIIa.

FIG. 6 shows the alignment of peptide sequences comprising cluster 4, based on Clustal Omega analysis of the representative sample of nerve-binding peptides. The corresponding ID number is indicated for each peptide, and the “majority rule consensus” residues at each position are highlighted to identify patterns reflected in corresponding Formula IV and Subformula IVa.

FIG. 7 shows the alignment of peptide sequences comprising cluster 5, based on Clustal Omega analysis of the representative sample of nerve-binding peptides. The corresponding ID number is indicated for each peptide, and the “majority rule consensus” residues at each position are highlighted to identify patterns reflected in corresponding Formula V and Subformula Va.

FIG. 8 shows the alignment of peptide sequences comprising cluster 6, based on Clustal Omega analysis of the representative sample of nerve-binding peptides. The corresponding ID number is indicated for each peptide, and the “majority rule consensus” residues at each position are highlighted to identify patterns, reflected in corresponding Formula VI and Subformula VIa.

FIG. 9 shows the alignment of peptide sequences comprising cluster 7, based on Clustal Omega analysis of the representative sample of nerve-binding peptides. The corresponding ID number is indicated for each peptide, and the “majority rule consensus” residues at each position are highlighted to identify patterns, reflected in corresponding Formula VII and Subformula VIIa.

FIG. 10 shows the alignment of peptide sequences comprising cluster 8, based on Clustal Omega analysis of the representative sample of nerve-binding peptides. The corresponding ID number is indicated for each peptide, and the “majority rule consensus” residues at each position are highlighted to identify patterns, reflected in corresponding Formula VIII and Subformula VIIIa.

FIG. 11 shows the alignment of peptide sequences comprising cluster 9, based on Clustal Omega analysis of the representative sample of nerve-binding peptides. The corresponding ID number is indicated for each peptide, and the “majority rule consensus” residues at each position are highlighted to identify patterns, reflected in corresponding Formula IX and Subformula IXa.

FIG. 12 shows the alignment of peptide sequences comprising cluster 10, based on Clustal Omega analysis of the representative sample of nerve-binding peptides. The corresponding ID number is indicated for each peptide, and the “majority rule consensus” residues at each position are highlighted to identify patterns, reflected in corresponding Formula X and Subformula Xa.

FIG. 13 shows the alignment of peptide sequences comprising cluster 11, based on Clustal Omega analysis of the representative sample of nerve-binding peptides. The corresponding ID number is indicated for each peptide, and the “majority rule consensus” residues at each position are highlighted to identify patterns, reflected in corresponding Formula XI and Subformula XIa.

FIG. 14 shows the alignment of peptide sequences comprising cluster 12, based on Clustal Omega analysis of the representative sample of nerve-binding peptides. The corresponding ID number is indicated for each peptide, and the “majority rule consensus” residues at each position are highlighted to identify patterns, reflected in corresponding Formula XII and Subformula XIIa.

FIG. 15 shows the alignment of peptide sequences comprising cluster 13, based on Clustal Omega analysis of the representative sample of nerve-binding peptides. The corresponding ID number is indicated for each peptide, and the “majority rule consensus” residues at each position are highlighted to identify patterns, reflected in corresponding Formula XIII and Subformula XIIIa.

FIG. 16 shows the alignment of peptide sequences comprising cluster 14, based on Clustal Omega analysis of the representative sample of nerve-binding peptides. The corresponding ID number is indicated for each peptide, and the “majority rule consensus” residues at each position are highlighted to identify patterns, reflected in corresponding Formula XIV and Subformula XIVa.

FIG. 17 shows the alignment of peptide sequences comprising cluster 15, based on Jalview tree analysis of the representative sample of nerve-binding peptides. The corresponding ID number is indicated for each peptide, and the “majority rule consensus” residues at each position are highlighted to identify patterns, reflected in corresponding Formula XV and Subformula XVa.

FIG. 18 shows the alignment of peptide sequences comprising cluster 16, based on Jalview tree analysis of the representative sample of nerve-binding peptides. The corresponding ID number is indicated for each peptide, and the “majority rule consensus” residues at each position are highlighted to identify patterns, reflected in corresponding Formula XVI and Subformula XVIa.

FIG. 19 shows the alignment of peptide sequences comprising cluster 17, based on Jalview tree analysis of the representative sample of nerve-binding peptides. The corresponding ID number is indicated for each peptide, and the “majority rule consensus” residues at each position are highlighted to identify patterns, reflected in corresponding Formula XVII and Subformula XVIIa.

FIG. 20 shows the alignment of peptide sequences comprising cluster 18, based on Jalview tree analysis of the representative sample of nerve-binding peptides. The corresponding ID number is indicated for each peptide, and the “majority rule consensus” residues at each position are highlighted to identify patterns, reflected in corresponding Formula XVIII and Subformula XVIIIa.

FIG. 21 shows the alignment of peptide sequences comprising cluster 19, based on Jalview tree analysis of the representative sample of nerve-binding peptides. The corresponding ID number is indicated for each peptide, and the “majority rule consensus” residues at each position are highlighted to identify patterns, reflected in corresponding Formula XIX and Subformula XIXa.

FIG. 22 shows the alignment of peptide sequences comprising cluster 20, based on Jalview tree analysis of the representative sample of nerve-binding peptides. The corresponding ID number is indicated for each peptide, and the “majority rule consensus” residues at each position are highlighted to identify patterns, reflected in corresponding Formula XX and Subformula XXa.

FIG. 23 shows the alignment of peptide sequences comprising cluster 21, based on Jalview tree analysis of the representative sample of nerve-binding peptides. The corresponding ID number is indicated for each peptide, and the “majority rule consensus” residues at each position are highlighted to identify patterns, reflected in corresponding Formula XXI and Subformula XXIa.

FIG. 24 shows the alignment of peptide sequences comprising cluster 22, based on Jalview tree analysis of the representative sample of nerve-binding peptides. The corresponding ID number is indicated for each peptide, and the “majority rule consensus” residues at each position are highlighted to identify patterns, reflected in corresponding Formula XXII and Subformula XXIIa.

FIG. 25 shows the alignment of peptide sequences comprising cluster 23, based on Jalview tree analysis of the representative sample of nerve-binding peptides. The corresponding ID number is indicated for each peptide, and the “majority rule consensus” residues at each position are highlighted to identify patterns, reflected in corresponding Formula XXIII and Subformula XXIIIa.

FIG. 26 shows the alignment of peptide sequences comprising cluster 24, based on Jalview tree analysis of the representative sample of nerve-binding peptides. The corresponding ID number is indicated for each peptide, and the “majority rule consensus” residues at each position are highlighted to identify patterns, reflected in corresponding Formula XXIV and Subformula XXIVa.

FIG. 27 shows the alignment of peptide sequences comprising cluster 25, based on Jalview tree analysis of the representative sample of nerve-binding peptides. The corresponding ID number is indicated for each peptide, and the “majority rule consensus” residues at each position are highlighted to identify patterns, reflected in corresponding Formula XXV and Subformula XXVa.

FIG. 28 shows the alignment of peptide sequences comprising cluster 26, based on Jalview tree analysis of the representative sample of nerve-binding peptides. The corresponding ID number is indicated for each peptide, and the “majority rule consensus” residues at each position are highlighted to identify patterns, reflected in corresponding Formula XVI and Subformula XVIa.

FIG. 29 shows the alignment of peptide sequences comprising cluster 27, based on Jalview tree analysis of the representative sample of nerve-binding peptides. The corresponding ID number is indicated for each peptide, and the “majority rule consensus” residues at each position are highlighted to identify patterns, reflected in corresponding Formula XVII and Subformula XVIIa.

FIG. 30 shows the alignment of peptide sequences comprising cluster 28, based on Jalview tree analysis of the representative sample of nerve-binding peptides. The corresponding ID number is indicated for each peptide, and the “majority rule consensus” residues at each position are highlighted to identify patterns, reflected in corresponding Formula XVIII and Subformula XVIIIa.

FIG. 31 shows the alignment of peptide sequences comprising cluster 29, based on Jalview tree analysis of the representative sample of nerve-binding peptides. The corresponding ID number is indicated for each peptide, and the “majority rule consensus” residues at each position are highlighted to identify patterns, reflected in corresponding Formula XIX and Subformula XIXa.

FIG. 32 shows the alignment of peptide sequences comprising cluster 30, based on Jalview tree analysis of the representative sample of nerve-binding peptides. The corresponding ID number is indicated for each peptide, and the “majority rule consensus” residues at each position are highlighted to identify patterns, reflected in corresponding Formula XXX and Subformula XXXa.

FIG. 33 shows the alignment of peptide sequences comprising cluster 31, based on Jalview tree analysis of the representative sample of nerve-binding peptides. The corresponding ID number is indicated for each peptide, and the “majority rule consensus” residues at each position are highlighted to identify patterns, reflected in corresponding Formula XXXI and Subformula XXXIa.

FIG. 34 shows the alignment of peptide sequences comprising cluster 32, based on Jalview tree analysis of the representative sample of nerve-binding peptides. The corresponding ID number is indicated for each peptide, and the “majority rule consensus” residues at each position are highlighted to identify patterns, reflected in corresponding Formula XXXII and Subformula XXXIIa.

FIG. 35 shows the alignment of peptide sequences comprising cluster 33, based on Jalview tree analysis of the representative sample of nerve-binding peptides. The corresponding ID number is indicated for each peptide, and the “majority rule consensus” residues at each position are highlighted to identify patterns, reflected in corresponding Formula XXXIII and Subformula XXXIIIa.

FIG. 36 shows the alignment of peptide sequences comprising cluster 34, based on Jalview tree analysis of the representative sample of nerve-binding peptides. The corresponding ID number is indicated for each peptide, and the “majority rule consensus” residues at each position are highlighted to identify patterns, reflected in corresponding Formula XXXIV and Subformula XXXIVa.

FIG. 37 shows the alignment of peptide sequences comprising cluster 35, based on Jalview tree analysis of the representative sample of nerve-binding peptides. The corresponding ID number is indicated for each peptide, and the “majority rule consensus” residues at each position are highlighted to identify patterns, reflected in corresponding Formula XXXV and Subformula XXXVa.

FIG. 38 shows the alignment of peptide sequences comprising cluster 36, based on Jalview tree analysis of the representative sample of nerve-binding peptides. The corresponding ID number is indicated for each peptide, and the “majority rule consensus” residues at each position are highlighted to identify patterns, reflected in corresponding Formula XXXVI and Subformula XXXVIa.

FIG. 39 shows the alignment of peptide sequences comprising cluster 37, based on Jalview tree analysis of the representative sample of nerve-binding peptides. The corresponding ID number is indicated for each peptide, and the “majority rule consensus” residues at each position are highlighted to identify patterns, reflected in corresponding Formula XXXVII and Subformula XXXVIIa.

FIG. 40 shows the alignment of peptide sequences comprising cluster 38, based on Jalview tree analysis of the representative sample of nerve-binding peptides. The corresponding ID number is indicated for each peptide, and the “majority rule consensus” residues at each position are highlighted to identify patterns, reflected in corresponding Formula XXXVIII and Subformula XXXVIIIa.

FIG. 41 shows the alignment of peptide sequences comprising cluster 39, based on Jalview tree analysis of the representative sample of nerve-binding peptides. The corresponding ID number is indicated for each peptide, and the “majority rule consensus” residues at each position are highlighted to identify patterns, reflected in corresponding Formula XXXIX and Subformula XXXIXa.

FIG. 42 shows the alignment of peptide sequences comprising cluster 40, based on Jalview tree analysis of the representative sample of nerve-binding peptides. The corresponding ID number is indicated for each peptide, and the “majority rule consensus” residues at each position are highlighted to identify patterns, reflected in corresponding Formula XL and Subformula XLa.

FIG. 43 shows the alignment of peptide sequences comprising cluster 41, based on Jalview tree analysis of the representative sample of nerve-binding peptides. The corresponding ID number is indicated for each peptide, and the “majority rule consensus” residues at each position are highlighted to identify patterns, reflected in corresponding Formula XLI and Subformula XLIa.

FIG. 44 shows the alignment of peptide sequences comprising cluster 42, based on Jalview tree analysis of the representative sample of nerve-binding peptides. The corresponding ID number is indicated for each peptide, and the “majority rule consensus” residues at each position are highlighted to identify patterns, reflected in corresponding Formula XLII and Subformula XLIIa.

FIG. 45 shows the alignment of peptide sequences comprising cluster 43, based on Jalview tree analysis of the representative sample of nerve-binding peptides. The corresponding ID number is indicated for each peptide, and the “majority rule consensus” residues at each position are highlighted to identify patterns, reflected in corresponding Formula XLIII and Subformula XLIIIa.

FIG. 46 shows the alignment of peptide sequences comprising cluster 44, based on Jalview tree analysis of the representative sample of nerve-binding peptides. The corresponding ID number is indicated for each peptide, and the “majority rule consensus” residues at each position are highlighted to identify patterns, reflected in corresponding Formula XLIV and Subformula XLIVa.

### DETAILED DESCRIPTION

Custom phage display libraries were generated from randomization of a parent peptide QVPWEEPYYVVKKS (SEQ ID NO:558) under conditions resulting in a mutation rate or about 50% per residue. The libraries were used to identify peptides specifically targeting nerve versus muscle. As described herein, these screens yielded a representative set of specific nerve-binding peptides set forth in SEQ ID NOS:1-557. Alignment and cluster analysis of these peptide sequences has further revealed multiple consensus sequences, corresponding to Formulas I to XLIV (and Subformulas Ia to XLIVa).

The targeting peptides described herein may bind to motor, sensory and autonomic nerves. Nerve targeting peptides can be used for nerve targeting peptide conjugates comprising a nerve targeting peptide comprising the amino acid sequence of any one of SEQ ID NOS: 1-557 and Formulas I-XLIV (including Subformulas Ia to XLIVa); and a cargo molecule, such as a drug, fluorescent moiety, or photosensitizing agent. Nerve targeting peptide conjugates of the present disclosure may be used for delivering a cargo molecule to a neuron or nerve, identifying a neuron or nerve, delivering a drug to a neuron or nerve, or delivering a photosensitizing agent to a neuron or nerve.

The invention may be more fully appreciated by reference to the following description, including the examples. Unless otherwise defined, all technical and scientific terms used herein have the same meaning as commonly understood by one of ordinary skill in the art. The use of headings and subheadings provided in the sections of this specification is solely for convenience of reference and does not limit the various embodiments herein, which are to be construed by reference to the specification as a whole.

### Terms and Definitions

Prior to setting forth this disclosure in more detail, it may be helpful to an understanding thereof to provide definitions of certain terms to be used herein. Additional definitions are set forth throughout this disclosure.

The term “consisting essentially of” limits the scope of a claim to the specified materials or steps and those that do not materially affect the basic and novel characteristics of the claimed invention. It should be understood that the terms “a” and “an” as used herein refer to “one or more” of the enumerated components.

The use of the alternative (e.g., “or”) should be understood to mean either one, both, or any combination thereof of the alternatives. Thus, in embodiments of any of the Formulas and Subformulas described herein, the selection of amino acids at any position in the peptide can be any subset or combination of choices at that position. For example, in the case of Subformula (XLIVa), the embodiments not only include those in which X₃ is A, L, T, or V; but also those in which X₃ is A; X₃ is L; X₃ is T; X₃ is V; X₃ is A or L, X₃ is A, L, or T; X₃ is L, T, or V, and so on. Moreover, the choice of amino acids, or combination or subset thereof, at one position, e.g., X₃, is independent of the choice of amino acids (or combination or subset thereof) at any other position, e.g., X₄ or X₅.

As used herein, the terms “include” and “have” are used synonymously, which terms and variants thereof are intended to be construed as non-limiting. The term “comprise” means the presence of the stated features, integers, steps, or components as referred to in the claims, but does not preclude the presence or addition of one or more other features, integers, steps, components, or groups thereof.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure

As used herein, the “central nervous system” or “CNS” refers to the brain and the spinal cord, as well as the retina.

As used herein, the “peripheral nervous system” or “PNS” refers to the nerves and ganglia outside the CNS. The PNS is divided into the somatic nervous system and the autonomic nervous system. The somatic nervous system controls all voluntary muscular systems within the body, and the process of voluntary reflex arcs and is composed of afferent or sensory nerves, which relay sensation from the body to the central nervous system, and efferent or motor nerves, which send out commands from the CNS to the body, stimulating muscle contraction. The autonomic nervous system supplies smooth muscle and glands, and thus influences the function of internal organs. The autonomic nervous system acts largely unconsciously and regulates bodily functions, such as the heart rate, digestion, respiratory rate, pupillary response, urination, and sexual arousal.

As used herein, a “neuron” refers to an electrically excitable cell that processes and transmits information by electrical and chemical signaling. A typical neuron possesses a cell body (often called the soma), dendrites, and an axon.

As used herein, a “nerve” refers to an enclosed, cable-like bundle of neural axons. Each nerve is a cordlike structure that contains many axons. Each axon within the nerve is an extension of an individual neuron. Within a nerve, each axon is surrounded by a layer of connective tissue called the endoneurium. The axons are bundled together into groups called fascicles, and each fascicle is wrapped in a layer of connective tissue called the perineurium. Each nerve is covered on the outside by a dense sheath of connective tissue, the epineurium.

As used herein the term “conjugate” refers to a peptide attached to at least one cargo molecule, optionally via a linker.

The terms “polypeptide,” “peptide,” and “protein” are used interchangeably herein to refer to a polymer of amino acid residues. The terms apply to naturally occurring amino acid polymers as well as amino acid polymers in which one or more amino acid residues is a non-naturally occurring amino acid (e.g., an amino acid analog). The terms encompass amino acid chains of any length, including full length proteins (i.e., antigens), wherein the amino acid residues are linked by covalent peptide bonds. As used herein, the term “peptide” refers to a polymer of amino acid residues typically ranging in length from 2 to about 50 residues. Where an amino acid sequence is provided herein, L-, D-, or beta amino acid versions of the sequence are also contemplated as well as retro, inversion, and retro-inversion isoforms. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other modified linkages (e.g., where the peptide bond is replaced by an α-ester, a /3-ester, a thioamide, phosphonamide, carbamate, hydroxylate, and the like (see, e.g., Spatola, 1983, Chem. Biochem. Amino Acids and Proteins 7, 267-357), where the amide is replaced with a saturated amine (see, e.g., Skiles et al., U.S. Pat. No. 4,496,542, which is incorporated herein by reference, and Kaltenbronn et al. 1990, pp. 969-970 in Proc. 11th American Peptide Symposium, ESCOM Science Publishers, The Netherlands, and the like).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. An amino acid may be an L- or D-amino acid. Amino acid analogs refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids are referred to herein by either their commonly known three letter symbols or by the one-letter symbols recommended by the IUP AC-IUB Biochemical Nomenclature Commission, as summarized here:

| | Three Letter | One Letter |
|---|---|---|
| Amino Acid | Symbol | Symbol |
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Nucleotides, likewise, may be referred to by their commonly accepted single-letter codes.

One of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to, and do not exclude, polymorphic variants, interspecies homologs, and alleles of the invention.

In some embodiments, naturally occurring amino acids in the peptides disclosed herein may be substituted with naturally occurring non-encoded amino acids and synthetic amino acids. For example, certain commonly encountered amino acids that may provide useful substitutions include, but are not limited to, β-alanine (β-Ala) and other omega-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); naphthylalanine (Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,4-diaminobutyric acid (Dbu); 2,3-diaminobutyric acid (Dab); p-aminophenylalanine (Phe(pNH₂)); N-methyl valine (MeVal); homocysteine (hCys), homophenylalanine (hPhe) and homoserine (hSer); hydroxyproline (Hyp), homoproline (hPro), N-methylated amino acids and peptoids (N-substituted glycines).

While in most instances, the amino acids of the nerve-binding peptides disclosed herein will be substituted with L-enantiomeric amino acids, the substitutions are not limited to L-enantiomeric amino acids. Thus, in some embodiments, an L-amino acid may be replaced with an identical D-amino acid or with a D-amino acid of the same category or subcategory.

“Sequence identity,” as used herein, refers to the percentage of amino acid residues in a single sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values can be generated using the NCBI BLAST 2.0 software as defined by Altschul et al. (1997), Nucl. Acids Res. 25:3389-3402, with the parameters set to default values.

As used herein, the terms “label” refers to a molecule that facilitates the visualization and/or detection of a targeting molecule disclosed herein. In some embodiments, the label is a fluorescent moiety.

The phrase “specifically binds” when referring to the interaction between a peptide disclosed herein and a target (e.g., purified protein, neuron or nerve tissue, neuron or nerves, cranial neuron or nerves, central neuron or nerves, myelinated or unmyelinated neuron or nerves, or connective tissue surrounding neuron or nerves), refers to the formation of a high affinity bond between the targeting molecule and the target. Further, the term means that the peptide has low affinity for non-targets.

“Selective binding,” “selectivity,” and the like refer to the preference of agent to interact with one molecule as compared to another. Preferably, interactions between a peptide disclosed herein and a target are both specific and selective. Note that in some embodiments an agent is designed to “specifically bind” and “selectively bind” two distinct, yet similar targets without binding to other undesirable targets.

The terms “individual,” “patient,” or “subject” are used interchangeably. As used herein, they mean any mammal (i.e., species of any orders, families, and genera within the taxonomic classification animalia: chordata: vertebrata: mammalia). In some embodiments, the mammal is a cow, horse, sheep, pig, cat, dog, goat, mouse, rat, rabbit, guinea pig, non-human primate, or human. In some embodiments, the mammal is a human.

The terms “administer,” “administering”, “administration,” and the like, as used herein, refer to the methods that may be used to enable delivery of agents or compositions to the desired site of biological action. These methods include, but are not limited to topical, oral, intrarectal, intravaginal, intranasal, inhalation, parenteral injection (e.g., intravenous, intraarterial, subcutaneous, intraperitoneal, intramuscular, intravascular, intrathecal, intravitreal, infusion, or local). Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa. Administration of agents may be local or systemic. In some embodiments, administration is via systemic intravenous injection into human patients.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

As used herein, “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, and having the properties of being nontoxic and non-inflammatory in a patient. Each excipient must be “acceptable” in the sense of being compatible with the other ingredients of the formulation and not injurious to the patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Examples of pharmaceutical excipients can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as excipients, particularly for injectable solutions. Suitable pharmaceutical excipients are described in “Remington's Pharmaceutical Sciences” by E. W. Martin.

The term “surgery” as used herein, refers to any methods that may be used to manipulate, change, or cause an effect by a physical intervention. These methods include, but are not limited to open surgery, endoscopic surgery, laparoscopic surgery, minimally invasive surgery, microscopic surgery, robotic surgery, any procedures that may affect any neuron or nerves such as placement of retractors during spinal surgery, microscopic procedures, cardiac neuron or nerve ablation, epidural injection, intrathecal injections, neuron or nerve blocks, implantation of devices such as neuron or nerve stimulators and implantation of pumps. In some embodiments, the subject of the surgery is a human subject or human patient.

### I. Peptide

In one aspect, the present disclosure provides peptides showing selective binding for nerves versus muscle and that can be used in nerve targeting peptide conjugates of the present disclosure.

In some embodiments, a peptide comprises or consists of an amino acid sequence set forth in any one of SEQ ID NOS:1-557. In some embodiments, the peptide comprises or consists of an amino acid sequence set forth in any one of SEQ ID NOS: 1-30. In some embodiments, the peptide comprises or consists of the amino acid sequence set forth in any one of SEQ ID NOS:31-36. In some embodiments, the peptide comprises or consists of the amino acid sequence set forth in any one of SEQ ID NOS:37-40. In some embodiments, the peptide comprises or consists of the amino acid sequence set forth in any one of SEQ ID NOS:41-240. In some embodiments, the peptide comprises or consists of the amino acid sequence set forth in any one of SEQ ID NOS:241-349. In some embodiments, the peptide comprises or consists of the amino acid sequence set forth in any one of SEQ ID NOS:350-449. In some embodiments, the peptide comprises or consists of the amino acid sequence set forth in any one of SEQ ID NOS:450-557.

In some embodiments, a peptide comprises or consists of the amino acid sequence having at least 75% identity to any one of SEQ ID NOS: 1-557. In some embodiments, the peptide comprises or consists of the amino acid sequence having at least 80% identity to any one of SEQ ID NOS: 1-557. In some embodiments, the peptide comprises or consists of the amino acid sequence having at least 85% identity to any one of SEQ ID NOS:1-557. In some embodiments, the peptide comprises or consists of the amino acid sequence having at least 90% identity to any one of SEQ ID NOS: 1-557. In some embodiments, the peptide comprises or consists of the amino acid sequence having at least 92% identity to any one of SEQ ID NOS: 1-557. In some embodiments, the peptide comprises or consists of the amino acid sequence having at least 95% identity to any one of SEQ ID NOS: 1-557.

In some embodiments, a peptide comprises or consists of the amino acid sequence set forth in any one of Formulas (I)-(XLIV), including Subformulas (Ia)-(XLIVa).

### Formula (I)

In some embodiments, a peptide comprises or consists of the amino acid sequence of Formula (I): X₁-X₂-X₃-X₄-X₅-X₆-X₇-X₈-X₉-X₁₀-X₁₁-X₁₂-X₁₃-X₁₄, wherein:
X₁ is A, E, H, L, Q, R, or Y;
X₂ is D, F, G, I, L, N, or V;
X₃ is A, E, H, I, L, P, Q, R, S, T, or W;
X₄ is C, F, G, K, L, Q, R, S, V, W, or Y;
X₅ is A, D, E, G, K, L, Q, T, V, or Y;
X₆ is A, D, E, G, K, N, Q, R, S, V, or Y;
X₇ is A, L, M, P, S, T, or W;
X₈ is C, E, H, L, N, P, R, S, V, or Y;
X₉ is A, C, D, E, H, L, N, P, S, or Y;
X₁₀ is A, D, E, F, G, H, I, L, N, P, S, T, V, or Y;
X₁₁ is A, D, E, I, L, Q, R, T, or V;
X₁₂ is D, I, K, L, N, Q, R, S, or T;
X₁₃ is A, C, D, E, H, I, K, M, N, Q, R, T, or V; and
X₁₄ is P, S, T, V, or Y.

In some embodiments, a peptide of Formula (I) comprises or consists of the amino acid sequence of any one of SEQ ID NOS: 25, 28, 37, 59, 60, 67, 78, 86, 99, 101, 140, 144, 149, 158, 159, 175, 182, 187, 188, 217, 233, 241, 248, 270, 282, 287, 290, 302, 317, 350, 359, 384, 407, 410, 422, 439, 448, 451, 454, 459, 460, 476, 495, 506, 513, 519, 520, 551, or 552.

In some embodiments, a peptide of Formula (I) comprises or consists of the amino acid sequence of Subformula (Ia): H-V-X₃-X₄-X₅-X₆-P-Y-Y-X₁₀-V-X₁₂-X₁₃-S, wherein:
X₃ is A, E, H, I, L, P, Q, R, S, T, or W;
X₄ is C, F, G, K, L, Q, R, S, V, W, or Y;
X₅ is A, D, E, G, K, L, Q, T, V, or Y;
X₆ is A, D, E, G, K, N, Q, R, S, V, or Y;
X₁₀ is A, D, E, F, G, H, I, L, N, P, S, T, V, or Y;
X₁₂ is D, I, K, L, N, Q, R, S, or T; and
X₁₃ is A, C, D, E, H, I, K, M, N, Q, R, T, or V.

In some embodiments, a peptide of Formula (I) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (Ia) does not comprise or consist of the amino acid sequence, wherein X₃ is P; X₄ is W; X₅ is E; X₆ is E; X₁₀ is V; X₁₂ is K; and X₁₃ is K.

More generally, in some embodiments, the selection of amino acids at any position of a peptide of Formula (I) and Subformula (Ia) can be any subset or combination of choices at that position. Thus, the embodiments not only include those in which X₃ is A, E, H, I, L, P, Q, R, S, T, or W; but also, for example, those in which X₃ is A; X₃ is E; X₃ is H; etc.; X₃ is A or E; X₃ is A, E, or H; X₃ is E or H; X₃ is E, H, L, or Q, and so on. Moreover, the choice of amino acids, or combination or subset thereof, at one position, e.g., X₃, is independent of the choice of amino acids (or combination or subset thereof) at any other position, e.g., X₅ or X₆, etc.

### Formula (II)

In some embodiments, a peptide comprises or consists of an amino acid sequence of Formula (II): X₁-X₂-X₃-X₄-X₅-X₆-X₇-X₈-X₉-X₁₀-X₁₁-X₁₂-X₁₃-X₁₄, wherein:
X₁ is A, D, E, H, K, L, N, Q, R, S, or T;
X₂ is A, F, H, I, L, T, or V;
X₃ is A, E, F, L, P, Q, S, or T;
X₄ is A, E, F, G, L, M, R, S, T, V, W, or Y;
X₅ is A, E, G, H, I, P, Q, T, V, or Y;
X₆ is A, D, E, G, H, I, K, N, Q, or R;
X₇ is A, H, L, P, Q, R, S, or T;
X₈ is G, H, L, R, S, V, or Y;
X₉ is D, E, F, L, M, N, T, V, or Y;
X₁₀ is A, D, F, G, H, I, L, N, R, S, V, or Y;
X₁₁ is E, F, G, K, L, M, R, or V;
X₁₂ is E, H, I, K, N, P, Q, R, S, T, or V,
X₁₃ is D, E, F, H, I, K, N, Q, R, S, T, or V; and
X₁₄ is A, F, H, P, R, S, or T.

In some embodiments, a peptide of Formula (II) comprises or consists of the amino acid sequence of any one of SEQ ID NOS: 26, 52, 68, 83, 89, 108, 114, 119, 139, 147, 174, 177, 179, 183, 195, 197, 198, 205, 209, 225, 232, 238, 279, 285, 303, 318, 321, 322, 325, 348, 363, 383, 397, 417, 462, 464, 488, 494, 502, 518, 545, or 546.

In some embodiments, a peptide of Formula (II) comprises or consists of the amino acid sequence of Subformula (IIa): X₁-X₂-X₃-X₄-E-X₆-X₇-Y-Y-X₁₀-V-X₁₂-X₁₃-X₁₄, wherein:
X₁ is A, D, E, H, K, L, N, Q, R, S, or T;
X₂ is A, F, H, I, L, T, or V;
X₃ is A, E, F, L, P, Q, S, or T;
X₄ is A, E, F, G, L, M, R, S, T, V, W, or Y;
X₆ is A, D, E, G, H, I, K, N, Q, or R;
X₇ is A, H, L, P, Q, R, S, or T;
X₁₀ is A, D, F, G, H, I, L, N, R, S, V, or Y;
X₁₂ is E, H, I, K, N, P, Q, R, S, T, or V,
X₁₃ is D, E, F, H, I, K, N, Q, R, S, T, or V; and
X₁₄ is A, F, H, P, R, S, or T.

In some embodiments, a peptide of Formula (II) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (IIa) does not comprise or consist of the amino acid sequence, wherein X₁ is Q; X₂ is V; X₃ is P; X₄ is W; X₆ is E; X₇ is P; X₁₀ is V; X₁₂ is K; X₁₃ is K; and X₁₄ is S.

More generally, in some embodiments, the selection of amino acids at any position of a peptide of Formula (II) and Subformula (IIa) can be any subset or combination of choices at that position. Thus, the embodiments not only include those in which X₃ is A, E, F, L, P, Q, S, or T; but also, for example, those in which X₃ is A; X₃ is E; X₃ is F; etc.; X₃ is A or E; X₃ is A, F, or P; X₃ is E or L; X₃ is E, F, L, or S, and so on. Moreover, the choice of amino acids, or combination or subset thereof, at one position, e.g., X₃, is independent of the choice of amino acids (or combination or subset thereof) at any other position, e.g., X₄ or X₇, etc.

### Formula (III):

In some embodiments, a peptide comprises or consists of the amino acid sequence of Formula (III): X₁-X₂-X₃-X₄-X₅-X₆-X₇-X₈-X₉-X₁₀-X₁₁-X₁₂-X₁₃-X₁₄, wherein:
X₁ is H, Q, R, or V;
X₂ is F, G, H, I, L, or V;
X₃ is A, H, P, or T;
X₄ is F, G, L, M, S, T, or W;
X₅ is A, D, G, I, K, P, Q, or V;
X₆ is D, E, G, or P;
X₇ is L, P, Q, R, S, or T;
X₈ is F, H, S, or Y;
X₉ is D, F, G, H, N, S, or Y;
X₁₀ is N or V;
X₁₁ is A, E, G, K, L, Q, S, V, or W;
X₁₂ is A, D, E, G, K, M, N, Q, R, or T;
X₁₃ is E, H, K, N, Q, R, T, or V; and
X₁₄ is F, H, N, P, S, T, or Y.

In some embodiments, a peptide of Formula (III) comprises or consists of the amino acid sequence of any one of SEQ ID NOS: 3, 4, 30, 80, 94, 118, 181, 214, 224, 240, 260, 286, 289, 312, 313, 357, 377, 405, or 550.

In some embodiments, a peptide of Formula (III) comprises or consists of the amino acid sequence of Subformula (IIIa): H-V-P-X₄-X₅-G-P-Y-X₉-V-X₁₁-K-X₁₃-X₁₄, wherein:
X₄ is F, G, L, M, S, T, or W;
X₅ is A, D, G, I, K, P, Q, or V;
X₉ is D, F, G, H, N, S, or Y;
X₁₁ is A, E, G, K, L, Q, S, V, or W;
X₁₃ is E, H, K, N, Q, R, T, or V; and
X₁₄ is F, H, N, P, S, T, or Y.

In some embodiments, a peptide of Formula (III) does not comprise or consist of the amino acid sequence, wherein X₁ is Q; X₂ is V; X₃ is P; X₄ is W; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (IIIa) does not comprise or consist of the amino acid sequence, wherein X₄ is W; X₉ is Y; X₁₁ is V; X₁₃ is K; and X₁₄ is S.

More generally, in some embodiments, the selection of amino acids at any position of a peptide of Formula (III) and Subformula (IIIa) can be any subset or combination of choices at that position. Thus, the embodiments not only include those in which X₄ is F, G, L, M, S, T, or W; but also, for example, those in which X₄ is F; X₄ is G; X₄ is L; etc.; X₄ is F or G; X₄ is F, G, or L; X₄ is G or L; and so on. Moreover, the choice of amino acids, or combination or subset thereof, at one position, e.g., X₄, is independent of the choice of amino acids (or combination or subset thereof) at any other position, e.g., X₅, X₉, or X₁₀, etc.

### Formula (IV)

In some embodiments, a peptide comprises or consists of the amino acid sequence of Formula (IV): X₁-X₂-X₃-X₄-X₅-X₆-X₇-X₈-X₉-X₁₀-X₁₁-X₁₂-X₁₃-X₁₄, wherein:
X₁ is E, H, K, L, Q, R, S, V, or Y;
X₂ is A, D, F, G, H, I, L, S, V, or Y;
X₃ is A, H, K, L, N, P, Q, R, S, T, V, or W;
X₄ is A, C, E, F, G, I, L, M, P, R, S, T, V, W, or Y;
X₅ is A, D, E, G, H, I, K, N, Q, S, T, V, or Y;
X₆ is A, D, E, G, H, K, N, P, Q, S, T, V, or Y;
X₇ is A, H, L, P, Q, R, S, or T;
X₈ is C, D, E, F, H, I, K, L, N, S, V, or Y;
X₉ is A, D, F, H, K, L, N, S, V, W, or Y;
X₁₀ is A, E, F, G, I, L, P, R, S, V, or Y;
X₁₁ is A, C, E, F, G, I, L, M, N, P, Q, S, T, V, or W;
X₁₂ is A, E, H, I, K, N, P, Q, R, T, V, or W;
X₁₃ is D, E, H, I, K, M, N, Q, R, S, T, or Y; and
X₁₄ is A, F, H, I, M, P, R, S, T, W, or Y.

In some embodiments, a peptide of Formula (IV) comprises or consists of the amino acid sequence of any one of SEQ ID NOS: 22, 36, 40, 64, 65, 69, 81, 85, 102, 111, 112, 116, 121, 129, 131, 135, 136, 142, 150, 216, 223, 234, 259, 261, 262, 266, 283, 288, 292, 296, 307, 315, 319, 345, 365, 368, 373, 378, 379, 389, 393, 416, 423, 485, 503, 504, 527, 531, 538, or 539.

In some embodiments, a peptide of Formula (IV) comprises or consists of the amino acid sequence of Subformula (IVa): H-V-X₃-X₄-X₅-X₆-X₇-X₈-Y-V-X₁₁-X₁₂-K-X₁₄, wherein:
X₃ is A, H, K, L, N, P, Q, R, S, T, V, or W;
X₄ is A, C, E, F, G, I, L, M, P, R, S, T, V, W, or Y;
X₅ is A, D, E, G, H, I, K, N, Q, S, T, V, or Y;
X₆ is A, D, E, G, H, K, N, P, Q, S, T, V, or Y;
X₇ is A, H, L, P, Q, R, S, or T;
X₈ is C, D, E, F, H, I, K, L, N, S, V, or Y;
X₁₁ is A, C, E, F, G, I, L, M, N, P, Q, S, T, V, or W;
X₁₂ is A, E, H, I, K, N, P, Q, R, T, V, or W; and
X₁₄ is A, F, H, I, M, P, R, S, T, W, or Y.

In some embodiments, a peptide of Formula (IV) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (IVa) does not comprise or consist of the amino acid sequence, wherein X₃ is P; X₄ is W; X₅ is E; X₆ is E; X₇ is P; X₈ is Y; X₁₁ is V; X₁₂ is K; and X₁₄ is S.

More generally, in some embodiments, the selection of amino acids at any position of a peptide of Formula (IV) and Subformula (IVa) can be any subset or combination of choices at that position. Thus, the embodiments not only include those in which X₇ is A, H, L, P, Q, R, S, or T; but also, for example, those in which X₇ is A; X₇ is H; X₇ is L; etc.; X₇ is A or H; X₇ is A, H, or L; X₇ is H or L; X₇ is H, L, P, or S, and so on. Moreover, the choice of amino acids, or combination or subset thereof, at one position, e.g., X₇, is independent of the choice of amino acids (or combination or subset thereof) at any other position, e.g., X₁, X₄, X₅, X₆, etc.

### Formula (V)

In some embodiments, a peptide comprises or consists of the amino acid sequence of Formula (V): X₁-X₂-X₃-X₄-X₅-X₆-X₇-X₈-X₉-X₁₀-X₁₁-X₁₂-X₁₃-X₁₄, wherein:
X₁ is D, F, G, H, N, Q, R, T, or Y;
X₂ is D, F, H, I, L, M, Q, or V;
X₃ is D, E, G, L, P, Q, R, S, or T;
X₄ is G, L, Q, R, S, or W;
X₅ is A, D, E, G, H, K, Q, R, or V;
X₆ is A, E, G, Q, or V;
X₇ is P or R;
X₈ is S or Y;
X₉ is G, H, N, Q, R, S, T, or Y;
X₁₀ is D, F, I, P, or V;
X₁₁ is A, D, F, G, I, L, Q, or V;
X₁₂ is A, E, H, K, L, N, R, S, or T;
X₁₃ is K, M, N, P, Q, T, or W; and
X₁₄ is A, F, I, L, P, R, S, or Y.

In some embodiments, a peptide of Formula (V) comprises or consists of the amino acid sequence of any one of SEQ ID NOS: 35, 75, 76, 107, 157, 196, 207, 215, 249, 251, 252, 272, 294, 305, 310, 311, 314, 316, 320, 347, 355, 362, 370, 385, 412, 413, 456, 498, or 549.

In some embodiments, a peptide of Formula (V) comprises or consists of the amino acid sequence of Subformula (Va): H-V-X₃-W-X₅-E-P-Y-Y-V-X₁₁-X₁₂-K-X₁₄, wherein:
X₃ is D, E, G, L, P, Q, R, S, or T;
X₅ is A, D, E, G, H, K, Q, R, or V;
X₁₁ is A, D, F, G, I, L, Q, or V;
X₁₂ is A, E, H, K, L, N, R, S, or T; and
X₁₄ is A, F, I, L, P, R, S, or Y.

In some embodiments, a peptide of Formula (V) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (Va) does not comprise or consist of the amino acid sequence, wherein X₃ is P; X₅ is E; X₁₁ is V; X₁₂ is K; and X₁₄ is S.

More generally, in some embodiments, the selection of amino acids at any position of a peptide of Formula (V) and Subformula (Va) can be any subset or combination of choices at that position. Thus, the embodiments not only include those in which X₅ is A, D, E, G, H, K, Q, R, or V; but also, for example, those in which X₅ is A; X₅ is D; X₅ is E; etc.; X₅ is A or D; X₅ is A, D, or E; X₅ is D or E; X₅ is A, D, E, or R, and so on. Moreover, the choice of amino acids, or combination or subset thereof, at one position, e.g., X₅, is independent of the choice of amino acids (or combination or subset thereof) at any other position, e.g., X₁₁ or X₁₂, etc.

### Formula (VI)

In some embodiments, a peptide comprises or consists of the amino acid sequence of Formula (VI): X₁-X₂-X₃-X₄-X₅-X₆-X₇-X₈-X₉-X₁₀-X₁₁-X₁₂-X₁₃-X₁₄, wherein:
X₁ is D, H, L, N, Q, or Y;
X₂ is A, D, F, G, H, I, L, R, or V;
X₃ is A, F, L, P, S, T, or V;
X₄ is A, G, L, S, V, W, or Y;
X₅ is A, D, E, I, K, Q, or V;
X₆ is A, E, L, N, P, Q, or R;
X₇ is A, G, H, L, P, Q, R, or S;
X₈ is A, D, F, K, N, R, S, or Y;
X₉ is D, E, F, K, L, N, S, or Y;
X₁₀ is A, E, F, G, I, L, N, P, T, or V;
X₁₁ is E, K, L, M, V, or W;
X₁₂ is E, H, K, L, Q, R, S, or T;
X₁₃ is E, H, K, M, N, Q, or Y; and
X₁₄ is F, G, I, S, or T.

In some embodiments, a peptide of Formula (VI) comprises or consists of the amino acid sequence of any one of SEQ ID NOS: 13, 33, 91, 96, 124, 167, 185, 199, 227, 235, 326, 360, 392, 409, 440, or 530.

In some embodiments, a peptide of Formula (VI) comprises or consists of the amino acid sequence of Subformula (VIa): X₁-X₂-X₃-X₄-X₅-E-X₇-X₈-X₉-X₁₀-X₁₁-X₁₂-X₁₃-S, wherein:
X₁ is D, H, L, N, Q, or Y;
X₂ is A, D, F, G, H, I, L, R, or V;
X₃ is A, F, L, P, S, T, or V;
X₄ is A, G, L, S, V, W, or Y;
X₅ is A, D, E, I, K, Q, or V;
X₇ is A, G, H, L, P, Q, R, or S;
X₈ is A, D, F, K, N, R, S, or Y;
X₉ is D, E, F, K, L, N, S, or Y;
X₁₀ is A, E, F, G, I, L, N, P, T, or V;
X₁₁ is E, K, L, M, V, or W;
X₁₂ is E, H, K, L, Q, R, S, or T; and
X₁₃ is E, H, K, M, N, Q, or Y.

In some embodiments, a peptide of Formula (VI) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (VIa) does not comprise or consist of the amino acid sequence, wherein X₁ is Q; X₂ is V; X₃ is P; X₄ is W; X₅ is E; X₇ is P; X₈ is Y; X₉ is Y; X₁₀ is V; X₁₁ is V; X₁₂ is K; and X₁₃ is K.

More generally, in some embodiments, the selection of amino acids at any position of a peptide of Formula (VI) and Subformula (VIa) can be any subset or combination of choices at that position. Thus, the embodiments not only include those in which X₁ is D, H, L, N, Q, or Y; but also, for example, those in which X₁ is D; X₁ is H; X₁ is L; etc.; X₁ is D or H; X₁ is D, H, or L; X₁ is H or L; X₁ is H, L, N, or Q, and so on. Moreover, the choice of amino acids, or combination or subset thereof, at one position, e.g., X₁, is independent of the choice of amino acids (or combination or subset thereof) at any other position, e.g., X₂ or X₃, etc.

### Formula (VII)

In some embodiments, a peptide comprises or consists of the amino acid sequence of Formula (VII): X₁-X₂-X₃-X₄-X₅-X₆-X₇-X₈-X₉-X₁₀-X₁₁-X₁₂-X₁₃-X₁₄, wherein:
X₁ is A, D, E, H, K, L, N, Q, R, or Y;
X₂ is A, C, D, E, F, G, I, L, R, S, or V;
X₃ is A, E, H, L, P, Q, R, S, T, W, or Y;
X₄ is C, F, G, I, L, M, S, T, V, or W;
X₅ is D, E, G, H, Q, S, or V;
X₆ is A, D, E, G, H, Q, T, V, or Y;
X₇ is H, L, P, Q, R, S, T, or V;
X₈ is C, D, E, F, H, L, N, Q, S, W, or Y;
X₉ is C, D, G, H, N, P, Q, S, W, or Y;
X₁₀ is A, D, G, I, L, P, T, or V;
X₁₁ is A, C, F, G, I, K, L, N, R, S, T, or V;
X₁₂ is D, K, N, Q, R, T, V, or Y;
X₁₃ is D, E, I, K, L, M, N, P, Q, R, T, or V; and
X₁₄ is C, F, S, V, or Y.

In some embodiments, a peptide of Formula (VII) comprises or consists of the amino acid sequence of any one of SEQ ID NOS: 5, 7, 10, 16, 21, 32, 53, 55, 61, 71, 84, 106, 110, 120, 123, 128, 130, 137, 151, 152, 204, 210, 237, 258, 268, 275, 281, 308, 323, 328, 330, 334, 367, 374, 375, 380, 395, 426, 433, 434, 474, 475, 486, 489, 490, 510, 525, 528, or 529.

In some embodiments, a peptide of Formula (VII) comprises or consists of the amino acid sequence of Subformula (VIIa): Q-X₂-X₃-W-E-X₆-P-X₈-Y-V-X₁₁-K-X₁₃-S, wherein:
X₂ is A, C, D, E, F, G, I, L, R, S, or V;
X₃ is A, E, H, L, P, Q, R, S, T, W, or Y;
X₆ is A, D, E, G, H, Q, T, V, or Y;
X₈ is C, D, E, F, H, L, N, Q, S, W, or Y;
X₁₁ is A, C, F, G, I, K, L, N, R, S, T, or V; and
X₁₃ is D, E, I, K, L, M, N, P, Q, R, T, or V.

In some embodiments, a peptide of Formula (VII) does not comprise or consist of an amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (VIIa) does not comprise or consist of an amino acid sequence, wherein X₂ is V; X₃ is P; X₆ is E; X₈ is Y; X₁₁ is V; and X₁₃ is K.

More generally, in some embodiments, the selection of amino acids at any position of a peptide of Formula (VII) and Subformula (VIIa) can be any subset or combination of choices at that position. Thus, the embodiments not only include those in which X₂ is A, C, D, E, F, G, I, L, R, S, or V; but also, for example, those in which X₂ is A; X₂ is C; X₂ is D; etc.; X₂ is A or C; X₂ is A, C, or D; X₂ is D or E; X₂ is A, C, D, or R, and so on. Moreover, the choice of amino acids, or combination or subset thereof, at one position, e.g., X₂, is independent of the choice of amino acids (or combination or subset thereof) at any other position, e.g., X₁, X₈, or X₁₁, etc.

### Formula (VIII)

In some embodiments, a peptide comprises or consists of the amino acid sequence of Formula (VIII): X₁-X₂-X₃-X₄-X₅-X₆-X₇-X₈-X₉-X₁₀-X₁₁-X₁₂-X₁₃-X₁₄, wherein:
X₁ is A, D, E, H, L, N, Q, R, S, T, Y;
X₂ is A, D, E, F, H, I, L, N, P, R, S, T, V, Y;
X₃ is C, I, L, P, Q, R, S, T, V;
X₄ is A, C, E, F, G, K, L, M, N, Q, R, S, T, V, W, Y;
X₅ is A, D, E, G, H, K, P, Q, R, S, T, V, Y;
X₆ is A, D, E, G, H, K, L, N, P, Q, R, S, T, V, W, Y;
X₇ is A, D, F, H, I, L, M, P, Q, S, T, W;
X₈ is C, F, G, H, I, K, L, N, P, Q, R, S, T, V, W, Y;
X₉ is A, C, D, E, F, G, H, N, S, T, Y;
X₁₀ is A, D, F, G, I, K, L, M, N, P, R, S, V;
X₁₁ is A, E, G, H, I, M, P, Q, V, Y;
X₁₂ is A, E, H, I, K, L, N, P, Q, R, T, V;
X₁₃ is A, D, E, I, K, M, N, P, R, S, T, V, Y; and
X₁₄ is A, C, F, G, H, L, N, P, S, T, Y.

In some embodiments, a peptide of Formula (VIII) comprises or consists of the amino acid sequence of any one of SEQ ID NOS: 1, 6, 12, 14, 19, 27, 38, 43, 44, 45, 50, 66, 90, 103, 104, 109, 132, 145, 160, 164, 166, 168, 170, 178, 191, 194, 213, 219, 220, 221, 247, 255, 257, 265, 267, 284, 298, 299, 336, 339, 340, 343, 349, 353, 372, 382, 406, 411, 415, 428, 431, 435, 445, 447, 453, 468, 471, 472, 491, 509, 511, 512, 522, 547 and 555.

In some embodiments, a peptide of Formula (VIII) comprises or consists of the amino acid sequence of Subformula (VIIIa): Q-X₂-P-X₄-X₅-X₆-P-X₈-Y-X₁₀-V-X₁₂-X₁₃-X₁₄, wherein:
X₂ is A, D, E, F, H, I, L, N, P, R, S, T, V, Y;
X₄ is A, C, E, F, G, K, L, M, N, Q, R, S, T, V, W, Y;
X₅ is A, D, E, G, H, K, P, Q, R, S, T, V, Y;
X₆ is A, D, E, G, H, K, L, N, P, Q, R, S, T, V, W, Y;
X₈ is C, F, G, H, I, K, L, N, P, Q, R, S, T, V, W, Y;
X₁₀ is A, D, F, G, I, K, L, M, N, P, R, S, V;
X₁₂ is A, E, H, I, K, L, N, P, Q, R, T, V;
X₁₃ is A, D, E, I, K, M, N, P, R, S, T, V, Y; and
X₁₄ is A, C, F, G, H, L, N, P, S, T, Y.

In some embodiments, a peptide of Formula (VIII) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (VIIIa) does not comprise or consist of the amino acid sequence, wherein X₂ is V; X₄ is W; X₅ is E; X₆ is E; X₈ is Y; X₁₀ is V; X₁₂ is K; X₁₃ is K; and X₁₄ is S.

More generally, in some embodiments, the selection of amino acids at any position of a peptide of Formula (VIII) and Subformula (VIIIa) can be any subset or combination of choices at that position. Thus, the embodiments not only include those in which X₂ is A, D, E, F, H, I, L, N, P, R, S, T, V, Y; but also, for example, those in which X₂ is A; X₂ is D; X₂ is E; etc.; X₂ is A or D; X₂ is A, D, or E; X₂ is D or E; X₂ is D, E, F, or V, and so on. Moreover, the choice of amino acids, or combination or subset thereof, at one position, e.g., X₂, is independent of the choice of amino acids (or combination or subset thereof) at any other position, e.g., X₁, X₁₂, or X₁₃, etc.

### Formula (IX)

In some embodiments, a peptide comprises or consists of the amino acid sequence of Formula (IX): X₁-X₂-X₃-X₄-X₅-X₆-X₇-X₈-X₉-X₁₀-X₁₁-X₁₂-X₁₃-X₁₄, wherein:
X₁ is D, E, H, N, Q, R, S, W, or Y;
X₂ is D, F, H, I, L, M, N, P, R, S, V, or Y;
X₃ is A, E, H, L, P, Q, R, S, T, or Y;
X₄ is A, G, H, I, L, N, P, R, S, or W;
X₅ is A, E, G, I, K, Q, S, or V;
X₆ is A, D, E, G, K, L, P, Q, R, T, or V;
X₇ is A, G, K, L, P, Q, R, S, or T;
X₈ is E, I, K, L, S, V, or Y;
X₉ is A, D, F, G, H, I, K, N, Q, S, T, W, or Y;
X₁₀ is A, D, F, H, I, L, N, T, or V;
X₁₁ is A, D, E, F, G, L, M, V, or W;
X₁₂ is E, K, N, Q, R, T, or V;
X₁₃ is E, H, K, L, N, Q, R, S, T, or Y; and
X₁₄ is E, P, R, S, T, or Y.

In some embodiments, a peptide of Formula (IX) comprises or consists of the amino acid sequence of any one of SEQ ID NOS: 8, 31, 79, 93, 127, 153, 155, 156, 169, 186, 189, 202, 212, 218, 226, 304, 329, 331, 335, 338, 352, 361, 366, 371, 376, 381, 390, 401, 408, 418, 424, 430, 436, 438, 443, 457, 493, 515, 521, 544.

In some embodiments, a peptide of Formula (IX) comprises or consists of the amino acid sequence of Subformula (IXa): Q-X₂-P-W-E-X₆-P-Y-X₉-X₁₀-X₁₁-X₁₂-X₁₃-S, wherein:
X₂ is D, F, H, I, L, M, N, P, R, S, V, or Y;
X₆ is A, D, E, G, K, L, P, Q, R, T, or V;
X₉ is A, D, F, G, H, I, K, N, Q, S, T, W, or Y;
X₁₀ is A, D, F, H, I, L, N, T, or V;
X₁₁ is A, D, E, F, G, L, M, V, or W;
X₁₂ is E, K, N, Q, R, T, or V; and
X₁₃ is E, H, K, L, N, Q, R, S, T, or Y.

In some embodiments, a peptide of Formula (IX) does not comprise or consist of the amino acid sequence, wherein X₁ is Q; X₂ is V; X₃ is P; X₄ is W; X₅ is E, X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (IXa) does not comprise or consist of the amino acid sequence, wherein X₂ is V; X₆ is E; X₇ is P; X₉ is Y; X₁₀ is V; Xu is V; X₁₂ is K; and X₁₃ is K.

More generally, in some embodiments, the selection of amino acids at any position of a peptide of Formula (IX) and Subformula (IXa) can be any subset or combination of choices at that position. Thus, the embodiments not only include those in which X₁₂ is E, K, N, Q, R, T, or V; but also, for example, those in which X₁₂ is E; X₁₂ is K; X₁₂ is N; etc.; X₁₂ is E or K; X₁₂ is E, K, or N; X₁₂ is K or N; X₁₂ is K, N, Q, or T, and so on. Moreover, the choice of amino acids, or combination or subset thereof, at one position, e.g., X₁₂, is independent of the choice of amino acids (or combination or subset thereof) at any other position, e.g., X₁, X₆, or X₁₃, etc.

### Formula (X)

In some embodiments, a peptide comprises or consists of the amino acid sequence of Formula (X): X₁-X₂-X₃-X₄-X₅-X₆-X₇-X₈-X₉-X₁₀-X₁₁-X₁₂-X₁₃-X₁₄, wherein:
X₁ is A, D, E, G, H, K, N, Q, R, S, T, or Y;
X₂ is A, D, F, H, I, L, M, Q, S, T, V, or Y;
X₃ is A, E, G, K, L, N, P, Q, R, S, T, V, or Y;
X₄ is C, F, G, H, L, M, R, S, T, W, or Y;
X₅ is A, D, E, G, K, L, N, Q, T, or V;
X₆ is A, D, E, G, H, K, L, N, Q, S, or V,
X₇ is A, L, P, R, S, T, or Y;
X₈ is A, C, D, F, G, N, R, S, T, or Y;
X₉ is A, D, F, H, L, N, Q, R, S, T, or Y;
X₁₀ is A, D, E, F, G, H, I, L, P, R, S, T, or V;
X₁₁ is A, D, E, F, G, H, I, L, M, P, Q, R, S, or V;
X₁₂ is A, D, E, G, H, K, N, Q, S, T, or V;
X₁₃ is D, E, F, H, I, K, L, M, N, Q, R, S, T, V, or Y; and
X₁₄ is A, F, H, I, K, L, N, P, R, S, T, V, or Y.

In some embodiments, a peptide of Formula (X) comprises or consists of the amino acid sequence of any one of SEQ ID NOS: 2, 17, 20, 23, 39, 41, 42, 56, 58, 63, 70, 97, 113, 125, 133, 134, 141, 148, 163, 165, 176, 180, 193, 203, 222, 228, 229, 243, 245, 254, 277, 295, 297, 309, 324, 342, 344, 346, 358, 387, 391, 396, 421, 425, 429, 432, 458, 461, 465, 467, 473, 477, 487, 514, 532, 537, 540, and 556.

In some embodiments, a peptide of Formula (X) comprises or consists of the amino acid sequence of Subformula (Xa): Q-X₂-X₃-X₄-X₅-E-P-Y-Y-X₁₀-X₁₁-K-X₁₃-X₁₄, wherein:
X₂ is A, D, F, H, I, L, M, Q, S, T, V, or Y;
X₃ is A, E, G, K, L, N, P, Q, R, S, T, V, or Y;
X₄ is C, F, G, H, L, M, R, S, T, W, or Y;
X₅ is A, D, E, G, K, L, N, Q, T, or V;
X₁₀ is A, D, E, F, G, H, I, L, P, R, S, T, or V;
X₁₁ is A, D, E, F, G, H, I, L, M, P, Q, R, S, or V;
X₁₃ is D, E, F, H, I, K, L, M, N, Q, R, S, T, V, or Y; and
X₁₄ is A, F, H, I, K, L, N, P, R, S, T, V, or Y.

In some embodiments, a peptide of Formula (X) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (Xa) does not comprise or consist of the amino acid sequence, wherein X₂ is V; X₃ is P; X₄ is W; X₅ is E; X₁₀ is V; X₁₁ is V; X₁₃ is K; and X₁₄ is S.

More generally, in some embodiments, the selection of amino acids at any position of a peptide of Formula (X) and Subformula (Xa) can be any subset or combination of choices at that position. Thus, the embodiments not only include those in which X₅ is A, D, E, G, K, L, N, Q, T, or V; but also, for example, those in which X₅ is A; X₅ is D; X₅ is E; etc.; X₅ is A or D; X₅ is A, D, or E; X₅ is D or E; X₅ is D, E, G, or K and so on. Moreover, the choice of amino acids, or combination or subset thereof, at one position, e.g., X₅, is independent of the choice of amino acids (or combination or subset thereof) at any other position, e.g., X₁, X₂, or X₃, etc.

### Formula (XI)

In some embodiments, a peptide comprises or consists of an amino acid sequence of Formula (XI): X₁-X₂-X₃-X₄-X₅-X₆-X₇-X₈-X₉-X₁₀-X₁₁-X₁₂-X₁₃-X₁₄, wherein:
X₁ is D, E, H, L, Q, or R;
X₂ F, G, H, I, L, N, Q, S, T, V, or Y;
X₃ is A, G, H, L, P, Q, R, S, T, or Y;
X₄ is A, C, F, G, L, P, Q, R, S, W, or Y;
X₅ is A, D, E, G, H, K, N, P, Q, S, T, V, or Y;
X₆ is A, D, E, G, H, K, L, N, Q, T, or V;
X₇ is A, E, H, K, L, P, Q, R, S, or T;
X₈ is C, D, E, F, G, H, K, L, N, P, Q, S, T, V, or Y;
X₉ is A, D, F, G, H, I, L, N, Q, S, T, V, W, or Y;
X₁₀ is A, D, F, G, H, I, L, P, R, S, T, V, or Y;
X₁₁ is A, D, E, F, G, H, L, M, Q, S, or V;
X₁₂ is A, E, H, I, K, L, N, P, Q, R, S, or T;
X₁₃ is A, D, H, I, K, L, M, N, P, Q, S, T, V, or Y; and
X₁₄ is A, F, H, L, P, S, T, V, or Y.

In some embodiments, a peptide of Formula (XI) comprises or consists of an amino acid sequence of any one of SEQ ID NOS: 11, 29, 46, 48, 51, 62, 77, 87, 88, 95, 98, 100, 162, 173, 201, 236, 253, 256, 263, 273, 278, 300, 327, 341, 351, 364, 386, 388, 399, 400, 403, 404, 414, 419, 420, 437, 441, 444, 449, 450, 466, 469, 478, 481, 483, 484, 496, 500, 516, 524, 533, 535, 536, 542, 543, 548, and 554.

In some embodiments, a peptide of Formula (XI) comprises or consists of an amino acid sequence of Subformula (XIa): Q-X₂-P-X₄-X₅-X₆-X₇-X₈-Y-X₁₀-X₁₁-K-X₁₃-S, wherein:
X₂ is F, G, H, I, L, N, Q, S, T, V, or Y;
X₄ is A, C, F, G, L, P, Q, R, S, W, or Y;
X₅ is A, D, E, G, H, K, N, P, Q, S, T, V, or Y;
X₆ is A, D, E, G, H, K, L, N, Q, T, or V;
X₇ is A, E, H, K, L, P, Q, R, S, or T;
X₈ is C, D, E, F, G, H, K, L, N, P, Q, S, T, V, or Y;
X₁₀ is A, D, F, G, H, I, L, P, R, S, T, V, or Y;
X₁₁ is A, D, E, F, G, H, L, M, Q, S, or V; and
X₁₃ is A, D, H, I, K, L, M, N, P, Q, S, T, V, or Y.

In some embodiments, a peptide of Formula (XI) does not comprise or consist of an amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XIa) does not comprise or consist of an amino acid sequence, wherein X₂ is V; X₄ is W; X₅ is E; X₆ is E; X₇ is P; X₈ is Y; X₁₀ is V; X₁₁ is V; and X₁₃ is K.

More generally, in some embodiments, the selection of amino acids at any position of a peptide of Formula (XI) and Subformula (XIa) can be any subset or combination of choices at that position. Thus, the embodiments not only include those in which X₇ is A, E, H, K, L, P, Q, R, S, or T; but also, for example, those in which X₇ is A; X₇ is H; X₇ is L; etc.; X₇ is E or H; X₇ is E, H, or L; X₇ is H or L; X₇ is H, L, P, or Q, and so on. Moreover, the choice of amino acids, or combination or subset thereof, at one position, e.g., X₇, is independent of the choice of amino acids (or combination or subset thereof) at any other position, e.g., X₁, X₁₀, or X₁₁, etc.

### Formula (XII)

In some embodiments, a peptide comprises or consists of an amino acid sequence of Formula (XII): X₁-X₂-X₃-X₄-X₅-X₆-X₇-X₈-X₉-X₁₀-X₁₁-X₁₂-X₁₃-X₁₄, wherein:
X₁ is E, H, K, L, N, Q, or R;
X₂ is A, C, D, F, I, K, L, M, N, S, V, or Y;
X₃ is A, L, P, R, S, or T;
X₄ is A, E, F, G, L, M, R, S, T, W, or Y;
X₅ is A, D, E, F, G, H, K, N, Q, R, T, V, or Y;
X₆ is A, D, E, G, Q, R, S, T, V, or Y;
X₇ is A, H, K, L, P, Q, R, S, or T;
X₈ is A, C, D, F, H, N, R, S, T, or Y;
X₉ is D, E, F, H, L, N, R, S, T, V, or Y;
X₁₀ is A, D, F, G, H, I, L, N, R, S, T, or V;
X₁₁ is A, D, F, G, L, M, P, Q, R, T, V, or W;
X₁₂ is A, D, E, H, K, L, M, N, P, Q, R, S, T, or Y;
X₁₃ is E, H, I, K, L, M, N, P, Q, R, S, T, or V; and
X₁₄ is A, F, P, or S.

In some embodiments, a peptide of Formula (XII) comprises or consists of the amino acid sequence of any one of SEQ ID NOS: 9, 34, 47, 57, 72, 73, 82, 92, 105, 115, 117, 122, 138, 143, 146, 161, 171, 172, 190, 200, 208, 211, 244, 246, 274, 276, 280, 293, 332, 333, 337, 354, 356, 369, 394, 398, 402, 442, 446, 452, 455, 463, 470, 479, 480, 482, 497, 499, 501, 507, 508, 517, 534, 541, 553, and 557.

In some embodiments, a peptide of Formula (XII) comprises or consists of the amino acid sequence of Subformula (XIIa): Q-X₂-P-X₄-X₅-E-P-Y-X₉-X₁₀-X₁₁-X₁₂-X₁₃-S, wherein:
X₂ is A, C, D, F, I, K, L, M, N, S, V, or Y;
X₄ is A, E, F, G, L, M, R, S, T, W, or Y;
X₅ is A, D, E, F, G, H, K, N, Q, R, T, V, or Y;
X₉ is D, E, F, H, L, N, R, S, T, V, or Y;
X₁₀ is A, D, F, G, H, I, L, N, R, S, T, or V;
X₁₁ is A, D, F, G, L, M, P, Q, R, T, V, or W;
X₁₂ is A, D, E, H, K, L, M, N, P, Q, R, S, T, or Y; and
X₁₃ is E, H, I, K, L, M, N, P, Q, R, S, T, or V.

In some embodiments, a peptide of Formula (XII) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XIIa) does not comprise or consist of the amino acid sequence, wherein X₂ is V; X₄ is W; X₅ is E; X₉ is Y; X₁₀ is V; X₁₁ is V; X₁₂ is K; and X₁₃ is K.

More generally, in some embodiments, the selection of amino acids at any position of a peptide of Formula (XII) and Subformula (XIIa) can be any subset or combination of choices at that position. Thus, the embodiments not only include those in which X₂ is A, C, D, F, I, K, L, M, N, S, V, or Y; but also, for example, those in which X₂ is A; X₂ is C; X₂ is D; etc.; X₂ is A or C; X₂ is A, C, or D; X₂ is C or D; X₂ is C, D, F, or L, and so on. Moreover, the choice of amino acids, or combination or subset thereof, at one position, e.g., X₂, is independent of the choice of amino acids (or combination or subset thereof) at any other position, e.g., X₁, X₄, or X₅, etc.

### Formula (XIII)

In some embodiments, a peptide comprises or consists of the amino acid sequence of Formula (XIII): X₁-X₂-X₃-X₄-X₅-E-X₇-X₈-X₉-X₁₀-X₁₁-X₁₂-X₁₃-X₁₄, wherein:
X₁ is E, F, H, or Q;
X₂ is A, F, I, M, S, T, or V;
X₃ is A, G, H, P, Q, R, or T;
X₄ is F, G, L, M, N, R, S, W, or Y;
X₅ is A, G, K, N, P, Q, or R;
X₇ is H, P, R, or T;
X₈ is D, F, H, K, L, N, or Y;
X₉ is F, G, H, K, Q, T, V, or Y;
X₁₀ is F, G, L, P, S, V, or Y;
X₁₁ is L, M, Q, or V;
X₁₂ is E, G, I, K, L, N, P, S, or Y;
X₁₃ is E, H, I, N, Q, S, or T; and
X₁₄ is I, P, S, or Y.

In some embodiments, a peptide of Formula (XIII) comprises or consists of the amino acid sequence of any one of SEQ ID NOS: 15, 54, 74, 154, 184, 192, 206, 230, 242, 269, 271, 306, 427, 492, or 505.

In some embodiments, a peptide of Formula (XIII) comprises or consists of the amino acid sequence of Subformula (XIIIa): H-X₂-A-X₄-X₅-E-P-X₈-X₉-X₁₀-V-X₁₂-X₁₃-S, wherein:
X₂ is A, F, I, M, S, T, or V;
X₄ is F, G, L, M, N, R, S, W, or Y;
X₅ is A, G, K, N, P, Q, or R;
X₈ is D, F, H, K, L, N, or Y;
X₉ is F, G, H, K, Q, T, V, or Y;
X₁₀ is F, G, L, P, S, V, or Y;
X₁₂ is E, G, I, K, L, N, P, S, or Y; and
X₁₃ is E, H, I, N, Q, S, or T.

In some embodiments, a peptide of Formula (XIII) does not comprise or consist of the amino acid sequence, wherein X₁ is Q; X₂ is V; X₃ is P; X₄ is W; X₇ is P; X₈ is Y; X₉ is Y; X₁₀ is V; X₁₁ is V; X₁₂ is K; and X₁₄ is S.

In some embodiments, a peptide of Subformula (XIIIa) does not comprise or consist of the amino acid sequence, wherein X₂ is V; X₄ is W; X₈ is Y; X₉ is Y; X₁₀ is V; and X₁₂ is K.

More generally, in some embodiments, the selection of amino acids at any position of a peptide of Formula (XIII) and Subformula (XIIIa) can be any subset or combination of choices at that position. Thus, the embodiments not only include those in which X₂ is A, F, I, M, S, T, or V; but also, for example, those in which X₂ is A; X₂ is F; X₂ is I; etc.; X₂ is A or F; X₂ is A, F, or I; X₂ is F or I; X₂ is F, I, M, or V, and so on. Moreover, the choice of amino acids, or combination or subset thereof, at one position, e.g., X₂, is independent of the choice of amino acids (or combination or subset thereof) at any other position, e.g., X₈ or X₉, etc.

### Formula (XIV)

In some embodiments, a peptide comprises or consists of the amino acid sequence of Formula (XIV): X₁-X₂-X₃-X₄-X₅-X₆-X₇-X₈-X₉-X₁₀-X₁₁-X₁₂-X₁₃-X₁₄, wherein:
X₁ is H, L, N, Q, or R;
X₂ is F, L, R, S, or V;
X₃ is P, S, or T;
X₄ is A, L, S, or T;
X₅ is A, D, E, H, Q, R, V, or Y;
X₆ is D, E, or K;
X₇ is A, P, Q, or W;
X₈ is H, R, W, or Y;
X₉ is H, K, L, S, or Y;
X₁₀ is D, E, F, L, T, or V;
X₁₁ is A, D, or L;
X₁₂ is D, E, G, H, K, N, Q, or R;
X₁₃ is G, K, N, Q, or T; and
X₁₄ is P or S.

In some embodiments, a peptide of Formula (XIV) comprises or consists of the amino acid sequence of any one of SEQ ID NOS: 18, 24, 49, 126, 231, 239, 250, 264, 291, 301, 523, and 526. In some embodiments, a peptide of Formula (XIV) comprises or consists of the amino acid sequence of Subformula (XIVa): H-V-X₃-S-X₅-E-P-Y-Y-X₁₀-L-X₁₂-X₁₃-S, wherein:
X₃ is P, S, or T;
X₅ is A, D, E, H, Q, R, V, or Y;
X₁₀ is D, E, F, L, T, or V;
X₁₂ is D, E, G, H, K, N, Q, or R; and
X₁₃ is G, K, N, Q, or T.

In some embodiments, a peptide of Formula (XIV) does not comprise or consist of the amino acid sequence, wherein X₁ is Q; X₂ is V; X₃ is P; X₅ is E; X₆ is E; X₇ is P; X₈ is Y; X₉ is Y; X₁₀ is V; X₁₂ is K; X₁₃ is K; and X₁₄ is S.

In some embodiments, a peptide of Subformula (XIVa) does not comprise or consist of the amino acid sequence, wherein X₃ is P; X₅ is E; X₁₀ is V; X₁₂ is K; and X₁₃ is K.

More generally, in some embodiments, the selection of amino acids at any position of a peptide of Formula (XIV) and Subformula (XIVa) can be any subset or combination of choices at that position. Thus, the embodiments not only include those in which X₃ is P, S, or T; but also, for example, those in which X₃ is P; X₃ is S; X₃ is T; etc.; X₃ is Por S; X₃ is P, S, or T; X₃ is S or T; and so on. Moreover, the choice of amino acids, or combination or subset thereof, at one position, e.g., X₃, is independent of the choice of amino acids (or combination or subset thereof) at any other position, e.g., X₁, X₁₂, or X₁₃, etc.

### Formula (XV)

In some embodiments, a peptide comprises or consists of the amino acid sequence of Formula (XV): X₁-X₂-X₃-X₄-X₅-X₆-X₇-Y-X₉-X₁₀-X₁₁-X₁₂-X₁₃-X₁₄, wherein:
X₁ is E, K, Q, or T;
X₂ is D, F, L, P, or V;
X₃ is C, L, P, T, or V;
X₄ is A, L, or T;
X₅ is D, E, H, or Q;
X₆ is A, D, K, or N;
X₇ is A, P, or S;
X₉ is C, H, M, or Y;
X₁₀ is F, L, or V;
X₁₁ is E, G, H, R, or V;
X₁₂ is K or T;
X₁₃ is I or Q; and
X₁₄ is A, F, or T.

In some embodiments, a peptide of Formula (XV) comprises or consists of the amino acid sequence of any one of SEQ ID NOS: 150, 238, 321, 397, and 415.

In some embodiments, a peptide of Formula (XV) comprises or consists of the amino acid sequence of Subformula (XVa): X₁-X₂-X₃-X₄-X₅-X₆-P-Y-X₉-X₁₀-X₁₁-T-Q-T. wherein:
X₁ is E, K, Q, or T;
X₂ is D, F, L, P, or V;
X₃ is C, L, P, T, or V;
X₄ is A, L, or T;
X₅ is D, E, H, or Q;
X₆ is A, D, K, or N;
X₉ is C, H, M, or Y;
X₁₀ is F, L, or V; and
X₁₁ is E, G, H, R, or V.

In some embodiments, a peptide of Formula (XV) does not comprise or consist of the amino acid sequence, wherein X₁ is Q; X₂ is V; X₃ is P; X₅ is E; X₇ is P; X₉ is Y; X₁₀ is V; Xu is V; and X₁₂ is K.

In some embodiments, a peptide of Subformula (XVa) does not comprise or consist of the amino acid sequence, wherein X₁ is Q; X₂ is V; X₃ is P; X₅ is E; X₉ is Y; X₁₀ is V; and X₁₁ is V.

More generally, in some embodiments, the selection of amino acids at any position of a peptide of Formula (XV) and Subformula (XVa) can be any subset or combination of choices at that position. Thus, the embodiments not only include those in which X₁ is E, K, Q, or T; but also, for example, those in which X₁ is E; X₁ is K; X₁ is Q; etc.; X₁ is E or K; X₁ is E, K, or Q; X₁ is E or K; X₁ is E, K, or T; and so on. Moreover, the choice of amino acids, or combination or subset thereof, at one position, e.g., X₁, is independent of the choice of amino acids (or combination or subset thereof) at any other position, e.g., X₂ or X₃, etc.

### Formula (XVI)

In some embodiments, a peptide comprises or consists of an amino acid sequence of Formula (XVI): X₁-X₂-X₃-X₄-X₅-X₆-X₇-X₈-X₉-X₁₀-X₁₁-X₁₂-X₁₃-X₁₄, wherein:
X₁ is D, H, K, or Q;
X₂ is F, I, or V;
X₃ is A, P, R, S, or T;
X₄ is G, L, M, Q, S, T, or W;
X₅ is D, G, Q, T, or V;
X₆ is D, E, H, Q, or S;
X₇ is A, P, R, or T;
X₈ is E, G, T, V, or Y;
X₉ is A, Q, S, or Y;
X₁₀ is A, D, E, G, I, L, R, T, V, or Y;
X₁₁ is A, E, F, G, or I;
X₁₂ is K, Q, or T;
X₁₃ is E, I, K, M, N, or S; and
X₁₄ is F, L, S, or T.

In some embodiments, a peptide of Formula (XVI) comprises or consists of an amino acid sequence of any one of SEQ ID NOS: 2, 48, 63, 70, 100, 141, 193, 228, 292, 403, 425, 473, and 477.

In some embodiments, the peptide comprises or consists of the amino acid sequence of Subformula (XVIa): Q-F-P-X₄-X₅-E-P-Y-Y-X₁₀-X₁₁-K-K-T, wherein:
X₄ is G, L, M, Q, S, T, or W;
X₅ is D, G, Q, T, or V;
X₁₀ is A, D, E, G, I, L, R, T, V, or Y; and
X₁₁ is A, E, F, G, or I.

In some embodiments, a peptide of Formula (XVI) does not comprise or consist of an amino acid sequence, wherein X₁ is Q; X₂ is V; X₃ is P; X₄ is W; X₆ is E; X₇ is P; X₈ is Y; X₉ is Y; X₁₀ is V; X₁₂ is K; X₁₃ is K; and X₁₄ is S.

In some embodiments, a peptide of Subformula (XVIa) does not comprise or consist of an amino acid sequence, wherein X₄ is W and X₁₀ is V.

More generally, in some embodiments, the selection of amino acids at any position of a peptide of Formula (XVI) and Subformula (XVIa) can be any subset or combination of choices at that position. Thus, the embodiments not only include those in which X₅ is D, G, Q, T, or V; but also, for example, those in which X₅ is D; X₅ is G; X₅ is Q; etc.; X₅ is D or G; X₅ is D, G, or Q; X₅ is Q or T; X₅ is D, Q, T, or V, and so on. Moreover, the choice of amino acids, or combination or subset thereof, at one position, e.g., X₅, is independent of the choice of amino acids (or combination or subset thereof) at any other position, e.g., X₁, X₁₀ or X₁₁, etc.

### Formula (XVII)

In some embodiments, a peptide comprises or consists of the amino acid sequence of Formula (XVII): Q-X₂-X₃-X₄-X₅-X₆-X₇-X₈-X₉-X₁₀-X₁₁-X₁₂-X₁₃-X₁₄, wherein:
X₂ is H, S, or T;
X₃ is P, Q, or T;
X₄ is F, M, P, or W;
X₅ is D, E, or Q;
X₆ is E or P;
X₇ is P, R, or T;
X₈ is A, K, or Y;
X₉ is H or Y;
X₁₀ is G, R, or V;
X₁₁ is L or V;
X₁₂ is I, L, S, or T;
X₁₃ is H, K, M, or Q; and
X₁₄ is A or S.

In some embodiments, a peptide of Formula (XVII) comprises or consists of the amino acid sequence of any one of SEQ ID NOS: 74, 265, 266, 296, and 482.

In some embodiments, a peptide of Formula (XVII) comprises or consists of the amino acid sequence of Subformula (XVIIa): Q-S-P-X₄-D-E-X₇-Y-Y-G-V-X₁₂-X₁₃-A, wherein:
X₄ is F, M, P, or W;
X₇ is P, R, or T;
X₁₂ is I, L, S, or T; and
X₁₃ is H, K, M, or Q.

In some embodiments, a peptide of Formula (XVII) does not comprise or consist of the amino acid sequence, wherein X₃ is P; X₄ is W; X₅ is E; X₆ is E; X₇ is P; X₈ is Y; X₉ is Y; X₁₀ is V; X₁₁ is V; X₁₃ is K; and X₁₄ is S.

In some embodiments, a peptide of Subformula (XVIIa) does not comprise or consist of the amino acid sequence, wherein X₄ is W; X₇ is P; and X₁₃ is K.

More generally, in some embodiments, the selection of amino acids at any position of a peptide of Formula (XVII) and Subformula (XVIIa) can be any subset or combination of choices at that position. Thus, the embodiments not only include those in which X₄ is F, M, P, or W; but also, for example, those in which X₄ is F; X₄ is M; X₄ is W; etc.; X₄ is F or M; X₄ is F, M, or W, and so on. Moreover, the choice of amino acids, or combination or subset thereof, at one position, e.g., X₄, is independent of the choice of amino acids (or combination or subset thereof) at any other position, e.g., X₁₂ or X₁₃, etc.

### Formula (XVIII)

In some embodiments, the peptide comprises or consists of the amino acid sequence of Formula (XVIII): X₁-X₂-X₃-X₄-X₅-X₆-X₇-X₈-X₉-X₁₀-X₁₁-X₁₂-X₁₃-X₁₄, wherein:
X₁ is K, Q, or R;
X₂ is F or L;
X₃ is G, P, or T;
X₄ is E, L, Q, S, or W;
X₅ is A, D, E, F, P, Q, V, or Y;
X₆ is A, D, E, K, N, Q, T, or Y;
X₇ is H, L, P, Q, R, or T;
X₈ is F, N, or Y;
X₉ is H, T, or Y;
X₁₀ is F, H, I, L, N, T, or Y;
X₁₁ is D, G, L, M, Q, or V;
X₁₂ is D, E, K, R, or T;
X₁₃ is A, E, K, M, N, P, Q, R, or T; and
X₁₄ is A, S, or P.

In some embodiments, the peptide of Formula (XVIII) comprises or consists of the amino acid of any one of SEQ ID NOS: 50, 88, 117, 143, 201, 211, 300, 303, 337, 420, 455, 463, 499, and 536.

In some embodiments, a peptide of Formula (XVIII) comprises or consists of the amino acid sequence of Subformula (XVIIIa): Q-L-P-L-X₅-X₆-X₇-N-Y-X₁₀-V-T-X₁₃-S, wherein:
X₅ is A, D, E, F, P, Q, V, or Y;
X₆ is A, D, E, K, N, Q, T, or Y;
X₇ is H, L, P, Q, R, or T;
X₁₀ is F, H, I, L, N, T, or Y; and
X₁₃ is A, E, K, M, N, P, Q, R, or T.

In some embodiments, a peptide of Formula (XVIII) does not comprise or consist of the amino acid sequence, wherein X₁ is Q; X₃ is P; X₄ is W; X₅ is E; X₆ is E; X₇ is P; X₈ is Y; X₉ is Y; X₁₁ is V; X₁₂ is K; X₁₃ is K; and X₁₄ is S.

In some embodiments, a peptide of Subformula (XVIIIa) does not comprise or consist of the amino acid sequence, wherein X₅ is E; X₆ is E; X₇ is P; and X₁₃ is K.

More generally, in some embodiments, the selection of amino acids at any position of a peptide of Formula (XVIII) and Subformula (XVIIIa) can be any subset or combination of choices at that position. Thus, the embodiments not only include those in which X₅ is A, D, E, F, P, Q, V, or Y; but also, for example, those in which X₅ is A; X₅ is D; X₅ is E; etc.; X₅ is A or D; X₅ is A, D, or E; X₅ is D or E; X₅ is D, E, P, or Q, and so on. Moreover, the choice of amino acids, or combination or subset thereof, at one position, e.g., X₅, is independent of the choice of amino acids (or combination or subset thereof) at any other position, e.g., X₁₀ or X₁₃, etc.

### Formula (XIX)

In some embodiments, the peptide comprises or consists of the amino acid sequence of Formula (XIX): X₁-X₂-X₃-X₄-X₅-X₆-X₇-X₈-X₉-X₁₀-X₁₁-X₁₂-X₁₃-X₁₄, wherein:
X₁ is Q or T;
X₂ is H, I, L, T, or V;
X₃ is P or T;
X₄ is A, C, F, L, R, S, T, or Y;
X₅ is A, E, G, H, or Q;
X₆ is A, E, K, Q, T, V, or Y;
X₇ is L, M, P, Q, or S;
X₈ is C, F, L, N, W, or Y;
X₉ is C or Y;
X₁₀ is A, L, P, S, or V;
X₁₁ is A, G, or V;
X₁₂ is K, N, Q, R, or V;
X₁₃ is A, E, K, T, V, or Y; and
X₁₄ is F, G, N, P, S, T, or Y.

In some embodiments, a peptide of Formula (XIX) comprises or consists of the amino acid sequence of any one of SEQ ID NOS: 1, 6, 12, 45, 103, 220, 267, 298, 299, 336, 343, 428, 491, and 522.

In some embodiments, a peptide of Formula (XIX) comprises or consists of the amino acid sequence of Subformula (XIXa): Q-X₂-P-X₄-E-X₆-P-X₈-Y-X₁₀-V-X₁₂-K-X₁₄, wherein:
X₂ is H, I, L, T, or V;
X₄ is A, C, F, L, R, S, T, or Y;
X₆ is A, E, K, Q, T, V, or Y;
X₈ is C, F, L, N, W, or Y;
X₁₀ is A, L, P, S, or V;
X₁₂ is K, N, Q, R, or V; and
X₁₄ is F, G, N, P, S, T, or Y.

In some embodiments, a peptide of Formula (XIX) does not comprise or consist of the amino acid sequence, wherein X₁ is Q; X₂ is V; X₃ is P; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XIXa) does not comprise or consist of the amino acid sequence, wherein X₂ is V; X₆ is E; X₈ is Y; X₁₀ is V; X₁₂ is K; and X₁₄ is S.

More generally, in some embodiments, the selection of amino acids at any position of a peptide of Formula (XIX) and Subformula (XIXa) can be any subset or combination of choices at that position. Thus, the embodiments not only include those in which X₂ is H, I, L, T, or V; but also, for example, those in which X₂ is H; X₂ is I; X₂ is L; etc.; X₂ is H or I; X₂ is H, I, or L; X₂ is I or L; X₂ is I, L, T, or V, and so on. Moreover, the choice of amino acids, or combination or subset thereof, at one position, e.g., X₂, is independent of the choice of amino acids (or combination or subset thereof) at any other position, e.g., X₁₂ or X₁₄, etc.

### Formula (XX)

In some embodiments, the peptide comprises or consists of an amino acid sequence of Formula (XX): X₁-X₂-X₃-X₄-X₅-X₆-X₇-X₈-X₉-X₁₀-X₁₁-X₁₂-X₁₃-X₁₄, wherein:
X₁ is E, H, N, Q, or S;
X₂ is H, I, L, or V;
X₃ is A, P, or T;
X₄ is G, L, M, or W;
X₅ is D, E, G, or V;
X₆ is A, D, E, G, H, L, or V;
X₇ is G, K, P, Q, R, or T;
X₈ is E, L, S, or Y;
X₉ is H, K, N, Q, R, S, V, or Y;
X₁₀ is A, F, I, L, or P;
X₁₁ is F, G, I, L, M, P, R, or V;
X₁₂ is H, K, N, or V;
X₁₃ is H, K, L, M, R, S, or T; and
X₁₄ is F, P, R, S, or Y.

In some embodiments, a peptide of Formula (XX) comprises or consists of the amino acid sequence of any one of SEQ ID NOS: 31, 58, 92, 105, 125, 169, 176, 245, 276, 293, 338, 381, 443, 452, 497, and 501.

In some embodiments, a peptide of Formula (XX) comprises or consists of the amino acid sequence of Subformula (XXa): Q-L-P-G-E-X₆-X₇-Y-X₉-L-X₁₁-K-K-S, wherein:
X₆ is A, D, E, G, H, L, or V;
X₇ is G, K, P, Q, R, or T;
X₉ is H, K, N, Q, R, S, V, or Y; and
X₁₁ is F, G, I, L, M, P, R, or V.

In some embodiments, a peptide of Formula (XX) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₁ is V; X₁₂ is K; X₁₃ is K; and X₁₄ is S.

In some embodiments, a peptide of Subformula (XXa) does not comprise or consist of an amino acid sequence, wherein X₆ is E; X₇ is P; X₉ is Y; and X₁₁ is V.

More generally, in some embodiments, the selection of amino acids at any position of a peptide of Formula (XX) and Subformula (XXa) can be any subset or combination of choices at that position. Thus, the embodiments not only include those in which X₁ is E, H, N, Q, or S; but also, for example, those in which X₁ is E; X₁ is H; X₁ is Q; etc.; X₁ is E or H; X₁ is E, H, or N; X₁ is N or Q; X₁ is N, Q, or S, and so on. Moreover, the choice of amino acids, or combination or subset thereof, at one position, e.g., X₁, is independent of the choice of amino acids (or combination or subset thereof) at any other position, e.g., X₆ or X₇, etc.

### Formula (XXI)

In some embodiments, the peptide comprises or consists of the amino acid sequence of Formula (XXI): X₁-X₂-X₃-X₄-X₅-E-X₇-X₈-X₉-X₁₀-X₁₁-K-X₁₃-X₁₄, wherein:
X₁ is D, Q, or R;
X₂ is A, F, H, I, L, V, or Y;
X₃ is A, E, L, P, or Q;
X₄ is A, L, R, S, or V;
X₅ is A, E, G, H, K, Q, or R;
X₇ is K, L, P, R, or T;
X₈ is D, N, R, or Y;
X₉ is E, F, H, N, R, S, or T;
X₁₀ is A, D, F, I, L, R, or V;
X₁₁ is A, F, L, M, P, Q, V, or W;
X₁₃ is E, H, K, M, or R; and
X₁₄ is P, F, or S.

In some embodiments, a peptide of Formula (XXI) comprises or consists of the amino acid sequence of any one of SEQ ID NOS: 9, 57, 138, 146, 163, 203, 227, 244, 274, 354, 442, 446, 487, and 532.

In some embodiments, a peptide of Formula (XXI) comprises or consists of the amino acid sequence of Subformula (XXIa): Q-X₂-P-S-X₅-E-P-Y-X₉-X₁₀-X₁₁-K-X₁₃-S, wherein:
X₂ is A, F, H, I, L, V, or Y;
X₅ is A, E, G, H, K, Q, or R;
X₉ is E, F, H, N, R, S, or T;
X₁₀ is A, D, F, I, L, R, or V;
X₁₁ is A, F, L, M, P, Q, V, or W; and
X₁₃ is E, H, K, M, or R.

In some embodiments, a peptide of Formula (XXI) does not comprise or consist of the amino acid sequence, wherein X₁ is Q; X₂ is V; X₃ is P; X₅ is E; X₇ is P; X₈ is Y; X₁₀ is V; X₁₁ is V; X₁₃ is K; and X₁₄ is S.

In some embodiments, a peptide of Subformula (XXI) does not comprise or consist of the amino acid sequence, wherein X₂ is V; X₅ is E; X₁₀ is V; X is V; and X₁₃ is K.

More generally, in some embodiments, the selection of amino acids at any position of a peptide of Formula (XXI) and Subformula (XXIa) can be any subset or combination of choices at that position. Thus, the embodiments not only include those in which X₂ is A, F, H, I, L, V, or Y; but also, for example, those in which X₂ is A; X₂ is F; X₂ is H; etc.; X₂ is A or F; X₂ is A, F, or H; X₂ is F or H; X₂ is F, H, I, or V, and so on. Moreover, the choice of amino acids, or combination or subset thereof, at one position, e.g., X₂, is independent of the choice of amino acids (or combination or subset thereof) at any other position, e.g., X₁₀ or X₁₁, etc.

### Formula (XXII)

In some embodiments, a peptide comprises or consists of the amino acid sequence of Formula (XXII): X₁-X₂-X₃-X₄-X₅-X₆-X₇-X₈-X₉-X₁₀-X₁₁-X₁₂-X₁₃-X₁₄, wherein:
X₁ is A, H, L, or Q;
X₂ is D, F, I, L, N, S, T, or Y;
X₃ is P, Q, S, or T;
X₄ is A, C, L, S, or W;
X₅ is A, D, E, H, K, Q, S, T, or V;
X₆ is A, D, E, G, H, K, Q, R, S, W, or Y;
X₇ is D, P, or S;
X₈ is C, F, G, H, L, N, Q, S, T, W, or Y;
X₉ is A, C, D, F, N, S, or Y;
X₁₀ is A, D, G, L, or V;
X₁₁ is A, G, L, Q, S, T, V, W, or Y;
X₁₂ is A, D, K, P, Q, S, T, or Y;
X₁₃ is I, K, M, N, P, R, or T; and
X₁₄ is A, C, F, P, S, or Y.

In some embodiments, a peptide of Formula (XXII) comprises or consists of the amino acid sequence of any one of SEQ ID NOS: 7, 16, 19, 27, 47, 66, 72, 166, 194, 236, 246, 278, 332, 340, 394, 453, 475, and 479.

In some embodiments, a peptide of Formula (XXII) comprises or consists of the amino acid sequence of Subformula (XXIIa): Q-X₂-P-X₄-X₅-X₆-P-X₈-S-X₁₀-V-X₁₂-X₁₃-X₁₄, wherein:
X₂ is D, F, I, L, N, S, T, or Y;
X₄ is A, C, L, S, or W;
X₅ is A, D, E, H, K, Q, S, T, or V;
X₆ is A, D, E, G, H, K, Q, R, S, W, or Y;
X₈ is C, F, G, H, L, N, Q, S, T, W, or Y;
X₁₀ is A, D, G, L, or V;
X₁₂ is A, D, K, P, Q, S, T, or Y;
X₁₃ is I, K, M, N, P, R, or T; and
X₁₄ is A, C, F, P, S, or Y.

In some embodiments, a peptide of Formula (XXII) does not comprise or consist of the amino acid sequence, wherein X₁ is Q; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XXIIa) does not comprise or consist of the amino acid sequence, wherein X₄ is W; X₅ is E; X₆ is E; X₈ is Y; X₁₀ is V; X₁₂ is K; X₁₃ is K; and X₁₄ is S.

More generally, in some embodiments, the selection of amino acids at any position of a peptide of Formula (XXII) and Subformula (XXIIa) can be any subset or combination of choices at that position. Thus, the embodiments not only include those in which X₄ is A, C, L, S, or W; but also, for example, those in which X₄ is C; X₄ is L; X₄ is S; etc.; X₄ is C or L; X₄ is C, L, or S; X₄ is L or S; X₄ is C, L, S, or W, and so on. Moreover, the choice of amino acids, or combination or subset thereof, at one position, e.g., X₄, is independent of the choice of amino acids (or combination or subset thereof) at any other position, e.g., X₆ or X₈, etc.

### Formula (XXIII)

In some embodiments, the peptide comprises or consists of the amino acid sequence of Formula (XXIII): X₁-X₂-X₃-X₄-X₅-X₆-X₇-X₈-X₉-X₁₀-X₁₁-X₁₂-X₁₃-X₁₄, wherein:
X₁ is D, E, or Q;
X₂ is L, M, N, P, R, or V;
X₃ is P, Q, R, or T;
X₄ is R, S, or W;
X₅ is E, G, H, Q, T, or Y;
X₆ is A, E, K, N, Q, or V;
X₇ is A, P, R, or S;
X₈ is D, F, H, K, N, or Y;
X₉ is D, F, H, N, S, T, W, or Y;
X₁₀ is G, I, R, or V;
X₁₁ is D, G, M, or V;
X₁₂ is E, K, N, R, or V;
X₁₃ is I, K, N, Q, R, S, T, V, or Y; and
X₁₄ is G, H, P, S, T, or Y.

In some embodiments, a peptide of Formula (XXIII) comprises or consists of the amino acid sequence of any one of SEQ ID NOS: 46, 87, 93, 98, 202, 218, 255, 339, 351, 352, 361, 376, 430, 436, 496, and 511.

In some embodiments, a peptide of Formula (XXIII) comprises or consists of the amino acid sequence of Subformula (XXIIIa): Q-L-P-W-X₅-X₆-P-Y-X₉-V-V-X₁₂-X₁₃-S, wherein:
X₅ is E, G, H, Q, T, or Y;
X₆ is A, E, K, N, Q, or V;
X₉ is D, F, H, N, S, T, W, or Y;
X₁₂ is E, K, N, R, or V; and
X₁₃ is I, K, N, Q, R, S, T, V, or Y.

In some embodiments, a peptide of Formula (XXIII) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XXIIIa) does not comprise or consist of the amino acid sequence, wherein X₅ is E; X₆ is E; X₉ is Y; X₁₂ is K; and X₁₃ is K.

More generally, in some embodiments, the selection of amino acids at any position of a peptide of Formula (XXIII) and Subformula (XXIIIa) can be any subset or combination of choices at that position. Thus, the embodiments not only include those in which X₅ is E, G, H, Q, T, or Y; but also, for example, those in which X₅ is E; X₅ is G; X₅ is H; etc.; X₅ is E or G; X₅ is E, G, or H; X₅ is G or H; X₅ is G, H, Q, or T, and so on. Moreover, the choice of amino acids, or combination or subset thereof, at one position, e.g., X₅, is independent of the choice of amino acids (or combination or subset thereof) at any other position, e.g., X₁₂ or X₁₃, etc.

### Formula (XXIV)

In some embodiments, the peptide comprises or consists of the amino acid sequence of Formula (XXIV): Q-X₂-P-X₄-X₅-X₆-X₇-X₈-X₉-X₁₀-X₁₁-X₁₂-X₁₃-S wherein:
X₂ is D, I, K, L, or M;
X₄ is F, G, L, S, or T;
X₅ is E, G, H, N, Q, V, or Y;
X₆ is A, D, E, G, or S;
X₇ is L, P, or S;
X₈ is N, T, or Y;
X₉ is D, N, or Y;
X₁₀ is A, G, I, S, or V;
X₁₁ is D, G, L, Q, or T;
X₁₂ is K, L, M, N, Q, R, S, or T; and
X₁₃ is E, K, M, N, P, S, or V.

In some embodiments, a peptide of Formula (XXIV) comprises or consists of the amino acid sequence of any one of SEQ ID NOS: 115, 172, 208, 333, 356, 398, 470, 480, 508, 517, 553, and 557.

In some embodiments, a peptide of Formula (XXIV) comprises or consists of the amino acid sequence of Subformula (XXIVa): Q-X₂-P-X₄-X₅-X₆-P-Y-Y-V-X₁₁-X₁₂-X₁₃-S, wherein:
X₂ is D, I, K, L, or M;
X₄ is F, G, L, S, or T;
X₅ is E, G, H, N, Q, V, or Y;
X₆ is A, D, E, G, or S;
X₁₁ is D, G, L, Q, or T;
X₁₂ is K, L, M, N, Q, R, S, or T; and
X₁₃ is E, K, M, N, P, S, or V.

In some embodiments, a peptide of Formula (XXIV) does not comprise or consist of the amino acid sequence, wherein X₅ is E; X₆ is E; X₇ is P; X₈ is Y; X₉ is Y; X₁₀ is V; X₁₂ is K; and X₁₃ is K.

In some embodiments, a peptide of Subformula (XXIVa) does not comprise or consist of the amino acid sequence, wherein X₅ is E; X₆ is E; X₁₂ is K; and X₁₃ is K.

More generally, in some embodiments, the selection of amino acids at any position of a peptide of Formula (XXIV) and Subformula (XXIVa) can be any subset or combination of choices at that position. Thus, the embodiments not only include those in which X₂ is D, I, K, L, or M; but also, for example, those in which X₂ is D; X₂ is I; X₂ is K; etc.; X₂ is D or I; X₂ is D, I, or K; X₂ is I or K; X₂ is D, I, K, or M, and so on. Moreover, the choice of amino acids, or combination or subset thereof, at one position, e.g., X₂, is independent of the choice of amino acids (or combination or subset thereof) at any other position, e.g., X₁₁ or X₁₂, etc.

### Formula (XXV)

In some embodiments, the peptide comprises or consists of the amino acid sequence of Formula (XXV): Q-X₂-P-X₄-X₅-X₆-X₇-X₈-X₉-X₁₀-X₁₁-X₁₂-X₁₃-X₁₄, wherein:
X₂ is F, I, L, N, S, or V;
X₄ is E, G, L, R, W, or Y;
X₅ is A, D, G, H, K, N, Q, S, T, V, or Y;
X₆ is A, D, E, H, N, Q, S, or T;
X₇ is A, K, L, P, or R;
X₈ is N, Q, S, T, or Y;
X₉ is E, F, H, N, V, or Y;
X₁₀ is S or V;
X₁₁ is F, H, L, M, S, or V;
X₁₂ is E, H, I, K, or P;
X₁₃ is K, N, Q, S, or Y; and
X₁₄ is F, S, V, or Y.

In some embodiments, a peptide of Formula (XXV) comprises or consists of the amino acid sequence of any one of SEQ ID NOS: 11, 29, 77, 82, 263, 280, 369, 402, 419, 449, 466, 478, 507, 534, 542, and 554.

In some embodiments, a peptide of Formula (XXV) comprises or consists of the amino acid sequence of Subformula (XXVa): Q-X₂-P-X₄-X₅-X₆-P-Y-X₉-V-V-X₁₂-K-S, wherein:
X₂ is F, I, L, N, S, or V;
X₄ is E, G, L, R, W, or Y;
X₅ is A, D, G, H, K, N, Q, S, T, V, or Y;
X₆ is A, D, E, H, N, Q, S, or T;
X₉ is E, F, H, N, V, or Y; and
X₁₂ is E, H, I, K, or P.

In some embodiments, a peptide of Formula (XXV) does not comprise or consist of the amino acid sequence, wherein X₂ is V; X₄ is W; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XXVa) does not comprise or consist of the amino acid sequence, wherein X₂ is V; X₄ is W; X₆ is E; X₉ is Y; and X₁₂ is K.

More generally, in some embodiments, the selection of amino acids at any position of a peptide of Formula (XXV) and Subformula (XXVa) can be any subset or combination of choices at that position. Thus, the embodiments not only include those in which X₂ is F, I, L, N, S, or V; but also, for example, those in which X₂ is F; X₂ is I; X₂ is L; etc.; X₂ is F or I; X₂ is F, I, or L; X₂ is I or L; X₂ is I, L, N, or V, and so on. Moreover, the choice of amino acids, or combination or subset thereof, at one position, e.g., X₂, is independent of the choice of amino acids (or combination or subset thereof) at any other position, e.g., X₅ or X₆, etc.

### Formula (XXVI)

In some embodiments, the peptide comprises or consists of the amino acid sequence of Formula (XXVI): X₁-X₂-X₃-X₄-X₅-X₆-X₇-X₈-X₉-X₁₀-X₁₁-X₁₂-X₁₃-X₁₄, wherein:
X₁ is N, Q, R, S, or V;
X₂ is A, D, F, H, I, L, N, or V;
X₃ is L, P, Q, R, or S;
X₄ is A, C, E, G, H, I, L, R, S, V, W, or Y;
X₅ is E, G, K, N, P, Q, or S;
X₆ is A, D, E, G, K, L, or R;
X₇ is H, L, Q, S, or T;
X₈ is F, H, K, or Y;
X₉ is D, F, I, L, N, or Y;
X₁₀ is A, F, G, L, M, or V;
X₁₁ is A, F, G, I, L, M, P, V, or W;
X₁₂ is A, K, N, T, or Y;
X₁₃ is H, K, L, M, N, Q, S, T, or Y; and
X₁₄ is C, E, G, N, P, or S.

In some embodiments, a peptide of Formula (XXVI) comprises or consists of the amino acid sequence of any one of SEQ ID NOS: 34, 38, 64, 127, 155, 156, 164, 213, 304, 329, 366, 371, 444, 445, 447, 493, and 555.

In some embodiments, a peptide of Formula XXVI comprises or consists of the amino acid sequence of Subformula (XXVIa): Q-X₂-X₃-X₄-X₅-X₆-Q-Y-X₉-V-X₁₁-N-X₁₃-S, wherein:
X₂ is A, D, F, H, I, L, N, or V;
X₃ is L, P, Q, R, or S;
X₄ is A, C, E, G, H, I, L, R, S, V, W, or Y;
X₅ is E, G, K, N, P, Q, or S;
X₆ is A, D, E, G, K, L, or R;
X₉ is D, F, I, L, N, or Y;
X₁₁ is A, F, G, I, L, M, P, V, or W; and
X₁₃ is H, K, L, M, N, Q, S, T, or Y.

In some embodiments, a peptide of Formula (XXVI) does not comprise or consist of the amino acid sequence, wherein X₁ is Q; X₂ is V; X₃ is P; X₄ is W; X₅ is E; X₆ is E; X₈ is Y; X₉ is Y; X₁₀ is V; X₁₁ is V; X₁₂ is K; X₁₃ is K; and X₁₄ is S.

In some embodiments, a peptide of Subformula (XXVIa) does not comprise or consist of the amino acid sequence, wherein X₂ is V; X₃ is P; X₄ is W; X₅ is E; X₆ is E; X₉ is Y; X₁₁ is V; and X₁₃ is K.

More generally, in some embodiments, the selection of amino acids at any position of a peptide of Formula (XXVI) and Subformula (XXVIa) can be any subset or combination of choices at that position. Thus, the embodiments not only include those in which X₃ is L, P, Q, R, or S; but also, for example, those in which X₃ is L; X₃ is P; X₃ is Q; etc.; X₃ is L or P; X₃ is L, P, or Q; X₃ is P or Q; X₃ is P, Q, R, or S, and so on. Moreover, the choice of amino acids, or combination or subset thereof, at one position, e.g., X₃, is independent of the choice of amino acids (or combination or subset thereof) at any other position, e.g., X₁₁ or X₁₃, etc.

### Formula (XXVII)

In some embodiments, the peptide comprises or consists of the amino acid sequence of Formula (XXVII): X₁-X₂-X₃-X₄-X₅-X₆-X₇-X₈-X₉-X₁₀-X₁₁-X₁₂-X₁₃-X₁₄, wherein:
X₁ is D, E, Q, or R;
X₂ is F, G, I, L, or V;
X₃ is A, H, P, Q, R, or S;
X₄ is C, L, R, S, T, W, or Y;
X₅ is A, E, G, H, P, Q, or V;
X₆ is A, D, E, G, K, Q, or T;
X₇ is A, H, Q, R, or S;
X₈ is C, E, F, N, Q, S, or Y;
X₉ is C, H, S, V, W, or Y;
X₁₀ is D, I, L, T, or V;
X₁₁ is A, E, G, or V;
X₁₂ is K, P, or T;
X₁₃ is K, M, N, Q, R, S, or T; and
X₁₄ is S or Y.

In some embodiments, a peptide of Formula (XXVII) comprises or consists of the amino acid sequence of any one of SEQ ID NOS: 53, 62, 204, 212, 275, 364, 374, 399, 437, 469, 474, 500, 524, 529, 533, and 548.

In some embodiments, a peptide of Formula (XXVII) comprises or consists of the amino acid sequence of Subformula (XXVIIa): Q-F-P-X₄-E-X₆-S-X₈-Y-X₁₀-V-K-X₁₃-S, wherein:
X₄ is C, L, R, S, T, W, or Y;
X₆ is A, D, E, G, K, Q, or T;
X₈ is C, E, F, N, Q, S, or Y;
X₁₀ is D, I, L, T, or V; and
X₁₃ is K, M, N, Q, R, S, or T.

In some embodiments, a peptide of Formula (XXVII) does not comprise or consist of the amino acid sequence, wherein X₁ is Q; X₂ is V; X₃ is P; X₄ is W; X₅ is E; X₆ is E; X₈ is Y; X₉ is Y; X₁₀ is V; X₁₁ is V; X₁₂ is K; X₁₃ is K; and X₁₄ is S.

In some embodiments, a peptide of Subformula (XXVIIa) does not comprise or consist of the amino acid sequence, wherein X₄ is W; X₆ is E; X₈ is Y; X₁₀ is V; and X₁₃ is K.

More generally, in some embodiments, the selection of amino acids at any position of a peptide of Formula (XXVII) and Subformula (XXVIIa) can be any subset or combination of choices at that position. Thus, the embodiments not only include those in which X₄ is C, L, R, S, T, W, or Y; but also, for example, those in which X₄ is C; X₄ is L; X₄ is R; etc.; X₄ is C or L; X₄ is C, L, or R; X₄ is L or R; X₄ is C, L, R, or W, and so on. Moreover, the choice of amino acids, or combination or subset thereof, at one position, e.g., X₄, is independent of the choice of amino acids (or combination or subset thereof) at any other position, e.g., X₈ or X₁₀, etc.

### Formula (XXVIII)

In some embodiments, the peptide comprises or consists of the amino acid sequence of Formula (XXVIII): X₁-X₂-X₃-X₄-X₅-X₆-X₇-X₈-X₉-X₁₀-X₁₁-X₁₂-X₁₃-X₁₄, wherein:
X₁ is H, Q, or S;
X₂ is C, F, G, I, V, or Y;
X₃ is A, P, Q, R, S, T, or Y;
X₄ is C, L, N, S, W, or Y;
X₅ is D, E, H, K, Q, S, or V;
X₆ is A, E, G, K, or Q;
X₇ is L or S;
X₈ is C, N, P, S, V, or Y;
X₉ is A, D, F, G, N, S, or Y;
X₁₀ is D, G, L, S, V, or Y;
X₁₁ is A, E, F, G, L, Q, S, or V;
X₁₂ is A, K, N, Q, R, S, or T;
X₁₃ is I, K, or T; and
X₁₄ is I, L, P, R, S, T, or W.

In some embodiments, a peptide of Formula (XXVIII) comprises or consists of the amino acid sequence of any one of SEQ ID NOS: 121, 128, 136, 186, 200, 256, 261, 331, 345, 386, 389, 404, 441, and 450.

In some embodiments, a peptide of Formula (XXVIII) comprises or consists of the amino acid sequence of Subformula (XXVIIIa): Q-X₂-X₃-X₄-X₅-X₆-S-X₈-X₉-X₁₀-X₁₁-X₁₂-K-S, wherein:
X₂ is C, F, G, I, V, or Y;
X₃ is A, P, Q, R, S, T, or Y;
X₄ is C, L, N, S, W, or Y;
X₅ is D, E, H, K, Q, S, or V;
X₆ is A, E, G, K, or Q;
X₈ is C, N, P, S, V, or Y;
X₉ is A, D, F, G, N, S, or Y;
X₁₀ is D, G, L, S, V, or Y;
X₁₁ is A, E, F, G, L, Q, S, or V; and
X₁₂ is A, K, N, Q, R, S, or T.

In some embodiments, a peptide of Formula (XXVIII) does not comprise or consist of the amino acid sequence, wherein X₁ is Q; X₂ is V; X₃ is P; X₄ is W; X₅ is E; X₆ is E; X₈ is Y; X₉ is Y; X₁₀ is V; X₁₁ is V; X₁₂ is K; X₁₃ is K; and X₁₄ is S.

In some embodiments, a peptide of Subformula (XXVIIIa) does not comprise or consist of the amino acid sequence, wherein X₂ is V, X₃ is P, X₄ is W; X₅ is E; X₆ is E; X₈ is Y; X₉ is Y; X₁₀ is V; X₁₁ is V; and X₁₂ is K.

More generally, in some embodiments, the selection of amino acids at any position of a peptide of Formula (XXVIII) and Subformula (XXVIIIa) can be any subset or combination of choices at that position. Thus, the embodiments not only include those in which X₄ is C, L, N, S, W, or Y; but also, for example, those in which X₄ is C; X₄ is L; X₄ is N; etc.; X₄ is C or L; X₄ is C, L, or W; X₄ is L or W; X₄ is L, N, S, or Y, and so on. Moreover, the choice of amino acids, or combination or subset thereof, at one position, e.g., X₄, is independent of the choice of amino acids (or combination or subset thereof) at any other position, e.g., X₈ or X₁₀, etc.

### Formula (XXIX)

In some embodiments, the peptide comprises or consists of the amino acid sequence of Formula (XXIX): X₁-X₂-X₃-X₄-X₅-X₆-X₇-X₈-X₉-X₁₀-X₁₁-X₁₂-X₁₃-X₁₄, wherein:
X₁ is D or E;
X₂ is F, G, H, I, R, or V;
X₃ is A, L, P, or T;
X₄ is F, G, L, R, S, T, W, or Y;
X₅ is A, D, E, G, I, P, R, or V;
X₆ is A, D, E, H, K, L, or R;
X₇ is L, P, Q, R, S, T, or W;
X₈ is F, H, N, R, S, V, or Y;
X₉ is F, K, or Y;
X₁₀ is A, D, F, G, I, L, N, R, or V;
X₁₁ is A, E, F, G, L, M, or V;
X₁₂ is E, K, P, Q, S, T, or V;
X₁₃ is D, F, H, I, K, or Q; and
X₁₄ is G, P, S, T, or Y.

In some embodiments, a peptide of Formula (XXIX) comprises or consists of the amino acid sequence of any one of SEQ ID NOS: 13, 108, 109, 139, 167, 183, 198, 205, 247, 285, 383, 440, 488, 502, and 546.

In some embodiments, a peptide of Formula (XXIX) comprises or consists of the amino acid sequence of Subformula (XXIXa): D-V-X₃-X₄-X₅-X₆-X₇-X₈-Y-X₁₀-X₁₁-X₁₂-X₁₃-X₁₄, wherein:
X₃ is A, L, P, or T;
X₄ is F, G, L, R, S, T, W, or Y;
X₅ is A, D, E, G, I, P, R, or V;
X₆ is A, D, E, H, K, L, or R;
X₇ is L, P, Q, R, S, T, or W;
X₈ is F, H, N, R, S, V, or Y;
X₁₀ is A, D, F, G, I, L, N, R, or V;
X₁₁ is A, E, F, G, L, M, or V;
X₁₂ is E, K, P, Q, S, T, or V;
X₁₃ is D, F, H, I, K, or Q; and
X₁₄ is G, P, S, T, or Y.

In some embodiments, a peptide of Formula (XXIX) does not comprise or consist of the amino acid sequence, wherein X₂ is V; X₃ is P; X₄ is W; X₅ is E; X₆ is E; X₇ is P; X₈ is Y; X₉ is Y; X₁₀ is V; X is V; X₁₂ is K; X₁₃ is K; and X₁₄ is S.

In some embodiments, a peptide of Subformula (XXIXa) does not comprise or consist of the amino acid sequence, wherein X₃ is P; X₄ is W; X₅ is E; X₆ is E; X₇ is P; X₈ is Y; X₁₀ is V; X₁₁ is V; X₁₂ is K; X₁₃ is K; and X₁₄ is S.

More generally, in some embodiments, the selection of amino acids at any position of a peptide of Formula (XXIX) and Subformula (XXIXa) can be any subset or combination of choices at that position. Thus, the embodiments not only include those in which X₄ is F, G, L, R, S, T, W, or Y; but also, for example, those in which X₄ is F; X₄ is G; X₄ is L; etc.; X₄ is F or G; X₄ is F, G, or L; X₄ is G or L; X₄ is G, L, R, or W, and so on. Moreover, the choice of amino acids, or combination or subset thereof, at one position, e.g., X₄, is independent of the choice of amino acids (or combination or subset thereof) at any other position, e.g., X₇ or X₈, etc.

### Formula (XXX)

In some embodiments, the peptide comprises or consists of the amino acid sequence of Formula (XXX): X₁-X₂-X₃-X₄-X₅-X₆-X₇-X₈-X₉-X₁₀-X₁₁-X₁₂-X₁₃-X₁₄, wherein:
X₁ is D, E, H, L, or R;
X₂ is F, G, H, I, L, S, or V;
X₃ is L, P, or Q;
X₄ is A, C, G, L, P, Q, R, S, T, V, or W;
X₅ is D, E, G, H, K, or Q;
X₆ is D, E, K, T, V, or Y;
X₇ is A, E, L, P, R, S, T, or W;
X₈ is D, E, F, H, S, W, or Y;
X₉ is I, W, or Y;
X₁₀ is A, D, I, P, or V;
X₁₁ is A, C, F, G, H, I, L, M, T, or V;
X₁₂ is D, E, H, K, T, or V;
X₁₃ is G, H, I, K, L, N, Q, R, S, V, or Y; and
X₁₄ is A, F, H, I, M, S, or Y.

In some embodiments, a peptide of Formula (XXX) comprises or consists of the amino acid sequence of any one of SEQ ID NOS: 21, 85, 95, 102, 112, 116, 129, 253, 259, 273, 327, 400, 423, 484, 486, 523, 526, and 539.

In some embodiments, a peptide of Formula (XXX) comprises or consists of the amino acid sequence of Subformula (XXXa): H-X₂-P-X₄-X₅-E-X₇-X₈-Y-V-X₁₁-K-X₁₃-S, wherein:
X₂ is F, G, H, I, L, S, or V;
X₄ is A, C, G, L, P, Q, R, S, T, V, or W;
X₅ is D, E, G, H, K, or Q;
X₇ is A, E, L, P, R, S, T, or W;
X₈ is D, E, F, H, S, W, or Y;
X₁₁ is A, C, F, G, H, I, L, M, T, or V; and
X₁₃ is G, H, I, K, L, N, Q, R, S, V, or Y.

In some embodiments, a peptide of Formula (XXX) does not comprise or consist of the amino acid sequence, wherein X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XXXa) does not comprise or consist of the amino acid sequence, wherein X₂ is V; X₄ is W; X₅ is E; X₇ is P; X₈ is Y; X₁₁ is V; and X₁₃ is K.

More generally, in some embodiments, the selection of amino acids at any position of a peptide of Formula (XXX) and Subformula (XXXa) can be any subset or combination of choices at that position. Thus, the embodiments not only include those in which X₂ is F, G, H, I, L, S, or V; but also, for example, those in which X₂ is F; X₂ is G; X₂ is H; etc.; X₂ is F or G; X₂ is F, G, or H; X₂ is G or H; X₂ is G, H, L, or V, and so on. Moreover, the choice of amino acids, or combination or subset thereof, at one position, e.g., X₂, is independent of the choice of amino acids (or combination or subset thereof) at any other position, e.g., X₇ or X₈, etc.

### Formula (XXXI)

In some embodiments, the peptide comprises or consists of the amino acid sequence of Formula (XXXI): X₁-X₂-X₃-X₄-X₅-X₆-X₇-X₈-X₉-X₁₀-X₁₁-X₁₂-X₁₃-X₁₄, wherein:
X₁ is D, E, H, L, Q, R, T, or Y;
X₂ is F, G, I, R, S, or V;
X₃ is A, G, K, L, P, R, S, or T;
X₄ is A, G, L, R, S, T, W, or Y;
X₅ is A, E, G, H, K, N, P, Q, T, V, or Y;
X₆ is A, D, E, G, or N;
X₇ is A, P, Q, S, or T;
X₈ is D, F, G, H, N, R, S, or Y;
X₉ is D, F, G, H, K, N, Q, R, S, T, V, or Y;
X₁₀ is E, I, L, N, T, V, or Y;
X₁₁ is A, F, G, K, L, M, Q, or V;
X₁₂ is I, K, L, N, Q, R, or T;
X₁₃ is E, H, I, K, M, N, Q, T, or W; and
X₁₄ is F, H, I, L, N, P, R, or S.

In some embodiments, a peptide of Formula (XXXI) comprises or consists of the amino acid sequence of any one of SEQ ID NOS: 18, 22, 23, 24, 49, 90, 111, 119, 131, 157, 222, 242, 264, 269, 301, 306, 344, 346, 347, 368, 373, 385, 414, 421, 429, and 492.

In some embodiments, a peptide of Formula (XXXI) peptide comprises or consists of the amino acid sequence of Subformula (XXXIa): X₁-V-S-X₄-X₅-E-P-X₈-X₉-V-L-X₁₂-X₁₃-X₁₄, wherein:
X₁ is D, E, H, L, Q, R, T, or Y;
X₄ is A, G, L, R, S, T, W, or Y;
X₅ is A, E, G, H, K, N, P, Q, T, V, or Y;
X₈ is D, F, G, H, N, R, S, or Y;
X₉ is D, F, G, H, K, N, Q, R, S, T, V, or Y;
X₁₂ is I, K, L, N, Q, R, or T;
X₁₃ is E, H, I, K, M, N, Q, T, or W; and
X₁₄ is F, H, I, L, N, P, R, or S.

In some embodiments, a peptide of Formula (XXXI) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XXXIa) does not comprise or consist of the amino acid sequence, wherein X₁ is Q; X₄ is W; X₅ is E; X₈ is Y; X₉ is Y; X₁₂ is K; X₁₃ is K; and X₁₄ is S.

More generally, in some embodiments, the selection of amino acids at any position of a peptide of Formula (XXXI) and Subformula (XXXIa) can be any subset or combination of choices at that position. Thus, the embodiments not only include those in which X₁₂ is I, K, L, N, Q, R, or T; but also, for example, those in which X₁₂ is I; X₁₂ is K; X₁₂ is L; etc.; X₁₂ is I or K; X₁₂ is I, K, or L; X₁₂ is K or L; X₁₂ is K, L, N, or Q, and so on. Moreover, the choice of amino acids, or combination or subset thereof, at one position, e.g., X₁₂, is independent of the choice of amino acids (or combination or subset thereof) at any other position, e.g., X₁, X₁₃, or X₁₄, etc.

### Formula (XXXII)

In some embodiments, the peptide comprises or consists of the amino acid sequence of Formula (XXXII): X₁-X₂-X₃-X₄-X₅-X₆-X₇-X₈-X₉-X₁₀-X₁₁-X₁₂-X₁₃-X₁₄, wherein:
X₁ is A, H, L, N, Q, or Y;
X₂ is C, D, F, H, I, L, R, S, T, V, or Y;
X₃ is A, G, L, N, P, Q, R, T, V, or Y;
X₄ is C, F, G, R, S, or W;
X₅ is A, E, or T;
X₆ is A, D, E, Q, S, or Y;
X₇ is A, L, P, T, or Y;
X₈ is C, E, F, G, N, Q, S, or Y;
X₉ is H, S, or Y;
X₁₀ is A, F, H, L, R, S, T, or V;
X₁₁ is A, C, D, E, F, G, H, L, M, N, Q, or V;
X₁₂ is A, D, E, G, H, K, N, Q, S, or T;
X₁₃ is D, E, F, I, K, L, M, N, P, Q, R, S, T, or Y; and
X₁₄ is A, F, K, L, P, S, V, or Y.

In some embodiments, a peptide of Formula (XXXII) comprises or consists of the amino acid sequence of any one of SEQ ID NOS: 10, 20, 41, 84, 97, 113, 122, 123, 130, 148, 165, 254, 277, 295, 297, 308, 342, 358, 432, 458, 461, 465, 514, 537, 540, 541, and 556.

In some embodiments, peptide of Formula (XXXII) comprises or consists of the amino acid sequence of Subformula (XXXIIa): Q-X₂-X₃-X₄-E-E-P-Y-Y-V-X₁₁-K-X₁₃-X₁₄, wherein:
X₂ is C, D, F, H, I, L, R, S, T, V, or Y;
X₃ is A, G, L, N, P, Q, R, T, V, or Y;
X₄ is C, F, G, R, S, or W;
X₁₁ is A, C, D, E, F, G, H, L, M, N, Q, or V;
X₁₃ is D, E, F, I, K, L, M, N, P, Q, R, S, T, or Y; and
X₁₄ is A, F, K, L, P, S, V, or Y.

In some embodiments, a peptide of Subformula (XXXII) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Formula (XXXIIa) does not comprise or consist of the amino acid sequence, wherein X₂ is V; X₃ is P; X₄ is W; X₁₃ is K; and X₁₄ is S.

More generally, in some embodiments, the selection of amino acids at any position of a peptide of Formula (XXXII) and Subformula (XXXIIa) can be any subset or combination of choices at that position. Thus, the embodiments not only include those in which X₄ is X₄ is C, F, G, R, S, or W; but also, for example, those in which X₄ is C; X₁ is F; X₄ is G; etc.; X₄ is C or F; X₄ is C, F, or G; X₄ is F or G; X₄ is F, R, S, or W, and so on. Moreover, the choice of amino acids, or combination or subset thereof, at one position, e.g., X₄, is independent of the choice of amino acids (or combination or subset thereof) at any other position, e.g., X₁ or X₃, etc.

### Formula (XXXIII)

In some embodiments, the peptide comprises or consists of the amino acid sequence of Formula (XXXIII): X₁-X₂-X₃-X₄-X₅-X₆-X₇-X₈-X₉-X₁₀-X₁₁-X₁₂-X₁₃-X₁₄, wherein:
X₁ is D, E, H, L, N, Q, R, or Y;
X₂ is A, D, E, F, I, L, T, V, or Y;
X₃ is A, E, L, P, Q, R, or S;
X₄ is F, G, P, R, S, or W;
X₅ is A, D, E, G, H, Q, S, or V;
X₆ is A, D, E, G, H, Q, or T;
X₇ is P, Q, R, S, or T;
X₈ is D, F, H, K, N, S, or Y;
X₉ is D, F, G, H, N, P, Q, S, or Y;
X₁₀ is A, D, F, G, I, L, P, R, or V;
X₁₁ is A, E, G, I, L, R, or V;
X₁₂ is D, E, K, N, Q, R, T, or V;
X₁₃ is D, E, I, K, L, Q, R, or T; and
X₁₄ is A, I, S, T, or Y.

In some embodiments, a peptide of Formula (XXXIII) comprises or consists of the amino acid sequence of any one of SEQ ID NOS: 5, 33, 71, 79, 106, 120, 151, 168, 219, 226, 229, 237, 268, 326, 330, 334, 375, 392, 395, 426, 433, 434, 472, 490, 528, and 544.

In some embodiments, the peptide comprises or consists of the amino acid sequence of Subformula (XXXIIIa): Q-X₂-X₃-W-E-X₆-P-X₈-Y-X₁₀-X₁₁-X₁₂-K-S, wherein:
X₂ is A, D, E, F, I, L, T, V, or Y;
X₃ is A, E, L, P, Q, R, or S;
X₆ is A, D, E, G, H, Q, or T;
X₈ is D, F, H, K, N, S, or Y;
X₁₀ is A, D, F, G, I, L, P, R, or V;
X₁₁ is A, E, G, I, L, R, or V; and
X₁₂ is D, E, K, N, Q, R, T, or V.

In some embodiments, a peptide of Formula (XXXIII) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XXXIIIa) does not comprise or consist of the amino acid sequence, wherein X₂ is V; X₃ is P; X₆ is E; X₈ is Y; X₁₀ is V; X₁₁ is V; and X₁₂ is K.

More generally, in some embodiments, the selection of amino acids at any position of a peptide of Formula (XXXIII) and Subformula (XXXIIIa) can be any subset or combination of choices at that position. Thus, the embodiments not only include those in which X₂ is A, D, E, F, I, L, T, V, or Y; but also, for example, those in which X₂ is A; X₂ is D; X₂ is E; etc.; X₂ is A or D; X₂ is D, E, or F; X₂ is D or E; X₂ is D, E, F, or T, and so on. Moreover, the choice of amino acids, or combination or subset thereof, at one position, e.g., X₂, is independent of the choice of amino acids (or combination or subset thereof) at any other position, e.g., X₁₀ or X₁₁, etc.

### Formula (XXXIV)

In some embodiments, the peptide comprises or consists of the amino acid sequence of Formula (XXXIV): X₁-X₂-X₃-X₄-X₅-X₆-X₇-X₈-X₉-X₁₀-X₁₁-X₁₂-X₁₃-X₁₄, wherein:
X₁ is A, D, E, K, L, N, Q, R, S, T, or Y;
X₂ is D, E, F, I, L, N, Q, R, S, T, or V;
X₃ is I, L, P, Q, or T;
X₄ is F, G, I, K, M, N, Q, R, S, V, W, or Y;
X₅ is A, D, E, G, P, Q, V, or Y;
X₆ is A, D, E, G, H, L, P, Q, S, or V;
X₇ is F or P;
X₈ is D, G, K, N, R, S, V, or Y;
X₉ is D, E, L, or Y;
X₁₀ is D, F, G, K, L, M, N, R, or V;
X₁₁ is A, E, F, G, M, P, Q, R, or V;
X₁₂ is K, L, N, or V;
X₁₃ is A, D, K, M, N, T, or V; and
X₁₄ is A, F, H, L, P, S, T, V, or Y.

In some embodiments, a peptide of Formula (XXXIV) comprises or consists of the amino acid sequence of any one of SEQ ID NOS: 14, 43, 44, 104, 132, 145, 160, 191, 210, 221, 284, 323, 324, 328, 353, 372, 387, 391, 411, 431, 435, 468, 471, 481, 509, 510, 516, 525, and 547.

In some embodiments, a peptide of Formula (XXXIV) or consists of the amino acid sequence of Subformula (XXXIVa): X₁-X₂-P-X₄-X₅-X₆-P-X₈-Y-X₁₀-V-X₁₂-N-S, wherein:
X₁ is A, D, E, K, L, N, Q, R, S, T, or Y;
X₂ is D, E, F, I, L, N, Q, R, S, T, or V;
X₄ is F, G, I, K, M, N, Q, R, S, V, W, or Y;
X₅ is A, D, E, G, P, Q, V, or Y;
X₆ is A, D, E, G, H, L, P, Q, S, or V;
X₈ is D, G, K, N, R, S, V, or Y;
X₁₀ is D, F, G, K, L, M, N, R, or V; and
X₁₂ is K, L, N, or V.

In some embodiments, a peptide of Formula (XXXIV) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XXXIVa) does not comprise or consist of the amino acid sequence, wherein X₁ is Q; X₂ is V; X₄ is W; X₅ is E; X₆ is E; X₈ is Y; X₁₀ is V; and X₁₂ is K.

More generally, in some embodiments, the selection of amino acids at any position of a peptide of Formula (XXXIV) and Subformula (XXXIVa) can be any subset or combination of choices at that position. Thus, the embodiments not only include those in which X₁ is A, D, E, K, L, N, Q, R, S, T, or Y; but also, for example, those in which X₁ is A; X₁ is D; X₁ is E; etc.; X₁ is A or D; X₁ is A, D, or E; X₁ is D or E; X₁ is D, E, Q, or R, and so on. Moreover, the choice of amino acids, or combination or subset thereof, at one position, e.g., X₁, is independent of the choice of amino acids (or combination or subset thereof) at any other position, e.g., X₅ or X₆, etc.

### Formula (XXXV)

In some embodiments, the peptide comprises or consists of the amino acid sequence of Formula (XXXV): X₁-X₂-X₃-X₄-X₅-X₆-X₇-X₈-X₉-X₁₀-X₁₁-X₁₂-X₁₃-X₁₄, wherein:
X₁ is A, E, H, L, N, Q, R, S, V, or Y;
X₂ is F, I, N, S, V, or Y;
X₃ is A, E, I, L, P, R, S, T, V, W, or Y;
X₄ is C, F, G, K, L, R, S, W, or Y;
X₅ is D, E, I, K, Q, V, or Y;
X₆ is A, D, E, G, N, P, Q, R, S, V, or Y;
X₇ is G, L, P, Q, or S;
X₈ is H, I, L, N, S, or Y;
X₉ is A, D, E, F, L, N, S, T, V, or Y;
X₁₀ is A, D, E, F, G, H, I, L, N, S, T, or V;
X₁₁ is K, L, M, or V;
X₁₂ is I, K, N, P, Q, R, S, or T;
X₁₃ is A, C, E, H, K, M, N, Q, R, S, T, or Y; and
X₁₄ is F, H, P, S, T, or V.

In some embodiments, a peptide of Formula (XXXV) comprises or consists of the amino acid sequence of any one of SEQ ID NOS: 25, 37, 68, 83, 101, 124, 147, 153, 158, 159, 182, 209, 232, 233, 235, 248, 262, 270, 283, 302, 384, 390, 401, 407, 409, 418, 448, 457, 515, 519, and 530.

In some embodiments, a peptide of Formula (XXXV) comprises or consists of the amino acid sequence of Subformula (XXXVa): X₁-X₂-X₃-X₄-E-X₆-P-Y-Y-X₁₀-V-X₁₂-X₁₃-S, wherein:
X₁ is A, E, H, L, N, Q, R, S, V, or Y;
X₂ is F, I, N, S, V, or Y;
X₃ is A, E, I, L, P, R, S, T, V, W, or Y;
X₄ is C, F, G, K, L, R, S, W, or Y;
X₆ is A, D, E, G, N, P, Q, R, S, V, or Y;
X₁₀ is A, D, E, F, G, H, I, L, N, S, T, or V;
X₁₂ is I, K, N, P, Q, R, S, or T; and
X₁₃ is A, C, E, H, K, M, N, Q, R, S, T, or Y.

In some embodiments, a peptide of Formula (XXXV) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XXXVa) does not comprise or consist of the amino acid sequence, wherein X₁ is Q; X₂ is V; X₃ is P; X₄ is W; X₆ is E; X₁₀ is V; X₁₂ is K; and X₁₃ is K.

More generally, in some embodiments, the selection of amino acids at any position of a peptide of Formula (XXXV) and Subformula (XXXVa) can be any subset or combination of choices at that position. Thus, the embodiments not only include those in which X₂ is F, I, N, S, V, or Y; but also, for example, those in which X₂ is F; X₂ is I; X₂ is N; etc.; X₂ is F or I; X₂ is F, I, or N; X₂ is I or N; X₂ is I, N, V, or Y, and so on. Moreover, the choice of amino acids, or combination or subset thereof, at one position, e.g., X₂, is independent of the choice of amino acids (or combination or subset thereof) at any other position, e.g., X₄ or X₆, etc.

### Formula (XXXVI)

In some embodiments, the peptide comprises or consists of the amino acid sequence of Formula (XXXVI): X₁-X₂-X₃-X₄-X₅-X₆-X₇-X₈-X₉-X₁₀-X₁₁-X₁₂-X₁₃-X₁₄, wherein:
X₁ is D, F, H, L, N, Q, R, S, T, or Y;
X₂ is A, D, F, G, I, L, Q, or V;
X₃ is A, D, E, F, G, H, K, L, P, Q, R, S, T, or V;
X₄ is A, C, E, F, G, I, L, P, Q, R, S, V, W, or Y;
X₅ is A, D, E, G, H, I, K, L, N, P, Q, R, S, T, V, or Y;
X₆ is A, D, E, G, K, N, P, Q, S, T, V, or Y;
X₇ is A, H, I, L, P, Q, R, S, or T;
X₈ is A, E, F, H, I, K, L, P, Q, R, S, V, or Y;
X₉ is A, C, D, F, G, H, K, L, N, P, Q, R, S, or Y;
X₁₀ is A, D, E, F, G, H, I, L, N, P, S, V, or Y;
X₁₁ is A, D, E, F, G, I, L, M, N, S, T, or V;
X₁₂ is D, E, G, H, I, K, N, Q, R, S, T, W, or Y;
X₁₃ is D, E, H, K, N, Q, R, S, T, or V; and
X₁₄ is A, I, P, S, or Y.

In some embodiments, a peptide of Formula (XXXVI) comprises or consists of the amino acid sequence of any one of SEQ ID NOS: 15, 67, 69, 75, 81, 86, 96, 99, 107, 126, 135, 140, 142, 144, 149, 170, 173, 175, 178, 188, 192, 196, 197, 206, 207, 217, 223, 231, 234, 239, 249, 250, 251, 257, 271, 272, 287, 291, 307, 314, 315, 317, 318, 319, 320, 349, 350, 359, 360, 362, 363, 365, 370, 378, 382, 406, 410, 427, 438, 456, 476, 485, 506, 512, 520, 527, 531, 538, and 545.

In some embodiments, a peptide of Formula (XXXVI) comprises or consists of the amino acid sequence of Subformula (XXXVIa): H-V-X₃-X₄-X₅-E-P-Y-X₉-X₁₀-V-X₁₂-K-S, wherein:
X₃ is A, D, E, F, G, H, K, L, P, Q, R, S, T, or V;
X₄ is A, C, E, F, G, I, L, P, Q, R, S, V, W, or Y;
X₅ is A, D, E, G, H, I, K, L, N, P, Q, R, S, T, V, or Y;
X₉ is A, C, D, F, G, H, K, L, N, P, Q, R, S, or Y;
X₁₀ is A, D, E, F, G, H, I, L, N, P, S, V, or Y; and
X₁₂ is D, E, G, H, I, K, N, Q, R, S, T, W, or Y.

In some embodiments, a peptide of Formula (XXXVI) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XXXVIa) does not comprise or consist of the amino acid sequence, wherein X₃ is P; X₄ is W; X₅ is E; X₉ is Y; X₁₀ is V; and X₁₂ is K.

More generally, in some embodiments, the selection of amino acids at any position of a peptide of Formula (XXXVI) and Subformula (XXXVIa) can be any subset or combination of choices at that position. Thus, the embodiments not only include those in which X₃ is A, D, E, F, G, H, K, L, P, Q, R, S, T, or V; but also, for example, those in which X₃ is A; X₃ is D; X₃ is F; etc.; X₃ is A or D; X₃ is A, D, or E; X₃ is D or E; X₃ is D, E, F, or P, and so on. Moreover, the choice of amino acids, or combination or subset thereof, at one position, e.g., X₃, is independent of the choice of amino acids (or combination or subset thereof) at any other position, e.g., X₉ or X₁₀, etc.

### Formula (XXXVII)

In some embodiments, the peptide comprises or consists of the amino acid sequence of Formula (XXXVII): X₁-X₂-X₃-X₄-X₅-X₆-X₇-X₈-X₉-X₁₀-X₁₁-X₁₂-X₁₃-X₁₄, wherein:
X₁ is A, E, H, Q, or R;
X₂ is D, F, G, L, M, or V;
X₃ is A, E, H, P, Q, R, S, T, or W;
X₄ is C, G, L, R, S, V, or W;
X₅ is A, D, E, G, I, K, L, or Y;
X₆ is A, D, E, G, K, L, N, Q, S, or V;
X₇ is A, H, L, M, P, S, T, or W;
X₈ is C, H, I, L, N, S, V, or Y;
X₉ is D, H, L, N, S, or Y;
X₁₀ is A, D, E, F, G, L, P, S, T, or V;
X₁₁ is A, D, E, G, I, M, Q, R, T, V, or W;
X₁₂ is H, I, K, L, N, Q, R, or T;
X₁₃ is A, E, H, I, K, M, N, R, or V; and
X₁₄ is F, P, S, or T.

In some embodiments, a peptide of Formula (XXXVII) comprises or consists of the amino acid sequence of any one of SEQ ID NOS: 28, 59, 60, 78, 91, 133, 187, 199, 241, 243, 281, 282, 290, 416, 422, 439, 451, 454, 459, 460, 495, 503, 513, 551, and 552.

In some embodiments, a peptide of Formula (XXXVII) comprises or consists of the amino acid sequence of Subformula (XXXVIIa): H-V-X₃-X₄-X₅-X₆-X₇-Y-Y-X₁₀-X₁₁-K-N-S, wherein:
X₃ is A, E, H, P, Q, R, S, T, or W;
X₄ is C, G, L, R, S, V, or W;
X₅ is A, D, E, G, I, K, L, or Y;
X₆ is A, D, E, G, K, L, N, Q, S, or V;
X₇ is A, H, L, M, P, S, T, or W;
X₁₀ is A, D, E, F, G, L, P, S, T, or V; and
X₁₁ is A, D, E, G, I, M, Q, R, T, V, or W.

In some embodiments, a peptide of Formula (XXXVII) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XXXVIIa) does not comprise or consist of the amino acid sequence, wherein X₃ is P; X₄ is W; X₅ is E; X₆ is E; X₇ is P; X₁₀ is V; and X₁₁ is V.

More generally, in some embodiments, the selection of amino acids at any position of a peptide of Formula (XXXVII) and Subformula (XXXVIIa) can be any subset or combination of choices at that position. Thus, the embodiments not only include those in which X₃ is A, E, H, P, Q, R, S, T, or W; but also, for example, those in which X₃ is D; X₁ is H; X₃ is L; etc.; X₃ is D or H; X₃ is D, H, or L; X₃ is H or L; X₃ is H, L, N, or Q, and so on. Moreover, the choice of amino acids, or combination or subset thereof, at one position, e.g., X₃, is independent of the choice of amino acids (or combination or subset thereof) at any other position, e.g., X₁₀ or X₁₁, etc.

### Formula (XXXVIII)

In some embodiments, a peptide comprises or consists of the amino acid sequence of Formula (XXXVIII): X₁-X₂-X₃-X₄-X₅-X₆-X₇-X₈-X₉-X₁₀-X₁₁-X₁₂-X₁₃-X₁₄, wherein:
X₁ is D, E, G, H, K, L, R, or S;
X₂ is A, F, I, or L;
X₃ is A, E, F, L, P, Q, R, or S;
X₄ is E, F, G, M, R, S, V, W, or Y;
X₅ is E, P, or Q;
X₆ is A, D, E, or G;
X₇ is H, L, P, R, S, or T;
X₈ G, L, S, or Y;
X₉ is D, H, K, L, T, or Y;
X₁₀ is A, F, G, I, L, or R;
X₁₁ L, P, or V;
X₁₂ is A, E, H, I, K, or R;
X₁₃ is E, I, K, N, Q, R, S, or T; and
X₁₄ is A, F, H, P, R, S, T, or Y.

In some embodiments, a peptide of Formula (XXXVIII) comprises or consists of the amino acid sequence of any one of SEQ ID NOS: 17, 26, 114, 174, 179, 184, 195, 309, 322, 325, 348, 417, 462, 464, 494, 505, and 518.

In some embodiments, a peptide of Formula (XXXVIII) comprises or consists of the amino acid sequence of Subformula (XXXVIIIa): H-L-P-X₄-E-E-X₇-Y-X₉-X₁₀-V-X₁₂-X₁₃-X₁₄, wherein:
X₄ is E, F, G, M, R, S, V, W, or Y;
X₇ is H, L, P, R, S, or T;
X₉ is D, H, K, L, T, or Y;
X₁₂ is A, E, H, I, K, or R;
X₁₃ is E, I, K, N, Q, R, S, or T; and
X₁₄ is A, F, H, P, R, S, T, or Y.

In some embodiments, a peptide of Formula (XXXVIII) does not comprise or consist of the amino acid sequence, wherein X₃ is P; X₄ is W; X₅ is E; X₆ is E; X₇ is P; X₈ is Y; X₉ is Y; X₁₁ is V; X₁₂ is K; X₁₃ is K; and X₁₄ is S.

In some embodiments, a peptide of Subformula (XXXVIIIa) does not comprise or consist of the amino acid sequence, wherein X₄ is W; X₇ is P; X₉ is Y; X₁₂ is K; X₁₃ is K; and X₁₄ is S.

More generally, in some embodiments, the selection of amino acids at any position of a peptide of Formula (XXXVIII) and Subformula (XXXVIIIa) can be any subset or combination of choices at that position. Thus, the embodiments not only include those in which X₄ is E, F, G, M, R, S, V, W, or Y; but also, for example, those in which X₄ is E; X₄ is F; X₄ is M; etc.; X₄ is E or F; X₄ is E, F, or G; X₄ is F or G; X₄ is F, G, M, or R, and so on. Moreover, the choice of amino acids, or combination or subset thereof, at one position, e.g., X₄, is independent of the choice of amino acids (or combination or subset thereof) at any other position, e.g., X₁₃ or X₁₄, etc.

### Formula (XXXIX)

In some embodiments, a peptide comprises or consists of the amino acid sequence of Formula (XXXIX): X₁-X₂-X₃-X₄-X₅-X₆-X₇-X₈-X₉-X₁₀-X₁₁-X₁₂-X₁₃-X₁₄, wherein:
X₁ is A, E, N, Q, R, or W;
X₂ is A, D, I, L, S, T, or V;
X₃ is E, H, L, P, T, or Y;
X₄ is L, S, or W;
X₅ is A, E, G, V, or Y;
X₆ is E, G, I, K, P, Q, or T;
X₇ is H, P, R, or S;
X₈ is S or Y;
X₉ is F, S, W, or Y;
X₁₀ is A, D, F, G, H, I, T, or V;
X₁₁ is L or V;
X₁₂ is E, H, K, or N;
X₁₃ is H, K, N, or V; and
X₁₄ is F, S, T, or Y.

In some embodiments, a peptide of Formula (XXXIX) comprises or consists of the amino acid sequence of any one of SEQ ID NOS: 8, 52, 89, 177, 189, 225, 279, 335, 379, 408, 424, 521, and 535.

In some embodiments, peptide of Formula (XXXIX) comprises or consists of the amino acid sequence of Subformula (XXXIXa): Q-X₂-X₃-W-X₅-X₆-P-Y-Y-X₁₀-V-K-K-X₁₄, wherein:
X₂ is A, D, I, L, S, T, or V;
X₃ is E, H, L, P, T, or Y;
X₅ is A, E, G, V, or Y;
X₆ is E, G, I, K, P, Q, or T;
X₁₀ is A, D, F, G, H, I, T, or V; and
X₁₄ is F, S, T, or Y.

In some embodiments, a peptide of Formula (XXXIX) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XXXIXa) does not comprise or consist of the amino acid sequence, wherein X₂ is V; X₃ is P; X₅ is E; X₆ is E; X₁₀ is V; and X₁₄ is S. More generally, in some embodiments, the selection of amino acids at any position of a peptide of Formula (XXXIX) and Subformula (XXXIXa) can be any subset or combination of choices at that position. Thus, the embodiments not only include those in which X₂ is A, D, I, L, S, T, or V; but also, for example, those in which X₂ is A; X₂ is D; X₂ is I; etc.; X₂ is A or D; X₂ is A, D, or I; X₂ is D or I; X₂ is D, I, L, or T, and so on. Moreover, the choice of amino acids, or combination or subset thereof, at one position, e.g., X₂, is independent of the choice of amino acids (or combination or subset thereof) at any other position, e.g., X₅ or X₆, etc.

### Formula (XL)

In some embodiments, a peptide comprises or consists of the amino acid sequence of Formula (XL): X₁-X₂-X₃-X₄-X₅-X₆-X₇-X₈-X₉-V-X₁₁-X₁₂-X₁₃-X₁₄, wherein:
X₁ is D, E, H, L, or Q;
X₂ is D, F, L, S, or V;
X₃ is A, H, L, P, Q, or S;
X₄ is E, G, M, R, or W;
X₅ is A, D, E, Q, or T;
X₆ is D or E;
X₇ is A, L, R, S, or V;
X₈ is F, L, N, or Y;
X₉ is F, G, H, L, N, S, or Y;
X₁₁ is K, M, or V;
X₁₂ is K or T;
X₁₃ is D, K, or N; and
X₁₄ is F, P, S, or T.

In some embodiments, a peptide of Formula (XL) comprises or consists of the amino acid sequence of any one of SEQ ID NOS: 32, 40, 51, 65, 134, 152, 185, 216, 258, 367, and 388.

In some embodiments, the peptide comprises or consists of the amino acid sequence of Subformula (XLa): X₁-X₂-X₃-W-X₅-E-X₇-X₈-X₉-V-V-K-N-X₁₄, wherein:
X₁ is D, E, H, L, or Q;
X₂ is D, F, L, S, or V;
X₃ is A, H, L, P, Q, or S;
X₅ is A, D, E, Q, or T;
X₇ is A, L, R, S, or V;
X₈ is F, L, N, or Y;
X₉ is F, G, H, L, N, S, or Y; and
X₁₄ is F, P, S, or T.

In some embodiments, a peptide of Formula (XL) does not comprise or consist of the amino acid sequence, wherein X₁ is Q; X₂ is V; X₃ is P; X₄ is W; X₅ is E; X₆ is E; X₈ is Y; X₉ is Y; Xu is V; X₁₂ is K; X₁₃ is K; and X₁₄ is S.

In some embodiments, a peptide of Subformula (XLa) does not comprise or consist of the amino acid sequence, wherein X₁ is Q; X₂ is V; X₃ is P; X₅ is E; X₈ is Y; X₉ is Y; and X₁₄ is S.

More generally, in some embodiments, the selection of amino acids at any position of a peptide of Formula (XL) and Subformula (XLa) can be any subset or combination of choices at that position. Thus, the embodiments not only include those in which X₃ is A, H, L, P, Q, or S; but also, for example, those in which X₃ is A; X₃ is H; X₃ is L; etc.; X₃ is A or H; X₃ is A, H, or L; X₃ is H or L; X₃ is H, L, P, or Q, and so on. Moreover, the choice of amino acids, or combination or subset thereof, at one position, e.g., X₃, is independent of the choice of amino acids (or combination or subset thereof) at any other position, e.g., X₇ or X₈, etc.

### Formula (XLI)

In some embodiments, a peptide comprises or consists of the amino acid sequence of Formula (XL1): X₁-X₂-X₃-X₄-X₅-X₆-X₇-X₈-X₉-V-X₁₁-X₁₂-X₁₃-X₁₄, wherein:
X₁ is H or Q;
X₂ is A, F, I, L, T, or V;
X₃ is H, L, P, R, S, or T;
X₄ is A, F, G, L, S, W, or Y;
X₅ is A, D, E, G, N, or T;
X₆ is A, E, or V;
X₇ is A, H, P, S, or T;
X₈ is A, D, H, N, Q, T, or Y;
X₉ is G, H, L, S, T, V, or Y;
X₁₁ is S or V;
X₁₂ is K, L, N, P, Q, R, T, or Y;
X₁₃ is E, H, I, K, N, or T; and
X₁₄ is F or S.

In some embodiments, a peptide of Formula (XLI) comprises or consists of the amino acid sequence of any one of SEQ ID NOS: 39, 55, 61, 73, 110, 137, 161, 171, 190, 230, 341, 380, 489, and 543.

In some embodiments, a peptide of Formula (XLI) comprises or consists of the amino acid sequence of Subformula (XLIa): Q-X₂-X₃-X₄-X₅-E-X₇-X₈-X₉-V-V-X₁₂-K-S, wherein:
X₂ is A, F, I, L, T, or V;
X₃ is H, L, P, R, S, or T;
X₄ is A, F, G, L, S, W, or Y;
X₅ is A, D, E, G, N, or T;
X₇ is A, H, P, S, or T;
X₈ is A, D, H, N, Q, T, or Y;
X₉ is G, H, L, S, T, V, or Y; and
X₁₂ is K, L, N, P, Q, R, T, or Y.

In some embodiments, a peptide of Formula (XLI) does not comprise or consist of the amino acid sequence, wherein X₁ is Q; X₂ is V; X₃ is P; X₄ is W; X₅ is E; X₆ is E; X₇ is P; X₈ is Y; X₉ is Y; X₁₁ is V; X₁₂ is K; X₁₃ is K; and X₁₄ is S.

In some embodiments, a peptide of Subformula (XLIa) does not comprise or consist of the amino acid sequence, wherein X₂ is V; X₃ is P; X₄ is W; X₅ is E; X₇ is P; X₈ is Y; X₉ is Y; and X₁₂ is K.

More generally, in some embodiments, the selection of amino acids at any position of a peptide of Formula (XLI) and Subformula (XLIa) can be any subset or combination of choices at that position. Thus, the embodiments not only include those in which X₂ is A, F, I, L, T, or V; but also, for example, those in which X₂ is A; X₂ is F; X₂ is I; etc.; X₂ is A or F; X₂ is A, F, or F; X₂ is F or I; X₂ is A, F, I, or T, and so on. Moreover, the choice of amino acids, or combination or subset thereof, at one position, e.g., X₂, is independent of the choice of amino acids (or combination or subset thereof) at any other position, e.g., X₄ or X₅, etc.

### Formula (XLII)

In some embodiments, a peptide comprises or consists of the amino acid sequence of Formula (XLII): X₁-X₂-X₃-X₄-X₅-X₆-X₇-X₈-X₉-X₁₀-X₁₁-X₁₂-X₁₃-X₁₄, wherein:
X₁ is H, L, Q, R, or V;
X₂ is F, G, H, I, L, or V;
X₃ is A, H, N, P, Q, or T;
X₄ is F, G, L, M, S, T, or W;
X₅ is A, D, G, I, K, P, Q, or V;
X₆ is D, E, G, K, or P;
X₇ is A, L, P, Q, R, S, or T;
X₈ is F, H, S, or Y;
X₉ is A, D, F, G, H, K, N, S, or Y;
X₁₀ is I, L, N, or V;
X₁₁ is A, E, G, K, L, Q, S, V, or W;
X₁₂ is A, D, E, G, I, K, M, N, Q, R, or T;
X₁₃ is E, H, K, N, Q, R, T, or V; and
X₁₄ is F, H, N, P, S, T, or Y.

In some embodiments, a peptide of Formula (XLII) comprises or consists of the amino acid sequence of any one of SEQ ID NOS: 3, 4, 30, 36, 80, 94, 118, 181, 214, 224, 240, 260, 286, 288, 289, 312, 313, 357, 377, 393, 405, 504, and 550.

In some embodiments, a peptide of Formula (XLII) comprises or consists of the amino acid sequence of Subformula (XLIIa): H-V-P-X₄-X₅-G-X₇-Y-X₉-V-X₁₁-X₁₂-X₁₃-X₁₄, wherein:
X₄ is F, G, L, M, S, T, or W;
X₅ is A, D, G, I, K, P, Q, or V;
X₇ is A, L, P, Q, R, S, or T;
X₉ is A, D, F, G, H, K, N, S, or Y;
X₁₁ is A, E, G, K, L, Q, S, V, or W;
X₁₂ is A, D, E, G, I, K, M, N, Q, R, or T;
X₁₃ is E, H, K, N, Q, R, T, or V; and
X₁₄ is F, H, N, P, S, T, or Y.

In some embodiments, a peptide of Formula (XLII) does not comprise or consist of the amino acid sequence, wherein X₁ is Q; X₂ is V; X₃ is P; X₄ is W; X₆ is E; X₇ is P; X₈ is Y; X₉ is Y; X₁₀ is V; X₁₁ is V; X₁₂ is K; X₁₃ is K; and X₁₄ is S.

In some embodiments, a peptide of Subformula (XLIIa) does not comprise or consist of the amino acid sequence, wherein X₄ is W; X₇ is P; X₉ is Y; X₁₁ is V; X₁₂ is K; X₁₃ is K; and X₁₄ is S.

More generally, in some embodiments, the selection of amino acids at any position of a peptide of Formula (XLII) and Subformula (XLIIa) can be any subset or combination of choices at that position. Thus, the embodiments not only include those in which X₄ is F, G, L, M, S, T, or W; but also, for example, those in which X₄ is F; X₄ is G; X₄ is L; etc.; X₄ is F or G; X₄ is F, G, or L; X₄ is G or L; X₄ is G, L, M, or W, and so on. Moreover, the choice of amino acids, or combination or subset thereof, at one position, e.g., X₄, is independent of the choice of amino acids (or combination or subset thereof) at any other position, e.g., X₁₁ or X₁₂, etc.

### Formula (XLIII)

In some embodiments, a peptide comprises or consists of the amino acid sequence of Formula (XLIII): X₁-X₂-X₃-X₄-X₅-X₆-X₇-Y-X₉-V-X₁₁-X₁₂-X₁₃-X₁₄, wherein:
X₁ is G, H, N, Q, R, or Y;
X₂ is F, H, I, L, M, or V;
X₃ is L, P, or Q;
X₄ is G, L, Q, R, S, or W;
X₅ is G, H, K, Q, R, or V;
X₆ is E or V;
X₇ is P or R;
X₉ is N, S, Y;
X₁₁ is L, Q, or V;
X₁₂ is A, E, K, L, or T;
X₁₃ is K, M, N, P, Q, or T; and
X₁₄ is A, F, I, L, P, S, or Y.

In some embodiments, a peptide of Formula (XLIII) comprises or consists of the amino acid sequence of any one of SEQ ID NOS: 35, 76, 215, 252, 294, 305, 310, 311, 316, 355, 412, 413, 498, and 549.

In some embodiments, the peptide comprises or consists of the amino acid sequence of Subformula (XLIIIa): H-V-P-X₄-Q-E-P-Y-Y-V-V-K-X₁₃-X₁₄, wherein:
X₄ is G, L, Q, R, S, or W;
X₁₃ is K, M, N, P, Q, or T; and
X₁₄ is A, F, I, L, P, S, or Y.

In some embodiments, a peptide of Formula (XLIII) does not comprise or consist of the amino acid sequence, wherein X₁ is Q; X₂ is V; X₃ is P; X₄ is W; X₆ is E; X₇ is P; X₉ is Y; X₁₁ is V; X₁₂ is K; X₁₃ is K; and X₁₄ is S.

In some embodiments, a peptide of Subformula (XLIIIa) does not comprise or consist of the amino acid sequence, wherein X₄ is W; X₁₃ is K; and X₁₄ is S.

More generally, in some embodiments, the selection of amino acids at any position of a peptide of Formula (XLIII) and Subformula (XLIIIa) can be any subset or combination of choices at that position. Thus, the embodiments not only include those in which X₄ is G, L, Q, R, S, or W; but also, for example, those in which X₄ is G; X₄ is L; X₄ is Q; etc.; X₄ is G or L; X₄ is G, L, or Q; X₄ is G or L; X₄ is L, Q, R, or S, and so on. Moreover, the choice of amino acids, or combination or subset thereof, at one position, e.g., X₄, is independent of the choice of amino acids (or combination or subset thereof) at any other position, e.g., X₁₃ or X₁₄, etc.

### Formula (XLIV)

In some embodiments, a peptide comprises or consists of the amino acid sequence of Formula (XLIV): Q-X₂-X₃-X₄-X₅-X₆-X₇-X₈-X₉-X₁₀-X₁₁-X₁₂-X₁₃-X₁₄, wherein:
X₂ is H, I, M, N, or V;
X₃ is A, L, T, or V;
X₄ is F, H, L, N, S, or W;
X₅ is A, D, K, N, or S;
X₆ is A, E, H, K, or Q;
X₇ is P or S;
X₈ is F, H, N, or Y;
X₉ is N, S, T, or Y;
X₁₀ is G, P, S, V, or Y;
X₁₁ is Q or V;
X₁₂ is E or K;
X₁₃ is D, N, P, R, or T; and
X₁₄ is S, T, or Y.

In some embodiments, a peptide of Formula (XLIV) comprises or consists of the amino acid sequence of any one of SEQ ID NOS: 42, 54, 56, 154, 162, 180, 396, 467, and 483.

In some embodiments, a peptide of Formula (XLIV) comprises or consists of the amino acid sequence of Subformula (XLIVa): Q-X₂-X₃-X₄-X₅-X₆-P-Y-X₉-X₁₀-V-K-X₁₃-S, wherein:
X₂ is H, I, M, N, or V;
X₃ is A, L, T, or V;
X₄ is F, H, L, N, S, or W;
X₅ is A, D, K, N, or S;
X₆ is A, E, H, K, or Q;
X₉ is N, S, T, or Y;
X₁₀ is G, P, S, V, or Y; and
X₁₃ is D, N, P, R, or T.

In some embodiments, a peptide of Formula (XLIV) does not comprise or consist of the amino acid sequence, wherein X₂ is V; X₄ is W; X₆ is E; X₇ is P; X₈ is Y; X₉ is Y; X₁₀ is V; X₁₁ is V; X₁₂ is K; and X₁₄ is S.

In some embodiments, a peptide of Subformula (XLIVa) does not comprise or consist of the amino acid sequence, wherein X₂ is V; X₄ is W; X₆ is E; X₉ is Y; and X₁₀ is V.

More generally, in some embodiments, the selection of amino acids at any position of a peptide of Formula (XLIV) and Subformula (XLIVa) can be any subset or combination of choices at that position. Thus, the embodiments not only include those in which X₄ is F, H, L, N, S, or W; but also, for example, those in which X₄ is F; X₄ is H; X₄ is L; etc.; X₄ is F or H; X₄ is F, H, or L; X₄ is H or L; X₄ is H, L, N, or W, and so on. Moreover, the choice of amino acids, or combination or subset thereof, at one position, e.g., X₄, is independent of the choice of amino acids (or combination or subset thereof) at any other position, e.g., X₉ or X₁₀, etc.

In some embodiments, a nerve targeting peptide of the present disclosure shows selective nerve binding, including a peptide consisting or comprising the amino acid sequence of any one of SEQ ID NOS: 1-557 or a peptide consisting or comprising the amino acid sequence of any one of Formulas I-XLIV, including Subformulas Ia-XLIVa.

In some embodiments, a nerve targeting peptide of the present disclosure show selective binding to nerves versus muscle, including a peptide consisting or comprising an amino acid sequence of any one of SEQ ID NOS: 1-557 or a peptide consisting or comprising an amino acid sequence of any one of Formulas I-XLIV, including Subformulas Ia-XLIVa.

In some embodiments, a nerve targeting peptide of the present disclosure, including one comprising the amino acid sequence of Formulas (I)-(XLIV) or Subformulas (Ia)-(XLIVa), as applicable, does not include the amino acid sequence of SGQVPWEEPYYVVKKSS (HNP-401, SEQ ID NO:559); SGQVPWEEPYYVVKKSSGGC (HNP-401 with GGC linker; SEQ ID NO:560); QVPWEEPYYVVKKSS (HNP401-N-2; SEQ ID NO: 561); QVPWEEPYYVVKKSSGG (HNP401-N-2 with GG linker; SEQ ID NO: 562) and QVPWEEPYYVVKKSSGGC (HNP401-N-2 with GGC linker; SEQ ID NO: 563).

In some embodiments, peptides of the present disclosure are acetylated at the N-terminus (“Ac” or “acetyl”), amidated at the C-terminus (“CONH₂” or “NH₂”), or both.

The peptides of the present disclosure may be synthesized by any suitable method. In some embodiments, the peptides are chemically synthesized, e.g., by solid phase peptide synthesis. Techniques for solid phase synthesis are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc, 85: 2149-2156, and Stewart et al. (1984) Solid Phase Peptide Synthesis, 2nd ed. Pierce Chem. Co., Rockford, 111. In some embodiments, the peptides are recombinantly produced via cell-based expression systems. A polynucleotide encoding a peptide of the present disclosure may be incorporated into a recombinant expression vector for production of the peptide in a host cell. The expression vector also comprises expression control sequences, such as a promoter, enhancer, initiation site, and the like. The polynucleotide encoding the peptide is operatively linked to at least one expression control sequence in the expression vector to direct mRNA synthesis. Host cells may be genetically engineered (transduced, transformed, or transfected) with the vectors. The vector may be in the form of a plasmid, a viral particle, a phage, etc. The engineered host cells can be cultured in appropriate media and under suitable conditions, such as temperature, pH, and the like, and for a suitable time to express a sufficient amount of the peptide. In some embodiments, the synthesized peptides are purified using methods such as preparative reversed phase chromatography, partition chromatography, gel filtration, gel electrophoresis, or ion-exchange chromatography or other methods used in the art.

### II. Cargo Molecule

In some embodiments, the peptide of a nerve targeting peptide conjugate is directly bound to a cargo molecule. In some embodiments, the peptide of a nerve targeting peptide conjugate is indirectly (e.g., via a linker) bound to a cargo molecule. In some embodiments, the peptide is bound to a cargo molecule at its N-terminus, at its C-terminus, or at an internal position (e.g., to an internal amino acid) of the peptide. In some embodiments, two, three, four or more peptides are directly or indirectly bound to a cargo molecule.

In certain embodiments, a cargo molecule comprises a drug, fluorescent moiety, photosensitizing agent, radiolabel, neurotrophic factor, or a combination thereof. In some embodiments, the cargo molecule comprises a drug. In some embodiments, the cargo molecule comprises a fluorescent moiety. In some embodiments, the cargo molecule comprises a photosensitizing agent. In some embodiments, the peptide is bound to two or more cargo molecules. The two or more cargo molecule may be the same molecule or different molecule, or be from the same class of cargo molecule (e.g., two drugs) or from different classes of cargo molecule (e.g., one drug and one fluorescent moiety).

In some embodiments, the peptide comprises or consists or the amino acid sequence set forth in any one of SEQ ID NOS: 1-557. In some embodiments, the peptide comprises or consists of the amino acid sequence set forth in any one of SEQ ID NOS:1-30. In some embodiments, the peptide comprises or consists of the amino acid sequence set forth in any one of SEQ ID NOS:31-36. In some embodiments, the peptide comprises or consists of the amino acid sequence set forth in any one of SEQ ID NOS:37-40. In some embodiments, the peptide comprises or consists of the amino acid sequence set forth in any one of SEQ ID NOS:41-240. In some embodiments, the peptide comprises or consists of the amino acid sequence set forth in any one of SEQ ID NOS:241-349. In some embodiments, the peptide comprises or consists of the amino acid sequence set forth in any one of SEQ ID NOS:350-449. In some embodiments, the peptide comprises or consists of the amino acid sequence set forth in any one of SEQ ID NOS:450-557.

In some embodiments, the peptide comprises or consists of the amino acid sequence set forth in any one of Formulas (I)-(XLIV) and Subformulas (Ia)-(XLIVa).

### Fluorescent Moieties

In some embodiments, the cargo molecule of the nerve targeting peptide conjugate comprises a fluorescent moiety (e.g., a fluorescent protein, fluorescent peptide, or fluorophore (fluorescent dye) molecule). All fluorescent moieties are encompassed within the term “fluorescent moiety.” Specific examples of fluorescent moieties given herein, are illustrative and are not meant to limit the fluorescent moieties for use with the targeting molecules disclosed herein. Common classes of fluorophores (fluorescent dyes) include, but are not limited to, xanthenes, such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives of such dyes. Fluorescent dyes are discussed, for example, in U.S. Pat. Nos. 4,452,720; 5,227,487; and 5,543,295.

In some embodiments, the fluorescent moiety is selected from the group consisting of a xanthene; a bimane; a coumarin; an aromatic amines; a benzofuran; a fluorescent cyanine; a carbazole; a dicyanomethylene pyrane; polymethine; oxabenzanthrane; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; FITC; Cy3; EGFP; cyan fluorescent protein (CFP); EGFP; 5-FAM; 6-FAM; FAM; fluorescein, IAEDANS, EDANS and BODIPY FL; TRITC; Cy5; Cy3; YFP; 6-FAM; LC Red 640; Alexa Fluor 546; fluorescein; tetramethylrhodamine; Dabcyl; BODIPY FL; QSY 7, QSY 9, QSY 21 and BBQ-650 dyes.

In some embodiments, the cargo molecule comprises a fluorescein dye. Typical fluorescein dyes include, but are not limited to, 5-carboxyfluorescein, fluorescein-5-isothiocyanate, 5(6)-carboxyfluorescein, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carboxyfluorescein quaternary ammonium, carboxyfluorescein phosphonate, carboxyfluorescein GABA, carboxyfluorescein-cys-Cy5,5′(6′)-carboxyfluorescein, fluorescein glutathione, and 6-carboxyfluorescein; examples of other fluorescein dyes can be found, for example, in U.S. Pat. Nos. 6,008,379, 5,750,409, 5,066,580, and 4,439,356.

In some embodiments, the cargo molecule comprises a rhodamine dye, such as, for example, 5-(and 6)-carboxy rhodamine 110, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®), and other rhodamine dyes. Other rhodamine dyes can be found, for example, in U.S. Pat. Nos. 6,080,852; 6,025,505; 5,936,087; 5,750,409. In some embodiments, a cargo moiety includes a cyanine dye, such as, for example, Cy3, Cy3B, Cy3.5, Cy5, Cy5.5, Cy 7, and indocyanine green.

In some embodiments, the fluorophore exhibits green fluorescence (such as for example 494 nm/519 nm), orange fluorescence (such as for example 554 nm/570 nm), red fluorescence (such as for example 590 nm/617 nm), or far red fluorescence (such as for example 651 nm/672 nm) excitation/emission spectra. In some embodiments, the fluorophore is a fluorophore with excitation and emission spectra in the range of about 350 nm to about 775 nm. In some embodiments the excitation and emission spectra are about 346 nm/446 nm, about 494 nm/519 nm, about 554 nm/570 nm, about 555 nm/572 nm, about 590 nm/617 nm, about 651 nm/672 nm, about 679 nm/702 nm or about 749 nm/775 nm.

In some embodiments, the fluorophore can include but is not limited to AlexaFluor 3, AlexaFluor 5, AlexaFluor 350, AlexaFluor 405, AlexaFluor 430, AlexaFluor 488, AlexaFluor 500, AlexaFluor 514, AlexaFluor 532, AlexaFluor 546, AlexaFluor 555, AlexaFluor 568, AlexaFluor 594, AlexaFluor 610, AlexaFluor 633, AlexaFluor 647, AlexaFluor 660, AlexaFluor 680, AlexaFluor 700, and AlexaFluor 750 (Molecular Probes AlexaFluor dyes, available from Life Technologies, Inc. (USA)). In some embodiments, the fluorophore can include but is not limited to Cy dyes, including Cy2, Cy3, Cy3B, Cy3.5, Cy5, Cy5.5 and Cy7 (available from GE Life Sciences or Lumiprobes). In some embodiments the fluorophore can include but is not limited to DyLight 350, DyLight 405, DyLight 488, DyLight 550, DyLight 594, DyLight 633, DyLight 650, DyLight 680, DyLight 750 and DyLight 800 (available from Thermo Scientific (USA)). In some embodiments, the fluorophore can include but is not limited to a FluoProbes 390, FluoProbes 488, FluoProbes 532, FluoProbes 547H, FluoProbes 594, FluoProbes 647H, FluoProbes 682, FluoProbes 752 and FluoProbes 782, AMCA, DEAC (7-Diethylaminocoumarin-3-carboxylic acid); 7-Hydroxy-4-methylcoumarin-3; 7-Hydroxycoumarin-3; MCA (7-Methoxycoumarin-4-acetic acid); 7-Methoxycoumarin-3; AMF (4′-(Aminomethyl)fluorescein); 5-DTAF (5-(4,6-Dichlorotriazinyl)aminofluorescein); 6-DTAF (6-(4,6-Dichlorotriazinyl)aminofluorescein); FAM; 6-FAM (6-Carboxyfluorescein), 5(6)-FAM cadaverine; 5-FAM cadaverine; 5(6)-FAM ethylenediamme; 5-FAM ethylenediamme; 5-FITC (FITC Isomer I; fluorescein-5-isothiocyanate); 5-FITC cadaverin; Fluorescein-5-maleimide; 5-IAF (5-Iodoacetamidofluorescein); 6-JOE (6-Carboxy-4′,5′-dichloro-2′,7′-dimethoxyfluorescein); 5-CRI 10 (5-Carboxyrhodamine 110); 6-CRI 10 (6-Carboxyrhodamine 110); 5-CR6G (5-Carboxyrhodamine 6G); 6-CR6G (6-Carboxyrhodamine 6G); 5(6)-Carboxyrhodamine 6G cadaverine; 5(6)-Caroxyrhodamine 6G ethylenediamme; 5-ROX (5-Carboxy-X-rhodamine); 6-ROX (6-Carboxy-X-rhodamine); 5-TAMRA (5-Carboxytetramethylrhodamine); 6-TAMRA (6-Carboxytetramethylrhodamine); 5-TAMRA cadaverine; 6-TAMRA cadaverine; 5-TAMRA ethylenediamme; 6-TAMRA ethylenediamme; 5-TMR C6 maleimide; 6-TMR C6 maleimide; TR C2 maleimide; TR cadaverine; 5-TRITC; G isomer (Tetramethylrhodamine-5-isothiocyanate); 6-TRITC; R isomer (Tetramethylrhodamine-6-isothiocyanate); Dansyl cadaverine (5-Dimethylaminonaphthalene-1-(N-(5-aminopentyl))sulfonamide); EDANS C2 maleimide; fluorescamine; NBD; and pyrromethene and derivatives thereof.

In some embodiments, a cargo comprises an environmentally sensitive fluorescent dye or fluorophore. Examples of environmentally sensitive fluorescent dyes or fluorophores include 5,6-carboxy-diethyl rhodol (pH sensitive), merocyanine (membrane potential sensitive), and Nile red carboxylic acid (lipid sensitive).

In some embodiments, the fluorescent moiety is a peptide or protein. In some embodiments, the fluorescent moiety is Green Fluorescent Protein (GFP). In some embodiments, the fluorescent moiety is a derivative or variant of GFP, including for example: EGFP, emerald, superfolder GFP, azami green mWasabi, TagGFP, TurboGFP, AcGFP, ZsGreen, T-Sapphire, EBFP, EBFP2, Azurite, mTagBFP, ECFP, mECFP, Cerulean, m Turquoise, CyPet, AmCyan1, Midori-Ishi Cyan, TagCFP, mTFP1 (Teal), EYFP, Topaz, Venus, mCitrine, YPet, TagYFP, PhiYFP, ZsYellow1, mBanana, Kusabira Orange, Kusbira Orange2, mOrange, mOrange2, dTomato, dTomato-Tandem, TagRFP, TagRFP-Y, DsRed, DsRed2, DsRed-Express (T1), DsRed-Monomer, mTangerine, mRuby, mApple, mStrawberry, AsRed2, mRFP1, JRed, mCherry, HcRed1, mRaspberry, dKeima-Tandem, HcRed-Tandem, mPlum, AQ143, mKalamal, YFP, and Citrine.

Fluorescent moieties are detected by any suitable method. For example, a fluorescent moiety may be detected by exciting the fluorochrome with the appropriate wavelength of light and detecting the resulting fluorescence, e.g., by microscopy, visual inspection, via photographic film, by the use of electronic detectors such as charge coupled devices (CCDs), photomultipliers, etc.

In some embodiments, the fluorescent moiety is conjugated to high molecular weight molecule, such as water soluble polymers including, but not limited to, dextran, PEG, serum albumin, or poly(amidoamine) dendrimer.

### Photosensitizing Agents

In some embodiments, a cargo molecule of a nerve targeting peptide conjugate comprises a photosensitizing agent. A photosensitizing agent is any agent or compound useful in light induced ablation therapy. Such agents, when exposed to a specific wavelength of light, react with molecular oxygen to produce singlet oxygen, which is highly cytotoxic. Thus, targeting molecules of the present invention comprising a photosensitizing agent may be used to focally injure nerves. In certain embodiments, a photosensitizing agent is a porphyrin, chlorin, or dye. Examples of photosensitizing agents include porphyrin, protoporfin IX, purlytin, verteporfin, HPPH, temoporfin, methylene blue, photofrin, protofrin, hematoporphyrin, Talaporfin, benzopophyrin derivative monoacid, 5-aminileuvolinic acid, Lutetium texaphyrin, metallophthalocyanine, metallo-naphthocyaninesulfobenzo-porphyrazines, metallo-naphthalocyanines, zinc tetrasulfophthalocyanine, bacteriochlorins, metallochlorins, chlorine derivative, Tetra(m-hydroxyphenyl)chlorin (mTHPC), pheophorbide, dibromofluorescein (DBF), IR700DX, naphthalocyanine, and porphyrin derivatives.

In some embodiments, the photosensitizing agent is conjugated to a C-terminal cysteine residue of the human neuron or nerve targeting molecule via maleimide mediated conjugation. Preferably, the photosensitizing agent of the present disclosure is activated by light having a wavelength of between 400 nm to 700 nm. Still more preferably, the photosensitizing agent in the present disclosure is activated at 627 nm and 660 nm. An optimal light dose can be identified to generate maximal nerve killing with minimal injury to adjacent tissue.

### Drugs

In some embodiments, a cargo molecule of a nerve targeting peptide conjugate comprises a drug. All drugs that act on a neuron or nerve (or a component thereof) are encompassed within the term “drug.” Specific examples of drug given herein, are illustrative and are not meant to limit the drugs for use with the targeting molecules disclosed herein.

In some embodiments, the drug is selected from a drug that: induces cell death (apoptotic or necrotic), inhibits cell death (apoptotic or necrotic), inhibits the transmission of a neuron or nerve signal (i.e., an electrochemical impulse), inhibits the release of a neurotransmitter, agonizes the activity of a GABA receptor, partially or fully inhibits the repolarization of a neuron, disrupts the conduction of an ion channel, or a combination thereof.

In some embodiments, the drug is an antihistamine, a GABA receptor modulator, a neurotransmitter reuptake inhibitor, a local anesthetic, an anticholinergic, a sodium channel blocker, a calcium channel blocker, a thyrotropin-releasing hormone, a y-secretase inhibitor, an AMPA receptor agonist or antagonist, an NMDA receptor agonist or antagonist, an mGlu receptor agonist or antagonist, a growth factor, an antiemetic agent, a corticosteroid; a cytotoxic agent; an antioxidant, an iron chelator, a mitochondrial modulator, a sirtuin modulator, a nitric oxide (NO) and/or nitric oxide synthase (NOS) modulator, a potassium channel agonist or antagonist, a purigenic receptor agonist or antagonist, or a combination thereof.

In some embodiments, the drug is meclizine, diphenhydramine, dimenhydrinate, loratadine, quetiapine, mepyramine, piperoxan, antazoline, carbinoxamine, doxylamine, clemastine, pheniramine, chlorphenamine, chlorpheniramine, dexchlorpheniramine, brompheniramine, triprolidine, cyclizine, chlorcyclizine, hydroxyzine, promethazine, alimemazine, trimeprazine, cyproheptadine, azatadine, ketotifen, oxatomide, meclizine hydrochloride, promethazine hydrochloride, cinnarizine, hydroxyzine pamoate, betahistine dihydrochloride, alprazolam, bromazepam, brotizolam, chlordiazepoxide, clonazepam, clorazepate, diazepam, estazolam, flunitrazepam, flurazepam, loprazolam, lorazepam, lormetazepam, idazolam, nimetazepam, nitrazepam, oxazepam, prazepam, temazepam, triazolam, clonazepam, diazepam, lorazepam, furosemide, bumetanide, ethacrynic acid, gabapentin, pregabalin, muscimol, baclofen, amitriptyline, nortriptyline, trimipramine, fluoxetine, paroxetine, sertraline, glycopyrrolate, homatropine, scopolamine, atropine, benzocaine, carticaine, cinchocaine, cyclomethycaine, lidocaine, prilocaine, propxycaine, proparacaine, tetracaine, tocainide, trimecaine, carbamazepine, oxcarbazepine, phenytein, valproic acid, sodium valproate, cinnarizine, flunarizine, nimodipine, thyrotropin-releasing hormone, amifostine (also known as WR-2721, or ETHYOL®); a carbamate compound (e.g., 2-phenyl-1,2-ethanediol monocarbomates and dicarbamates); LY450139 (hydroxylvaleryl monobenzocaprolactam); L685458 (1S-benzyl-4R[1-[1-S-carbamoyl-2-phenethylcarbamoyl)-1S-3-methylbutylcarbamoyl]-2R-hydroxy-5-phenylpentyl}carbamic acid tert-butyl ester); LY411575 (N2-[(2S)-2-(3,5-difluorophenyl)-2-hydroxyethanoyl]-N1[(7S)-5-methyl-6-oxo-6,7-dihydro-5H-dibenzo[bid]azepin-7yl]-L-alaninamide); MK-0752; tarenflurbil; BMS-299897 (2-[(1R)-1-[[(4-chlorophenyl) sulfony](2,5-difluorophenyl)amino]ethyl]-5-fluorobenzenepropanoic acid; CNQX (6-cyano-7-nitroquinoxaline-2,3-dione); NBQX (2,3-dihydroxy-6-nitro-7-sulfamoylbenzo[f]quinoxaline-2,3-dione); DNQX (6,7-dinitroquinoxaline-2,3-dione); kynurenic acid; 2,3-dihydroxy-6-nitro-7-sulfamoylbenzo-[fjquinoxaline; 1-aminoadamantane; dextromethorphan; dextrorphan; ibogaine; ketamine; nitrous oxide; phencyclidine; riluzole; tiletamine; memantine; dizocilpine; aptiganel; remacimide; 7-chlorokynurenate; DCKA (5,7-dichlorokynurenic acid); kynurenic acid; 1-aminocyclopropanecarboxylic acid (ACPC); AP7 (2-amino-7-phosphonoheptanoic acid); APV (R-2-amino-5-phosphonopentanoate); CPPene (3-[(R)-2-carboxypiperazin-4-yl]-prop-2-enyl-1-phosphonic acid); (+)-(1S,2S)-1-(4-hydroxy-phenyl)-2-(4-hydroxy-4-phenylpiperidino)-1-pro-panol; (1S,2S)-1-(4-hydroxy-3-methoxyphenyl)-2-(4-hydroxy-4-phenylpiperi-dino)-1-propanol; (3R,4S)-3-(4-(4-fluorophenyl)-4-hydroxypiperidin-1-yl-)-chroman-4;7-diol; (1R*,2R*)-1-(4-hydroxy-3-memylphenyl)-2-(4-(4-fluoro-phenyl)-4-hydroxypiperidin-1-yl)-propan-1-ol-mesylate); LY389795 ((−)-2-thia-4-aminobicyclo-hexane-4,6-dicarboxylate); LY379268 ((−)-2-oxa-4-aminobicyclo-hexane-4,6-dicarboxylate); LY354740 ((+)-2-aminobicyclo-hexane-2,6dicarboxylate); DCG-IV ((2S,2′R,3′R)-2-(2′,31-dicarboxycyclopropyl)glycine); 2R,4R-APDC (2R,4R-4-aminopyrrolidine-2,4-dicarboxylate); (S)-3C4HPG ((S)-3-carboxy-4-hydroxyphenylglycine); (S)-4C3HPG ((S)-4-carboxy-3-hydroxyphenylglycine); L-CCG-I ((2S,1′S,2′S)-2-(carboxycyclopropyl)glycine); ACPT-I ((1S,3R,4S)-1-aminocyclopentane-1,3,4-tricarboxylic acid); L-AP4 (L-(+)-2-Amino-4-phosphonobutyric acid); (S)-3,4-DCPG ((S)-3,4-dicarboxyphenylglycine); (RS)-3,4-DCPG ((RS)-3,4-dicarboxyphenylglycine); (RS)-4-phosphonophenylglycine ((RS)PPG); AMN082 (,N′-bis(diphenylmethyl)-1,2-ethanediamine dihydrochloride); DCG-IV ((2S,2′R,3′R)-2-(2′,3′-dicarboxycyclopropyl)glycine); AMN082; brain-derived neurotrophic factor (BDNF); ciliary neurotrophic factor (CNTF); glial cell-line derived neurotrophic factor (GDNF); neurotrophin-3; neurotrophin-4; fibroblast growth factor (FGF) receptor; insulin-like growth factor (IGF); an aminoglycoside antibiotic (e.g., gentamicin and amikacin); a macrolide antibiotic (e.g, erythromycin); a glycopeptide antibiotic (e.g. vancomycin); salicylic acid; nicotine; Eburnamenine-14-carboxylic acid ethyl ester; sipatrigine (2-(4-Methylpiperazin-1-yl)-5-(2,3,5-trichlorophenyl)-pyrimidin-4-amine); amiloride (3,5-diamino-N-(aminoiminomethyl)-6-chloropyrazinecarbox amide hydrochloride); carbamazepine (5H-dibenzo[b,f]azepine-5-carboxamide); TTX (octahydro-12-(hydroxymethyl)-2-imino-5,9:7,10a-dimethano-10aH-[1,3]dioxocino[6,5-d]pyrimidine-4,7,10,11,12-pentol); RS100642 (1-(2,6-dimethyl-phenoxy)-2-ethylaminopropane hydrochloride); mexiletine ((1-(2,6-dimethylphenoxy)-2-aminopropane hydrochloride)); QX-314 (N-(2,6-Dimethylphenylcarbamoylmethyl)triethylammonium bromide); phenytoin (5,5-diphenylimidazolidine-2,4-dione); lamotrigine (6-(2,3-dichlorophenyl)-1,2,4-triazine-3,5-diamine); 4030W92 (2,4-diamino-5-(2,3-dichlorophenyl)-6-fluoromethylpyrimidine); BW1003C87 (5-(2,3,5-trichlorophenyl)pyrimidine-2,4-1.1 ethanesulphonate); QX-222 (2-[(2,6-dimethylphenyl)amino]-N,N,N-trimethyl-2-oxoetha niminium chloride); ambroxol (trans-4-[[(2-Amino-3,5-dibromophenyl)methyl]amino]cyclo hexanol hydrochloride); R56865 (N-[1-(4-(4-fluorophenoxy)butyl]-4-piperidinyl-N-methyl-2-benzo-thiazolamine); lubeluzole; ajmaline ((17R,21alpha)-ajmalan-17,21-diol); procainamide (4-amno-N-(2-diethylaminoethyljbenzamide hydrochloride); flecainide; riluzoleor; triamicinolone actenoide; Dexamethasone; promethazine; prochlorperazine; trimethobenzamide; triethylperazine; dolasetron; granisetron; ondansetron; tropisetron; and palonosetron; droperidol; meclizine; perphenazine; thiethyl perazine; domperidone; properidol; haloperidol; chlorpromazine; promethazine; prochlorperazine; metoclopramide; dronabinol; nabilone; sativex; scopolamine; dexamethasone; trimethobenzamine; emetrol; propofol; muscimol; acridine carboxamide; actinomycin; 17-N-allylamino-17-demethoxygeldanamycin; amcinonide; amsacrine; aminopterin; anthracycline; antineoplastic; antineoplaston; 5-azacytidine; azathioprine; BL22; beclometasone; bendamustine; betamethasone; biricodar; bleomycin; bortezomib; bryostatin; budesonide; busulfan; calyculin; camptothecin; capecitabine; carboplatin; chlorambucil; cisplatin; cladribine; clofarabine; cortisone acetate; cytarabine; dacarbazine; dasatinib; daunorubicin; decitabine; desonide; dexamethasone; dichloroacetic acid; discodermolide; docetaxel; doxorubicin; epirubicin; epothilone; eribulin; estramustine; etoposide; exatecan; exisulind; ferruginol; floxuridine; fludarabine; fluocinonide; fluocortolone; fluorouracil; fosfestrol; fotemustine; gemcitabine; halometasone; halcinonide; hydrocortisone; hydrocortisone acetate; hydroxyurea; IT-101; idarubicin; ifosfamide; imiquimod; irinotecan; irofulven; ixabepilone; laniquidar; lapatinib; lenalidomide; lomustine; lurtotecan; mafosfamide; masoprocol; mechlorethamine; melphalan; mercaptopurine; methylprednisone; mitomycin; mitotane; mitoxantrone; mometasone; nelarabine; nilotinib; oblimersen; oxaliplatin; PAC-I; methotrexate (RHEUMATREX®, Amethopterin); cyclophosphamide (CYTOXAN®) thalidomide (THALID OMID®); paclitaxel; pemetrexed; pentostatin; pipobroman; pixantrone; plicamycin; prednisolone; prednisone; procarbazine; proteasome inhibitors (e.g., bortezomib); raltitrexed; rebeccamycin; rubitecan; SN-38; salinosporamide A; satraplatin; streptozotocin; swainsonine; tariquidar; taxane; tegafur-uracil; temozolomide; testolactone; thioTEPA; tioguanine; topotecan; trabectedin; triamcinolone acetonide; tretinoin; triplatin tetranitrate; tris(2-chloroethyl)amme; troxacitabine; uracil mustard; valrubicin; vinblastine; vincristine; vinorelbine; vorinostat; zosuquidar; N-acetylcysteine; vitamin E; vitamin C; vitamin A; lutein; selenium glutathione; melatonin; a polyphenol; a carotenoid; coenzyme Q-IO; Ebselen (2-phenyl-1,2-benzisoselenazol-3(2H)-one (also called PZ 51 or DR3305); L-methionine; azulenyl nitrones; L-(+)-Ergothioneine; CAPE (caffeic acid phenethyl ester); dimethylthiourea; dimethylsulfoxide; disufenton sodium; pentoxifylline; MCI-186; Ambroxol; U-83836E; MitoQ (mitoquinone mesylate); Idebenone (2-(10-hydroxydecyl)-5,6-dimethoxy-3-methyl-cyclohexa-2,5-diene-1,4-dione); desferrioxamine; hydroxybenzyl ethylene diamine; fullerenol-1, pyrrolidine dithiocarbamate; acetylcarnitine; lipoic acid; a stilbene; a chalcone; a flavone; an isoflavone; a flavanones; an anthocyanidin; a catechin; isonicotinamide; dipyridamole; ZM 336372; camptothecin; coumestrol; nordihydroguaiaretic acid; esculetin; SRT-1720; SRT-1460; SRT-2183; aminoguanidine; 1-Amino-2-hydroxyguanidine p-toluensulfate; GED; bromocriptine mesylate; dexamethasone; SDMA; ADMA; L-NMMA; L-NMEA; D-MMA; L-NIL; L-NNA; L-NPA; L-NAME; L-VNIO; diphenyleneiodonium chloride; 2-ethyl-2-thiopseudourea; haloperidol; L-NIO; MEG; SMT; SMTC; 7-Ni; nNOS inhibitor; 1,3-PBITU; L-thiocitrulline; TRIM; MTR-105; BBS-I; BBS-2; ONO-1714; GW273629; GW 274150; PPA250; AR-R17477; AR-R18512; spiroquinazolone; 1400W; S-NC; NTG; SNP; thapsigargin; VEGF; bradykinin; ATP; sphingosine-1-phosphate; estrogen; angiopoietin; acetylcholine; SIN-I; GEA 3162; GEA; GEA 5024; GEA 5538; SNAP; molsidomine; CNO-4; CNO-5; DEA/NO; IPA/NO; SPER/NO; SULFI/NO; OXI/NO; DETA/NO; nicorandil; minoxidil, levcromakalim; lemakalim; cromakalim; L-735,334; retigabine; flupirtine; BMS-204352; DMP-543; linopirdine; XE991; 4-AP; 3,4-DAP; E-4031; DIDS; Way 123,398; CGS-12066 A; dofetilide; sotalol; apamin; amiodarone; azimilide; bretylium; clofilium; tedisamil; ibutilide; sematilide; nifekalant; tamulustoxin; ATP; ADP; UTP; UDP; UDP-glucose; adenosine; 2-MESATP; 2-MESADP; ABMEATP; DATPAS; ATPrS; BZ-ATP; MRS2703; DENUFOSOL TETRASODIUM; MRS2365; MRS 2690; PSB 0474; A-317491; RO-3 (Roche); SURAMIN; PPADS; PPNDS; DIDS; pyridoxal-5-phosphate; 5-(3-bromophenyl)-1,3-dihydro-2H-benzofuro-[3,2-e]-1,4-diazepin-2-one; cibacron blue; basilen blue; ivermectin; A-438079; A-740003; NF023; NF449; NFI 10; NF157; MRS 2179; NF279; MRS 2211; MRS 2279; MRS 2500 tetrasodium salt; TNP-ATP; tetramethylpyrazine; Ip51; jQy-carboxymethylene ATP; βγ-chlorophosphomethylene ATP; KN-62; spinorphin; minocycline; SB-203580 (4-(4-Fluorophenyl)-2-(4-methylsulfmyl phenyl)-5-(4-pyridyl) 1H-imidazole); PD 169316 (4-(4-Fluorophenyl)-2-(4-nitrophenyl)-5-(4-pyridyl)-1H-imidazole); SB 202190 (4-(4-Fluorophenyl)-2-(4-hydroxyphenyl)-5-(4-pyridyl)1H-imidazole); RWJ 67657 (4-[4-(4-fluorophenyl)-1-(3-phenylpropyl)-5-(4-pyridinyl)-1H-imidazol-2-yl]-3-butyn-1-ol); SB 220025 (5-(2-Amino-4-pyrimidinyl)-4-(4-fluorophenyl)-1-(4-piperidinlyl)imidazole); D-JNKI-I ((D)-hJIPi75-i57-DPro-DPro-(D)-HIV-T AT57-48); AM-111 (Auris); SP600125 (anthra[1,9-cd]pyrazol-6(2H)-one); JNK Inhibitor I ((L)-HIV-TAT48-57-PP-JBD20); JNK Inhibitor III ((L)-HIV-TAT47-57-gaba-c-Jun833-57); AS601245 (1,3-benzothiazol-2-yl (2-[[2-(3-pyridinyl)ethyl]amino]-4 pyrimidinyl) acetonitrile); JNK Inhibitor VI (H2N-RPKRPTTLNLF-NH2) (SEQ ID NO:564); JNK Inhibitor VIII (N-(4-Amino-5-cyano-6-ethoxypyridin-2-yl)-2-(2,5-dimethoxyphenyl)acetamide); JNK Inhibitor IX (N-(3-Cyano-4,5,6,7-tetrahydro-1-benzothien-2-yl)-1-naphthamide); dicumarol (3,3′-Methylenebis(4-hydroxycoumarin)); SC-236 (4-[5-(4-chlorophenyl)-3-(trifluoromethyl)-1H-pyrazol-1-yl]benzene-sulfonamide); CEP-1347 (Cephalon); CEP-11004 (Cephalon); an artificial protein comprising at least a portion of a Bcl-2 polypeptide; a recombinant FNK; V5 (also known as Bax inhibitor peptide V5); Bax channel blocker ((+)-1-(3,6-Dibromocarbazol-9-yl)-3-piperazin-1-yl-propan-2-ol); Bax inhibiting peptide P5 (also known as Bax inhibitor peptide P5); Kp7-6; FAIM(S) (Fas apoptosis inhibitory molecule-short); FAIM(L) (Fas apoptosis inhibitory molecule-long); Fas:Fc; FAP-I; NOK2; F2051; F1926; F2928; ZB4; Fas M3 mAb; EGF; 740 Y-P; SC 3036 (KKHTDDGYMPMSPGVA) (SEQ ID NO:565); PI 3-kinase Activator (Santa Cruz Biotechnology, Inc.); Pam3Cys ((S)-(2,3-bis(palmitoyloxy)-(2RS)-propyl)-N-palmitoyl-(R)-Cys-(S)-Ser(S)-Lys4-OH, trihydrochloride); Actl (NF-kB activator 1); an anti-DcB antibody; Acetyl-11-keto-b-Boswellic Acid; Andrographolide; Caffeic Acid Phenethyl Ester (CAPE); Gliotoxin; Isohelenin; NEMO-Binding Domain Binding Peptide (DRQIKIWFQNRRMKWKKTALDWSWLQTE) (SEQ ID NO:566); NF-kB Activation Inhibitor (6-Amino-4-(4-phenoxyphenylethylamino)quinazoline); NF-kB Activation Inhibitor II (4-Methyl-Nl-(3-phenylpropyl)benzene-1,2-diamine); NF-kB Activation Inhibitor III (3-Chloro-4-nitro-N-(5-nitro-2-thiazolyl)-benzamide); NF-kB Activation Inhibitor IV ((E)-2-Fluoro-4′-methoxystilbene); NF-kB Activation Inhibitor V (5-Hydroxy-(2,6-diisopropylphenyl)-1H-isoindole-1,3-dione); NF-kB SN50 (AAV ALLP A VLLALL AP VQRKRQKLMP) (SEQ ID NO:567); Oridonin; Parthenolide; PPM-18 (2-Benzoylamino-1,4-naphthoquinone); Rol06-9920; Sulfasalazine; TIRAP Inhibitor Peptide (RQIKiWFNRRMKWKKLQLRD AAPGGAIVS) (SEQ ID NO:568); Withaferin A; Wogonin; BAY 11-7082 ((E)3-[(4-Methylphenyl)sulfonyl]-2-propenenitrile); BAY 11-7085 ((E)3-[(4-t-Butylphenyl)sulfonyl]-2-propenenitrile); (E)-Capsaicin; Aurothiomalate (ATM or AuTM); Evodiamine; Hypoestoxide; IKK Inhibitor III (BMS-345541); IKK Inhibitor VII; IKK Inhibitor X; IKK Inhibitor II; IKK-2 Inhibitor IV; IKK-2 Inhibitor V; IKK-2 Inhibitor VI; IKK-2 Inhibitor (SC-514); IkB Kinase Inhibitor Peptide; IKK-3 Inhibitor IX; ARRY-797 (Array BioPharma); SB-220025 (5-(2-Amino-4-pyrimidinyl)-4-(4-fluorophenyl)-1-(4-piperidinlyl)imidazole); SB-239063 (trans-4-[4-(4-Fluorophenyl)-5-(2-methoxy-4-pyrimidinyl)-1H-imidazol-1-ylJcyclohexanol); SB-202190 (4-(4-Fluorophenyl)-2-(4-hydroxyphenyl)-5-(4-pyridyl)1H-imidazole); JX-401 (-[2-Methoxy-4-(methylthio)benzoyl]-4-(phenylmethyl)piperidine); PD-169316 (4-(4-Fluorophenyl)-2-(4-nitrophenyl)-5-(4-pyridyl)-1H-imidazole); SKF-86002 (6-(4-Fluorophenyl)-2,3-dihydro-5-(4-pyridinyl)imidazo[2,1-b]thiazole dihydrochloride); SB-200646 (N-(1-Methyl-1H-indol-5-yl)-N′-3-pyridinylurea); CMPD-I (2′-Fluoro-N-(4-hydroxyphenyl)-[1,1′-biphenyl]-4-butanamide); EO-1428 ((2-Methylphenyl)-[4-[(2-amino-4-bromophenyl)amino]-2-chlorophenyl]methanone); SB-253080 (4-[5-(4-Fluorophenyl)-2-[4-(methylsulfonyl)phenyl]-1H-imidazol-4-yl]pyridine); SD-169 (1H-Indole-5-carboxamide); SB-203580 (4-(4-Fluorophenyl)-2-(4-methylsulfinyl phenyl)-5-(4-pyridyl) 1H-imidazole); TZP-101 (Tranzyme Pharma); TZP-102 (Tranzyme Pharma); GHRP-6 (growth hormone-releasing peptide-6); GHRP-2 (growth hormone-releasing peptide-2); EX-1314 (Elixir Pharmaceuticals); MK-677 (Merck); L-692,429 (Butanamide, 3-amino-3-methyl-N-(2,3,4,5-tetrahydro-2-oxo-1-((2′-(1H-tetrazol-5-yl)(1,1′-biphenyl)-4-yl)methyl)-1H-1-benzazepin-3-yl)-, (R)-); EP1572 (Aib-DTrp-DgTrp-CHO); diltiazem; metabolites of diltiazem; BRE (Brain and Reproductive organ-Expressed protein); verapamil; nimodipine; diltiazem; omega-conotoxin; GVIA; amlodipine; felodipine; lacidipine; mibefradil; NPPB (5-Nitro-2-(3-phenylpropylamino)benzoic Acid); flunarizine; erythropoietin; pipeline; hemin; brazilin; z-VAD-FMK (Benzyloxycarbonyl-Val-Ala-Asp(OMe)-fluoromethylketone); z-LEHD-FMK (benzyloxycarbonyl-Leu-Glu(OMe)-His-Asp(OMe)-fluoromethylketone) (SEQ ID NO:569); B-D-FMK (boc-aspartyl(Ome)-fluoromethylketone); Ac-LEHD-CHO (N-acetyl-Leu-Glu-His-Asp-CHO) (SEQ ID NO:569); Ac-IETD-CHO (N-acetyl-Ile-Glu-Thr-Asp-CHO) (SEQ ID NO:570); z-IETD-FMK (benzyloxycarbonyl-Ile-Glu(OMe)-Thr-Asp(OMe)-fluoromethylketone) (SEQ ID NO:570); FAM-LEHD-FMK (benzyloxycarbonyl Leu-Glu-His-Asp-fluoromethyl ketone) (SEQ ID NO:569); FAM-LETD-FMK (benzyloxycarbonyl Leu-Glu-Thr-Asp-fluoromethyl ketone) (SEQ ID NO:571); Q-VD-OPH (Quinoline-Val-ASp-CH2-O-Ph); XIAP; cIAP-1; cIAP-2; ML-IAP; ILP-2; NAIP; Survivin; Bruce; IAPL-3; fortilin; leupeptine; PD-150606 (3-(4-Iodophenyl)-2-mercapto-(Z)-2-propenoic acid); MDL-28170 (Z-Val-Phe-CHO); calpeptin; acetyl-calpastatin; MG 132 (N-[(phenylmethoxy)carbonyl]-L-leucyl-N-[(1S)-1-formyl-3-methylbutyl]-L-leucinamide); MYODUR; BN 82270 (Ipsen); BN 2204 (Ipsen); AHLi-11 (Quark Pharmaceuticals), an mdm2 protein, pifithrin-α (1-(4-Methylphenyl)-2-(4,5,6,7-tetrahydro-2-imino-3(2H)-benzothiazolyl)ethanone); trans-stilbene; cis-stilbene; resveratrol; piceatannol; rhapontin; deoxyrhapontin; butein; chalcon; isoliquirtigen; butein; 4,2′,4′-trihydroxychalcone; 3,4,2′,4′,6′-pentahydroxychalcone; flavone; morin; fisetin; luteolin; quercetin; kaempferol; apigenin; gossypetin; myricetin; 6-hydroxyapigenin; 5-hydroxyflavone; 5,7,3′,4′,5′-pentahydroxyflavone; 3,7,3′,4′,5′-pentahydroxyflavone; 3,6,3′,4′-tetrahydroxyflavone; 7,3′,4′,5′-tetrahydroxyflavone; 3,6,2′,4′-tetrahydroxyflavone; 7,4′-dihydroxyflavone; 7,8,3′,4′-tetrahydroxy flavone; 3,6,2′,3′-tetrahydroxyflavone; 4′-hydroxyflavone; 5-hydroxyflavone; 5,4′-dihydroxyflavone; 5,7-dihydroxyflavone; daidzein; genistein; naringenin; flavanone; 3,5,7,3′,4′-pentahydroxyflavanone; pelargonidin chloride; cyanidin chloride; delphinidin chloride; (−)-epicatechin (Hydroxy Sites: 3,5,7,3′,4\hat{ }; (−)-catechin (Hydroxy Sites: 3,5,7,3′,40; (−)-gallocatechin (Hydroxy Sites: 3,5,7,3′,4′,5O (+)-catechin (Hydroxy Sites: 3,5,7,3′,4\hat{ }; (+)-epicatechin (Hydroxy Sites: 3,5,7,3′,41J; Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole4-ethanaminium inner salt); Caffeic Acid Phenyl Ester; MCI-186 (3-Methyl-1-phenyl-2-pyrazolin-5-one); HBED (N,N′-Di-(2-hydroxybenzy\hat{ }ethylenediamine-HN′-diacetic acid<<H2O); Ambroxol (trans-4-(2-Amino-3,5-dibromobenzylamino)cyclohexane-HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol*2HCl); /5-1-5-methyl-nicotinamide-2′-deoxyribose; /S-D-I′-5-methyl-nico-tinamide-2′-deoxyribofuranoside; /3-1′-4,5-dimethyl-nicotinamide-2′-de-oxyribose; /3-D-1′-4,5-dimethyl-nicotinamide-2′-deoxyribofuranoside; 1-Naphthyl PP 1 (1-(1,1-Dimethyl ethyl)-3-(1-naphthalenyl)-1H-pyrazolo[3,4-d]pyrimidin-4-amine); Lavendustin A (5-[[(2,5-Dihydroxyphenyl)methyl][(2-hydroxyphenyl)methyl]amino]-2-hydroxybenzoic acid); MNS (3,4-Methylenedioxy-b-nitrostyrene); PP 1 (1-(1,1-Dimethylethyl)-1-(4-methylphenyl)-1H-pyrazolo[3,4-d]pyrimidin-4-amine); PP2 (3-(4-chlorophenyl) 1-(1,1-dimethylethyl)-1H-pyrazolo[3,4-d]pyrimidin-4-amine); KX1-004 (Kinex); KX1-005 (Kinex); KX1-136 (Kinex); KX1-174 (Kinex); KX1-141 (Kinex); KX2-328 (Kinex); KX1-306 (Kinex); KX1-329 (Kinex); KX2-391 (Kinex); KX2-377 (Kinex); ZD4190 (Astra Zeneca; N-(4-bromo-2-fluorophenyl)-6-methoxy-7-(2-(1H-1,2,3-triazol-1-yl)ethoxy)quinazolin-4-amine); AP22408 (Ariad Pharmaceuticals); AP23236 (Ariad Pharmaceuticals); AP23451 (Ariad Pharmaceuticals); AP23464 (Ariad Pharmaceuticals); AZD0530 (Astra Zeneca); AZM475271 (M475271; Astra Zeneca); Dasatinib (N-(2-chloro-6-methylphneyl)-2-(6-(4-(2-hydroxyethyl)-piperazin-1-yl)-2-methylpyrimidin-4-ylamino) thiazole-5-carboxamide); GN963 (trans-4-(6,7-dimethoxyquinoxalin-2ylamino)cyclohexanol sulfate); Bosutinib (4-((2,4-dichloro-5-methoxyphenyl)amino)-6-methoxy-7-(3-(4-methyl-1-piperazinyl)propoxy)-3-quinolinecarbonitrile); or combinations thereof.

### Radiolabels

In some embodiments, a cargo molecule comprises a radiolabel. As used herein, “radiolabel” refers to a moiety comprising a radioactive isotope of at least one element. In some embodiments, a radiolabel is one used in positron emission tomography (PET). In some embodiments, a radiolabel is one used in single-photon emission computed tomography (SPECT). In some embodiments, radioisotopes comprise ^{99m}Tc, ¹¹¹In, ⁶⁴Cu, ⁶⁷Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹¹C, ³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ⁶⁷Cu, ¹⁰⁵Rh, ¹¹¹Ag, ⁸⁹Zr, ²²⁵Ac, and ¹⁹²Ir.

### Neurotropic Factors

In some embodiments, a cargo molecule comprises a factor having neurotrophic properties (e.g., neurotrophic proteins such as nerve growth factor (NGF), brain-derived neurotrophic factor (BDNF), neurotrophin-3 (NT-3), neurotrophin-4 (NT-4), glial cell line-derived neurotrophic factor (GDNF), ciliary neurotrophic factor (CNTF) as well as non-protein small molecules with neurotrophic properties).

### III. Linkers

In some embodiments, a cargo molecule (e.g., a fluorescent moiety, photosensitizing agent, or drug) is directly attached to the peptides disclosed herein, e.g. at the end of the targeting peptide. Alternatively, in some embodiments, a cargo molecule (e.g., a fluorescent moiety, photosensitizing agent, or drug) is indirectly attached to a peptide disclosed herein, e.g., via a linker.

In some embodiments of the nerve targeting peptide conjugates (with or without a linker) described herein, the peptide comprises or consists or the amino acid sequence set forth in any one of SEQ ID NOS: 1-557. In some embodiments, the peptide comprises or consists of the amino acid sequence set forth in any one of SEQ ID NOS: 1-30. In some embodiments, the peptide comprises or consists of the amino acid sequence set forth in any one of SEQ ID NOS:31-36. In some embodiments, the peptide comprises or consists of the amino acid sequence set forth in any one of SEQ ID NOS:37-40. In some embodiments, the peptide comprises or consists of the amino acid sequence set forth in any one of SEQ ID NOS:41-240. In some embodiments, the peptide comprises or consists of the amino acid sequence set forth in any one of SEQ ID NOS:241-349. In some embodiments, the peptide comprises or consists of the amino acid sequence set forth in any one of SEQ ID NOS:350-449. In some embodiments, the peptide comprises or consists of the amino acid sequence set forth in any one of SEQ ID NOS:450-557.

In some embodiments, the peptide comprises or consists of the amino acid sequence set forth in any one of Formulas (I)-(XLIV) or Subformulas (Ia)-(XLIVa).

As used herein, a “linker” is any molecule capable of binding (e.g., covalently) to a targeting molecule disclosed herein. Linkers include, but are not limited to, straight or branched-chain carbon linkers, heterocyclic carbon linkers, amino acid linkers (e.g., D- or L-amino acid), lipophilic residues, peptide linkers, peptide nucleic acid linkers, hydrazone linkers, SPDB disulfide, sulfo-SPDB, maleimidomethyl cyclohexane-1-carboxylate (MCC), aminohexanoic acid linkers, and polyether linkers (e.g., PEG). For example, poly(ethylene glycol) linkers are available from Quanta Biodesign, Powell, OH. These linkers optionally have amide linkages, sulfhydryl linkages, or hetero functional linkages.

In some embodiments, the linker binds to a targeting molecule disclosed herein by a covalent linkage. A linker may connect a cargo molecule to the peptide by forming a covalent linkage to the cargo molecule at one location and a covalent linkage to the peptide at another location. The covalent linkages can be formed by reaction between functional groups on the linker and functional groups on the peptide and on the cargo molecule. In some embodiments, the covalent linkage comprises an ether bond, thioether bond, amine bond, amide bond, carbon-carbon bond, carbon-nitrogen bond, carbon-oxygen bond, or carbon-sulfur bond.

In some embodiments, the linker is flexible. In some embodiments, the linker is rigid. In some embodiments, the linker has segment(s) of flexibility and segment(s) of rigidity.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the linker is an alkyl. In some embodiments, the linker is a heteroalkyl.

In some embodiments, the linker is an alkylene. In some embodiments, the linker is an alkenylene. In some embodiments, the linker is an alkynylene. In some embodiments, the linker is a heteroalkylene.

An “alkyl” group refers to an aliphatic hydrocarbon group. The alkyl moiety may be a saturated alkyl or an unsaturated alkyl. Depending on the structure, an alkyl group can be a monoradical or a diradical (i.e., an alkylene group).

The “alkyl” moiety may have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group could also be a “lower alkyl” having 1 to 6 carbon atoms. The alkyl group of the compounds described herein may be designated as “Ci-C4 alkyl” or similar designations. By way of example only, “C1-C4 alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

In some embodiments, the linker comprises a ring structure (e.g., an aryl). As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyl groups include groups having from 3 to 10 ring atoms. Cycloalkyls include, but are not limited to, cyclopropyl, cyclobutyl, cyclopentyl, cyclohexyl, cycloheptyl, and cyclooctyl.

In some embodiments, the ring is a cycloalkane. In some embodiments, the ring is a cycloalkene.

In some embodiments, the ring is an aromatic ring. The term “aromatic” refers to a planar ring having a delocalized π-electron system containing 4n+2 π electrons, where n is an integer. Aromatic rings can be formed from five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

In some embodiments, the ring is a heterocycle. The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and faropyridinyl. The foregoing groups, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

In some embodiments, the ring is fused. The term “fused” refers to structures in which two or more rings share one or more bonds, hi some embodiments, the ring is a dimer. In some embodiments, the ring is a trimer. In some embodiments, the ring is a substituted.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e., a heteroatom). Heterocycle includes heteroaryl and heterocycloalkyl. Carbocycles and heterocycles can be optionally substituted.

In some embodiments, the linker is substituted. The term “optionally substituted” or “substituted” means that the referenced group may be substituted with one or more additional group(s) individually and independently selected from Ci-Cealkyl, C3-Cgcycloalkyl, aryl, heteroaryl, C2-C6heteroalicyclic, hydroxy, Ci-C6alkoxy, aryloxy, Ci-C6alkylthio, arylthio, Ci-C6alkylsulfoxide, arylsulfoxide, Ci-C6alkylsulfone, arylsulfone, cyano, halo, C2-C8acyl, C2-C8acyloxy, nitro, Ci-C6haloalkyl, Ci-C6fluoroalkyl, and amino, including Ci-C6alkylamino, and the protected derivatives thereof. By way of example, an optional substituents may be LSRS, wherein each Ls is independently selected from a bond, —O—, —C(═O)—, —S—, —S(═O)—, —S(O)2-, —NH—, —NHC(O)—, —C(O)NH—, S(O)2NH—, —NHS(O)2-, —OC(O)NH—, —NHC(O)O—, -(CpC6alkyl)-, or —(C2-C6alkenyl)-; and each Rs is independently selected from H, (Ci-C4alkyl), (C3-C8cycloalkyl), heteroaryl, aryl, and Ci-C6heteroalkyl. Optionally substituted non-aromatic groups may be substituted with one or more oxo (═O). The protecting groups that may form the protective derivatives of the above substituents are known to those of skill in the art.

In some embodiments, a bifunctional linker having one functional group reactive with a group on one molecule (e.g., a targeting molecule), and another group reactive on the other molecule (e.g., a fluorescent moiety or a drug), is used to form the desired conjugate. Alternatively, in some embodiments, derivatization is performed to provide functional groups. Thus, for example, procedures for the generation of free sulfhydryl groups on peptides are also known (See U.S. Pat. No. 4,659,839). A linker may alternatively comprise a heterobifunctional crosslinker comprising two or more different reactive groups that form a heterocyclic ring that can interact with a targeting molecule. For example, a heterobifunctional crosslinker such as cysteine may comprise an amine reactive group and a thiol-reactive group can interact with an aldehyde on a derivatized targeting molecule. Additional combinations of reactive groups suitable for heterobifunctional crosslinkers include, for example, amine- and sulfhydryl reactive groups; carbonyl and sulfhydryl reactive groups; amine and photoreactive groups; sulfhydryl and photoreactive groups; carbonyl and photoreactive groups; carboxylate and photoreactive groups; and arginine and photoreactive groups. Examples of a heterobifunctional crosslinker include N-Succinimidyl 4-(2-pyridyldithio)butanoate (SPDB) and maleimidomethyl cyclohexane-1-carboxylate (MCC).

In some embodiments, a linker is cleavable. In some embodiments, the linker is non-cleavable. A linker can be chemically stable to extracellular environments, for example, chemically stable in the blood stream, and/or may include linkages that are not stable. A linker can include linkages that are designed to cleave and/or immolate or otherwise breakdown specifically or non-specifically inside cells. A cleavable linker can be sensitive to enzymes at a specific site, such as by extracellular proteases.

A cleavable linker can include a valine-citrulline peptide, a valine-alanine peptide, a phenylalanine-lysine or other peptide, such as a peptide that forms a protease recognition and cleavage site. Such a peptide-containing linker can contain a pentafluorophenyl group. A peptide-containing linker can include a succimide or a maleimide group. A peptide-containing linker can include a para aminobenzoic acid (PABA) group. A peptide-containing linker can include an aminobenzyloxycarbonyl (PABC) group. A peptide-containing linker can include a PABA or PABC group and a pentafluorophenyl group. A peptide-containing linker can include a PABA or PABC group and a succinimide group. A peptide-containing linker can include a PABA or PABC group and a maleimide group.

A non-cleavable linker is generally protease-insensitive and insensitive to intracellular processes. A non-cleavable linker can include a maleimide group. A non-cleavable linker can include a succinimide group. A non-cleavable linker can be maleimido-alkyl-C(O)— linker. A non-cleavable linker can be maleimidocaproyl linker. A maleimidocaproyl linker can be N-maleimidomethylcyclohexane-1-carboxylate. A maleimidocaproyl linker can include a succinimide group. A maleimidocaproyl linker can include pentafluorophenyl group.

In some embodiments, a peptide linker consisting of one or more amino acids is used to join the targeting molecule and a fluorescent moiety or drug. Generally, the peptide linker will have no specific biological activity other than to join the molecules or to preserve some minimum distance or other spatial relationship between them. However, the constituent amino acids of the linker may be selected to influence some property of the molecule such as the folding, net charge, or hydrophobicity. In some embodiments the peptide linker is relatively short, typically less than about 10 amino acids, preferably less than about 8 amino acids and more preferably less than 5 amino acids. Non-limiting illustrative examples include glycine and glycine-serine linkers which can be added to the C-terminus of a peptide. In some embodiments, a peptide linker comprises a cysteine residue or an unnatural amino acid residue (e.g., selenocysteine (Sec), p-acetophenylalanine (pAcF), p-azidomethyl-L-phenylalanine (pAMF), and azido-lysine (AzK)) for site specific conjugation. In some embodiments, a peptide linker is a glycine-glycine-glycine-cysteine (GGGC) (SEQ ID NO:572) linker, a glycine-glycine-cysteine (GGC) linker, a glycine-glycine (GG) linker, or a cysteine (C) linker. In some embodiments, the GGGC (SEQ ID NO:572), GGC, GG, or C linker is added to the N-terminus or C-terminus of a peptide.

### IV. Further Modifications

In some embodiments, the peptides of the present disclosure are optionally conjugated to high molecular weight molecules that increase the multivalency and avidity of labeling. In some embodiments, the high molecular weight molecules are water-soluble polymers. Examples of suitable water-soluble polymers include, but are not limited to, peptides, saccharides, poly(vinyls), poly(ethers), poly(amines), poly(carboxylic acids) and the like. In some embodiments, the water-soluble polymers is dextran, polyethylene glycol (PEG), polyoxyalkylene, polysialic acid, starch, or hydroxyethyl starch. Any suitable method is used to conjugate peptides to water-soluble polymers (see, Hermanson G., Bioconjugate Techniques 2nd Ed., Academic Press, Inc. 2008). In some embodiments, the peptide-high molecular weight molecule conjugate further comprises a cargo molecule.

In some embodiments, one or more additional amino acid residues can be added to the N-terminus, the C-terminus, or both N-terminus and C-terminus of the peptides disclosed herein, thus producing a longer peptide. In some embodiments, 1, 2, 3, 4, 5, 6, 7, 8, 9, 10, 11, 12, 13, 14, 15 or more amino acid residues are added to the peptides disclosed herein. In some embodiments, about 1-15, 1-10, 1-5, or 1-3 amino acids are added to the peptides disclosed herein. In some embodiments, additional amino acids that are added to the peptides of the present disclosure may act as a spacer to provide a variable distance between the peptide and the cargo molecule.

In some embodiments, the nerve targeting peptide conjugates of the present invention are modified to increase solubility. Peptide modifications that increase solubility include addition of hydrophilic amino acid(s), a PEG moiety, or both. In some embodiments, a PEG moiety is 8-Amino-3,6-dioxaoctanoic acid (AEEA); 12-amino-4,7,10-trioxadodecanoic acid; or 15-amino-4,7,10,13-tetraoxapenta-decanoic acid. In some embodiments, about one to ten (e.g., one, two, three, four, five, six, seven, eight, nine, or ten) hydrophilic amino acids may be added to the N-terminus, C-terminus, an internal position, or any combination thereof, of the peptide component of the nerve targeting peptide conjugate to increase solubility. Hydrophilic amino acids include D, E, H, K, N, Q, R, S, T, and G. In some embodiments, the peptide comprises a K, KK, KKK, KKKK (SEQ ID NO:573), G, GG, GGG, or GGGG (SEQ ID NO:574) at the N-terminus or C-terminus.

### V. Pharmaceutical Compositions

Disclosed herein, in certain embodiments, are pharmaceutical compositions comprising a nerve targeting peptide conjugate disclosed herein. Pharmaceutical compositions herein are formulated using one or more physiologically acceptable carriers including excipients and auxiliaries which facilitate processing of the active agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N. Y., 1980; and Pharmaceutical Dosage Forms and Drug Delivery Systems, Seventh Ed. (Lippincott Williams & Wilkins, 1999).

In some embodiments of pharmaceutical compositions comprising the nerve targeting peptide conjugates described herein, the peptide comprises or consists or the amino acid sequence set forth in any one of SEQ ID NOS: 1-557. In some embodiments, the peptide comprises or consists of the amino acid sequence set forth in any one of SEQ ID NOS: 1-30. In some embodiments, the peptide comprises or consists of the amino acid sequence set forth in any one of SEQ ID NOS:31-36. In some embodiments, the peptide comprises or consists of the amino acid sequence set forth in any one of SEQ ID NOS:37-40. In some embodiments, the peptide comprises or consists of the amino acid sequence set forth in any one of SEQ ID NOS:41-240. In some embodiments, the peptide comprises or consists of the amino acid sequence set forth in any one of SEQ ID NOS:241-349. In some embodiments, the peptide comprises or consists of the amino acid sequence set forth in any one of SEQ ID NOS:350-449. In some embodiments, the peptide comprises or consists of the amino acid sequence set forth in any one of SEQ ID NOS:450-557.

In embodiments of pharmaceutical compositions comprising the nerve targeting peptide conjugates described herein, the peptide comprises or consists of an amino acid sequence set forth in any one of Formulas (I)-(XLIV) or Subformulas (Ia)-(XLIVa).

In certain embodiments, a pharmaceutical composition disclosed herein further comprises a pharmaceutically acceptable diluent(s), excipient(s), or carrier(s). In some embodiments, the pharmaceutical compositions include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

In certain embodiments, nerve targeting peptide conjugates disclosed herein are delivered to a subject via a drug delivery vehicle or carrier. In some embodiments, a delivery vehicle is made from natural or synthetic materials or both. In some embodiments, a delivery vehicle is a nanoparticle, microparticle, polymeric micelle, nanocapsule, dendrimer, large PEG, nanogel, liposome, fullerene, nanostructured lipid carrier, nanoshell, quantum dot, protein-based nanocarriers (e.g., albumin, elastin, gliadin, legumin, zein, soy protein, milk protein, whey based nanocarriers), organic nanocarrier (e.g., gelatin, dextran, guar gum, chitosan, collagen), polysaccharide based carrier (e.g., dextran, chitosan, pectin), lipid emulsion, or a combination thereof.

In certain embodiments, a pharmaceutical composition disclosed herein is administered to a subject by any suitable administration route, including but not limited to, topical, oral, intrarectal, intravaginal, intranasal, inhalation, parenteral (intravenous, intraarterial, subcutaneous, intraperitoneal, intramuscular, intravascular, intrathecal, intravitreal, infusion) administration. In certain embodiments, a pharmaceutical composition disclosed herein is administered to a subject is administered locally or systemically. In embodiments, a pharmaceutical composition disclosed herein is administered intravenously.

Formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Bioavailability enhancers may include penetration or permeation enhancers. See, e.g., Muheem et al., 2016, Saudi Pharm J. 24, 413-428; Brayden et al 2020, Adv Drug Deliv Rev. 157, 2-36; Ibrahim et al 2020, J Pham. Sci. 28, 403-416. In some formulations, proper fluidity can be maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents, as are known in the art. In embodiments, formulations suitable for intravenous injection may be prepared in aqueous solutions, such as saline buffers and other physiologically compatible buffers known in the art.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as sterile suspensions, solutions or emulsions in oily or aqueous vehicles, and contain formulating agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

In embodiments, a pharmaceutical composition herein is administered orally. Dosage forms suitable for oral administration can be solid or liquid and may include for example, a pill, capsule, troche, tablet, caplet, gel caplet (gel cap), syrup, an aqueous suspension or solution, a chewable form, a swallowable form, a dissolvable form, an effervescent, a granulated form, and an oral liquid solution. In a specific embodiment, the dosage form is a solid dosage form, and more specifically, comprises a tablet or capsule, or is orally administered by a vehicle or carrier as disclosed herein.

In some embodiments, the pharmaceutical composition described herein is in unit dosage forms suitable for single administration of precise dosages. In unit dosage form, the formulation is divided into unit doses containing appropriate quantities of an active agent disclosed herein. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

In some embodiments, the nerve targeting peptide conjugate is administered via systemic intravenous injection into human patients.

### VI. Methods of Use

In another aspect, the present disclosure provides methods of delivering a cargo molecule to a neuron or nerve, comprising contacting the neuron or nerve with the nerve targeting peptide conjugates or pharmaceutical compositions described herein. Such methods may be used to label or identify a neuron or nerve, delivery a drug to a neuron or nerve, or delivery a photosensitizing agent to a neuron or nerve.

In some embodiments, the contacting occurs in vivo. In some embodiments, the contacting occurs in vitro.

In some embodiments of the methods described herein, the nerve targeting peptide conjugates are administered to a subject, as described herein, including humans and mammals (e.g., mice, rats, pigs, cats, dogs, and horses). In some embodiments, subjects are mammals. In some embodiments, subjects are primates. In some embodiments, subjects are humans. In some embodiments, human subjects are pediatric subjects (age 21 years and younger), adult subjects (age 22 years to 65 years), or geriatric subjects (age 65 years and above).

In some embodiments, the peptide comprises or consists of the amino acid sequence set forth in any one of SEQ ID NOS: 1-30. In some embodiments, the peptide comprises or consists of the amino acid sequence set forth in any one of SEQ ID NOS:31-36. In some embodiments, the peptide comprises or consists of the amino acid sequence set forth in any one of SEQ ID NOS:37-40. In some embodiments, the peptide comprises or consists of the amino acid sequence set forth in any one of SEQ ID NOS:41-240. In some embodiments, the peptide comprises or consists of the amino acid sequence set forth in any one of SEQ ID NOS:241-349. In some embodiments, the peptide comprises or consists of the amino acid sequence set forth in any one of SEQ ID NOS:350-449. In some embodiments, the peptide comprises or consists of the amino acid sequence set forth in any one of SEQ ID NOS:450-557.

In some embodiments, a peptide comprises or consists of an amino acid sequence set forth in any one of Formulas (I)-(XLIV), including Subformulas (Ia)-(XLIVa).

In some embodiments, the present disclosure provides methods of labeling or identifying a neuron or nerve (or component of either) by contacting a neuron or nerve with nerve targeting peptide conjugate described herein.

In some embodiments, the cargo molecule of the nerve targeting peptide conjugate comprises a fluorescent moiety described herein. In some embodiments, the fluorescent moiety comprises a fluorescent protein, a fluorescent peptide, a fluorophore, or any combination thereof.

In some embodiments, a nerve targeting peptide conjugate is administered in combination (simultaneously, concurrently, or serially) with a fluorescent moiety (e.g., fluorescent moiety that is not conjugated to the nerve target peptide conjugate, or “free” fluorescent moiety). In some embodiments, the fluorescent moiety is a fluorescein, e.g., carboxyfluorescein.

In some embodiments of the methods of labeling or identifying a neuron or nerve described herein, the neuron or nerve is labeled for identification during surgery on the subject. In some embodiments, the surgery is related to a traumatic injury in the subject. In some embodiments, the surgery is related to an infection in the subject. In some embodiments, the surgery is plastic surgery e.g., cosmetic or reconstructive surgery, in the subject.

In some embodiments, the surgery is head, neck, skull base, spinal, prostate, heart, kidney, hand, arm, foot, leg, lower abdominal, or gynecological surgery.

In some embodiments, the surgery is head and neck surgery.

In some embodiments, the surgery is gastrointestinal tract (GI) surgery or genitourinary tract (GU) surgery.

In some embodiments the surgery is cancer surgery. In some embodiments the cancer is selected from the group consisting of prostate cancer, liver cancer (HCC), colorectal cancer, ovarian cancer, endometrial cancer, breast cancer, pancreatic cancer, stomach cancer, cervical cancer, head and neck cancer, thyroid cancer, testis cancer, urothelial cancer, lung cancer, melanoma, testicular germ cell tumors, mesothelioma, and esophageal cancer. In some embodiments, the cancer is prostate cancer.

In some embodiments, the method comprises administering a nerve targeting peptide conjugate disclosed herein to a subject that will undergo surgery. In some embodiments, the method comprises administering a nerve targeting peptide conjugate disclosed herein to a subject that is currently undergoing surgery. In some embodiments, a nerve targeting peptide conjugate disclosed herein is administered to a patient systemically, for example, by intravenous injection. In some embodiments, a nerve targeting peptide conjugate disclosed herein is administered to a patient locally.

Disclosed herein, in certain embodiments, are methods of targeted drug delivery. In some embodiments, a nerve targeting peptide conjugate disclosed herein delivers a drug to a specific target. In some embodiments, a nerve targeting peptide conjugate disclosed herein delivers a drug to a neuron or nerve.

In some embodiments, the drug is an agent that reduces pain (either the perception of pain or activity of a painful stimulant). In some embodiments, the drug is an anesthetic. In some embodiments, the drug is benzocaine; carticaine; cinchocaine; cyclomethycaine; lidocaine; prilocaine; propxycaine; proparacaine; tetracaine; tocainide; and trimecaine; or a combination thereof.

In some embodiments, the drug is an agent that modulates death (e.g., via apoptosis or necrosis) of a neuron or nerve. In some embodiments, the drug is a cytotoxic agent. In some embodiments, the drug is methotrexate (RHEUMATREX®, Amethopterin); cyclophosphamide (CYTOXAN®); thalidomide (THALIDOMID®); paclitaxel; pemetrexed; pentostatin; pipobroman; pixantrone; plicamycin; procarbazine; proteasome inhibitors (e.g.; bortezomib); raltitrexed; rebeccamycin; rubitecan; SN-38; salinosporamide A; satraplatin; streptozotocin; swainsonine; tariquidar; taxane; tegafur-uracil; temozolomide; testolactone; tbioTEPA; tioguanine; topotecan; trabectedin; tretinoin; triplatin tetranitrate; tris(2-chloroethyl)amine; troxacitabine; uracil mustard; valrubicin; vinblastine; vincristine; vinorelbine; vorinostat; zosuquidar; or a combination thereof. In some embodiments, the drug is a pro-apoptotic agent. In some embodiments, the drug is an anti-apoptotic agent. In some embodiments, the drug is selected from minocycline; SB-203580 (4-(4-Fluorophenyl)-2-(4-methylsulfmyl phenyl)-5-(4-pyridyl) 1H-imidazole); PD 169316 (4-(4-Fluorophenyl)-2-(4-nitrophenyl)-5-(4-pyridyl)-1H-imidazole); SB 202190 (4-(4-Fluorophenyl)-2-(4-hydroxyphenyl)-5-(4-pyridyl)1H-imidazole); RWJ 67657 (4-[4-(4-fluorophenyl)-1-(3-phenylpropyl)-5-(4-pyridinyl)-1H-imidazol-2-yl]-3-butyn-1-ol); SB 220025 (5-(2-Amino-4-pyrimidinyl)-4-(4-fluorophenyl)-1-(4-piperidinlyl)imidazole); D-JNKI-1 ((D)-hJIP 175_i 57-DPrO-DPrO-(D)-HIV-TAT57-48); AM-111 (Auris); SP600125 (anthra[1,9-cd]pyrazol-6(2H)-one); JNK Inhibitor I ((L)-HIV-T AT48-57-PP-JBD20); JNK Inhibitor III ((L)-HIV-TAT47-57-gaba-c-Jun833-57); AS601245 (1,3-benzothiazol-2-yl (2-[[2-(3-pyridinyl)ethyl]amino]-4 pyrimidinyl) acetonitrile); JNK Inhibitor VI (H2N-RPKRPTTLNLF-NH2) (SEQ ID NO:564); JNK Inhibitor VIII (N-(4-Amino-5-cyano-6-ethoxypyridin-2-yl)-2-(2,5-dimethoxyphenyl)acetamide); JNK Inhibitor IX (N-(3-Cyano-4,5,6,7-tetrahydro-1-benzothien-2-yl)-1-naphthamide); dicumarol (3,3′-Methylenebis(4-hydroxycoumarin)); SC-236 (4-[5-(4-chlorophenyl)-3-(trifluoromethyl)-1H-pyrazol-1-yl]benzene-sulfonamide); CEP-1347 (Cephalon); CEP-11004 (Cephalon); an artificial protein comprising at least a portion of a Bcl-2 polypeptide; a recombinant FNK; V5 (also known as Bax inhibitor peptide V5); Bax channel blocker ((+)-1-(3,6-Dibromocarbazol-9-yl)-3-piperazin-1-yl-propan-2-ol); Bax inhibiting peptide P5 (also known as Bax inhibitor peptide P5); Kp7-6; FAIM(S) (Fas apoptosis inhibitory molecule-short); FAIM(L) (Fas apoptosis inhibitory molecule-long); Fas: Fc; FAP-1; NOK2; F2051; F1 926; F2928; ZB4; Fas M3 mAb; EGF; 740 Y-P; SC 3036 (KKHTDDGYMPMSPGVA) (SEQ ID NO:565); PI 3-kinase Activator (Santa Cruz Biotechnology, Inc.); Pam3Cys ((S)-(2,3-bis(palmitoyloxy)-(2RS)-propyl)-N-palmitoyl-(R)-Cys-(S)-Ser(S)-Lys4-OH, trihydrochloride); Actl (NF-kB activator 1); an anti-DcB antibody; Acetyl-11-keto-b-Boswellic Acid; Andrographolide; Caffeic Acid Phenethyl Ester (CAPE); Gliotoxin; Isohelenin; NEMO-Binding Domain Binding Peptide (DRQIKIWFQNRRMKWKKTALDWSWLQTE) (SEQ ID NO:566); NF-kB Activation Inhibitor (6-Amino-4-(4-phenoxyphenylethylamino)quinazoline); NF-kB Activation Inhibitor II (4-Methyl-Nl-(3-phenylpropyl)benzene-1,2-diamine); NF-kB Activation Inhibitor III (3-Chloro-4-nitro-N-(5-nitro-2-thiazolyl)-benzamide); NF-kB Activation hihibitor IV ((E)-2-Fluoro-4′-methoxystilbene); NF-kB Activation Inhibitor V (5-Hydroxy-(2,6-diisopropylphenyl)-1H-isoindole-1,3-dione); NF-kB SN50 (AA VALLP A VLLALLAP VQRKRQKLMP) (SEQ ID NO:567); Oridonin; Parthenolide; PPM-18 (2-Benzoylamino-1,4-naphthoquinone); Rol06-9920; Sulfasalazine; TIRAP Inhibitor Peptide (RQIKIWFNRRMKWKKLQLRD AAPGGAIVS) (SEQ ID NO:568); Withaferin A; Wogonin; BAY 11-7082 ((E)3-[(4-Methylphenyl)sulfonyl]-2-propenenitrile); BAY 11-7085 ((E)3-[(4-t-Butylphenyl)sulfonyl]-2-propenenitrile); (E)-Capsaicin; Aurothiomalate (ATM or AuTM); Evodiamine; Hypoestoxide; IKK Inhibitor III (BMS-345541); IKK Inhibitor VII; IKK Inhibitor X; IKK Inhibitor II; IKK-2 Inhibitor IV; IKK-2 Inhibitor V; IKK-2 Inhibitor VI; IKK-2 Inhibitor (SC-514); IkB Kinase Inhibitor Peptide; IKK-3 Inhibitor LX; ARRY-797 (Array BioPharma); SB-220025 (5-(2-Amino-4-pyrimidinyl)-4-(4-fluorophenyl)-1-(4-piperidinlyl)imidazole); SB-239063 (trans-4-[4-(4-Fluorophenyl)-5-(2-methoxy-4-pyrimidinyl)-1H-imidazol-1-yl]cyclohexanol); SB-202190 (4-(4-Fluorophenyl)-2-(4-hydroxyphenyl)-5-(4-pyridyl) 1H-imidazole); JX-401 (-[2-Methoxy-4-(methylthio)benzoyl]-4-(phenylmethyl)piperidine); PD-169316 (4-(4-Fluorophenyl)-2-(4-nitrophenyl)-5-(4-pyridyl)-1H-imidazole); SKF-86002 (6-(4-Fluorophenyl)-2,3-dihydro-5-(4-pyridinyl)imidazo[2,1-b]thiazole dihydrochloride); SB-200646 (N-(1-Methyl-1H-indol-5-yl)-N′-3-pyridinylurea); CMPD-I (2′-Fluoro-N-(4-hydroxyphenyl)-[1,1′-biphenyl]-4-butanamide); EO-1428 ((2-Methylphenyl)-[4-[(2-amino-4-bromophenyl)amino]-2-chlorophenyl]methanone); SB-253080 (4-[5-(4-Fluorophenyl)-2-[4-(methylsulfonyl)phenyl]-1H-imidazol-4-yl]pyridine); SD-169 (1H-Indole-5-carboxamide); SB-203580 (4-(4-Fluorophenyl)-2-(4-methylsulfmyl phenyl)-5-(4-pyridyl) 1H-imidazole); TZP-101 (Tranzyme Pharma); TZP-102 (Tranzyme Pharma); GHRP-6 (growth hormone-releasing peptide-6); GHRP-2 (growth hormone-releasing peptide-2); EX-1314 (Elixir Pharmaceuticals); MK-677 (Merck); L-692,429 (Butanamide, 3-amino-3-methyl-N-(2,3,4,5-tetrahydro-2-oxo-1-((2′-(1H-tetrazol-5-yl)(1,1′-biphenyl)-4-yl)methyl)-1H-1-benzazepin-3-yl)-, (R)-); EP1572 (Aib-DTrp-DgTo-CHO); diltiazem; metabolites of diltiazem; BRE (Brain and Reproductive organ-Expressed protein); verapamil; nimodipine; diltiazem; omega-conotoxin; GVIA; amlodipine; felodipine; lacidipine; mibefradil; NPPB (5-Nitro-2-(3-phenylpropylamino)benzoic Acid); flunarizine; erythropoietin; piperine; hemin; brazilin; z-V AD-FMK (Benzyloxycarbonyl-Val-Ala-Asp(OMe)-fluoromethylketone); z-LEHD-FMK (benzyloxycarbonyl-Leu-Glu(OMe)-His-Asp(OMe)-fluoromethylketone) (SEQ ID NO:569); B-D-FMK (boc-aspartyl(Ome)-fluoromethylketone); Ac-LEHD-CHO (N-acetyl-Leu-Glu-His-Asp-CHO) (SEQ ID NO:569); Ac-IETD-CHO (N-acetyl-Ile-Glu-Thr-Asp-CHO) (SEQ ID NO:570); z-IETD-FMK (benzyloxycarbonyl-Ile-Glu(OMe)-Thr-Asp(OMe)-fluoromethyl ketone) (SEQ ID NO:570); FAM-LEHD-FMK (benzyloxycarbonyl Leu-Glu-His-Asp-fluoromethyl ketone) (SEQ ID NO:569); FAM-LETD-FMK (benzyloxycarbonyl Leu-Glu-Thr-Asp-fluoromethyl ketone) (SEQ ID NO:571); Q-VD-OPH (Quinoline-Val-ASp-CH2-O-Ph); XIAP; cIAP-1; cIAP-2; ML-IAP; ILP-2; NAIP; Survivin; Brace; IAPL-3; fortilin; leupeptine; PD-150606 (3-(4-Iodophenyl)-2-mercapto-(Z)-2-propenoic acid); MDL-28170 (Z-Val-Phe-CHO); calpeptin; acetyl-calpastatin; MG 132 (N-[(phenylmethoxy)carbonyl]-L-leucyl-N-[(1S)-1-formyl-3-methylbutyl]-L-leucinamide); MYODUR; BN 82270 (Ipsen); BN 2204 (Ipsen); AHLi-11 (Quark Pharmaceuticals), an mdm2 protein, pifithrin-α (1-(4-Methylphenyl)-2-(4,5,6,7-tetrahydro-2-imino-3(2H)-benzothiazolyl)ethanone); trans-stilbene; cis-stilbene; resveratrol; piceatannol; rhapontin; deoxyrhapontin; butein; chalcon; isoliquirtigen; butein; 4,2′,4′-trihydroxychalcone; 3,4,2′,4′,6′-pentahydroxychalcone; flavone; morin; fisetin; luteolin; quercetin; kaempferol; apigenin; gossypetin; myricetin; 6-hydroxyapigenin; 5-hydroxyflavone; 5,7,3′,4′,5′-pentahydroxyflavone; 3,7,3′,4′,5′-pentahydroxyflavone; 3,6,3′,4′-tetrahydroxyflavone; 7,3′,4′,5′-tetrahydroxyflavone; 3,6,2′,4′-tetrahydroxyflavone; 7,4′-dihydroxyflavone; 7,8,3′,4′-tetrahydroxyflavone; 3,6,2′,3′-tetrahydroxyflavone; 4′-hydroxyflavone; 5-hydroxyflavone; 5,4′-dihydroxyflavone; 5,7-dihydroxyflavone; daidzein; genistein; naringenin; flavanone; 3,5,7,3′,4′-pentahydroxyflavanone; pelargonidin chloride; cyanidin chloride; delphinidin chloride; (−)-epicatechin (Hydroxy Sites: 3,5,7,3′,4\hat{ }; (−)-catechin (Hydroxy Sites: 3,5,7,3′,4); (−)-gallocatechin (Hydroxy Sites: 3,5,7,3′,4′,5) (+)-catechin (Hydroxy Sites: 3,5,7,3′,4\hat{ }; (+)-epicatechin (Hydroxy Sites: 3,5,7,3′,40; Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-iniidazole4-ethanaminium inner salt); Caffeic Acid Phenyl Ester; MCI-186 (3-Methyl-1-phenyl-2-pyrazolin-5-one); HBED (N,N′-Di-(2-hydroxybenzyl)ethylenediamine-N,N′-diacetic acid*H2O); Ambroxol (trans-4-(2-Amino-3,5-dibromobenzylamino)cyclohexane-HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol>>2HCl); β-1-5-methyl-nicotinamide-2′-deoxyribose; /3-D-1′-5-methyl-nicotinamide-2′-deoxyribofuranoside; /3-1′-4,5-dimethyl-nicotinamide-2′-de-oxyribose; /3-D-1′-4,5-dimethyl-nicotmamide-2′-deoxyribofuranoside; 1-Naphthyl PPI (1-(1,1-Dimethylethyl)-3-(1-naphthalenyl)-1H-pyrazolo[3,4-d]pyrimidin-4-amine); Lavendustin A (5-[[(2,5-Dihydroxyphenyl)methyl][(2-hydroxyphenyl)methy 1]amino]-2-hydroxybenzoic acid); MNS (3,4-Methylenedioxy-b-nitrostyrene); PPI (1-(1,1-Dimethylethyl)-1-(4-methylphenyl)-1H-pyrazolo[3,4-d]pyrimidin-4-amine); PP2 (3-(4-chlorophenyl) 1-(1,1-dimethylethyl)-1H-pyrazolo[3,4-d]pyrimidin-4-amine); KX1-004 (Kinex); KX1-005 (Kinex); KX1-136 (Kinex); KX1-174 (Kinex); KX1-141 (Kinex); KX2-328 (Kinex); KX1-306 (Kinex); KX1-329 (Kinex); KX2-391 (Kinex); KX2-377 (Kinex); ZD4190 (Astra Zeneca; N-(4-bromo-2-fluorophenyl)-6-methoxy-7-(2-(1H-1,2,3-triazol-1-yl)ethoxy)quinazolin-4-amine); AP22408 (Ariad Pharmaceuticals); AP23236 (Ariad Pharmaceuticals); AP23451 (Ariad Pharmaceuticals); AP23464 (Ariad Pharmaceuticals); AZD0530 (Astra Zeneca); AZM475271 (M475271; Astra Zeneca); Dasatinib (N-(2-chloro-6-methylphneyl)-2-(6-(4-(2-hydroxyethyl)-piperazin-1-yl)-2-methylpyrimidin-4-ylamino) thiazole-5-carboxamide); GN963 (trans-4-(6,7-dimethoxyquinoxalin-2ylamino)cyclohexanol sulfate); Bosutinib (4-((2,4-dichloro-5-methoxyphenyl)amino)-6-methoxy-7-(3-(4-methyl-1-piperazinyl)propoxy)-3-quinolinecarbonitrile); or combinations thereof.

In some embodiments, the drug is an agent that reduces undesired neuron or nerve impulses. In some embodiments, the drug reduces one or more symptoms of dyskinesia or synkinesia. In some embodiments, the drug is carbamazepine, oxcarbazepine, phenytein, valproic acid, sodium valproate, cinnarizine, flunarizine, or nimodipine, or combinations thereof.

In some embodiments, the drug is an agent that promotes regeneration of neuron or nerve tissue. In some embodiments, the drug is a growth factor. In some embodiments, the drug is selected from brain-derived neurotrophic factor (BDNF); ciliary neurotrophic factor (CNTF); glial cell-line derived neurotrophic factor (GDNF); neurotrophin-3; neurotrophin-4; fibroblast growth factor (FGF) receptor; insulin-like growth factor (IGF); or a combination thereof.

Disclosed herein, in certain embodiments, are methods of delivering a photosensitizing agent to a human neuron or nerve comprising: contacting the human neuron or nerve with a nerve targeting peptide conjugate comprising (a) a nerve targeting peptide comprising the amino acid sequence of any one of SEQ ID NOS: 1-557, and (b) a photosensitizing agent. In some embodiments, the method further comprises exposing the human neuron or nerve with a light source that activates the photosensitizing agent, wherein the activated photosensitizing agent induces ablation or killing of the human neuron or nerve. Upon exposure to a specific wavelength of light, a photosensitizing agent reacts with molecular oxygen to produce singlet oxygen, which is cytotoxic. In certain embodiments, a photosensitizing agent is a porphyrin, chlorin, or dye. Examples of photosensitizing agents include porphyrin, protoporfin IX, purlytin, verteporfin, HPPH, temoporfin, methylene blue, photofrin, protofrin, hematoporphyrin, Talaporfin, benzopophyrin derivative monoacid, 5-aminileuvolinic acid, Lutetium texaphyrin, metallophthalocyanine, metallo-naphthocyaninesulfobenzo-porphyrazines, metallo-naphthalocyanines, zinc tetrasulfophthalocyanine, bacteriochlorins, metallochlorins, chlorine derivative, Tetra(m-hydroxyphenyl)chlorin (mTHPC), pheophorbide, dibromofluorescein (DBF), IR700DX, naphthalocyanine, and porphyrin derivatives.

Human neuron or nerve targeting molecules comprising a photosensitizing agent as disclosed herein can be used in methods of localized nerve killing in a subject. In some embodiments, human neuron or nerve targeting molecules comprising a photosensitizing agent are used for treating chronic pain (e.g., back, neck, or joint pain) in subject. In some embodiments, human neuron or nerve targeting molecules comprising a photosensitizing agent are used for treating prostate cancer in a subject. Autonomic innervation may contribute to prostate cancer growth and metastasis by light induced ablation of local autonomic nerves. Thus, local autonomic nerves may be a viable target for prostate cancer therapy. In some embodiments, human neuron or nerve targeting molecules comprising a photosensitizing agent are used for treating renovascular hypertension in a subject by light induced ablation of sympathetic nerves in the renal vessels. In some embodiments, human neuron or nerve targeting molecules comprising a photosensitizing agent are used for treating excessive sweating. In some embodiments, human neuron or nerve targeting molecules comprising a photosensitizing agent are used for treating cardiac arrhythmias. In some embodiments, human neuron or nerve targeting molecules comprising a photosensitizing agent are used for treating pathological muscle spasms (e.g., Meige syndrome, hemifacial spasm, torticollis).

In some embodiments, a first nerve targeting peptide conjugate is administered in combination (simultaneously, concurrently, or serially) with a second nerve targeting peptide conjugate. In yet further embodiments, the cargo molecule of the first nerve targeting peptide conjugate and the cargo molecule of the second nerve targeting peptide conjugate are each independently selected from a fluorescently moiety, drug, photosensitizing agent, neurotrophic factor, and radiolabel. In some embodiments, the cargo molecule of the first nerve targeting peptide conjugate and the cargo molecule of the second nerve targeting peptide conjugate are the same or different.

### EXAMPLES

### Example 1: Phage Display Library Construction, Screening, and Identification of Nerve-Binding Peptides

Screens of phage display libraries were used to identify peptides with selective binding for nerves versus muscle and therefore may be useful for systemic in-vivo labeling of nerves during fluorescence assisted surgery, as well as other nerve targeting applications.

### Construction of Library

Several m13 phage libraries were constructed expressing randomized variants of a parent peptide (QVPWEEPYYVVKKS-SGGL; SEQ ID NO: 576). In particular, the first 14 amino acids of the parent peptide were randomized using degenerate oligonucleotide synthesis to obtain a diverse set of peptides with a mutation rate of about 50% per residue.

To accomplish this, synthesis was carried out by the XXK codon design approach, where X is any base and K is G or T. To bias synthesis towards the parent sequence, synthesis was carried out at each X position with a mixture of about 70%-85% of the parent nucleotide and 15-30% of the remaining 3 nucleotides. The resulting molecules were inserted into fADL-1e (Antibody Design Laboratories), which is a type 3 phage display vector having a cloning site on the N-terminal side of the full-length gene III protein. The fusion proteins are directed to the periplasm by the PelB leader peptide, which is inserted in the vector in place of the wild-type gene III protein. During the phage assembly process, the peptides are displayed on the surface of the nascent phage particles.

Collectively, the phage display libraries used in the screens comprised about 600 million peptides, with sequencing analysis confirming a mutation rate of about 50% per residue.

### Screening and Sequencing

Phage libraries were independently processed, as follows, with five biological samples: two independent samples of excised human nerve tissue, one sample of a human nerve extract, and two independent samples of human muscle tissue, as follows:

Excised Human Nerve Tissue: For each of the two independent samples, phage were processed through multiple cycles of selection, with representative phage being isolated and sequenced after each selection cycle. Specifically, for selection using human nerve tissue, a sample of nerve muscle tissue (2 mm by 2 mm) was washed prior to mixing and incubation with a phage library. After incubation, the mixture (containing mostly intact nerves with phage particles that had variable affinity for nerves) was centrifuged and the nerve pellet washed multiple times with PBS to remove phage with little or no affinity for nerve tissue. The pellet was homogenized and plated for titering and reamplification. Phage that were bound at each round were sequenced. NextGen sequencing of pools after 2, 3 and 4 rounds of selection were used to isolate candidate sequences that were further analyzed using bioinformatics.

Human Nerve Extracts: Phage libraries were incubated for up to 24 hours at 4° C. in 96 well plates that had been coated with a human nerve extract. Plates were then washed with PBS and phage were eluted using 0.2M glycine pH 2.0. Phage supernatants where then neutralized with Tris pH 9.0 prior to plating on TG1 bacteria and LB agar plates. Colonies were counted to determine titer followed by DNA preparation of pools of phage and NextGen sequencing. After rounds of selection, phage were pooled and amplified for iterative selection. Phage that eluted by 0.2M glycine pH 2.0 (the nerve binders) at each round were sequenced. NextGen sequencing of pools after 2, 3 and 4 rounds of selection were used to isolate candidate sequences that were further analyzed using bioinformatics.

Human Muscle Tissue: For each sample, phage were processed through multiple cycles of selection, with representative phage being isolated and sequenced after each selection cycle. Specifically, for selection using human skeletal muscle tissue, a sample of skeletal muscle tissue (2 mm by 2 mm) was washed prior to mixing and incubation with a phage library. After incubation, the mixture (containing mostly intact muscle with phage particles that had variable affinity for muscle) was centrifuged and the muscle pellet washed multiple times with PBS to remove phage with little or no affinity for muscle tissue. The pellet was then homogenized and plated for titering and reamplification. Phage that were bound at each round were sequenced.

NextGen sequencing of pools after 2, 3 and 4 rounds of selection were used to isolate candidate sequences from each of the five samples. These five data sets were then analyzed as described in the next section.

### Processing of Peptide Sequences

A python script was used to translate, trim, and filter the peptide sequences:

Peptide and vector sequences were translated starting at the first start codon in the nucleotide sequence. The leader sequence, comprising the first 22 amino acids, was trimmed from the translated protein, the following 14 amino acids were saved as the displayed binding peptide, and the remaining amino acids after the peptide (derived from the vector) were also trimmed. Trimmed peptides with identical sequences were combined and quantified according to their frequency (count).

The script then removed peptide sequences that were likely artifacts (or otherwise not useful) by filtering out: 1) sequences without a start codon, 2) sequences with more than two changes in the leader sequence, 3) C-terminal sequences (after the peptide) that did not start with amino acids “SGGGG” (SEQ ID NO:575) or were of incorrect length except if they had an extra or missing “SGGGG” (SEQ ID NO:575), 4) peptides that had a single or an odd number of Cysteines, 5) peptide sequences that were truncated by stop codons, and 6) peptide sequences that occurred less than six times in a dataset.

Table 1 summarizes the number of sequences corresponding to these processing steps for each of the 5 data sets:

TABLE 1

| | ABMR2 | ABMCR3 | ABNCR2 | ABNCR3 | ABNPR2 |
|---|---|---|---|---|---|
| | (Muscle2) | (Muscle3) | (Nerve2) | (Nerve3) | (Plated Nerve2) |
| seqs | 52215 | 25520 | 52632 | 26498 | 34022 |
| no_start | 9 | 3 | 21 | 5 | 10 |
| incorrect_leader | 690 | 533 | 592 | 525 | 602 |
| length_error | 1887 | 1184 | 1840 | 1129 | 1464 |
| C-terminal error | 2715 | 2074 | 2647 | 1895 | 2259 |
| odd cysteines | 2773 | 395 | 2902 | 1134 | 1441 |
| has stop codon | 318 | 263 | 268 | 218 | 241 |
| combined peptide | 24106 | 4215 | 33093 | 6629 | 10484 |
| total | | | | | |
| unique peptides | 4307 | 258 | 2064 | 366 | 2809 |
| (count of at least 6) | | | | | |

### Assessing Nerve Versus Muscle Binding

Following processing, an R script was written to merge the unique peptide sequences (with counts 6 or greater) from the 5 different data sets to analyze the overlap between nerve and muscle binding activity.

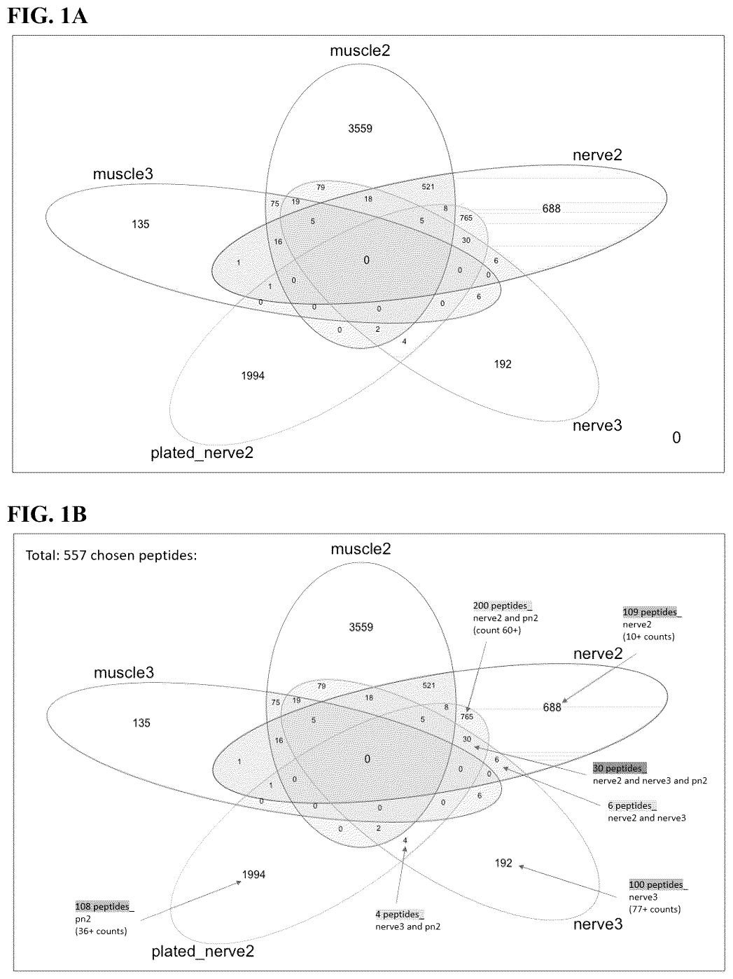

FIG. 1A shows a Venn diagram, showing a high resolution overlap between the five data sets. Collectively, the analysis revealed 2679 unique peptides binding to nerve versus muscle, 3769 unique peptides binding to muscle versus nerve, and 681 unique peptides binding to both muscle and nerve. Peptide sequences within each group were ranked by unique sequence count, that is, their relative enrichment in each set.

### Obtaining a Representative Set of Nerve-Selective Peptides

A representative group of 557 nerve-specific peptides was obtained from all 7 subsets of binding data, as shown in FIG. 1B. For the single Nerve2, Nerve3, and Plated Nerve2 (pn2) subsets, the number of counts for each group was set at a threshold giving rise to at least 100 peptides. As highlighted in gray in FIG. 1B, this resulted in 109 unique nerve 2 peptides (having at least 10 counts); 100 unique nerve 3 peptides (having at least 77 counts); and 108 plated nerve 2 peptides (having at least 36 counts). As highlighted in yellow in FIG. 1B, the representative group also included 6 unique peptides overlapping between nerve2 and nerve3, 200 unique peptides overlapping between nerve2 and plated nerve2 (at least 60 counts), 4 unique peptides overlapping between nerve3 and plated nerve2. Finally, as highlighted in green in FIG. 1B, the representative group also included 30 unique peptides common to the nerve2, nerve3, and plated nerve2 sets.

Tables 2A-2G show the peptide sequences and corresponding counts of the 7 groups making up the representative sample of 557 peptides.

TABLE 2A

| SEQ ID | Nerve2_Nerve3_ | |
|---|---|---|
| NO: # | Plated Nerve 2 | Count |
| 1 | TIPYAEPYYVVRKY | 711 |
| 2 | DFTMQEPYYAEKKT | 593 |
| 3 | HVTWQEQYYVLKTP | 371 |
| 4 | HVPGVDPYSVVKKF | 368 |
| 5 | HVAFEQPDNVLVLY | 357 |
| 6 | QVPTEVQFYLVKEP | 337 |
| 7 | QLPSEQPCCVVKKY | 329 |
| 8 | QDPWVTRYYIVKKS | 304 |
| 9 | QVPAKEPYNFMKKS | 299 |
| 10 | YLTWEEPEHVNKQS | 272 |
| 11 | QVPRQELYYVSHQF | 245 |
| 12 | QHPFEQSFYVVNKF | 227 |
| 13 | DILLDELRFAEKHG | 203 |
| 14 | YNLWDAPSYLVKNS | 201 |
| 15 | HVAGGERYFSVKES | 185 |
| 16 | HLPSDQPLSVTKIS | 173 |
| 17 | GFLGEEPYHFLAKR | 167 |
| 18 | LRSTADAHYTLKKS | 152 |
| 19 | ATPCSQPCAVVPKF | 142 |
| 20 | QYAWEEPYYLEKKL | 140 |
| 21 | HLPVEYPFYVFKNS | 131 |
| 22 | RGSLEGPFKLGKTH | 130 |
| 23 | QVSSQEPNFIVKNN | 116 |
| 24 | HFSAYEQYYVLRNS | 111 |
| 25 | YVIKYAPYYAVTRS | 93 |
| 26 | HLPEEELYTIVRKT | 85 |
| 27 | QDPCVWPYNGVKKC | 79 |
| 28 | HVHWDATYYAVTNS | 77 |
| 29 | QFPYDHKNYVVKYS | 69 |
| 30 | RFHMVGPYNVATTS | 68 |

TABLE 2B

| SEQ ID | | |
|---|---|---|
| NO: # | Nerve2_Nerve3 | Count |
| 31 | HLPWEGQLNIGNKS | 357 |
| 32 | DDLWQDLYGVVKKS | 106 |
| 33 | NLAWAESKFLLEKI | 71 |
| 34 | QLRYQEHYYLGYNS | 56 |
| 35 | YVPWQEPYYVQKNL | 49 |
| 36 | LVPWDETHKVVQRH | 14 |

TABLE 2C

| SEQ ID | Nerve3_Plated | |
|---|---|---|
| NO: # | Nerve 2 | Count |
| 37 | HVALEQPYYAVKES | 342 |
| 38 | QVQCKEQYYGVTNC | 195 |
| 39 | QALYAEPAYVSKKF | 19 |
| 40 | HVSMDERYYVVKKT | 17 |

TABLE 2D

| SEQ ID | Nerve2_Plated | |
|---|---|---|
| NO: # | Nerve2 | Count |
| 41 | HSYREEPYYFGKRS | 632 |
| 42 | QHTWKEPYNGQKRT | 577 |
| 43 | QLPMGGPYERVNNT | 380 |
| 44 | SVPKVHPYYVVNNS | 333 |
| 45 | QVPTGEPLYPVQKT | 323 |
| 46 | QLPSYQAHDVMKVH | 308 |
| 47 | QIPSQRPSSVGAIA | 292 |
| 48 | QFPGDERYYLATST | 283 |
| 49 | HVSSQEPYYVLRNS | 252 |
| 50 | RLPSDDHNTFGTRS | 249 |
| 51 | QLHGDELLSVVKNP | 232 |
| 52 | ATEWEESYFHVKKF | 223 |
| 53 | RVPWEQSSWVVKRS | 220 |
| 54 | QIANKEPHTYVENS | 213 |
| 55 | QIRSEVTHSVVRES | 213 |
| 56 | QHTLNHPYSVVKTS | 205 |
| 57 | QVPSHELYFRPKES | 204 |
| 58 | EVPGGVPYYPMKKF | 201 |
| 59 | HVSGGQWVHGQKNS | 200 |
| 60 | HVPSKQMNSLAKKS | 196 |
| 61 | QIHFGEPNYVVKKS | 194 |
| 62 | QFPWEGSYYTAKQS | 193 |
| 63 | QIPWGEAYQAEKIT | 191 |
| 64 | VVPSNAQHLAPNMS | 191 |
| 65 | HVAEAESYNVVKKT | 187 |
| 66 | QIPCEGPYSGVQMC | 186 |
| 67 | HITGGVPYPVVRDS | 185 |
| 68 | HVLWEDSSYHVTHH | 179 |
| 69 | HVTYDEQKYFVKKS | 179 |
| 70 | QVPSVQPYYIGKNT | 175 |
| 71 | QARFEQPYSGLRDS | 167 |
| 72 | LIPSAYPHSLWSKS | 166 |
| 73 | QLRSNEAYHVVKKS | 166 |
| 74 | QTQMQEPKYGVSHS | 163 |
| 75 | DVRWDEPYYVDEKI | 161 |
| 76 | HVPRVEPYNVVTNY | 158 |
| 77 | QFPGQALSFVHEKS | 152 |
| 78 | HFPSKALNNTAKNT | 151 |
| 79 | QFPPEQPYGLANKS | 150 |
| 80 | HVPWAGRHYVAKHS | 149 |
| 81 | RVAIDELYLIAHKS | 147 |
| 82 | QIPEQQPYNVFKKS | 142 |
| 83 | SIPGEDSLYAVTEH | 142 |
| 84 | QCPSEQPNYVCKIF | 140 |
| 85 | HGQTQEPSYVTVYY | 139 |
| 86 | HVHWQVPSNIVRNS | 139 |
| 87 | QLPWYEADYRMEYY | 139 |
| 88 | QLPSQAQNYYVTAS | 137 |
| 89 | QVTSEEHYFAVKKF | 136 |
| 90 | TSLSGAPFHNANTF | 134 |
| 91 | HDASIALYDPVLMS | 133 |
| 92 | QLPGDVTYNLGHKS | 133 |
| 93 | QPTWEQPYHVDRNS | 131 |
| 94 | HIPFPGLYSNLDQS | 127 |
| 95 | DHPWDVRHYAHKKS | 126 |
| 96 | DVPWKESFNIVENS | 126 |
| 97 | QFARTAPYYVQTEY | 125 |
| 98 | QNPWQKPYTVMKTS | 123 |
| 99 | HLSFEEPLYGVKKS | 122 |
| 100 | QFPSGHRVYTGKMT | 122 |
| 101 | YNLYEQPYSVLKRS | 122 |
| 102 | HLPSQVTHYVITSF | 115 |
| 103 | QVPSHEPFYLGKKY | 114 |
| 104 | RVTQGSPVYGENNA | 114 |
| 105 | QIPMEETYSLRKMS | 113 |
| 106 | QIPWEAPSYGATIS | 113 |
| 107 | HVGWEQPYYVLRKS | 112 |
| 108 | DFATEERRYAVPKY | 111 |
| 109 | DVPLVKPHYGAVDS | 111 |
| 110 | QFPSEASHYVVRNS | 111 |
| 111 | HSRLHETSYVFNKP | 110 |
| 112 | HVQRQETDYAAKRI | 110 |
| 113 | NVLSEEPSSRDANV | 110 |
| 114 | HLPFEAPYYAVKRS | 109 |
| 115 | QLPGEEPTYVGSNS | 109 |
| 116 | EVPVGDLSYVGKKY | 108 |
| 117 | QFPLVQPNYLVEMP | 108 |
| 118 | HVPGQDSYNVVNKF | 107 |
| 119 | HVTYTEASVYKRTF | 107 |
| 120 | QLEWEEPHYAARKS | 107 |
| 121 | HYQSVKSYSVQKKI | 105 |
| 122 | QLTSAEASYVGKQS | 105 |
| 123 | HRPWEEPYYVAKQS | 104 |
| 124 | LFASENPYNVKSKS | 103 |
| 125 | SVPGEDPSQFGKTP | 103 |
| 126 | HVPSEEPYKLDEQS | 102 |
| 127 | QFRIEDSYDVANQS | 102 |
| 128 | QIYWHESNYLVTKS | 102 |
| 129 | HIPGDETSYVMKKY | 100 |
| 130 | QSLSEEPQYVVTPS | 98 |
| 131 | HILLEEPSNVLNKR | 96 |
| 132 | TLPVYVPYYVVNNT | 96 |
| 133 | HMRSEQPYYGGKVS | 95 |
| 134 | EVPRTERNFVVKNT | 94 |
| 135 | HGAWEETHYVVIQS | 94 |
| 136 | HGRSVESYDLVNKR | 94 |
| 137 | QVTWGEPQYVVYKS | 94 |
| 138 | QLPSGEPYSFVKES | 93 |
| 139 | DVPLEDTYYRVKKY | 91 |
| 140 | HVPFDKSSHLVKKY | 90 |
| 141 | QFAWDSPYSRGKKT | 90 |
| 142 | HVTWQQTKYVTNRS | 89 |
| 143 | RFPLEEPNYFQTKS | 89 |
| 144 | HVTWGKTYHLVTDS | 88 |
| 145 | LVPWEEPGYFONTS | 88 |
| 146 | QYPREEPRHALKMS | 88 |
| 147 | NVTFEQSSYVVTSP | 87 |
| 148 | YVTSESPYYVHGQP | 87 |
| 149 | HVSGTKPYHLVKKS | 86 |
| 150 | EDVLHNAYHVETQF | 85 |
| 151 | QFAWEHRDYVLQRS | 85 |
| 152 | QVLWEERLYVVKNS | 83 |
| 153 | RSTWEQSYTIVKES | 83 |
| 154 | QMASKEPNYPVKNS | 82 |
| 155 | QNPAEEQYIVWNNS | 82 |
| 156 | SHPWEKQYDVFNNS | 81 |
| 157 | DVPWEEPYHVGNWR | 80 |
| 158 | HVASEQPYYGVNRP | 80 |
| 159 | HVLGEEPYYFVQHV | 80 |
| 160 | QEPWDEFSYNVNAL | 80 |
| 161 | QFRSGEAYVVVQTS | 80 |
| 162 | QNAFSKPYNGVKPS | 80 |
| 163 | QIESGEPYHDQKHP | 79 |
| 164 | QVRWPEQYYVVNMG | 79 |
| 165 | QYAREEPYYFMSYK | 79 |
| 166 | QIPCQSDHNVVTNC | 78 |
| 167 | DRLSDEPFKGMTHS | 77 |
| 168 | DVRWSDPHDRITKS | 77 |
| 169 | HLPWEGPEYLGKKS | 77 |
| 170 | HVPRDEPQSFVHRS | 77 |
| 171 | QFSSAEAYTVVNES | 77 |
| 172 | QIPSHSPYYALNSS | 77 |
| 173 | QQRLSASYQHVIKS | 77 |
| 174 | HFEGEDLYDRVKSR | 76 |
| 175 | HVTLEKPYDVATKS | 76 |
| 176 | QLPGEHPYYPIKKP | 76 |
| 177 | EALWYKHYYGVHKT | 75 |
| 178 | HVVRHYPPHFVHKS | 75 |
| 179 | LIPSEERSYAVINT | 75 |
| 180 | QHTHKQPFNVVKNY | 75 |
| 181 | VVPSQGTYHVGMTS | 75 |
| 182 | AVAWERPYYVVNHS | 74 |
| 183 | DVLLARTYYFGEFP | 74 |
| 184 | HFAGQEHLHFVEIS | 74 |
| 185 | LFAWAEANNVMKNS | 74 |
| 186 | SITNKQSVYVLKKS | 73 |
| 187 | HVQSANPYYDVRNS | 72 |
| 188 | HVSFDEPYYYVKKS | 72 |
| 189 | QDYWAKPSYIVKHS | 72 |
| 190 | QISLTEATLVVRKS | 72 |
| 191 | DVLWEEPRYFVNNS | 71 |
| 192 | HAHGQETYFLVKQS | 71 |
| 193 | HFALQDPYQVFKKT | 71 |
| 194 | QYPAERPTFDVTNA | 71 |
| 195 | SLPMEERYYAVRTA | 71 |
| 196 | DVEWEEPYYFLEKS | 70 |
| 197 | DVTWEEPYYAVKNS | 69 |
| 198 | EHPRVESVYLFPHT | 69 |
| 199 | HDSLDLHYYAMHES | 69 |
| 200 | QCPLEESCFDGQKS | 69 |
| 201 | QLTWPKQNYTMTQS | 69 |
| 202 | QLPWGAPYYVVKNS | 68 |
| 203 | RLPSEEPYTVAKRP | 68 |
| 204 | DFPWEQRSSTVKQY | 67 |
| 205 | DHTRVHPYYDFEIP | 67 |
| 206 | HATLQEPYFVVNHY | 67 |
| 207 | HDTWKGPYYPANKS | 67 |
| 208 | QLPSQEPYNVQRKS | 67 |
| 209 | SVTRYDLSTDVTKS | 67 |
| 210 | ESQWEQPSDVFKVV | 66 |
| 211 | KLPLEEPFYIVTPS | 66 |
| 212 | QIRWEDASYVEKTS | 66 |
| 213 | QVPWKGQYYVVASS | 66 |
| 214 | HGPTDGPYYVSKKS | 65 |
| 215 | HILRQEPYYVLTNY | 65 |
| 216 | HSHGQELFYVVTNT | 65 |
| 217 | HVPWGEPEALVKKS | 65 |
| 218 | QPPREQPYHIMKNS | 65 |
| 219 | QTSWAGPHDRVTKA | 65 |
| 220 | QVPCEEPWCLVKVN | 65 |
| 221 | QVPWQDPVYDVNNA | 65 |
| 222 | YVKTVNPDSVLKIS | 65 |
| 223 | HVHFTDPHYVMKKS | 64 |
| 224 | HVPLQGPYYVKQKP | 64 |
| 225 | NVEWEIPYYFVKKF | 64 |
| 226 | QFAWVQPYYFAKKS | 64 |
| 227 | QHPVKERDEFWKES | 64 |
| 228 | QIPWGETTAGIKKL | 64 |
| 229 | EFSREQPNDFLNKS | 63 |
| 230 | HIPFAEHDGVVPIS | 63 |
| 231 | HVTSHEARYLAQNS | 63 |
| 232 | EFTWEGLSEGVNEP | 62 |
| 233 | HVSSEYPYYVVKHT | 62 |
| 234 | HVPAEPLVYENQKS | 61 |
| 235 | LIVSQPSYLAVQYS | 61 |
| 236 | QLPLAKPNSVVATS | 61 |
| 237 | RLAWEEPHYVVKKS | 61 |
| 238 | KFTTQDSYYFGTQT | 60 |
| 239 | NVTSREPYHLLEKS | 60 |
| 240 | QLPFIGPYHVAKKS | 60 |

TABLE 2E

| SEQ ID | | |
|---|---|---|
| NO: # | Nerve2 | Count |
| 241 | HDASGVSYYDRRES | 21 |
| 242 | HVGLPEPHHVMIHS | 20 |
| 243 | EVPSLQPYHGGKKS | 19 |
| 244 | QIPSQEPYHRFKES | 19 |
| 245 | QIPGEAPYSFGKKY | 18 |
| 246 | QNPWHYPYSLVSTA | 17 |
| 247 | DVPFRLWNFAVVDS | 15 |
| 248 | HFAWVVPNDAVKAS | 15 |
| 249 | HFSWEERYRVVHKS | 15 |
| 250 | RVPSDEPYSFLRQS | 15 |
| 251 | TQRWAEPYHVFHKS | 15 |
| 252 | HVLRHEPYYVVAQA | 14 |
| 253 | LLPSDEAHYIVKHS | 14 |
| 254 | NVLSEQPGYVMEYL | 14 |
| 255 | QLPWTNPFHVVNKY | 14 |
| 256 | QYSLDESPYGAKKS | 14 |
| 257 | YVPGAAPIGIVHKS | 14 |
| 258 | HFAWEEVFHVKKNF | 13 |
| 259 | HSPSKESHYVLENM | 13 |
| 260 | HVPFGGTYSVEGES | 13 |
| 261 | HYRCVGSCYVVAKS | 13 |
| 262 | LFRSQAQNVLVISH | 13 |
| 263 | QFPGSELTVSVIQV | 13 |
| 264 | QFSSQEAHYELNTP | 13 |
| 265 | QSPWDEPYYGVLMA | 13 |
| 266 | QSTPEERYHRVIQA | 13 |
| 267 | QVPLGAPFYAVRKS | 13 |
| 268 | YERWEAPFYDVQKS | 13 |
| 269 | HVAWNEPHHVLLQI | 12 |
| 270 | HVLWEDPYYVVRMP | 12 |
| 271 | HVQLREHYFVVYSS | 12 |
| 272 | HVSSEAPYYVLNKS | 12 |
| 273 | LFPLDETYYVAKVS | 12 |
| 274 | QALLEEKYTALKKS | 12 |
| 275 | QFPWEQRNYDVKKY | 12 |
| 276 | QIPLDARSYLLKRP | 12 |
| 277 | QIQREEPFYVLKLY | 12 |
| 278 | QLPSKASNSVQQTF | 12 |
| 279 | QLPWEKPYYDVEVT | 12 |
| 280 | QNPGKSPYYVVHKS | 12 |
| 281 | QVWGEVPLSGAKRF | 12 |
| 282 | RVPSAAAYYGAKIS | 12 |
| 283 | VFSSDSQYLFVPNF | 12 |
| 284 | DRPVEVPSDFVNNS | 11 |
| 285 | DVLGIERYYAVKDP | 11 |
| 286 | HHPGVGPFHVVKRS | 11 |
| 287 | HLSQYEPYDPVDKS | 11 |
| 288 | HVAWDGAYNLVQTT | 11 |
| 289 | HVPGQDPYDVQATY | 11 |
| 290 | HVPLEGSYLDDINS | 11 |
| 291 | HVTLAEPHLLLGKS | 11 |
| 292 | KFSLVHPEYYGKET | 11 |
| 293 | NLPGVEQYNLPNSS | 11 |
| 294 | NVPWQVPYSVLKMP | 11 |
| 295 | QFAGEVPYYFATDL | 11 |
| 296 | QHPMDPTYHVVIKA | 11 |
| 297 | QILSEEPYYRVDMP | 11 |
| 298 | QIPLAYSNYVVNKY | 11 |
| 299 | QITFEVPCCLVRKG | 11 |
| 300 | QLGLDKLNHLVKKS | 11 |
| 301 | QSSSVEPYYVLQNS | 11 |
| 302 | QVACEQPYYNVNCS | 11 |
| 303 | RLPEAEPYYLMTQP | 11 |
| 304 | RVPGERQYDALNLS | 11 |
| 305 | YVPLQEPYYVVKQS | 11 |
| 306 | ESALKEPHQVLKHS | 10 |
| 307 | HAPADQLYYPVWKP | 10 |
| 308 | HFPWEEPYHVAKRS | 10 |
| 309 | HFQSEEPYHIPHKH | 10 |
| 310 | HHPLQEPYYVVEKS | 10 |
| 311 | HMPGREPYYVVTTS | 10 |
| 312 | HVAWKGPYYVLETH | 10 |
| 313 | HVHGAPSYSVLKHS | 10 |
| 314 | HVLWVEPYYVINKY | 10 |
| 315 | HVPRVDHLYVETKS | 10 |
| 316 | HVPSGERYYVVKTI | 10 |
| 317 | HVPYVGAYNGVKKS | 10 |
| 318 | HVQWEGPYYDVKKS | 10 |
| 319 | HVRGAELSYVLTKS | 10 |
| 320 | HVTWGEPYQVLNKS | 10 |
| 321 | KLLTEAPYMFRTQA | 10 |
| 322 | KLPWQESYDIVRKS | 10 |
| 323 | KLQWQDPDYVVKNS | 10 |
| 324 | KQLGVLPDYVRKNT | 10 |
| 325 | LAPFEERSYALKSP | 10 |
| 326 | LASWDEPSSVLRES | 10 |
| 327 | LLPPDEPYYPMKKS | 10 |
| 328 | NLQWEQPSYVAKDY | 10 |
| 329 | NVPWSEQYNALKTS | 10 |
| 330 | QFASSHTDYVADKS | 10 |
| 331 | QIQWQGSYNDLNKS | 10 |
| 332 | QISSEEPTSLLDRP | 10 |
| 333 | QKPTGASYYGTKPS | 10 |
| 334 | QLAFDGPSPILTTS | 10 |
| 335 | QLHWGQPYSILNKS | 10 |
| 336 | QLPCEVPCYLVQYS | 10 |
| 337 | QLPLQYRYYFVDQS | 10 |
| 338 | QLPWEGKYQLGKKR | 10 |
| 339 | QLRWHEPYHVVNRY | 10 |
| 340 | QSQCAGPGSAYKKC | 10 |
| 341 | QTLAGETNSVVLKF | 10 |
| 342 | QTQCEEPCYVVKFA | 10 |
| 343 | QVPSEEQFYAAVKS | 10 |
| 344 | QVPSENPFRVLKNL | 10 |
| 345 | QVRYVASSDLVRIP | 10 |
| 346 | QVSLQEPGDVLKEF | 10 |
| 347 | QVSREEPSGVVNKP | 10 |
| 348 | RLPSEALYYLVKRS | 10 |
| 349 | YVPVEGIQHVVEKS | 10 |

TABLE 2F

| SEQ ID | | |
|---|---|---|
| NO: # | Nerve3 | Count |
| 350 | HVRCEEPYCHVRKS | 755 |
| 351 | ELPWQKRKDVMKYP | 638 |
| 352 | QMPWEVPYYVVKRS | 548 |
| 353 | DDIWEVPKYRPKNS | 475 |
| 354 | QIASEERDNIVKKS | 462 |
| 355 | RVPWQEPYYVVLKP | 419 |
| 356 | QLPGYEPYYVGTVS | 412 |
| 357 | RIPSVGPYGVVKTF | 391 |
| 358 | QDVWAEPYYAGKQP | 390 |
| 359 | HVAWQEPRYFVKKS | 359 |
| 360 | YLFAEELAYNLTKS | 359 |
| 361 | QRPWEQAYFVVRST | 357 |
| 362 | FVSREEPYYVVKKS | 341 |
| 363 | DVAREKPYYDMKKS | 335 |
| 364 | QFPSVGSYYVVPQS | 331 |
| 365 | HVVWESSHFVVIDS | 329 |
| 366 | QLRLGDLYNVMTKE | 328 |
| 367 | QFQWQELLNVVKDS | 327 |
| 368 | HVPLQEADYVLKKF | 315 |
| 369 | QFPLYDPYEVMPKS | 310 |
| 370 | HFRWDEPYYIANKA | 300 |
| 371 | RDRHEDSKFVMNHS | 290 |
| 372 | QVPFAHPYYVVNMS | 288 |
| 373 | HVRWGEPSYVLKNF | 272 |
| 374 | QGQTETHSHDAKRY | 270 |
| 375 | QLRFEGPDNPGVQS | 269 |
| 376 | QLPWEVPYSVVRQS | 267 |
| 377 | HVPLPGPYFVLKNY | 263 |
| 378 | HVKRIETYYVFTKS | 258 |
| 379 | RIHLYQPYSILKNY | 258 |
| 380 | QFTSEETHYVVNKS | 257 |
| 381 | QHPWEDPYKIFNKY | 240 |
| 382 | DVPWPNAYNFGIKS | 238 |
| 383 | DVPFEHSYYGEQQY | 237 |
| 384 | EFLGEAPYYVVSRS | 236 |
| 385 | HVSGEEPYTVGNTS | 234 |
| 386 | QFQLDQSYYSFNTL | 231 |
| 387 | QILRVHPDYVVVNP | 231 |
| 388 | HFPWDEAYLVVKNP | 230 |
| 389 | HFSSQESNSGGKKW | 230 |
| 390 | HISGEDPYFILQNS | 212 |
| 391 | TFQYEQPNLVVKNA | 207 |
| 392 | LLAWDEQDYFLRKT | 199 |
| 393 | HINWDKRYAVVITY | 197 |
| 394 | HFPSEYPFDLVSKS | 196 |
| 395 | QFLWQQPDHVRRKS | 196 |
| 396 | QIVLNAPYYVVKTS | 189 |
| 397 | TVPAHNPYYLVKQT | 185 |
| 398 | QDPSVDPYYVDRSS | 181 |
| 399 | QFHCHGACYIVKNS | 180 |
| 400 | RLPQHESYIVGKIS | 174 |
| 401 | RVYWIDPYYIVNES | 174 |
| 402 | QIPLKTPYNVVKKF | 171 |
| 403 | QFPQGEAGYIAKSF | 163 |
| 404 | QFYWDQSNYGEQKP | 161 |
| 405 | HVPWAGSSDVLKVT | 160 |
| 406 | QVPWEKPVSFVQKS | 159 |
| 407 | HVWWEDPYYVVRKT | 158 |
| 408 | WLHWEQPYWTVKKS | 143 |
| 409 | LFAGENGYFTVRQF | 139 |
| 410 | QVSWVYPPNDVIVS | 133 |
| 411 | NDPWPVPYYMVKNS | 130 |
| 412 | QVQWQVPYYVVKKP | 128 |
| 413 | HFPLQEPYYVVKNS | 127 |
| 414 | QGSSVATSSVLTML | 123 |
| 415 | QPCADKPYCVHTIT | 123 |
| 416 | HVWWEEAHYSWKHS | 120 |
| 417 | DLFVEEPYLGVIKF | 117 |
| 418 | YSLWEDLIFNMQNS | 116 |
| 419 | QIPWVEASNVLISF | 115 |
| 420 | QFGQENHNHFVRKS | 113 |
| 421 | QVSLVESRSVLKIL | 110 |
| 422 | AVQRAKPYYAVONS | 109 |
| 423 | HLLGDYRFWAFTNA | 109 |
| 424 | ESPWAPPYYVVKKS | 107 |
| 425 | QIPTVEPYYEGKKS | 105 |
| 426 | QVRGGQPDQVLRLS | 105 |
| 427 | FVARNEPFYVVGTS | 104 |
| 428 | QIPLEEMYYVVRTY | 103 |
| 429 | QVSSVGPNHVVKKI | 102 |
| 430 | QLPWGKPYNVVVYS | 101 |
| 431 | QTPWQVPGYNMLNS | 99 |
| 432 | AYLSEQLNYVMKNS | 98 |
| 433 | QASSEAPSYPVVKS | 95 |
| 434 | LDQWHTPNYVVRKS | 92 |
| 435 | QLLWDPPSYFVNNS | 92 |
| 436 | DVPWEQPYFVVRNS | 90 |
| 437 | QVPYGQSFVVGTSS | 90 |
| 438 | QLRWEDQYAHVEKS | 89 |
| 439 | HVAWKAPYHFTKNS | 87 |
| 440 | DGLYVRSFYVLQHS | 86 |
| 441 | QFALKGLNGDFQKS | 83 |
| 442 | QFPSAETYRIVKKF | 83 |
| 443 | QLTWELGYHAFVKP | 83 |
| 444 | QLLRNLSYDGAKYS | 82 |
| 445 | QLRVKLQYDVIANN | 81 |
| 446 | QVPAREPNHIVKKS | 80 |
| 447 | SAREQGTFYFVNNS | 80 |
| 448 | LFPKYAPHESVKMS | 78 |
| 449 | QVPGNELYFVLKQY | 77 |

TABLE 2G

| SEQ ID | | |
|---|---|---|
| NO: # | Plated Nerve2 | Count |
| 450 | QFAWSQSYAYSSTT | 107 |
| 451 | QGQWDKPYDAVQNS | 88 |
| 452 | QIPGDAQYRLVKRS | 80 |
| 453 | QIPWTQPQYDVTPP | 79 |
| 454 | HVELESSYYFVNNS | 78 |
| 455 | QFPLAEPYYHLTES | 77 |
| 456 | HFDWEEPYYDASKA | 73 |
| 457 | SYEWEDPYYDLKYS | 73 |
| 458 | NVTGAEYYYRMNNY | 67 |
| 459 | HVSCEVPCYGALAS | 66 |
| 460 | HDASEGAYYVERNS | 65 |
| 461 | QFGWEEPYYVLHTV | 65 |
| 462 | HLSREGTYYAVKQP | 64 |
| 463 | QLPLYDPYYTVRTS | 63 |
| 464 | ELPYEEPGTFVEKA | 62 |
| 465 | QFPWEEPYYVFTNS | 62 |
| 466 | QIPGGERSYVLENS | 62 |
| 467 | QITSDKPYYVVKNS | 60 |
| 468 | YDPSGEPYYRVNNH | 59 |
| 469 | QVPRPASSYIGKTS | 58 |
| 470 | QIPLEGPTYVTNPS | 57 |
| 471 | AIPSGAPYYKVNKY | 56 |
| 472 | EFRSEQPYYFVNKY | 55 |
| 473 | QIRWDETYYDIQKT | 55 |
| 474 | QLALEQQSYDAKMS | 55 |
| 475 | ALPWEHPWCVSKNC | 54 |
| 476 | HVPVLEPYDLVKKS | 54 |
| 477 | QIRSTEPYYVFKKT | 54 |
| 478 | QSPGTNPQNVLIKS | 54 |
| 479 | QITSVDPSSLAYRS | 53 |
| 480 | QMPSGAPYDATLNS | 53 |
| 481 | QNPWDEPSYVVKNV | 53 |
| 482 | QSPFDETAYGLTHS | 53 |
| 483 | QVLFAKSYYSVKDS | 53 |
| 484 | RFPADEEHYVGKLS | 53 |
| 485 | YVPYEEQKYVVTKS | 53 |
| 486 | HSQGQTTEYVGKQS | 52 |
| 487 | DFQSEETRNLAKMP | 51 |
| 488 | DVPWPAPYYAVSKY | 51 |
| 489 | QFPGEESHYVVRNS | 51 |
| 490 | QFQWETPSHVINDS | 51 |
| 491 | QTPRQTLYYSVKAY | 51 |
| 492 | HVAYQEPFVYQKTS | 50 |
| 493 | QISRGDTYYVMNKS | 50 |
| 494 | HIPGEEPSDGVRSY | 49 |
| 495 | HLSVAEPYYAVRNS | 49 |
| 496 | QLQWQKSNWVGKIT | 49 |
| 497 | EIPWEGTYRLVKHS | 48 |
| 498 | HVPQKEPYYVVKTF | 48 |
| 499 | QFPLFTTFYNVEES | 47 |
| 500 | QFPWETSYYIVKNS | 47 |
| 501 | QLAGGDPYVLPKLS | 47 |
| 502 | DVASGEQSYIVVKY | 46 |
| 503 | HVWLEDTISAEKKS | 46 |
| 504 | QVQWDGASSIVQKT | 46 |
| 505 | HFRGPEPYKFVENP | 45 |
| 506 | HVPWEGPEYIVTQS | 45 |
| 507 | QFPRHDPNEVVHKS | 45 |
| 508 | QIPSEEPNYILKSS | 45 |
| 509 | QRPGDEPSYFVKNS | 45 |
| 510 | ELTMEQPSYVGNVS | 44 |
| 511 | QLPWHEAFFGVNNG | 44 |
| 512 | QVLEKEPSSNVHKS | 44 |
| 513 | HVPSYAPNYEIKES | 43 |
| 514 | QHLGEEPSYSGEYA | 43 |
| 515 | SISSKDPYALVKYS | 43 |
| 516 | QFPWDEPNYVVKNF | 42 |
| 517 | QLPFNEPYYSQQVS | 42 |
| 518 | HLSMEAPYYGVKEY | 41 |
| 519 | HVLGEQPYSVVKTS | 41 |
| 520 | HVSWEEPRYIVKDS | 41 |
| 521 | QLPWEGPYYIVKKS | 41 |
| 522 | QTPAEKPYYVVKKY | 41 |
| 523 | HVPSQEWYYDLHKS | 40 |
| 524 | QFPWHKQFYVGKSS | 40 |
| 525 | DFQIVGPSYVGKNS | 39 |
| 526 | HLPSEKPWYDLDGS | 39 |
| 527 | HVSWSTSKYVVNHS | 39 |
| 528 | QISWEDPDYGVRKS | 39 |
| 529 | QVPCEQQSCVVKNS | 39 |
| 530 | LFTGQQSYYEVQES | 38 |
| 531 | LVSPTGPIYVMNKS | 38 |
| 532 | QFASEEPYHVMKKP | 38 |
| 533 | QFSRADSQYLVKNS | 38 |
| 534 | QIPLQDPYHVVEKS | 38 |
| 535 | QLLWGESYSIVKNY | 38 |
| 536 | QLPSQTQNYYDTNA | 38 |
| 537 | QLRSEDTYYTMQSY | 38 |
| 538 | SVAGQQHYYVSTKP | 38 |
| 539 | HLPCKKSFYACKKH | 37 |
| 540 | HVRFAEPYYAGKTS | 37 |
| 541 | LLTGAEPYYLGKRS | 37 |
| 542 | QLPGAEAQNVVKYS | 37 |
| 543 | QLRFDEHHSVVTHS | 37 |
| 544 | QYPWEQRYYFERKS | 37 |
| 545 | RVTWEGPYNSVKKS | 37 |
| 546 | DVALGESHYNVKKY | 36 |
| 547 | DVPNDLPSYLVKNS | 36 |
| 548 | EFPYQESEYIVKQS | 36 |
| 549 | GLPSKEPYYVVKPA | 36 |
| 550 | HVPWDDQYYVWRTN | 36 |
| 551 | HVQWDQTSYVVKNS | 36 |
| 552 | HVTSDDSYYAVKNP | 36 |
| 553 | QDPFGDLYYVTKMS | 36 |
| 554 | QFPYDEPYNVVKKS | 36 |
| 555 | QHPWKEQYYMATKP | 36 |
| 556 | QLNSEEPYYHFKNY | 36 |
| 557 | QLPTNDPYYVLMES | 36 |

### Example 2: Prevalence of Amino Acid Residues in Nerve-Binding Peptides

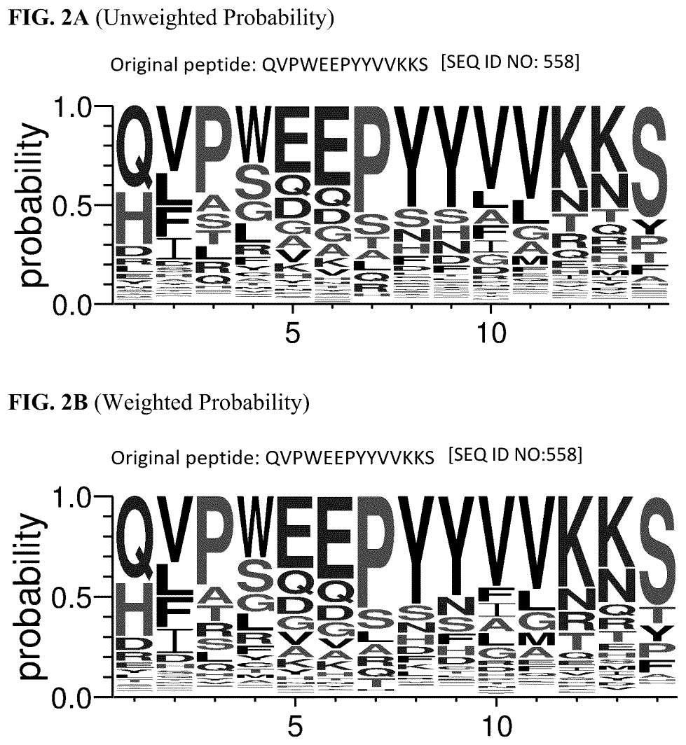

FIG. 2 depicts the most prevalent amino acids at each position of the sample set of peptides identified in Example 1 (and listed in Table 2), based on unweighted (FIG. 2A) or weighted (FIG. 2B) probabilities. In an unweighted analysis, the probability of each amino acid at each position is based on its representation in the unique peptide set without considering the number of counts. In a weighted analysis, the probability of each amino acid in the sequence is based on its representation in the unique peptide set as a function of the total number of counts.

As shown in FIG. 2 and quantified in Table 3, by either analysis, the most frequent amino acid at each position corresponds to that in the original peptide sequences. However, some differences were noted with respect to less frequent amino acids at certain positions. For example, at position 10, the most frequently present amino acid was valine—the same as the parent peptide—in the unweighted and weighted alignments. However, the second (and third) most frequent amino acid at position 10 was leucine (and glycine) in the unweighted alignment but phenylalanine (and isoleucine) in the weighted analysis.

TABLE 3
Unweighted Probabilities

| position | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| AA | Q | V | P | W | E | E | P | Y | Y | V | V | K | K | S |
| percent | 43.63 | 33.75 | 44.70 | 29.44 | 34.65 | 41.11 | 54.58 | 51.71 | 52.06 | 42.91 | 47.76 | 42.37 | 34.29 | 57.27 |
| | H | L | A | S | Q | Q | S | S | S | L | L | N | N | Y |
| | 27.11 | 17.24 | 9.52 | 18.85 | 11.85 | 10.77 | 11.49 | 10.59 | 8.44 | 9.52 | 12.75 | 12.03 | 18.13 | 8.44 |
| | D | F | S | G | D | D | T | N | H | A | G | T | T | P |
| | 6.10 | 15.80 | 8.62 | 10.95 | 11.31 | 8.80 | 6.82 | 7.18 | 7.90 | 7.90 | 8.08 | 10.05 | 7.18 | 7.90 |

Weighted Probabilities

| position | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| AA | Q | V | P | W | E | E | P | Y | Y | V | V | K | K | S |
| percent | 43.28 | 33.73 | 44.92 | 31.25 | 36.67 | 41.89 | 56.36 | 54.41 | 50.13 | 45.57 | 47.09 | 46.03 | 36.10 | 55.05 |
| | H | L | A | S | Q | Q | S | S | N | F | L | N | N | T |
| | 27.12 | 16.43 | 9.38 | 17.52 | 13.42 | 12.36 | 10.65 | 8.55 | 10.02 | 7.76 | 10.61 | 11.50 | 17.67 | 9.19 |
| | D | F | T | G | D | D | L | N | S | I | G | R | Q | Y |
| | 7.09 | 15.74 | 8.76 | 9.67 | 9.50 | 8.71 | 6.41 | 5.99 | 8.25 | 7.50 | 10.17 | 10.18 | 6.79 | 8.88 |

### Example 3: Alignments and Cluster Analysis of Nerve-Binding Peptides and

To further characterize the 557 distinct peptides and identify nerve-binding consensus sequences and corresponding formulas, clustering analyses were carried out using established platforms for multiple sequence alignments. See, e.g., Sievers and Higgins, 2014, Clustal Omega, accurate alignment of very large numbers of sequences, Methods Mol. Biol. 1079, 105-116; Sievers and Higgins, 2018, Clustal Omega for making accurate alignments of many protein sequences, Protein Sci. 27, 135-145; Sievers and Higgins, 2021, The Clustal Omega multiple alignment package, Methods Mol. Biol. 2231, 3-16; Larsson, 2014, AliView: a fast and lightweight alignment viewer and editor for large data sets, Bioinformatics. 30, 3276-3278; Waterhouse et al., 2009, Jalview Version 2—a multiple sequence alignment editor and analysis workbench, Bioinformatics. 25, 1189-1191; Procter et al. 2021, Alignment of biological Sequences with Jalview, Methods Mol Biol. 2231, 203-224.

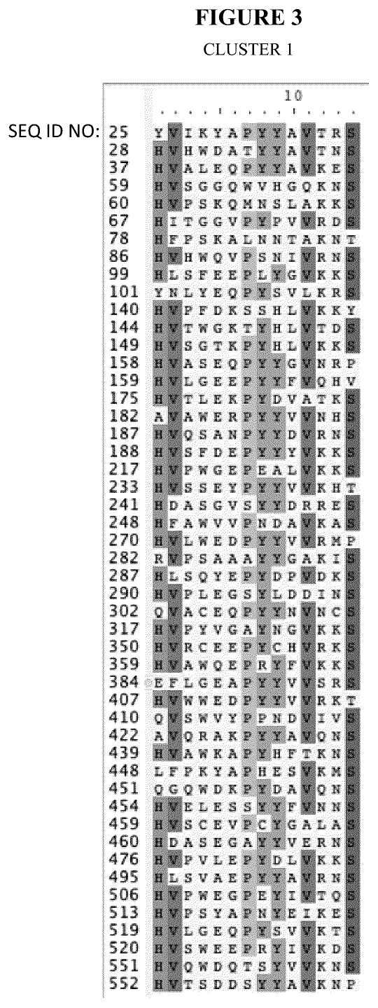

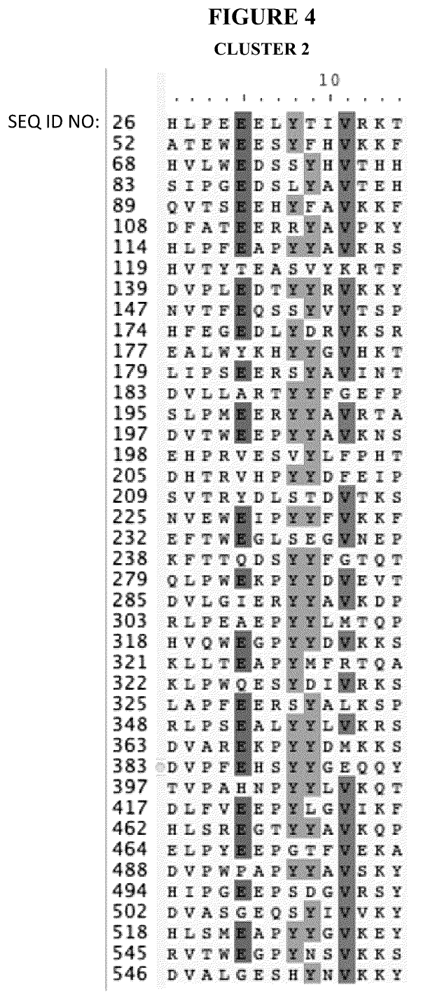

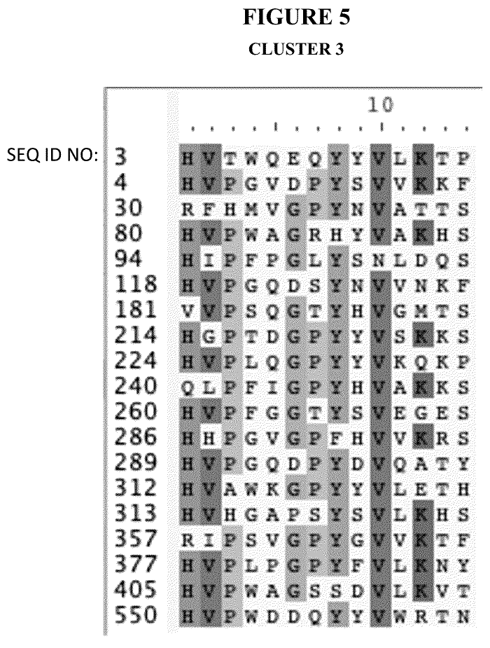

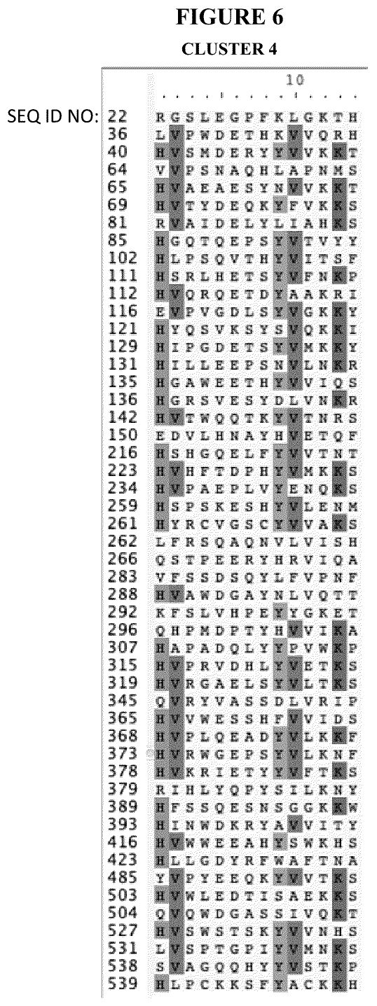

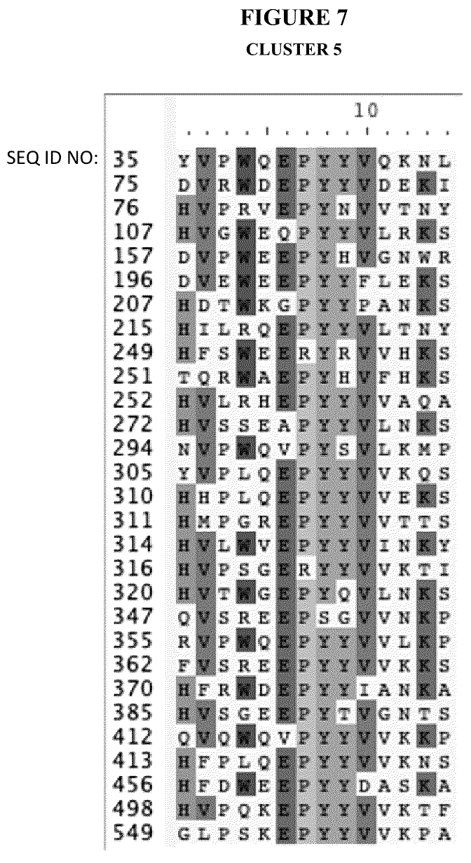

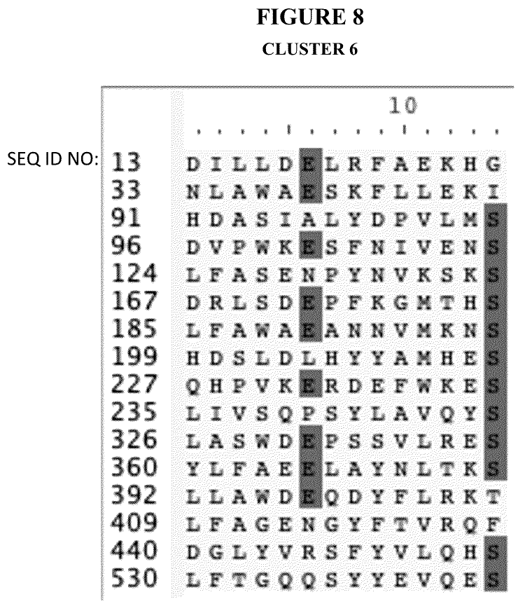

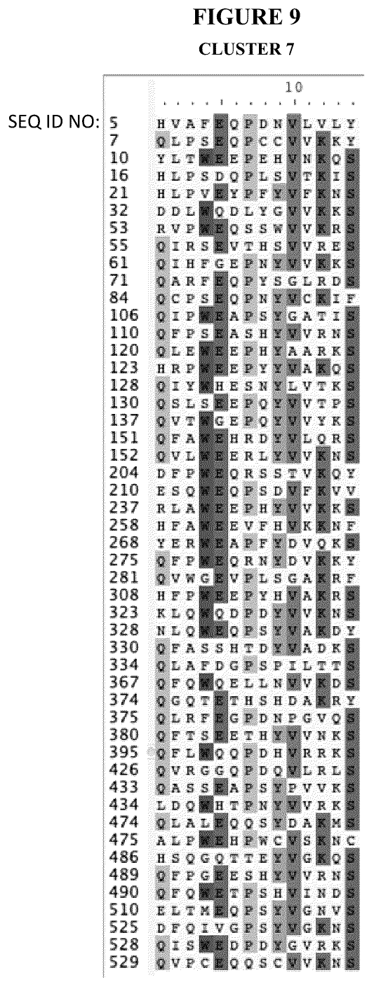

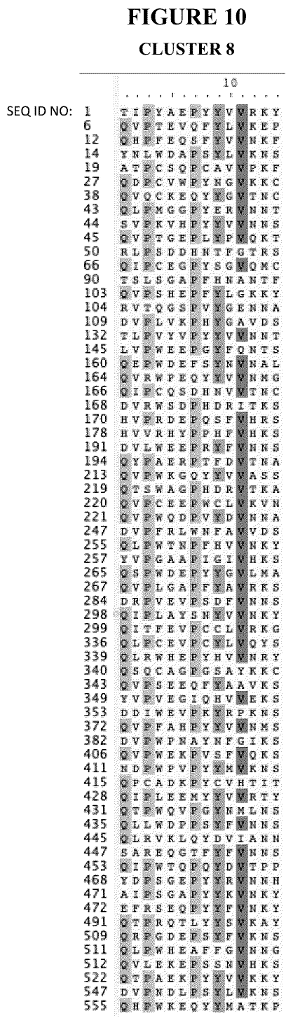

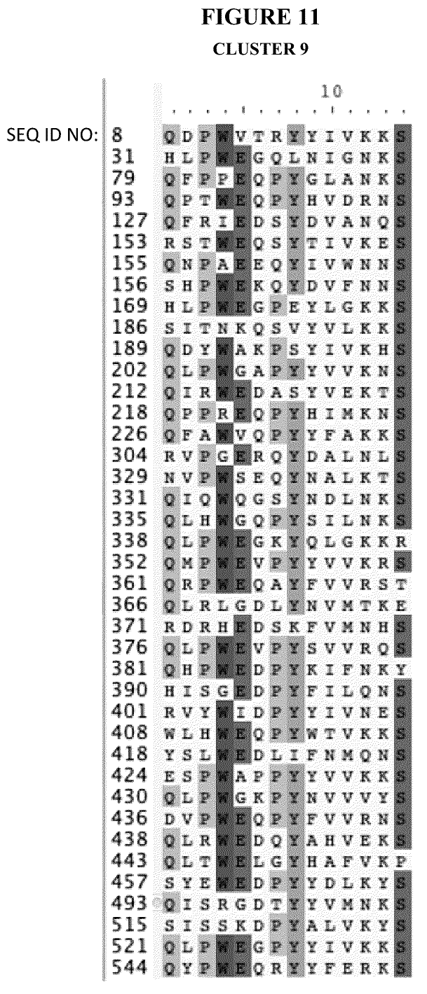

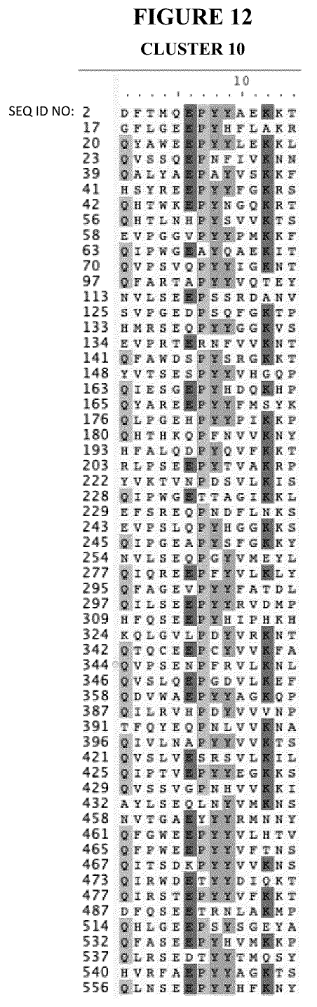

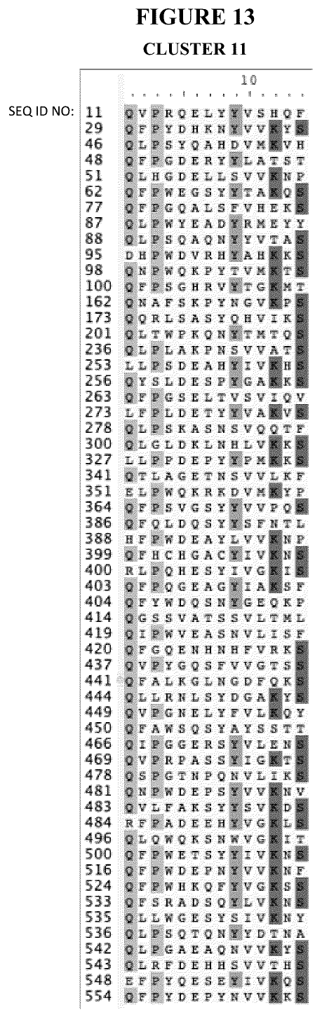

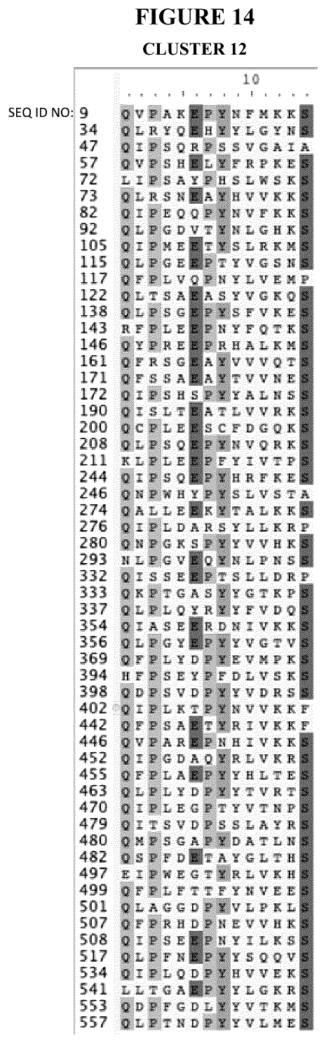

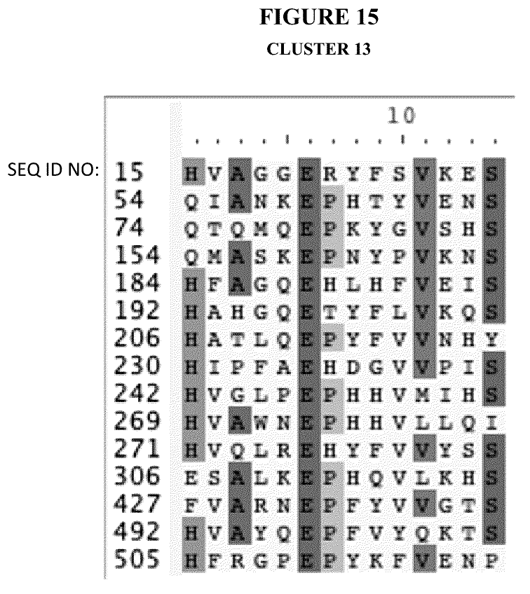

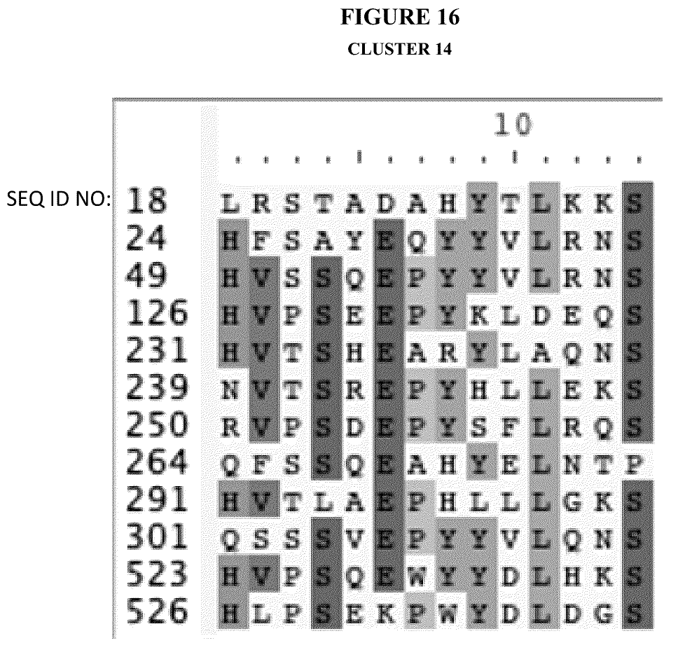

In the first approach, Clustal Omega was used to create an initial phylogenetic tree, and 14 highly related clusters were chosen to visualize individually. The 14 clusters were each visualized with AliView software and the “majority rule consensus” residues at each position were highlighted in order to identify patterns in each cluster. These 14 clusters correspond to Formulas I to XIV (and Subformulas Ia to XIVa), respectively, and are depicted in FIGS. 3 to 16, respectively. Table 4 shows the “majority rule” consensus sequence for these 14 clusters, which are each based on different, non-overlapping sets of peptides in the representative sample.

TABLE 4

| Position | 1 | 2 | 3 | 4 | 5 | 6 | 7 | 8 | 9 | 10 | 11 | 12 | 13 | 14 | |
|---|---|---|---|---|---|---|---|---|---|---|---|---|---|---|---|
| Parent | Q | V | P | W | E | E | P | Y | Y | V | V | K | K | S | (SEQ ID |
| Peptide | | | | | | | | | | | | | | | NO: 558) |
| Cluster 1 | H | V | — | — | — | — | P | Y | Y | — | V | — | — | S | |
| Cluster 2 | — | — | — | — | E | — | — | Y | Y | — | V | — | — | — | |
| Cluster 3 | H | V | P | — | — | G | P | Y | — | V | — | K | — | — | |
| Cluster 4 | H | V | — | — | — | — | — | — | Y | V | — | K | — | — | |
| Cluster 5 | H | V | — | W | — | E | P | Y | Y | V | — | — | K | — | |
| Cluster 6 | — | — | — | — | — | E | — | — | — | — | — | — | — | S | |
| Cluster 7 | Q | — | — | W | E | — | P | — | Y | V | — | K | — | S | |
| Cluster 8 | Q | — | P | — | — | — | P | — | Y | — | V | — | — | — | |
| Cluster 9 | Q | — | P | W | E | — | P | Y | — | — | — | — | — | S | |
| Cluster 10 | Q | — | — | — | — | E | P | Y | Y | — | — | K | — | — | |
| Cluster 11 | Q | — | P | — | — | — | — | — | Y | — | — | K | — | S | |
| Cluster 12 | Q | — | P | — | — | E | P | Y | — | — | — | — | — | S | |
| Cluster 13 | H | — | A | — | — | E | P | — | — | — | V | — | — | S | |
| Cluster 14 | H | V | — | S | — | E | P | Y | Y | — | L | — | — | S | |

In the second approach, an alternate relational tree was created with Jalview software, and 30 smaller clusters (clusters 15 through 44) were chosen. These 30 clusters were each visualized with AliView software and the “majority rule consensus” residues at each position were highlighted in order to identify patterns in each cluster.

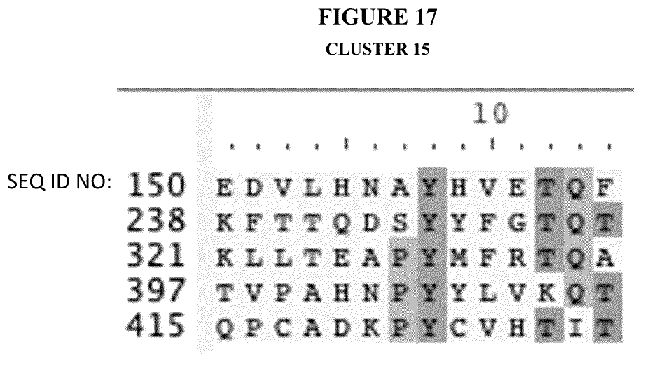

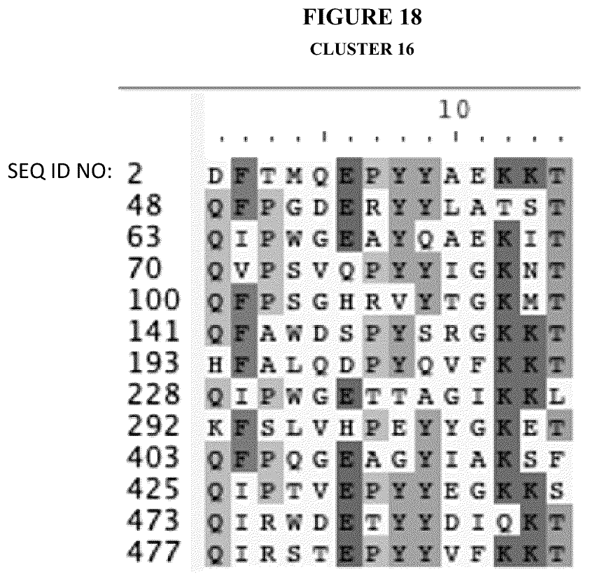

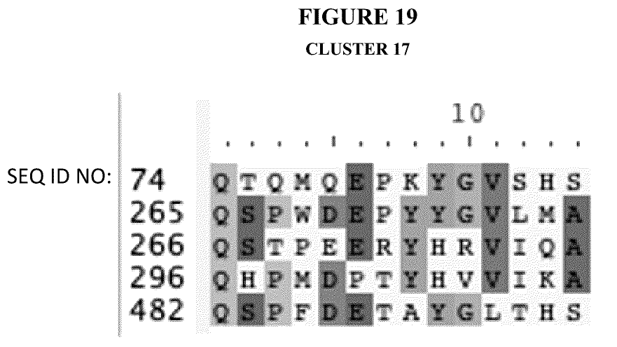

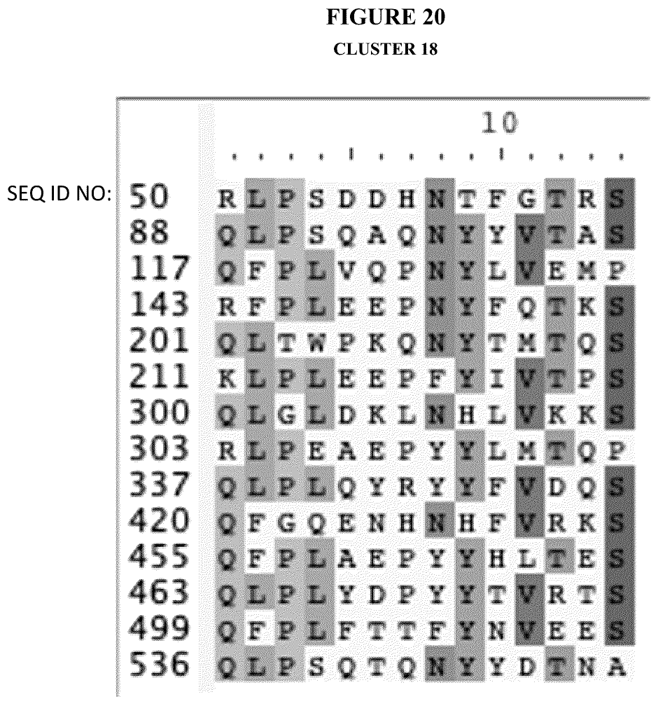

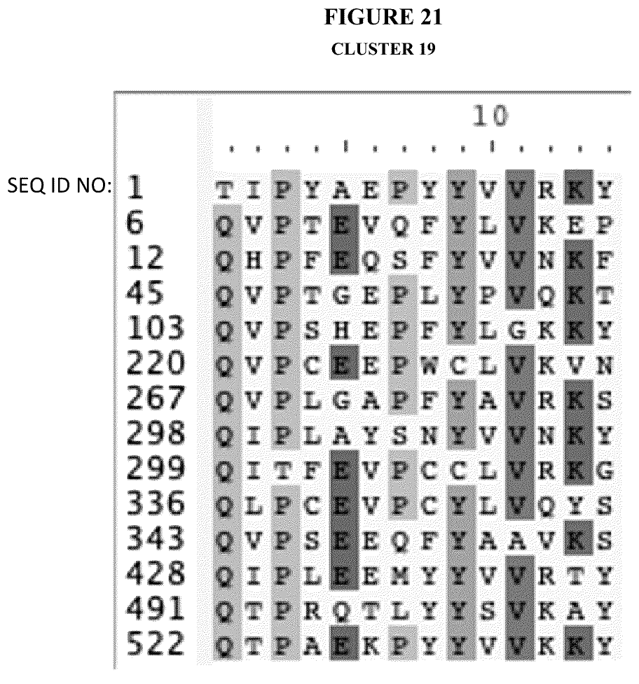

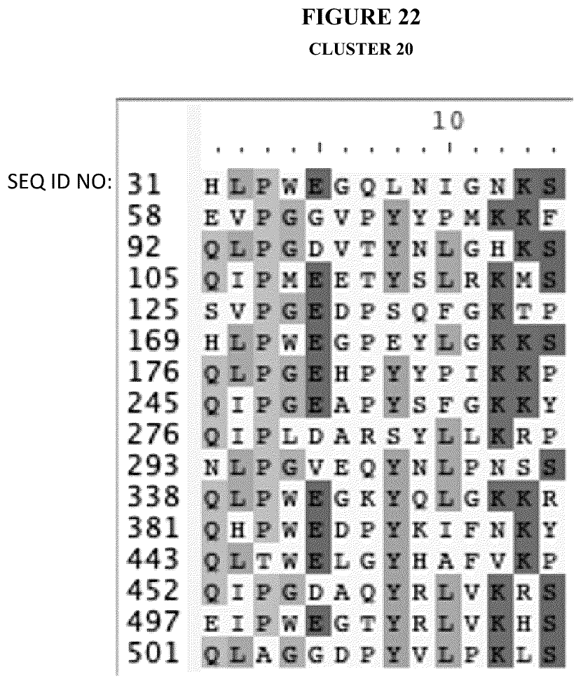

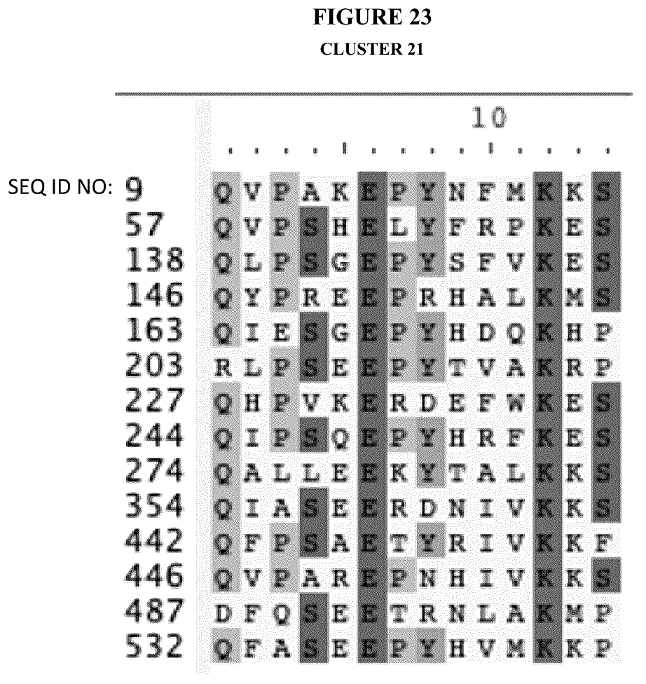

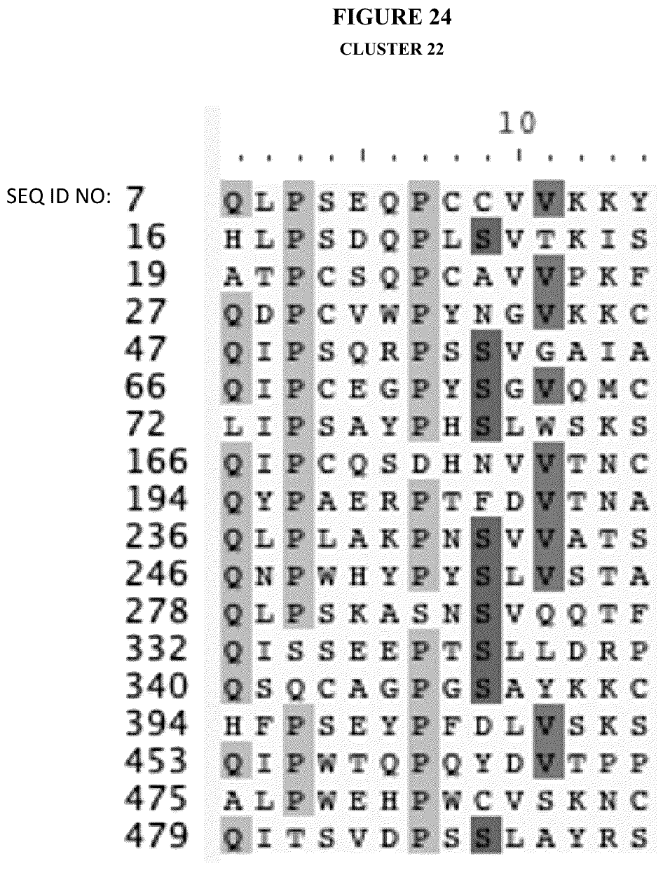

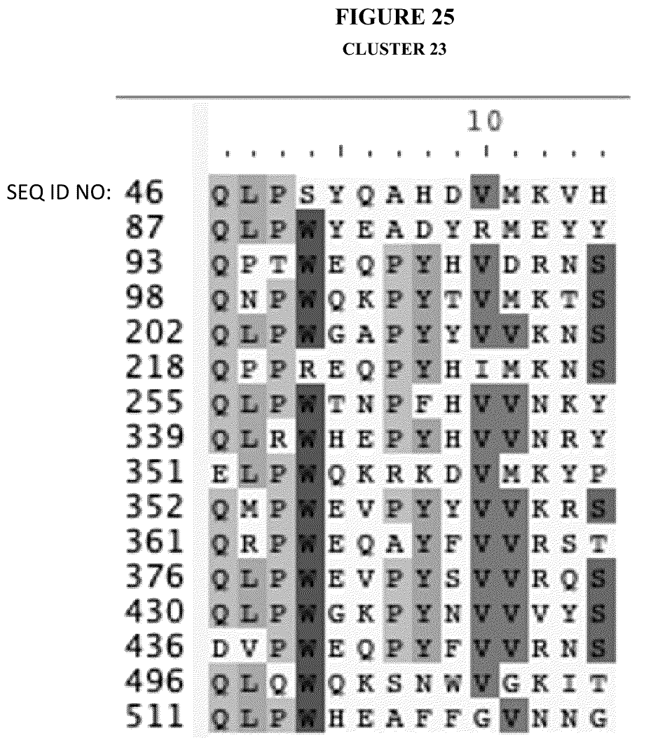

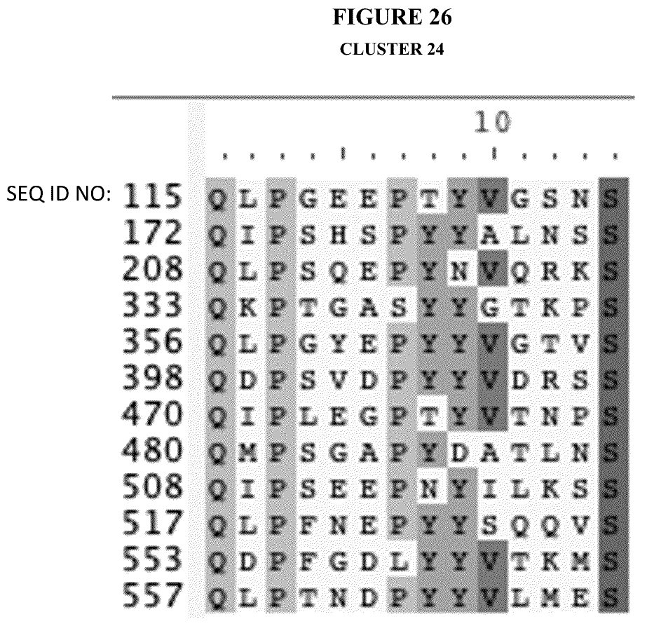

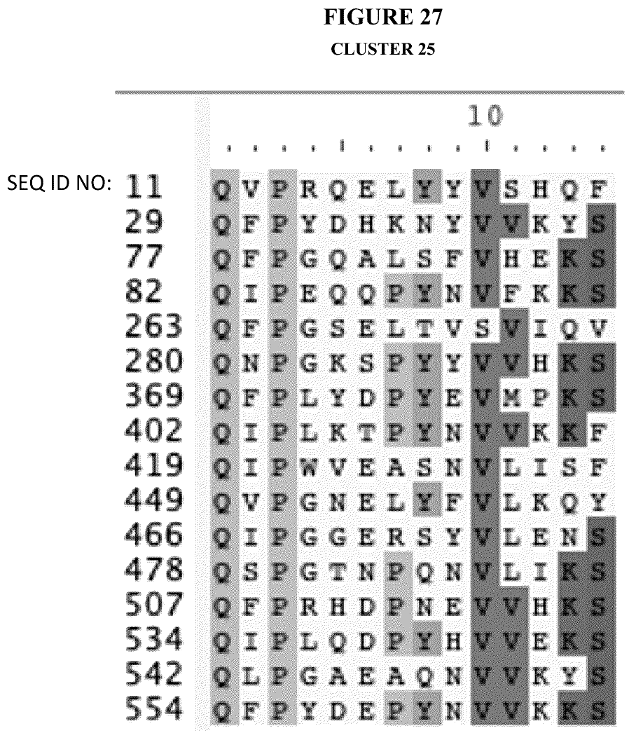

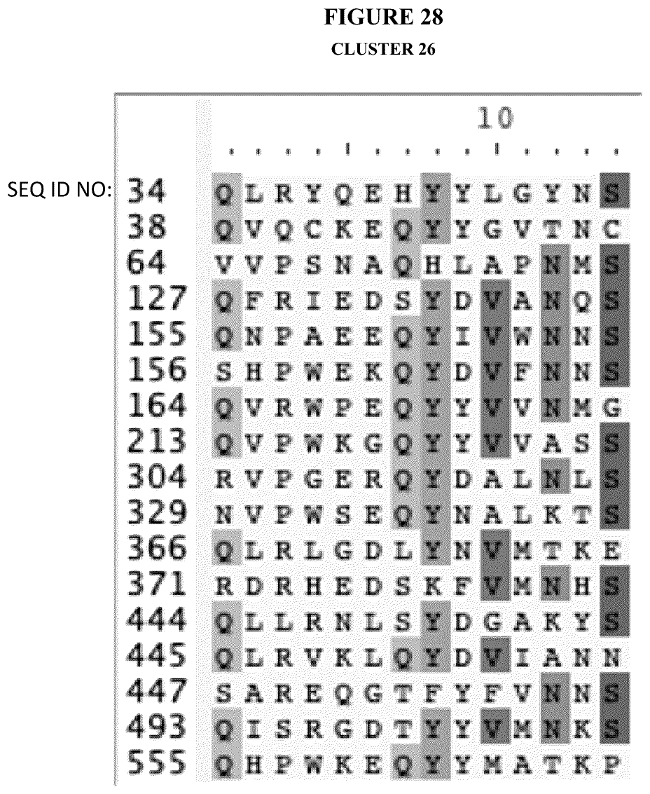

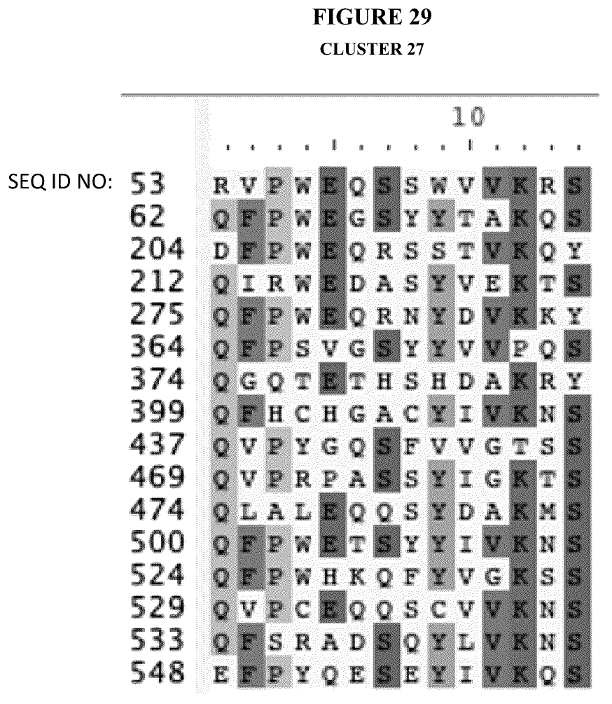

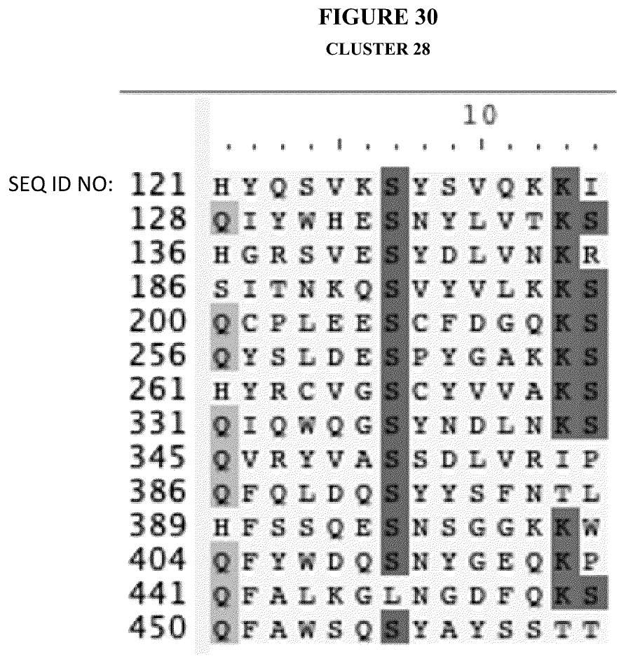

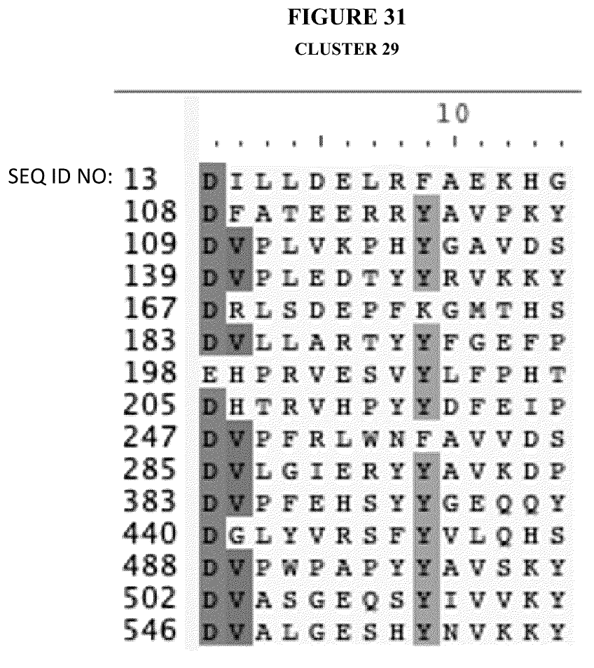

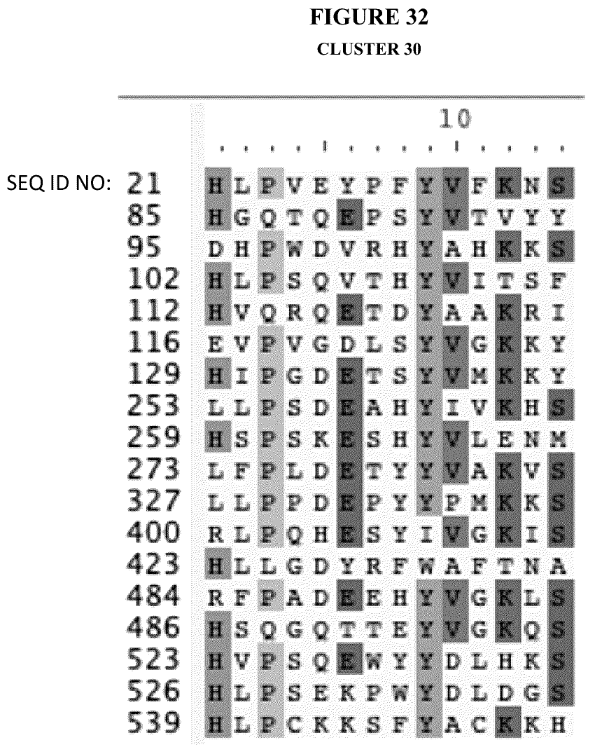

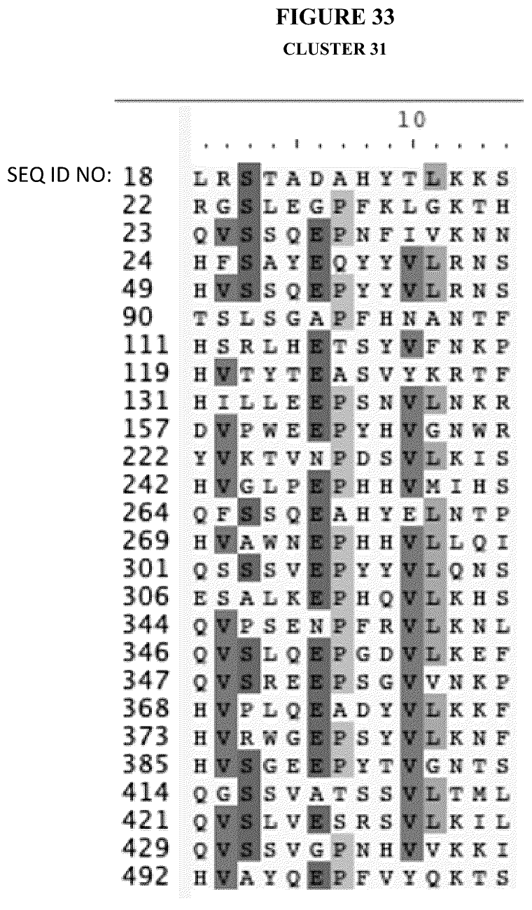

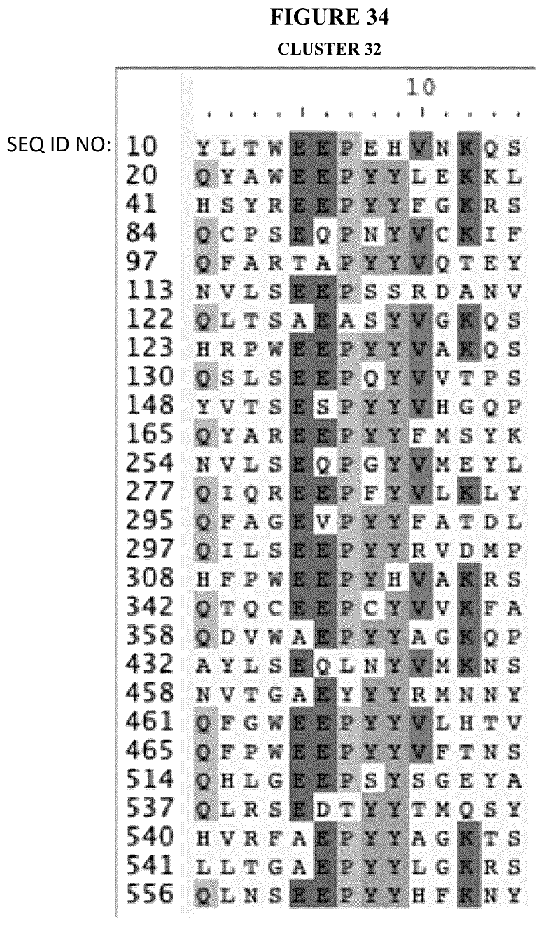

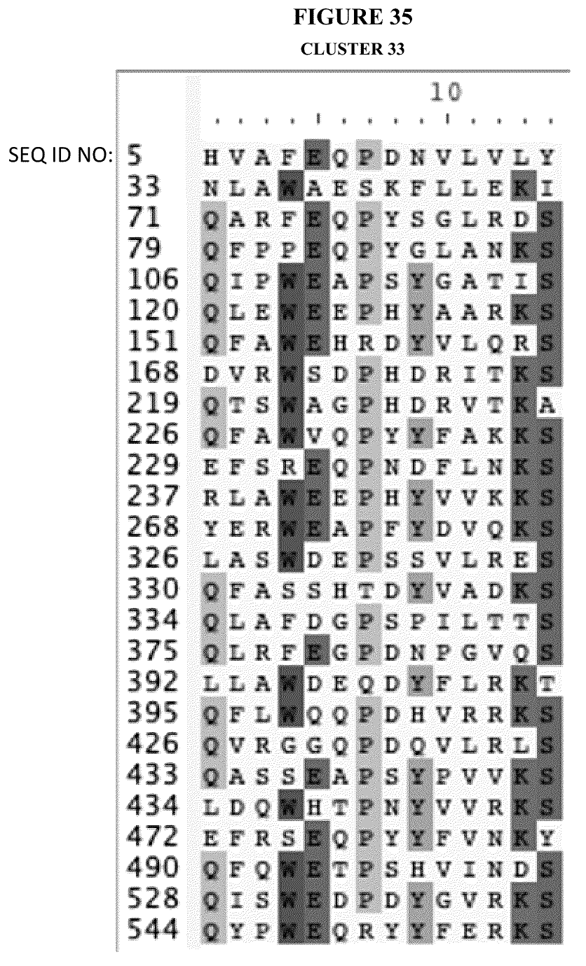

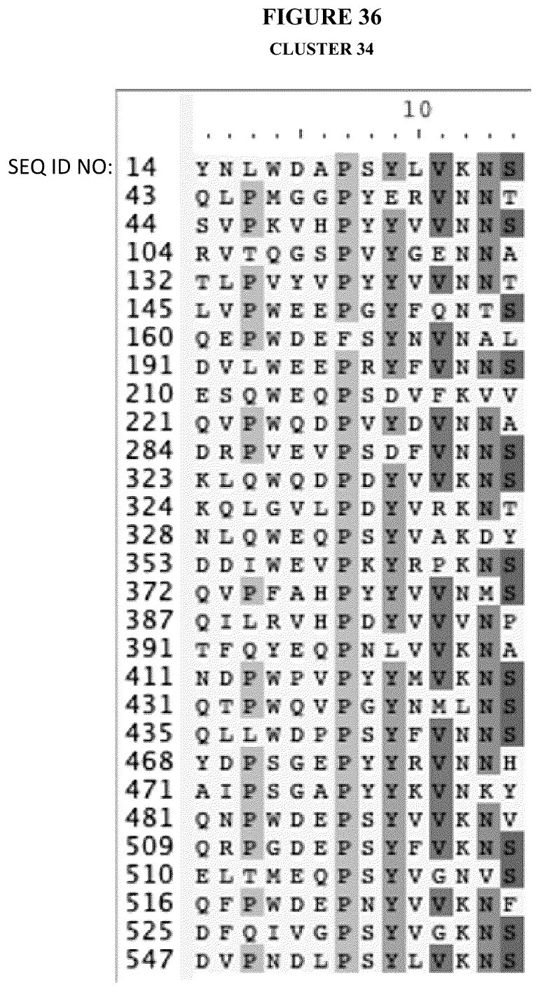

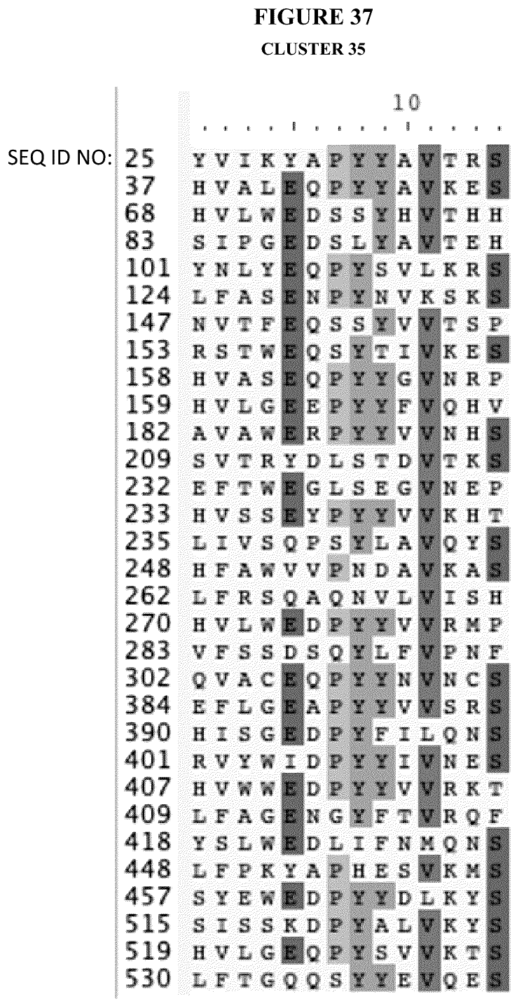

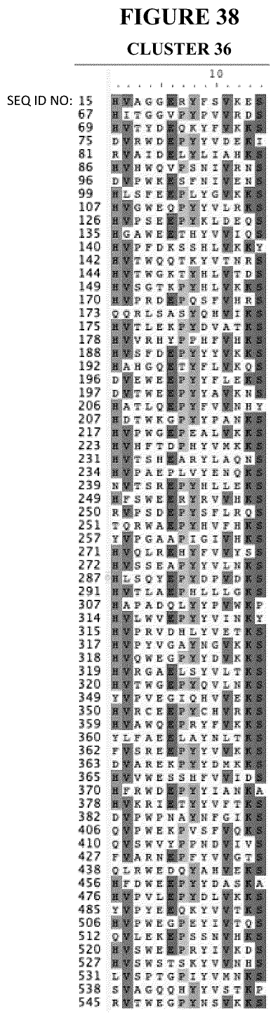

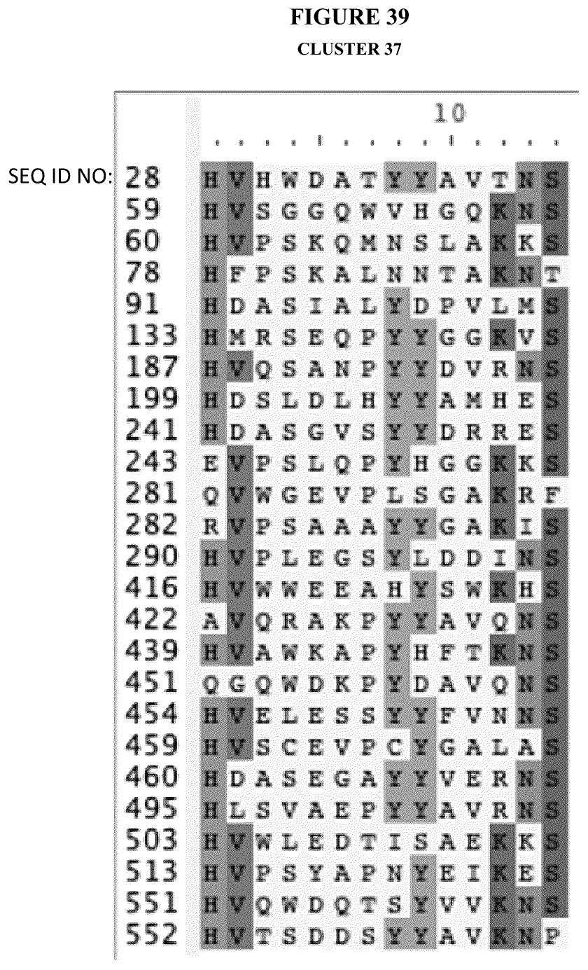

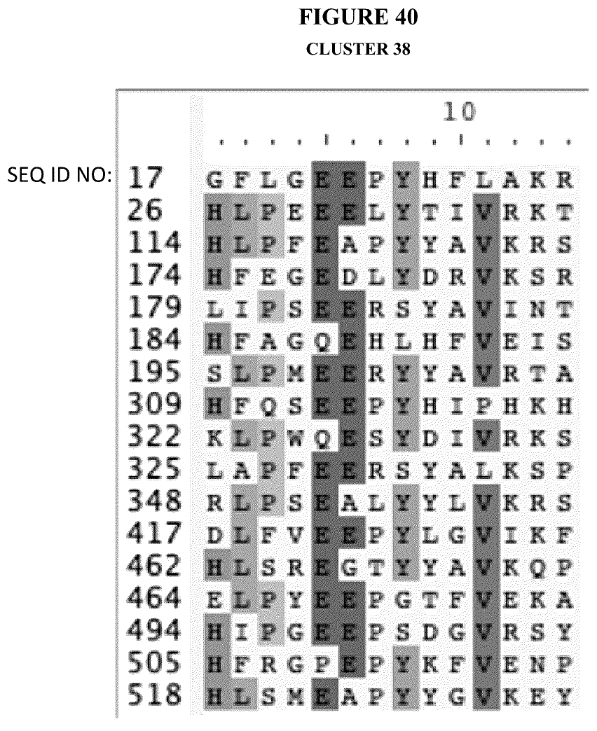

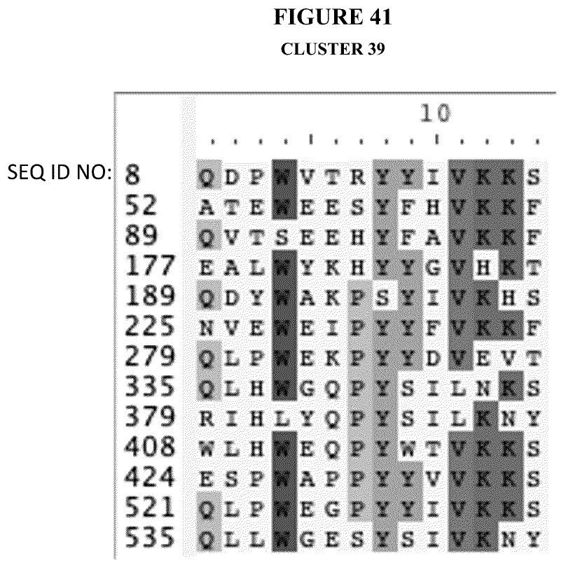

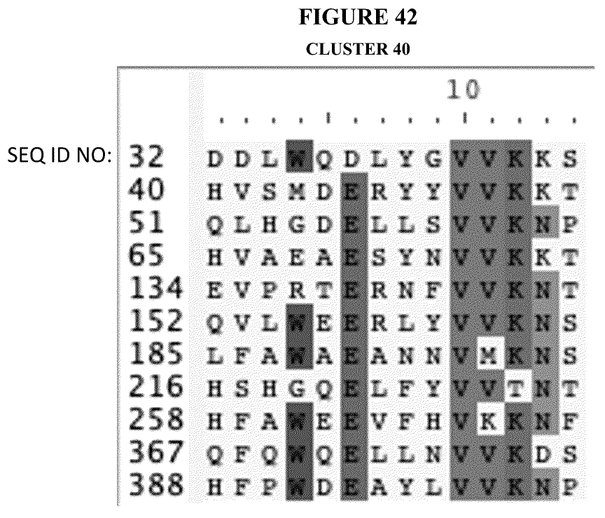

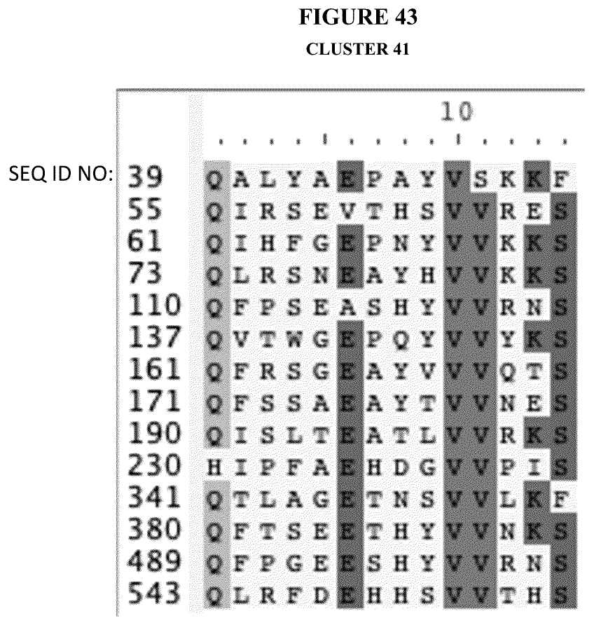

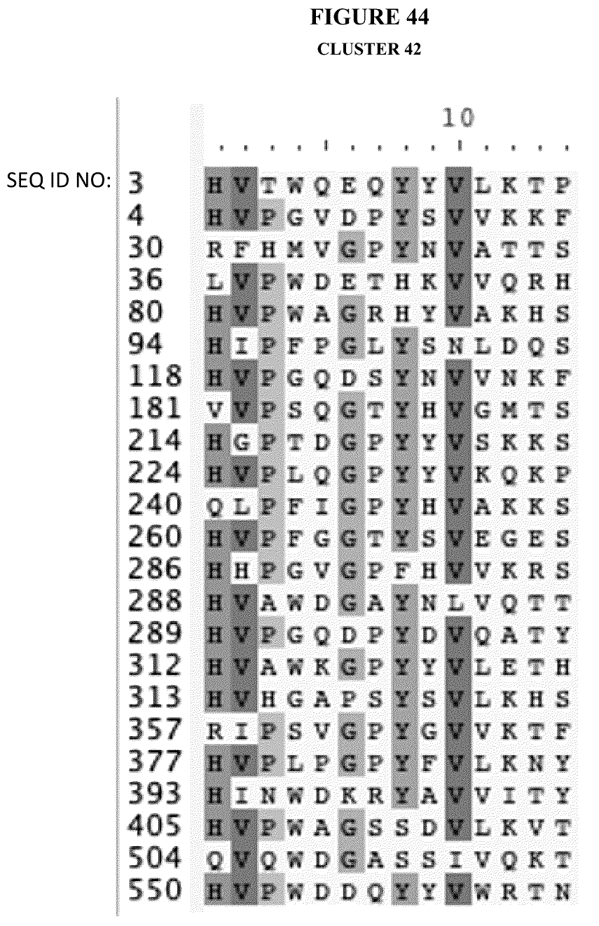

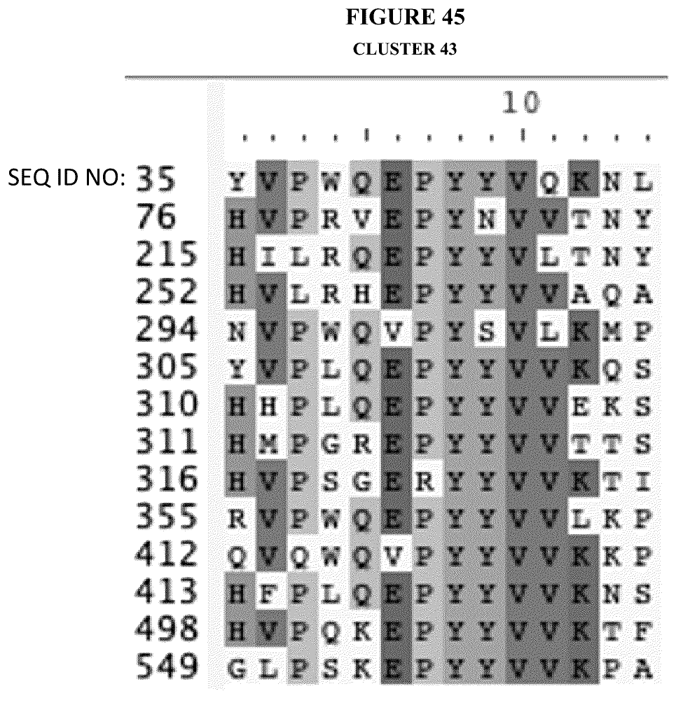

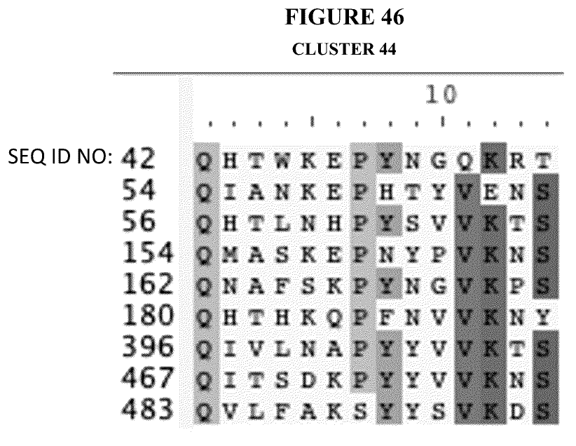

These 30 clusters correspond to Formulas XV to XLIV (and Subformulas XVa to XLIVa), respectively, and are depicted in FIGS. 17 to 46, respectively. Table 5 shows the “majority rule” consensus sequence for these clusters (numbered 15 to 44), which are each based on different, non-overlapping sets of peptides in the representative sample.

TABLE 5

| Position | 1 | 2 | 3 | 4 | 5 | 6 | 7 | 8 | 9 | 10 | 11 | 12 | 13 | 14 | |
|---|---|---|---|---|---|---|---|---|---|---|---|---|---|---|---|
| Parent | Q | V | P | W | E | E | P | Y | Y | V | V | K | K | S | (SEQ ID |
| Peptide | | | | | | | | | | | | | | | NO: 558) |
| Cluster 15 | — | — | — | — | — | — | P | Y | — | — | — | T | Q | T | |
| Cluster 16 | Q | F | P | — | — | E | P | Y | Y | — | — | K | K | T | |
| Cluster 17 | Q | S | P | — | D | E | — | Y | Y | G | V | — | — | A | |
| Cluster 18 | Q | L | P | L | — | — | — | N | Y | — | V | T | — | S | |
| Cluster 19 | Q | — | P | — | E | — | P | — | Y | — | V | — | K | — | |
| Cluster 20 | Q | L | P | G | E | — | — | Y | — | L | — | K | K | S | |
| Cluster 21 | Q | — | P | S | — | E | P | Y | — | — | — | K | — | S | |
| Cluster 22 | Q | — | P | — | — | — | P | — | S | — | V | — | — | — | — |
| Cluster 23 | Q | L | P | W | — | — | P | Y | — | V | V | — | — | S | |
| Cluster 24 | Q | — | P | — | — | — | P | Y | Y | V | — | — | — | S | |
| Cluster 25 | Q | — | P | — | — | — | P | Y | — | V | V | — | K | S | |
| Cluster 26 | Q | — | — | — | — | — | Q | Y | — | V | — | N | — | S | |
| Cluster 27 | Q | F | P | — | E | — | S | — | Y | — | V | K | — | S | |
| Cluster 28 | Q | — | — | — | — | — | S | — | — | — | — | — | K | S | |
| Cluster 29 | D | V | — | — | — | — | — | — | Y | — | — | — | — | — | |
| Cluster 30 | H | — | P | — | — | E | — | — | Y | V | — | K | — | S | |
| Cluster 31 | — | V | S | — | — | E | P | — | — | V | L | — | — | — | |
| Cluster 32 | Q | — | — | — | E | E | P | Y | Y | V | — | K | — | — | |
| Cluster 33 | Q | — | — | W | E | — | P | — | Y | — | — | — | K | S | |
| Cluster 34 | — | — | P | — | — | — | P | — | Y | — | V | — | N | S | |
| Cluster 35 | — | — | — | — | E | — | P | Y | Y | — | V | — | — | S | |
| Cluster 36 | H | V | — | — | — | E | P | Y | — | — | V | — | K | S | |
| Cluster 37 | H | V | — | — | — | — | — | Y | Y | — | — | K | N | S | |
| Cluster 38 | H | L | P | — | E | E | — | Y | — | — | V | — | — | — | |
| Cluster 39 | Q | — | — | W | — | — | P | Y | Y | — | V | K | K | — | |
| Cluster 40 | — | — | — | W | — | E | — | — | — | V | V | K | N | — | |
| Cluster 41 | Q | — | — | — | — | E | — | — | — | V | V | — | K | S | |
| Cluster 42 | H | V | P | — | — | G | — | Y | — | V | — | — | — | — | |
| Cluster 43 | H | V | P | — | Q | E | P | Y | Y | V | V | K | — | — | |
| Cluster 44 | Q | — | — | — | — | — | P | Y | — | — | V | K | — | S | |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments. Moreover, in certain instances, well-known methods, procedures, or other specific details have not been described to avoid unnecessarily obscuring aspects of the invention defined by the appended claims

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A nerve targeting peptide conjugate comprising:
(a) a peptide comprising the amino acid sequence of any one of SEQ ID NOS:1-557; and
(b) a cargo molecule.

2.-8. (canceled)

9. A nerve targeting peptide conjugate comprising:
(a) a peptide comprising the amino acid sequence of Formula (I):
X₁-X₂-X₃-X₄-X₅-X₆-X₇-X₈-X₉-X₁₀-X₁₁-X₁₂-X₁₃-X₁₄, wherein:
X₁ is A, E, H, L, Q, R, or Y;
X₂ is D, F, G, I, L, N, or V;
X₃ is A, E, H, I, L, P, Q, R, S, T, or W;
X₄ is C, F, G, K, L, Q, R, S, V, W, or Y;
X₅ is A, D, E, G, K, L, Q, T, V, or Y;
X₆ is A, D, E, G, K, N, Q, R, S, V, or Y;
X₇ is A, L, M, P, S, T, or W;
X₈ is C, E, H, L, N, P, R, S, V, or Y;
X₉ is A, C, D, E, H, L, N, P, S, or Y;
X₁₀ is A, D, E, F, G, H, I, L, N, P, S, T, V, or Y;
X₁₁ is A, D, E, I, L, Q, R, T, or V;
X₁₂ is D, I, K, L, N, Q, R, S, or T;
X₁₃ is A, C, D, E, H, I, K, M, N, Q, R, T, or V; and
X₁₄ is P, S, T, V, or Y;
and
(b) a cargo molecule.

10. (canceled)

11. (canceled)

12. A nerve targeting peptide conjugate comprising:
(a) a peptide comprising the amino acid sequence of Formula (II):
X₁-X₂-X₃-X₄-X₅-X₆-X₇-X₈-X₉-X₁₀-X₁₁-X₁₂-X₁₃-X₁₄, wherein:
X₁ is A, D, E, H, K, L, N, Q, R, S, or T;
X₂ is A, F, H, I, L, T, or V;
X₃ is A, E, F, L, P, Q, S, or T;
X₄ is A, E, F, G, L, M, R, S, T, V, W, or Y;
X₅ is A, E, G, H, I, P, Q, T, V, or Y;
X₆ is A, D, E, G, H, I, K, N, Q, or R;
X₇ is A, H, L, P, Q, R, S, or T;
X₈ is G, H, L, R, S, V, or Y;
X₉ is D, E, F, L, M, N, T, V, or Y;
X₁₀ is A, D, F, G, H, I, L, N, R, S, V, or Y;
X₁₁ is E, F, G, K, L, M, R, or V;
X₁₂ is E, H, I, K, N, P, Q, R, S, T, or V,
X₁₃ is D, E, F, H, I, K, N, Q, R, S, T, or V; and
X₁₄ is A, F, H, P, R, S, or T; and
(b) a cargo molecule.

13. (canceled)

14. (canceled)

15. A nerve targeting peptide conjugate comprising:
(a) a peptide comprising an amino acid sequence of Formula (III):
X₁-X₂-X₃-X₄-X₅-X₆-X₇-X₈-X₉-X₁₀-X₁₁-X₁₂-X₁₃-X₁₄, wherein:
X₁ is H, Q, R, or V;
X₂ is F, G, H, I, L, or V;
X₃ is A, H, P, or T;
X₄ is F, G, L, M, S, T, or W;
X₅ is A, D, G, I, K, P, Q, or V;
X₆ is D, E, G, or P;
X₇ is L, P, Q, R, S, or T;
X₈ is F, H, S, or Y;
X₉ is D, F, G, H, N, S, or Y;
X₁₀ is N or V;
X₁₁ is A, E, G, K, L, Q, S, V, or W;
X₁₂ is A, D, E, G, K, M, N, Q, R, or T;
X₁₃ is E, H, K, N, Q, R, T, or V; and
X₁₄ is F, H, N, P, S, T, or Y; and
(b) a cargo molecule.

16. (canceled)

17. (canceled)

18. A nerve targeting peptide conjugate comprising:
(a) a peptide comprising the amino acid sequence of Formula (IV):
X₁-X₂-X₃-X₄-X₅-X₆-X₇-X₈-X₉-X₁₀-X₁₁-X₁₂-X₁₃-X₁₄, wherein:
X₁ is E, H, K, L, Q, R, S, V, or Y;
X₂ is A, D, F, G, H, I, L, S, V, or Y;
X₃ is A, H, K, L, N, P, Q, R, S, T, V, or W;
X₄ is A, C, E, F, G, I, L, M, P, R, S, T, V, W, or Y;
X₅ is A, D, E, G, H, I, K, N, Q, S, T, V, or Y;
X₆ is A, D, E, G, H, K, N, P, Q, S, T, V, or Y;
X₇ is A, H, L, P, Q, R, S, or T;
X₈ is C, D, E, F, H, I, K, L, N, S, V, or Y;
X₉ is A, D, F, H, K, L, N, S, V, W, or Y;
X₁₀ is A, E, F, G, I, L, P, R, S, V, or Y;
X₁₁ is A, C, E, F, G, I, L, M, N, P, Q, S, T, V, or W;
X₁₂ is A, E, H, I, K, N, P, Q, R, T, V, or W;
X₁₃ is D, E, H, I, K, M, N, Q, R, S, T, or Y; and
X₁₄ is A, F, H, I, M, P, R, S, T, W, or Y; and
(b) a cargo molecule.

19. (canceled)

20. (canceled)

21. A nerve targeting peptide conjugate comprising:
(a) a peptide comprising the amino acid sequence of Formula (V):
X₁-X₂-X₃-X₄-X₅-X₆-X₇-X₈-X₉-X₁₀-X₁₁-X₁₂-X₁₃-X₁₄, wherein:
X₁ is D, F, G, H, N, Q, R, T, or Y;
X₂ is D, F, H, I, L, M, Q, or V;
X₃ is D, E, G, L, P, Q, R, S, or T;
X₄ is G, L, Q, R, S, or W;
X₅ is A, D, E, G, H, K, Q, R, or V;
X₆ is A, E, G, Q, or V;
X₇ P or R;
X₈ is S or Y;
X₉ is G, H, N, Q, R, S, T, or Y;
X₁₀ is D, F, I, P, or V;
X₁₁ is A, D, F, G, I, L, Q, or V;
X₁₂ is A, E, H, K, L, N, R, S, or T;
X₁₃ is K, M, N, P, Q, T, or W; and
X₁₄ is A, F, I, L, P, R, S, or Y;
and
(b) a cargo molecule.

22. (canceled)

23. (canceled)

24. A nerve targeting peptide conjugate, comprising:
(a) a peptide comprising the amino acid sequence of Formula (VI):
X₁-X₂-X₃-X₄-X₅-X₆-X₇-X₈-X₉-X₁₀-X₁₁-X₁₂-X₁₃-X₁₄, wherein:
X₁ is D, H, L, N, Q, or Y;
X₂ is A, D, F, G, H, I, L, R, or V;
X₃ is A, F, L, P, S, T, or V;
X₄ is A, G, L, S, V, W, or Y;
X₅ is A, D, E, I, K, Q, or V;
X₆ is A, E, L, N, P, Q, or R;
X₇ is A, G, H, L, P, Q, R, or S;
X₈ A, D, F, K, N, R, S, or Y;
X₉ is D, E, F, K, L, N, S, or Y;
X₁₀ is A, E, F, G, I, L, N, P, T, or V;
X₁₁ is E, K, L, M, V, or W;
X₁₂ is E, H, K, L, Q, R, S, or T;
X₁₃ is E, H, K, M, N, Q, or Y; and
X₁₄ is F, G, I, S, or T;
and
(b) a cargo molecule.

25. (canceled)

26. (canceled)

27. A nerve targeting peptide conjugate, comprising:
(a) a peptide comprises or consists of the amino acid sequence of Formula (VII):
X₁-X₂-X₃-X₄-X₅-X₆-X₇-X₈-X₉-X₁₀-X₁₁-X₁₂-X₁₃-X₁₄, wherein:
X₁ is A, D, E, H, K, L, N, Q, R, or Y;
X₂ is A, C, D, E, F, G, I, L, R, S, or V;
X₃ is A, E, H, L, P, Q, R, S, T, W, or Y;
X₄ is C, F, G, I, L, M, S, T, V, or W;
X₅ is D, E, G, H, Q, S, or V;
X₆ is A, D, E, G, H, Q, T, V, or Y;
X₇ is H, L, P, Q, R, S, T, or V;
X₈ is C, D, E, F, H, L, N, Q, S, W, or Y;
X₉ is C, D, G, H, N, P, Q, S, W, or Y;
X₁₀ is A, D, G, I, L, P, T, or V;
X₁₁ is A, C, F, G, I, K, L, N, R, S, T, or V;
X₁₂ is D, K, N, Q, R, T, V, or Y;
X₁₃ is D, E, I, K, L, M, N, P, Q, R, T, or V; and
X₁₄ is C, F, S, V, or Y;
and
(b) a cargo molecule.

28. (canceled)

29. (canceled)

30. A nerve targeting peptide conjugate, comprising:
(a) a peptide comprising the amino acid sequence of Formula (VIII):
X₁-X₂-X₃-X₄-X₅-X₆-X₇-X₈-X₉-X₁₀-X₁₁-X₁₂-X₁₃-X₁₄, wherein:
X₁ is A, D, E, H, L, N, Q, R, S, T, Y;
X₂ is A, D, E, F, H, I, L, N, P, R, S, T, V, Y;
X₃ is C, I, L, P, Q, R, S, T, V;
X₄ is A, C, E, F, G, K, L, M, N, Q, R, S, T, V, W, Y;
X₅ is A, D, E, G, H, K, P, Q, R, S, T, V, Y;
X₆ is A, D, E, G, H, K, L, N, P, Q, R, S, T, V, W, Y;
X₇ is A, D, F, H, I, L, M, P, Q, S, T, W;
X₈ is C, F, G, H, I, K, L, N, P, Q, R, S, T, V, W, Y;
X₉ is A, C, D, E, F, G, H, N, S, T, Y;
X₁₀ is A, D, F, G, I, K, L, M, N, P, R, S, V;
X₁₁ is A, E, G, H, I, M, P, Q, V, Y;
X₁₂ is A, E, H, I, K, L, N, P, Q, R, T, V;
X₁₃ is A, D, E, I, K, M, N, P, R, S, T, V, Y; and
X₁₄ is A, C, F, G, H, L, N, P, S, T, Y;
and
(b) a cargo molecule.

31. (canceled)

32. (canceled)

33. A nerve targeting peptide conjugate, comprising:
(a) a peptide comprising the amino acid sequence of Formula (IX):
X₁-X₂-X₃-X₄-X₅-X₆-X₇-X₈-X₉-X₁₀-X₁₁-X₁₂-X₁₃-X₁₄, wherein:
X₁ is D, E, H, N, Q, R, S, W, or Y;
X₂ is D, F, H, I, L, M, N, P, R, S, V, or Y;
X₃ is A, E, H, L, P, Q, R, S, T, or Y;
X₄ is A, G, H, I, L, N, P, R, S, or W;
X₅ is A, E, G, I, K, Q, S, or V;
X₆ is A, D, E, G, K, L, P, Q, R, T, or V;
X₇ is A, G, K, L, P, Q, R, S, or T;
X₈ is E, I, K, L, S, V, or Y;
X₉ is A, D, F, G, H, I, K, N, Q, S, T, W, or Y;
X₁₀ is A, D, F, H, I, L, N, T, or V;
X₁₁ is A, D, E, F, G, L, M, V, or W;
X₁₂ is E, K, N, Q, R, T, or V;
X₁₃ is E, H, K, L, N, Q, R, S, T, or Y; and
X₁₄ is E, P, R, S, T, or Y;
and
(b) a cargo molecule.

34. (canceled)

35. (canceled)

36. A nerve targeting peptide conjugate, comprising:
(a) a peptide comprising the amino acid sequence of Formula (X):
X₁-X₂-X₃-X₄-X₅-X₆-X₇-X₈-X₉-X₁₀-X₁₁-X₁₂-X₁₃-X₁₄, wherein:
X₁ is A, D, E, G, H, K, N, Q, R, S, T, or Y;
X₂ is A, D, F, H, I, L, M, Q, S, T, V, or Y;
X₃ is A, E, G, K, L, N, P, Q, R, S, T, V, or Y;
X₄ is C, F, G, H, L, M, R, S, T, W, or Y;
X₅ is A, D, E, G, K, L, N, Q, T, or V;
X₆ is A, D, E, G, H, K, L, N, Q, S, or V,
X₇ is A, L, P, R, S, T, or Y;
X₈ is A, C, D, F, G, N, R, S, T, or Y;
X₉ is A, D, F, H, L, N, Q, R, S, T, or Y;
X₁₀ is A, D, E, F, G, H, I, L, P, R, S, T, or V;
X₁₁ is A, D, E, F, G, H, I, L, M, P, Q, R, S, or V;
X₁₂ is A, D, E, G, H, K, N, Q, S, T, or V;
X₁₃ is D, E, F, H, I, K, L, M, N, Q, R, S, T, V, or Y; and
X₁₄ is A, F, H, I, K, L, N, P, R, S, T, V, or Y;
and
(b) a cargo molecule.

37. (canceled)

38. (canceled)

39. A nerve targeting peptide conjugate of Formula (XI), comprising:
(a) a peptide comprising the amino acid sequence of Formula (XI):
X₁-X₂-X₃-X₄-X₅-X₆-X₇-X₈-X₉-X₁₀-X₁₁-X₁₂-X₁₃-X₁₄, wherein:
X₁ is D, E, H, L, Q, or R;
X₂ is F, G, H, I, L, N, Q, S, T, V, or Y;
X₃ is A, G, H, L, P, Q, R, S, T, or Y;
X₄ is A, C, F, G, L, P, Q, R, S, W, or Y;
X₅ is A, D, E, G, H, K, N, P, Q, S, T, V, or Y;
X₆ is A, D, E, G, H, K, L, N, Q, T, or V;
X₇ is A, E, H, K, L, P, Q, R, S, or T;
X₈ is C, D, E, F, G, H, K, L, N, P, Q, S, T, V, or Y;
X₉ is A, D, F, G, H, I, L, N, Q, S, T, V, W, or Y;
X₁₀ is A, D, F, G, H, I, L, P, R, S, T, V, or Y;
X₁₁ is A, D, E, F, G, H, L, M, Q, S, or V;
X₁₂ is A, E, H, I, K, L, N, P, Q, R, S, or T;
X₁₃ is A, D, H, I, K, L, M, N, P, Q, S, T, V, or Y; and
X₁₄ is A, F, H, L, P, S, T, V, or Y;
and
(b) a cargo molecule.

40. (canceled)

41. (canceled)

42. A nerve targeting peptide conjugate, comprising:
(a) the amino acid sequence of Formula (XII):
X₁-X₂-X₃-X₄-X₅-X₆-X₇-X₈-X₉-X₁₀-X₁₁-X₁₂-X₁₃-X₁₄, wherein:
X₁ is E, H, K, L, N, Q, or R;
X₂ is A, C, D, F, I, K, L, M, N, S, V, or Y;
X₃ is A, L, P, R, S, or T;
X₄ is A, E, F, G, L, M, R, S, T, W, or Y;
X₅ is A, D, E, F, G, H, K, N, Q, R, T, V, or Y;
X₆ is A, D, E, G, Q, R, S, T, V, or Y;
X₇ is A, H, K, L, P, Q, R, S, or T;
X₈ is A, C, D, F, H, N, R, S, T, or Y;
X₉ is D, E, F, H, L, N, R, S, T, V, or Y;
X₁₀ is A, D, F, G, H, I, L, N, R, S, T, or V;
X₁₁ is A, D, F, G, L, M, P, Q, R, T, V, or W;
X₁₂ is A, D, E, H, K, L, M, N, P, Q, R, S, T, or Y;
X₁₃ is E, H, I, K, L, M, N, P, Q, R, S, T, or V; and
X₁₄ is A, F, P, or S;
and
(b) a cargo molecule.

43. (canceled)

44. (canceled)

45. A nerve targeting peptide conjugate, comprising:
(a) a peptide comprising the amino acid sequence of Formula (XIII):
X₁-X₂-X₃-X₄-X₅-E-X₇-X₈-X₉-X₁₀-X₁₁-X₁₂-X₁₃-X₁₄, wherein:
X₁ is E, F, H, or Q;
X₂ is A, F, I, M, S, T, or V;
X₃ is A, G, H, P, Q, R, or T;
X₄ is F, G, L, M, N, R, S, W, or Y;
X₅ is A, G, K, N, P, Q, or R;
X₇ is H, P, R, or T;
X₈ is D, F, H, K, L, N, or Y;
X₉ is F, G, H, K, Q, T, V, or Y;
X₁₀ is F, G, L, P, S, V, or Y;
X₁₁ is L, M, Q, or V;
X₁₂ is E, G, I, K, L, N, P, S, or Y;
X₁₃ is E, H, I, N, Q, S, or T; and
X₁₄ is I, P, S, or Y;
and
(b) a cargo molecule.

46. (canceled)

47. (canceled)

48. A nerve targeting peptide conjugate, comprising:
(a) a peptide comprising the amino acid sequence of Formula (XIV):
X₁-X₂-X₃-X₄-X₅-X₆-X₇-X₈-X₉-X₁₀-X₁₁-X₁₂-X₁₃-X₁₄, wherein:
X₁ is H, L, N, Q, or R;
X₂ is F, L, R, S, or V;
X₃ is P, S, or T;
X₄ is A, L, S, or T;
X₅ is A, D, E, H, Q, R, V, or Y;
X₆ is D, E, or K;
X₇ is A, P, Q, or W;
X₈ is H, R, W, or Y;
X₉ is H, K, L, S, or Y;
X₁₀ is D, E, F, L, T, or V;
X₁₁ is A, D, or L;
X₁₂ is D, E, G, H, K, N, Q, or R;
X₁₃ is G, K, N, Q, or T; and
X₁₄ is P or S;
and
(b) a cargo molecule.

49. (canceled)

50. (canceled)

51. A nerve targeting peptide conjugate, comprising:
(a) a peptide comprising the amino acid sequence of Formula (XV):
X₁-X₂-X₃-X₄-X₅-X₆-X₇-Y-X₉-X₁₀-X₁₁-X₁₂-X₁₃-X₁₄, wherein:
X₁ is E, K, Q, or T;
X₂ is D, F, L, P, or V;
X₃ is C, L, P, T, or V;
X₄ is A, L, or T;
X₅ is D, E, H, or Q;
X₆ is A, D, K, or N;
X₇ is A, P, or S;
X₉ is C, H, M, or Y;
X₁₀ is F, L, or V;
X₁₁ is E, G, H, R, or V;
X₁₂ is K or T;
X₁₃ is I or Q; and
X₁₄ is A, F, or T;
and
(b) a cargo molecule.

52. (canceled)

53. (canceled)

54. A nerve targeting peptide conjugate, comprising:
(a) a peptide comprising the amino acid sequence of Formula (XVI):
X₁-X₂-X₃-X₄-X₅-X₆-X₇-X₈-X₉-X₁₀-X₁₁-X₁₂-X₁₃-X₁₄, wherein:
X₁ is D, H, K, or Q;
X₂ is F, I, or V;
X₃ is A, P, R, S, or T;
X₄ is G, L, M, Q, S, T, or W;
X₅ is D, G, Q, T, or V;
X₆ is D, E, H, Q, or S;
X₇ is A, P, R, or T;
X₈ is E, G, T, V, or Y;
X₉ is A, Q, S, or Y;
X₁₀ is A, D, E, G, I, L, R, T, V, or Y;
X₁₁ is A, E, F, G, or I;
X₁₂ is K, Q, or T;
X₁₃ is E, I, K, M, N, or S; and
X₁₄ is F, L, S, or T;
and;
(b) a cargo molecule.

55. (canceled)

56. (canceled)

57. A nerve targeting peptide conjugate, comprising:
(a) a peptide comprising the amino acid sequence of Formula (XVII):
Q-X₂-X₃-X₄-X₅-X₆-X₇-X₈-X₉-X₁₀-X₁₁-X₁₂-X₁₃-X₁₄, wherein:
X₂ is H, S, or T;
X₃ is P, Q, or T;
X₄ is F, M, P, or W;
X₅ is D, E, or Q;
X₆ is E or P;
X₇ is P, R, or T;
X₈ is A, K, or Y;
X₉ is H or Y;
X₁₀ is G, R, or V;
X₁₁ is L or V;
X₁₂ is I, L, S, or T;
X₁₃ is H, K, M, or Q; and
X₁₄ is A or S;
and
(b) a cargo molecule.

58. (canceled)

59. (canceled)

60. A nerve targeting peptide conjugate, comprising:
(a) a peptide comprising an amino acid sequence of Formula (XVIII):
X₁-X₂-X₃-X₄-X₅-X₆-X₇-X₈-X₉-X₁₀-X₁₁-X₁₂-X₁₃-X₁₄, wherein:
X₁ is K, Q, or R;
X₂ is F or L;
X₃ is G, P, or T;
X₄ is E, L, Q, S, or W;
X₅ is A, D, E, F, P, Q, V, or Y;
X₆ is A, D, E, K, N, Q, T, or Y;
X₇ is H, L, P, Q, R, or T;
X₈ is F, N, or Y;
X₉ is H, T, or Y;
X₁₀ is F, H, I, L, N, T, or Y;
X₁₁ is D, G, L, M, Q, or V;
X₁₂ is D, E, K, R, or T;
X₁₃ is A, E, K, M, N, P, Q, R, or T; and
X₁₄ is A, S, or P;
and
(b) a cargo molecule.

61. (canceled)

62. (canceled)

63. A nerve targeting peptide conjugate, comprising:
(a) a peptide comprising the amino acid sequence of Formula (XIX):
X₁-X₂-X₃-X₄-X₅-X₆-X₇-X₈-X₉-X₁₀-X₁₁-X₁₂-X₁₃-X₁₄, wherein:
X₁ is Q or T;
X₂ is H, I, L, T, or V;
X₃ is P or T;
X₄ is A, C, F, L, R, S, T, or Y;
X₅ is A, E, G, H, or Q;
X₆ is A, E, K, Q, T, V, or Y;
X₇ is L, M, P, Q, or S;
X₈ is C, F, L, N, W, or Y;
X₉ is C or Y;
X₁₀ is A, L, P, S, or V;
X₁₁ is A, G, or V;
X₁₂ is K, N, Q, R, or V;
X₁₃ is A, E, K, T, V, or Y; and
X₁₄ is F, G, N, P, S, T, or Y.

64. (canceled)

65. (canceled)

66. A nerve targeting peptide conjugate, comprising:
(a) the amino acid sequence of Formula (XX):
X₁-X₂-X₃-X₄-X₅-X₆-X₇-X₈-X₉-X₁₀-X₁₁-X₁₂-X₁₃-X₁₄, wherein:
X₁ is E, H, N, Q, or S;
X₂ is H, I, L, or V;
X₃ is A, P, or T;
X₄ is G, L, M, or W;
X₅ is D, E, G, or V;
X₆ is A, D, E, G, H, L, or V;
X₇ is G, K, P, Q, R, or T;
X₈ is E, L, S, or Y;
X₉ is H, K, N, Q, R, S, V, or Y;
X₁₀ is A, F, I, L, or P;
X₁₁ is F, G, I, L, M, P, R, or V;
X₁₂ is H, K, N, or V;
X₁₃ is H, K, L, M, R, S, or T; and
X₁₄ is F, P, R, S, or Y;
and
(b) a cargo molecule.

67. (canceled)

68. (canceled)

69. A nerve targeting peptide conjugate, comprising:
(a) a peptide comprising the amino acid sequence of Formula (XXI):
X₁-X₂-X₃-X₄-X₅-E-X₇-X₈-X₉-X₁₀-X₁₁-K-X₁₃-X₁₄, wherein:
X₁ is D, Q, or R;
X₂ is A, F, H, I, L, V, or Y;
X₃ is A, E, L, P, or Q;
X₄ is A, L, R, S, or V;
X₅ is A, E, G, H, K, Q, or R;
X₇ is K, L, P, R, or T;
X₈ is D, N, R, or Y;
X₉ is E, F, H, N, R, S, or T;
X₁₀ is A, D, F, I, L, R, or V;
X₁₁ is A, F, L, M, P, Q, V, or W;
X₁₃ is E, H, K, M, or R; and
X₁₄ is P, F, or S;
and
(b) a cargo molecule.

70. (canceled)

71. (canceled)

72. A nerve targeting peptide conjugate, comprising:
(a) a peptide comprising the amino acid sequence of Formula (XXII):
X₁-X₂-X₃-X₄-X₅-X₆-X₇-X₈-X₉-X₁₀-X₁₁-X₁₂-X₁₃-X₁₄, wherein:
X₁ is A, H, L, or Q;
X₂ is D, F, I, L, N, S, T, or Y;
X₃ is P, Q, S, or T;
X₄ is A, C, L, S, or W;
X₅ is A, D, E, H, K, Q, S, T, or V;
X₆ is A, D, E, G, H, K, Q, R, S, W, or Y;
X₇ is D, P, or S;
X₈ is C, F, G, H, L, N, Q, S, T, W, or Y;
X₉ is A, C, D, F, N, S, or Y;
X₁₀ is A, D, G, L, or V;
X₁₁ is A, G, L, Q, S, T, V, W, or Y;
X₁₂ is A, D, K, P, Q, S, T, or Y;
X₁₃ is I, K, M, N, P, R, or T; and
X₁₄ is A, C, F, P, S, or Y;
and
(b) a cargo molecule.

73. (canceled)

74. (canceled)

75. A nerve targeting peptide conjugate, comprising:
(a) a peptide comprising the amino acid sequence of Formula (XXIII):
X₁-X₂-X₃-X₄-X₅-X₆-X₇-X₈-X₉-X₁₀-X₁₁-X₁₂-X₁₃-X₁₄, wherein:
X₁ is D, E, or Q;
X₂ is L, M, N, P, R, or V;
X₃ is P, Q, R, or T;
X₄ is R, S, or W;
X₅ is E, G, H, Q, T, or Y;
X₆ is A, E, K, N, Q, or V;
X₇ is A, P, R, or S;
X₈ is D, F, H, K, N, or Y;
X₉ is D, F, H, N, S, T, W, or Y;
X₁₀ is G, I, R, or V;
X₁₁ is D, G, M, or V;
X₁₂ is E, K, N, R, or V;
X₁₃ is I, K, N, Q, R, S, T, V, or Y; and
X₁₄ is G, H, P, S, T, or Y;
and
(b) a cargo molecule.

76. (canceled)

77. (canceled)

78. A nerve targeting peptide conjugate, comprising:
(a) a peptide comprising the amino acid sequence of Formula (XXIV):
Q-X₂-P-X₄-X₅-X₆-X₇-X₈-X₉-X₁₀-X₁₁-X₁₂-X₁₃-S wherein:
X₂ is D, I, K, L, or M;
X₄ is F, G, L, S, or T;
X₅ is E, G, H, N, Q, V, or Y;
X₆ is A, D, E, G, or S;
X₇ is L, P, or S;
X₈ is N, T, or Y;
X₉ is D, N, or Y;
X₁₀ is A, G, I, S, or V;
X₁₁ is D, G, L, Q, or T;
X₁₂ is K, L, M, N, Q, R, S, or T; and
X₁₃ is E, K, M, N, P, S, or V;
and
(b) a cargo molecule.

79. (canceled)

80. (canceled)

81. A nerve targeting peptide conjugate, comprising:
(a) a peptide comprising the amino acid sequence of Formula (XXV):
Q-X₂-P-X₄-X₅-X₆-X₇-X₈-X₉-X₁₀-X₁₁-X₁₂-X₁₃-X₁₄, wherein:
X₂ is F, I, L, N, S, or V;
X₄ is E, G, L, R, W, or Y;
X₅ is A, D, G, H, K, N, Q, S, T, V, or Y;
X₆ is A, D, E, H, N, Q, S, or T;
X₇ is A, K, L, P, or R;
X₈ is N, Q, S, T, or Y;
X₉ is E, F, H, N, V, or Y;
X₁₀ is S or V;
X₁₁ is F, H, L, M, S, or V;
X₁₂ is E, H, I, K, or P;
X₁₃ is K, N, Q, S, or Y; and
X₁₄ is F, S, V, or Y; and
(b) a cargo molecule.

82. (canceled)

83. (canceled)

84. A nerve targeting peptide conjugate, comprising:
(a) a peptide comprising the amino acid sequence of Formula (XXVI):
X₁-X₂-X₃-X₄-X₅-X₆-X₇-X₈-X₉-X₁₀-X₁₁-X₁₂-X₁₃-X₁₄, wherein:
X₁ is N, Q, R, S, or V;
X₂ is A, D, F, H, I, L, N, or V;
X₃ is L, P, Q, R, or S;
X₄ is A, C, E, G, H, I, L, R, S, V, W, or Y;
X₅ is E, G, K, N, P, Q, or S;
X₆ is A, D, E, G, K, L, or R;
X₇ is H, L, Q, S, or T;
X₈ is F, H, K, or Y;
X₉ is D, F, I, L, N, or Y;
X₁₀ is A, F, G, L, M, or V;
X₁₁ is A, F, G, I, L, M, P, V, or W;
X₁₂ is A, K, N, T, or Y;
X₁₃ is H, K, L, M, N, Q, S, T, or Y; and
X₁₄ is C, E, G, N, P, or S;
and
(b) a cargo molecule.

85. (canceled)

86. (canceled)

87. A nerve targeting peptide conjugate, comprising:
(a) a peptide comprising the amino acid sequence of Formula (XXVII):
X₁-X₂-X₃-X₄-X₅-X₆-X₇-X₈-X₉-X₁₀-X₁₁-X₁₂-X₁₃-X₁₄, wherein:
X₁ is D, E, Q, or R;
X₂ is F, G, I, L, or V;
X₃ is A, H, P, Q, R, or S;
X₄ is C, L, R, S, T, W, or Y;
X₅ is A, E, G, H, P, Q, or V;
X₆ is A, D, E, G, K, Q, or T;
X₇ is A, H, Q, R, or S;
X₈ is C, E, F, N, Q, S, or Y;
X₉ is C, H, S, V, W, or Y;
X₁₀ is D, I, L, T, or V;
X₁₁ is A, E, G, or V;
X₁₂ is K, P, or T;
X₁₃ is K, M, N, Q, R, S, or T; and
X₁₄ is S or Y.

88. (canceled)

89. (canceled)

90. A nerve targeting peptide conjugate, comprising:
(a) a peptide comprising the amino acid sequence of Formula (XXVIII):
X₁-X₂-X₃-X₄-X₅-X₆-X₇-X₈-X₉-X₁₀-X₁₁-X₁₂-X₁₃-X₁₄, wherein:
X₁ is H, Q, or S;
X₂ is C, F, G, I, V, or Y;
X₃ is A, P, Q, R, S, T, or Y;
X₄ is C, L, N, S, W, or Y;
X₅ is D, E, H, K, Q, S, or V;
X₆ is A, E, G, K, or Q;
X₇ is L or S;
X₈ is C, N, P, S, V, or Y;
X₉ is A, D, F, G, N, S, or Y;
X₁₀ is D, G, L, S, V, or Y;
X₁₁ is A, E, F, G, L, Q, S, or V;
X₁₂ is A, K, N, Q, R, S, or T;
X₁₃ is I, K, or T; and
X₁₄ is I, L, P, R, S, T, or W;
and
(b) a cargo molecule.

91. (canceled)

92. (canceled)

93. A nerve targeting peptide conjugate, comprising:
(a) a peptide comprising the amino acid sequence of Formula (XXIX):
X₁-X₂-X₃-X₄-X₅-X₆-X₇-X₈-X₉-X₁₀-X₁₁-X₁₂-X₁₃-X₁₄, wherein:
X₁ is D or E;
X₂ is F, G, H, I, R, or V;
X₃ is A, L, P, or T;
X₄ is F, G, L, R, S, T, W, or Y;
X₅ is A, D, E, G, I, P, R, or V;
X₆ is A, D, E, H, K, L, or R;
X₇ is L, P, Q, R, S, T, or W;
X₈ is F, H, N, R, S, V, or Y;
X₉ is F, K, or Y;
X₁₀ is A, D, F, G, I, L, N, R, or V;
X₁₁ is A, E, F, G, L, M, or V;
X₁₂ is E, K, P, Q, S, T, or V;
X₁₃ is D, F, H, I, K, or Q; and
X₁₄ is G, P, S, T, or Y;
and
(b) a cargo molecule.

94. (canceled)

95. (canceled)

96. A nerve targeting peptide conjugate, comprising:
(a) a peptide comprising the amino acid sequence of Formula (XXX):
X₁-X₂-X₃-X₄-X₅-X₆-X₇-X₈-X₉-X₁₀-X₁₁-X₁₂-X₁₃-X₁₄, wherein:
X₁ is D, E, H, L, or R;
X₂ is F, G, H, I, L, S, or V;
X₃ is L, P, or Q;
X₄ is A, C, G, L, P, Q, R, S, T, V, or W;
X₅ is D, E, G, H, K, or Q;
X₆ is D, E, K, T, V, or Y;
X₇ is A, E, L, P, R, S, T, or W;
X₈ is D, E, F, H, S, W, or Y;
X₉ is I, W, or Y;
X₁₀ is A, D, I, P, or V;
X₁₁ is A, C, F, G, H, I, L, M, T, or V;
X₁₂ is D, E, H, K, T, or V;
X₁₃ is G, H, I, K, L, N, Q, R, S, V, or Y; and
X₁₄ is A, F, H, I, M, S, or Y.

97. (canceled)

98. (canceled)

99. A nerve targeting peptide conjugate, comprising:
(a) a peptide comprises or consists of the amino acid sequence of Formula (XXXI):
X₁-X₂-X₃-X₄-X₅-X₆-X₇-X₈-X₉-X₁₀-X₁₁-X₁₂-X₁₃-X₁₄, wherein:
X₁ is D, E, H, L, Q, R, T, or Y;
X₂ is F, G, I, R, S, or V;
X₃ is A, G, K, L, P, R, S, or T;
X₄ is A, G, L, R, S, T, W, or Y;
X₅ is A, E, G, H, K, N, P, Q, T, V, or Y;
X₆ is A, D, E, G, or N;
X₇ is A, P, Q, S, or T;
X₈ is D, F, G, H, N, R, S, or Y;
X₉ is D, F, G, H, K, N, Q, R, S, T, V, or Y;
X₁₀ is E, I, L, N, T, V, or Y;
X₁₁ is A, F, G, K, L, M, Q, or V;
X₁₂ is I, K, L, N, Q, R, or T;
X₁₃ is E, H, I, K, M, N, Q, T, or W; and
X₁₄ is F, H, I, L, N, P, R, or S;
and
(b) a cargo molecule.

100. (canceled)

101. (canceled)

102. A nerve targeting peptide conjugate, comprising:
(a) a peptide comprising the amino acid sequence of Formula (XXXII):
X₁-X₂-X₃-X₄-X₅-X₆-X₇-X₈-X₉-X₁₀-X₁₁-X₁₂-X₁₃-X₁₄, wherein:
X₁ is A, H, L, N, Q, or Y;
X₂ is C, D, F, H, I, L, R, S, T, V, or Y;
X₃ is A, G, L, N, P, Q, R, T, V, or Y;
X₄ is C, F, G, R, S, or W;
X₅ is A, E, or T;
X₆ is A, D, E, Q, S, or Y;
X₇ is A, L, P, T, or Y;
X₈ is C, E, F, G, N, Q, S, or Y;
X₉ is H, S, or Y;
X₁₀ is A, F, H, L, R, S, T, or V;
X₁₁ is A, C, D, E, F, G, H, L, M, N, Q, or V;
X₁₂ is A, D, E, G, H, K, N, Q, S, or T;
X₁₃ is D, E, F, I, K, L, M, N, P, Q, R, S, T, or Y; and
X₁₄ is A, F, K, L, P, S, V, or Y;
and
(b) a cargo molecule.

103. (canceled)

104. (canceled)

105. A nerve targeting peptide conjugate, comprising:
(a) a peptide comprising the amino acid sequence of Formula (XXXIII):
X₁-X₂-X₃-X₄-X₅-X₆-X₇-X₈-X₉-X₁₀-X₁₁-X₁₂-X₁₃-X₁₄, wherein:
X₁ is D, E, H, L, N, Q, R, or Y;
X₂ is A, D, E, F, I, L, T, V, or Y;
X₃ is A, E, L, P, Q, R, or S;
X₄ is F, G, P, R, S, or W;
X₅ is A, D, E, G, H, Q, S, or V;
X₆ is A, D, E, G, H, Q, or T;
X₇ is P, Q, R, S, or T;
X₈ is D, F, H, K, N, S, or Y;
X₉ is D, F, G, H, N, P, Q, S, or Y;
X₁₀ is A, D, F, G, I, L, P, R, or V;
X₁₁ is A, E, G, I, L, R, or V;
X₁₂ is D, E, K, N, Q, R, T, or V;
X₁₃ is D, E, I, K, L, Q, R, or T; and
X₁₄ is A, I, S, T, or Y;
and
(b) a cargo molecule.

106. (canceled)

107. (canceled)

108. A nerve targeting peptide conjugate, comprising:
(a) a peptide comprising the amino acid sequence of Formula (XXXIV):
X₁-X₂-X₃-X₄-X₅-X₆-X₇-X₈-X₉-X₁₀-X₁₁-X₁₂-X₁₃-X₁₄, wherein:
X₁ is A, D, E, K, L, N, Q, R, S, T, or Y;
X₂ is D, E, F, I, L, N, Q, R, S, T, or V;
X₃ is I, L, P, Q, or T;
X₄ is F, G, I, K, M, N, Q, R, S, V, W, or Y;
X₅ is A, D, E, G, P, Q, V, or Y;
X₆ is A, D, E, G, H, L, P, Q, S, or V;
X₇ is F or P;
X₈ is D, G, K, N, R, S, V, or Y;
X₉ is D, E, L, or Y;
X₁₀ is D, F, G, K, L, M, N, R, or V;
X₁₁ is A, E, F, G, M, P, Q, R, or V;
X₁₂ is K, L, N, or V;
X₁₃ is A, D, K, M, N, T, or V; and
X₁₄ is A, F, H, L, P, S, T, V, or Y; and
(b) a cargo molecule.

109. (canceled)

110. (canceled)

111. A nerve targeting peptide conjugate, comprising:
(a) a peptide comprising the amino acid sequence of Formula (XXXV):
X₁-X₂-X₃-X₄-X₅-X₆-X₇-X₈-X₉-X₁₀-X₁₁-X₁₂-X₁₃-X₁₄, wherein:
X₁ is A, E, H, L, N, Q, R, S, V, or Y;
X₂ is F, I, N, S, V, or Y;
X₃ is A, E, I, L, P, R, S, T, V, W, or Y;
X₄ is C, F, G, K, L, R, S, W, or Y;
X₅ is D, E, I, K, Q, V, or Y;
X₆ is A, D, E, G, N, P, Q, R, S, V, or Y;
X₇ is G, L, P, Q, or S;
X₈ is H, I, L, N, S, or Y;
X₉ is A, D, E, F, L, N, S, T, V, or Y;
X₁₀ is A, D, E, F, G, H, I, L, N, S, T, or V;
X₁₁ is K, L, M, or V;
X₁₂ is I, K, N, P, Q, R, S, or T;
X₁₃ is A, C, E, H, K, M, N, Q, R, S, T, or Y; and
X₁₄ is F, H, P, S, T, or V; and
(b) a cargo molecule.

112. (canceled)

113. (canceled)

114. A nerve targeting peptide conjugate, comprising:
(a) a peptide comprising the amino acid sequence of Formula (XXXVI):
X₁-X₂-X₃-X₄-X₅-X₆-X₇-X₈-X₉-X₁₀-X₁₁-X₁₂-X₁₃-X₁₄, wherein:
X₁ is D, F, H, L, N, Q, R, S, T, or Y;
X₂ is A, D, F, G, I, L, Q, or V;
X₃ is A, D, E, F, G, H, K, L, P, Q, R, S, T, or V;
X₄ is A, C, E, F, G, I, L, P, Q, R, S, V, W, or Y;
X₅ is A, D, E, G, H, I, K, L, N, P, Q, R, S, T, V, or Y;
X₆ is A, D, E, G, K, N, P, Q, S, T, V, or Y;
X₇ is A, H, I, L, P, Q, R, S, or T;
X₈ is A, E, F, H, I, K, L, P, Q, R, S, V, or Y;
X₉ is A, C, D, F, G, H, K, L, N, P, Q, R, S, or Y;
X₁₀ is A, D, E, F, G, H, I, L, N, P, S, V, or Y;
X₁₁ is A, D, E, F, G, I, L, M, N, S, T, or V;
X₁₂ is D, E, G, H, I, K, N, Q, R, S, T, W, or Y;
X₁₃ is D, E, H, K, N, Q, R, S, T, or V; and
X₁₄ is A, I, P, S, or Y.

115. (canceled)

116. (canceled)

117. A nerve targeting peptide conjugate, comprising:
(a) a peptide comprising the amino acid sequence of Formula (XXXVII):
X₁-X₂-X₃-X₄-X₅-X₆-X₇-X₈-X₉-X₁₀-X₁₁-X₁₂-X₁₃-X₁₄, wherein:
X₁ is A, E, H, Q, or R;
X₂ is D, F, G, L, M, or V;
X₃ is A, E, H, P, Q, R, S, T, or W;
X₄ is C, G, L, R, S, V, or W;
X₅ is A, D, E, G, I, K, L, or Y;
X₆ is A, D, E, G, K, L, N, Q, S, or V;
X₇ is A, H, L, M, P, S, T, or W;
X₈ is C, H, I, L, N, S, V, or Y;
X₉ is D, H, L, N, S, or Y;
X₁₀ is A, D, E, F, G, L, P, S, T, or V;
X₁₁ is A, D, E, G, I, M, Q, R, T, V, or W;
X₁₂ is H, I, K, L, N, Q, R, or T;
X₁₃ is A, E, H, I, K, M, N, R, or V; and
X₁₄ is F, P, S, or T;
and
(b) a cargo molecule.

118. (canceled)

119. (canceled)

120. A nerve targeting peptide conjugate, comprising:
(a) a peptide comprising the amino acid sequence of Formula (XXXVIII):
X₁-X₂-X₃-X₄-X₅-X₆-X₇-X₈-X₉-X₁₀-X₁₁-X₁₂-X₁₃-X₁₄, wherein:
X₁ is D, E, G, H, K, L, R, or S;
X₂ is A, F, I, or L;
X₃ is A, E, F, L, P, Q, R, or S;
X₄ is E, F, G, M, R, S, V, W, or Y;
X₅ is E, P, or Q;
X₆ is A, D, E, or G;
X₇ is H, L, P, R, S, or T;
X₈ G, L, S, or Y;
X₉ is D, H, K, L, T, or Y;
X₁₀ is A, F, G, I, L, or R;
X₁₁ L, P, or V;
X₁₂ is A, E, H, I, K, or R;
X₁₃ is E, I, K, N, Q, R, S, or T; and
X₁₄ is A, F, H, P, R, S, T, or Y;
and
(b) a cargo molecule.

121. (canceled)

122. (canceled)

123. A nerve targeting peptide conjugate, comprising:
(a) a peptide comprising the amino acid sequence of Formula (XXXIX):
X₁-X₂-X₃-X₄-X₅-X₆-X₇-X₈-X₉-X₁₀-X₁₁-X₁₂-X₁₃-X₁₄, wherein:
X₁ is A, E, N, Q, R, or W;
X₂ is A, D, I, L, S, T, or V;
X₃ is E, H, L, P, T, or Y;
X₄ is L, S, or W;
X₅ is A, E, G, V, or Y;
X₆ is E, G, I, K, P, Q, or T;
X₇ is H, P, R, or S;
X₈ is S or Y;
X₉ is F, S, W, or Y;
X₁₀ is A, D, F, G, H, I, T, or V;
X₁₁ is L or V;
X₁₂ is E, H, K, or N;
X₁₃ is H, K, N, or V; and
X₁₄ is F, S, T, or Y;
and
(b) a cargo molecule.

124. (canceled)

125. (canceled)

126. A nerve targeting peptide conjugate, comprising:
(a) a peptide comprising the amino acid sequence of Formula (XL):
X₁-X₂-X₃-X₄-X₅-X₆-X₇-X₈-X₉-V-X₁₁-X₁₂-X₁₃-X₁₄, wherein:
X₁ is D, E, H, L, or Q;
X₂ is D, F, L, S, or V;
X₃ is A, H, L, P, Q, or S;
X₄ is E, G, M, R, or W;
X₅ is A, D, E, Q, or T;
X₆ is D or E;
X₇ is A, L, R, S, or V;
X₈ is F, L, N, or Y;
X₉ is F, G, H, L, N, S, or Y;
X₁₁ is K, M, or V;
X₁₂ is K or T;
X₁₃ is D, K, or N; and
X₁₄ is F, P, S, or T;
and
(b) a cargo molecule.

127. (canceled)

128. (canceled)

129. A nerve targeting peptide conjugate, comprising:
(a) a peptide comprising the amino acid sequence of Formula (XL1):
X₁-X₂-X₃-X₄-X₅-X₆-X₇-X₈-X₉-V-X₁₁-X₁₂-X₁₃-X₁₄, wherein:
X₁ is H or Q;
X₂ is A, F, I, L, T, or V;
X₃ is H, L, P, R, S, or T;
X₄ is A, F, G, L, S, W, or Y;
X₅ is A, D, E, G, N, or T;
X₆ is A, E, or V;
X₇ is A, H, P, S, or T;
X₈ is A, D, H, N, Q, T, or Y;
X₉ is G, H, L, S, T, V, or Y;
X₁₁ is S or V;
X₁₂ is K, L, N, P, Q, R, T, or Y;
X₁₃ is E, H, I, K, N, or T; and
X₁₄ is F or S;
and
(b) a cargo molecule.

130. (canceled)

131. (canceled)

132. A nerve targeting peptide conjugate, comprising:
(a) a peptide comprising the amino acid sequence of Formula (XLII):
X₁-X₂-X₃-X₄-X₅-X₆-X₇-X₈-X₉-X₁₀-X₁₁-X₁₂-X₁₃-X₁₄, wherein:
X₁ is H, L, Q, R, or V;
X₂ is F, G, H, I, L, or V;
X₃ is A, H, N, P, Q, or T;
X₄ is F, G, L, M, S, T, or W;
X₅ is A, D, G, I, K, P, Q, or V;
X₆ is D, E, G, K, or P;
X₇ is A, L, P, Q, R, S, or T;
X₈ is F, H, S, or Y;
X₉ is A, D, F, G, H, K, N, S, or Y;
X₁₀ is I, L, N, or V;
X₁₁ is A, E, G, K, L, Q, S, V, or W;
X₁₂ is A, D, E, G, I, K, M, N, Q, R, or T;
X₁₃ is E, H, K, N, Q, R, T, or V; and
X₁₄ is F, H, N, P, S, T, or Y;
and
(b) a cargo molecule.

133. (canceled)

134. (canceled)

135. A nerve targeting peptide conjugate, comprising
(a) a peptide comprising the amino acid sequence of Formula (XLIII):
X₁-X₂-X₃-X₄-X₅-X₆-X₇-Y-X₉-V-X₁₁-X₁₂-X₁₃-X₁₄, wherein
X₁ is G, H, N, Q, R, or Y;
X₂ is F, H, I, L, M, or V;
X₃ is L, P, or Q;
X₄ is G, L, Q, R, S, or W;
X₅ is G, H, K, Q, R, or V;
X₆ is E or V;
X₇ is P or R;
X₉ is N, S, Y;
X₁₁ is L, Q, or V;
X₁₂ is A, E, K, L, or T;
X₁₃ is K, M, N, P, Q, or T; and
X₁₄ is A, F, I, L, P, S, or Y;
and
(b) a cargo molecule.

136. (canceled)

137. (canceled)

138. A nerve targeting peptide conjugate, comprising:
(a) a peptide comprising the amino acid sequence of Formula (XLIV):
Q-X₂-X₃-X₄-X₅-X₆-X₇-X₈-X₉-X₁₀-X₁₁-X₁₂-X₁₃-X₁₄, wherein:
X₂ is H, I, M, N, or V;
X₃ is A, L, T, or V;
X₄ is F, H, L, N, S, or W;
X₅ is A, D, K, N, or S;
X₆ is A, E, H, K, or Q;
X₇ is P or S;
X₈ is F, H, N, or Y;
X₉ is N, S, T, or Y;
X₁₀ is G, P, S, V, or Y;
X₁₁ is Q or V;
X₁₂ is E or K;
X₁₃ is D, N, P, R, or T; and
X₁₄ is S, T, or Y;
and
(b) a cargo molecule.

139. (canceled)

140. (canceled)

141. The nerve targeting peptide conjugate of claim 1, wherein the cargo molecule comprises a drug, a fluorescent moiety, or a photosensitizing agent.

142. The nerve targeting peptide conjugate of claim 1, wherein the cargo molecule is joined to the N-terminus or C-terminus of the peptide.

143. The nerve targeting peptide conjugate of claim 1, wherein the cargo molecule is joined to the peptide via a linker.

144.-154. (canceled)

155. A pharmaceutical composition comprising the nerve targeting peptide conjugate of claim 1 and a pharmaceutically acceptable carrier.

156. A method of delivering a cargo molecule to a neuron or nerve comprising contacting the neuron or nerve with the nerve targeting peptide conjugate of claim 1.

157. A method of identifying a neuron or nerve comprising contacting the neuron or nerve with the nerve targeting peptide conjugate of claim 141, wherein the cargo molecule comprises a fluorescent moiety.

158. A method of delivering a photosensitizing agent to a neuron or nerve comprising contacting the neuron or nerve with the nerve targeting peptide conjugate of claim 141, wherein the cargo molecule comprises a photosensitizing agent.

159. (canceled)

160. A method of delivering a drug to a neuron or nerve comprising contacting the neuron or nerve with the nerve targeting peptide conjugate of claim 141, wherein the cargo molecule comprises a drug.

161.-169. (canceled)


SEQUENCE LISTING (576 sequences)

SEQ ID NO: 1 - protein, 14 aa, synthetic construct
TIPYAEPYYVVRKY

SEQ ID NO: 2 - protein, 14 aa, synthetic construct
DFTMQEPYYAEKKT

SEQ ID NO: 3 - protein, 14 aa, synthetic construct
HVTWQEQYYVLKTP

SEQ ID NO: 4 - protein, 14 aa, synthetic construct
HVPGVDPYSVVKKF

SEQ ID NO: 5 - protein, 14 aa, synthetic construct
HVAFEQPDNVLVLY

SEQ ID NO: 6 - protein, 14 aa, synthetic construct
QVPTEVQFYLVKEP

SEQ ID NO: 7 - protein, 14 aa, synthetic construct
QLPSEQPCCVVKKY

SEQ ID NO: 8 - protein, 14 aa, synthetic construct
QDPWVTRYYIVKKS

SEQ ID NO: 9 - protein, 14 aa, synthetic construct
QVPAKEPYNFMKKS

SEQ ID NO: 10 - protein, 14 aa, synthetic construct
YLTWEEPEHVNKQS

SEQ ID NO: 11 - protein, 14 aa, synthetic construct
QVPRQELYYVSHQF

SEQ ID NO: 12 - protein, 14 aa, synthetic construct
QHPFEQSFYVVNKF

SEQ ID NO: 13 - protein, 14 aa, synthetic construct
DILLDELRFAEKHG

SEQ ID NO: 14 - protein, 14 aa, synthetic construct
YNLWDAPSYLVKNS

SEQ ID NO: 15 - protein, 14 aa, synthetic construct
HVAGGERYFSVKES

SEQ ID NO: 16 - protein, 14 aa, synthetic construct
HLPSDQPLSVTKIS

SEQ ID NO: 17 - protein, 14 aa, synthetic construct
GFLGEEPYHFLAKR

SEQ ID NO: 18 - protein, 14 aa, synthetic construct
LRSTADAHYTLKKS

SEQ ID NO: 19 - protein, 14 aa, synthetic construct
ATPCSQPCAVVPKF

SEQ ID NO: 20 - protein, 14 aa, synthetic construct
QYAWEEPYYLEKKL

SEQ ID NO: 21 - protein, 14 aa, synthetic construct
HLPVEYPFYVFKNS

SEQ ID NO: 22 - protein, 14 aa, synthetic construct
RGSLEGPFKLGKTH

SEQ ID NO: 23 - protein, 14 aa, synthetic construct
QVSSQEPNFIVKNN

SEQ ID NO: 24 - protein, 14 aa, synthetic construct
HFSAYEQYYVLRNS

SEQ ID NO: 25 - protein, 14 aa, synthetic construct
YVIKYAPYYAVTRS

SEQ ID NO: 26 - protein, 14 aa, synthetic construct
HLPEEELYTIVRKT

SEQ ID NO: 27 - protein, 14 aa, synthetic construct
QDPCVWPYNGVKKC

SEQ ID NO: 28 - protein, 14 aa, synthetic construct
HVHWDATYYAVTNS

SEQ ID NO: 29 - protein, 14 aa, synthetic construct
QFPYDHKNYVVKYS

SEQ ID NO: 30 - protein, 14 aa, synthetic construct
RFHMVGPYNVATTS

SEQ ID NO: 31 - protein, 14 aa, synthetic construct
HLPWEGQLNIGNKS

SEQ ID NO: 32 - protein, 14 aa, synthetic construct
DDLWQDLYGVVKKS

SEQ ID NO: 33 - protein, 14 aa, synthetic construct
NLAWAESKFLLEKI

SEQ ID NO: 34 - protein, 14 aa, synthetic construct
QLRYQEHYYLGYNS

SEQ ID NO: 35 - protein, 14 aa, synthetic construct
YVPWQEPYYVQKNL

SEQ ID NO: 36 - protein, 14 aa, synthetic construct
LVPWDETHKVVQRH

SEQ ID NO: 37 - protein, 14 aa, synthetic construct
HVALEQPYYAVKES

SEQ ID NO: 38 - protein, 14 aa, synthetic construct
QVQCKEQYYGVTNC

SEQ ID NO: 39 - protein, 14 aa, synthetic construct
QALYAEPAYVSKKF

SEQ ID NO: 40 - protein, 14 aa, synthetic construct
HVSMDERYYVVKKT

SEQ ID NO: 41 - protein, 14 aa, synthetic construct
HSYREEPYYFGKRS

SEQ ID NO: 42 - protein, 14 aa, synthetic construct
QHTWKEPYNGQKRT

SEQ ID NO: 43 - protein, 14 aa, synthetic construct
QLPMGGPYERVNNT

SEQ ID NO: 44 - protein, 14 aa, synthetic construct
SVPKVHPYYVVNNS

SEQ ID NO: 45 - protein, 14 aa, synthetic construct
QVPTGEPLYPVQKT

SEQ ID NO: 46 - protein, 14 aa, synthetic construct
QLPSYQAHDVMKVH

SEQ ID NO: 47 - protein, 14 aa, synthetic construct
QIPSQRPSSVGAIA

SEQ ID NO: 48 - protein, 14 aa, synthetic construct
QFPGDERYYLATST

SEQ ID NO: 49 - protein, 14 aa, synthetic construct
HVSSQEPYYVLRNS

SEQ ID NO: 50 - protein, 14 aa, synthetic construct
RLPSDDHNTFGTRS

SEQ ID NO: 51 - protein, 14 aa, synthetic construct
QLHGDELLSVVKNP

SEQ ID NO: 52 - protein, 14 aa, synthetic construct
ATEWEESYFHVKKF

SEQ ID NO: 53 - protein, 14 aa, synthetic construct
RVPWEQSSWVVKRS

SEQ ID NO: 54 - protein, 14 aa, synthetic construct
QIANKEPHTYVENS

SEQ ID NO: 55 - protein, 14 aa, synthetic construct
QIRSEVTHSVVRES

SEQ ID NO: 56 - protein, 14 aa, synthetic construct
QHTLNHPYSVVKTS

SEQ ID NO: 57 - protein, 14 aa, synthetic construct
QVPSHELYFRPKES

SEQ ID NO: 58 - protein, 14 aa, synthetic construct
EVPGGVPYYPMKKF

SEQ ID NO: 59 - protein, 14 aa, synthetic construct
HVSGGQWVHGQKNS

SEQ ID NO: 60 - protein, 14 aa, synthetic construct
HVPSKQMNSLAKKS

SEQ ID NO: 61 - protein, 14 aa, synthetic construct
QIHFGEPNYVVKKS

SEQ ID NO: 62 - protein, 14 aa, synthetic construct
QFPWEGSYYTAKQS

SEQ ID NO: 63 - protein, 14 aa, synthetic construct
QIPWGEAYQAEKIT

SEQ ID NO: 64 - protein, 14 aa, synthetic construct
VVPSNAQHLAPNMS

SEQ ID NO: 65 - protein, 14 aa, synthetic construct
HVAEAESYNVVKKT

SEQ ID NO: 66 - protein, 14 aa, synthetic construct
QIPCEGPYSGVQMC

SEQ ID NO: 67 - protein, 14 aa, synthetic construct
HITGGVPYPVVRDS

SEQ ID NO: 68 - protein, 14 aa, synthetic construct
HVLWEDSSYHVTHH

SEQ ID NO: 69 - protein, 14 aa, synthetic construct
HVTYDEQKYFVKKS

SEQ ID NO: 70 - protein, 14 aa, synthetic construct
QVPSVQPYYIGKNT

SEQ ID NO: 71 - protein, 14 aa, synthetic construct
QARFEQPYSGLRDS

SEQ ID NO: 72 - protein, 14 aa, synthetic construct
LIPSAYPHSLWSKS

SEQ ID NO: 73 - protein, 14 aa, synthetic construct
QLRSNEAYHVVKKS

SEQ ID NO: 74 - protein, 14 aa, synthetic construct
QTQMQEPKYGVSHS

SEQ ID NO: 75 - protein, 14 aa, synthetic construct
DVRWDEPYYVDEKI

SEQ ID NO: 76 - protein, 14 aa, synthetic construct
HVPRVEPYNVVTNY

SEQ ID NO: 77 - protein, 14 aa, synthetic construct
QFPGQALSFVHEKS

SEQ ID NO: 78 - protein, 14 aa, synthetic construct
HFPSKALNNTAKNT

SEQ ID NO: 79 - protein, 14 aa, synthetic construct
QFPPEQPYGLANKS

SEQ ID NO: 80 - protein, 14 aa, synthetic construct
HVPWAGRHYVAKHS

SEQ ID NO: 81 - protein, 14 aa, synthetic construct
RVAIDELYLIAHKS

SEQ ID NO: 82 - protein, 14 aa, synthetic construct
QIPEQQPYNVFKKS

SEQ ID NO: 83 - protein, 14 aa, synthetic construct
SIPGEDSLYAVTEH

SEQ ID NO: 84 - protein, 14 aa, synthetic construct
QCPSEQPNYVCKIF

SEQ ID NO: 85 - protein, 14 aa, synthetic construct
HGQTQEPSYVTVYY

SEQ ID NO: 86 - protein, 14 aa, synthetic construct
HVHWQVPSNIVRNS

SEQ ID NO: 87 - protein, 14 aa, synthetic construct
QLPWYEADYRMEYY

SEQ ID NO: 88 - protein, 14 aa, synthetic construct
QLPSQAQNYYVTAS

SEQ ID NO: 89 - protein, 14 aa, synthetic construct
QVTSEEHYFAVKKF

SEQ ID NO: 90 - protein, 14 aa, synthetic construct
TSLSGAPFHNANTF

SEQ ID NO: 91 - protein, 14 aa, synthetic construct
HDASIALYDPVLMS

SEQ ID NO: 92 - protein, 14 aa, synthetic construct
QLPGDVTYNLGHKS

SEQ ID NO: 93 - protein, 14 aa, synthetic construct
QPTWEQPYHVDRNS

SEQ ID NO: 94 - protein, 14 aa, synthetic construct
HIPFPGLYSNLDQS

SEQ ID NO: 95 - protein, 14 aa, synthetic construct
DHPWDVRHYAHKKS

SEQ ID NO: 96 - protein, 14 aa, synthetic construct
DVPWKESFNIVENS

SEQ ID NO: 97 - protein, 14 aa, synthetic construct
QFARTAPYYVQTEY

SEQ ID NO: 98 - protein, 14 aa, synthetic construct
QNPWQKPYTVMKTS

SEQ ID NO: 99 - protein, 14 aa, synthetic construct
HLSFEEPLYGVKKS

SEQ ID NO: 100 - protein, 14 aa, synthetic construct
QFPSGHRVYTGKMT

SEQ ID NO: 101 - protein, 14 aa, synthetic construct
YNLYEQPYSVLKRS

SEQ ID NO: 102 - protein, 14 aa, synthetic construct
HLPSQVTHYVITSF

SEQ ID NO: 103 - protein, 14 aa, synthetic construct
QVPSHEPFYLGKKY

SEQ ID NO: 104 - protein, 14 aa, synthetic construct
RVTQGSPVYGENNA

SEQ ID NO: 105 - protein, 14 aa, synthetic construct
QIPMEETYSLRKMS

SEQ ID NO: 106 - protein, 14 aa, synthetic construct
QIPWEAPSYGATIS

SEQ ID NO: 107 - protein, 14 aa, synthetic construct
HVGWEQPYYVLRKS

SEQ ID NO: 108 - protein, 14 aa, synthetic construct
DFATEERRYAVPKY

SEQ ID NO: 109 - protein, 14 aa, synthetic construct
DVPLVKPHYGAVDS

SEQ ID NO: 110 - protein, 14 aa, synthetic construct
QFPSEASHYVVRNS

SEQ ID NO: 111 - protein, 14 aa, synthetic construct
HSRLHETSYVFNKP

SEQ ID NO: 112 - protein, 14 aa, synthetic construct
HVQRQETDYAAKRI

SEQ ID NO: 113 - protein, 14 aa, synthetic construct
NVLSEEPSSRDANV

SEQ ID NO: 114 - protein, 14 aa, synthetic construct
HLPFEAPYYAVKRS

SEQ ID NO: 115 - protein, 14 aa, synthetic construct
QLPGEEPTYVGSNS

SEQ ID NO: 116 - protein, 14 aa, synthetic construct
EVPVGDLSYVGKKY

SEQ ID NO: 117 - protein, 14 aa, synthetic construct
QFPLVQPNYLVEMP

SEQ ID NO: 118 - protein, 14 aa, synthetic construct
HVPGQDSYNVVNKF

SEQ ID NO: 119 - protein, 14 aa, synthetic construct
HVTYTEASVYKRTF

SEQ ID NO: 120 - protein, 14 aa, synthetic construct
QLEWEEPHYAARKS

SEQ ID NO: 121 - protein, 14 aa, synthetic construct
HYQSVKSYSVQKKI

SEQ ID NO: 122 - protein, 14 aa, synthetic construct
QLTSAEASYVGKQS

SEQ ID NO: 123 - protein, 14 aa, synthetic construct
HRPWEEPYYVAKQS

SEQ ID NO: 124 - protein, 14 aa, synthetic construct
LFASENPYNVKSKS

SEQ ID NO: 125 - protein, 14 aa, synthetic construct
SVPGEDPSQFGKTP

SEQ ID NO: 126 - protein, 14 aa, synthetic construct
HVPSEEPYKLDEQS

SEQ ID NO: 127 - protein, 14 aa, synthetic construct
QFRIEDSYDVANQS

SEQ ID NO: 128 - protein, 14 aa, synthetic construct
QIYWHESNYLVTKS

SEQ ID NO: 129 - protein, 14 aa, synthetic construct
HIPGDETSYVMKKY

SEQ ID NO: 130 - protein, 14 aa, synthetic construct
QSLSEEPQYVVTPS

SEQ ID NO: 131 - protein, 14 aa, synthetic construct
HILLEEPSNVLNKR

SEQ ID NO: 132 - protein, 14 aa, synthetic construct
TLPVYVPYYVVNNT

SEQ ID NO: 133 - protein, 14 aa, synthetic construct
HMRSEQPYYGGKVS

SEQ ID NO: 134 - protein, 14 aa, synthetic construct
EVPRTERNFVVKNT

SEQ ID NO: 135 - protein, 14 aa, synthetic construct
HGAWEETHYVVIQS

SEQ ID NO: 136 - protein, 14 aa, synthetic construct
HGRSVESYDLVNKR

SEQ ID NO: 137 - protein, 14 aa, synthetic construct
QVTWGEPQYVVYKS

SEQ ID NO: 138 - protein, 14 aa, synthetic construct
QLPSGEPYSFVKES

SEQ ID NO: 139 - protein, 14 aa, synthetic construct
DVPLEDTYYRVKKY

SEQ ID NO: 140 - protein, 14 aa, synthetic construct
HVPFDKSSHLVKKY

SEQ ID NO: 141 - protein, 14 aa, synthetic construct
QFAWDSPYSRGKKT

SEQ ID NO: 142 - protein, 14 aa, synthetic construct
HVTWQQTKYVTNRS

SEQ ID NO: 143 - protein, 14 aa, synthetic construct
RFPLEEPNYFQTKS

SEQ ID NO: 144 - protein, 14 aa, synthetic construct
HVTWGKTYHLVTDS

SEQ ID NO: 145 - protein, 14 aa, synthetic construct
LVPWEEPGYFQNTS

SEQ ID NO: 146 - protein, 14 aa, synthetic construct
QYPREEPRHALKMS

SEQ ID NO: 147 - protein, 14 aa, synthetic construct
NVTFEQSSYVVTSP

SEQ ID NO: 148 - protein, 14 aa, synthetic construct
YVTSESPYYVHGQP

SEQ ID NO: 149 - protein, 14 aa, synthetic construct
HVSGTKPYHLVKKS

SEQ ID NO: 150 - protein, 14 aa, synthetic construct
EDVLHNAYHVETQF

SEQ ID NO: 151 - protein, 14 aa, synthetic construct
QFAWEHRDYVLQRS

SEQ ID NO: 152 - protein, 14 aa, synthetic construct
QVLWEERLYVVKNS

SEQ ID NO: 153 - protein, 14 aa, synthetic construct
RSTWEQSYTIVKES

SEQ ID NO: 154 - protein, 14 aa, synthetic construct
QMASKEPNYPVKNS

SEQ ID NO: 155 - protein, 14 aa, synthetic construct
QNPAEEQYIVWNNS

SEQ ID NO: 156 - protein, 14 aa, synthetic construct
SHPWEKQYDVFNNS

SEQ ID NO: 157 - protein, 14 aa, synthetic construct
DVPWEEPYHVGNWR

SEQ ID NO: 158 - protein, 14 aa, synthetic construct
HVASEQPYYGVNRP

SEQ ID NO: 159 - protein, 14 aa, synthetic construct
HVLGEEPYYFVQHV

SEQ ID NO: 160 - protein, 14 aa, synthetic construct
QEPWDEFSYNVNAL

SEQ ID NO: 161 - protein, 14 aa, synthetic construct
QFRSGEAYVVVQTS

SEQ ID NO: 162 - protein, 14 aa, synthetic construct
QNAFSKPYNGVKPS

SEQ ID NO: 163 - protein, 14 aa, synthetic construct
QIESGEPYHDQKHP

SEQ ID NO: 164 - protein, 14 aa, synthetic construct
QVRWPEQYYVVNMG

SEQ ID NO: 165 - protein, 14 aa, synthetic construct
QYAREEPYYFMSYK

SEQ ID NO: 166 - protein, 14 aa, synthetic construct
QIPCQSDHNVVTNC

SEQ ID NO: 167 - protein, 14 aa, synthetic construct
DRLSDEPFKGMTHS

SEQ ID NO: 168 - protein, 14 aa, synthetic construct
DVRWSDPHDRITKS

SEQ ID NO: 169 - protein, 14 aa, synthetic construct
HLPWEGPEYLGKKS

SEQ ID NO: 170 - protein, 14 aa, synthetic construct
HVPRDEPQSFVHRS

SEQ ID NO: 171 - protein, 14 aa, synthetic construct
QFSSAEAYTVVNES

SEQ ID NO: 172 - protein, 14 aa, synthetic construct
QIPSHSPYYALNSS

SEQ ID NO: 173 - protein, 14 aa, synthetic construct
QQRLSASYQHVIKS

SEQ ID NO: 174 - protein, 14 aa, synthetic construct
HFEGEDLYDRVKSR

SEQ ID NO: 175 - protein, 14 aa, synthetic construct
HVTLEKPYDVATKS

SEQ ID NO: 176 - protein, 14 aa, synthetic construct
QLPGEHPYYPIKKP

SEQ ID NO: 177 - protein, 14 aa, synthetic construct
EALWYKHYYGVHKT

SEQ ID NO: 178 - protein, 14 aa, synthetic construct
HVVRHYPPHFVHKS

SEQ ID NO: 179 - protein, 14 aa, synthetic construct
LIPSEERSYAVINT

SEQ ID NO: 180 - protein, 14 aa, synthetic construct
QHTHKQPFNVVKNY

SEQ ID NO: 181 - protein, 14 aa, synthetic construct
VVPSQGTYHVGMTS

SEQ ID NO: 182 - protein, 14 aa, synthetic construct
AVAWERPYYVVNHS

SEQ ID NO: 183 - protein, 14 aa, synthetic construct
DVLLARTYYFGEFP

SEQ ID NO: 184 - protein, 14 aa, synthetic construct
HFAGQEHLHFVEIS

SEQ ID NO: 185 - protein, 14 aa, synthetic construct
LFAWAEANNVMKNS

SEQ ID NO: 186 - protein, 14 aa, synthetic construct
SITNKQSVYVLKKS

SEQ ID NO: 187 - protein, 14 aa, synthetic construct
HVQSANPYYDVRNS

SEQ ID NO: 188 - protein, 14 aa, synthetic construct
HVSFDEPYYYVKKS

SEQ ID NO: 189 - protein, 14 aa, synthetic construct
QDYWAKPSYIVKHS

SEQ ID NO: 190 - protein, 14 aa, synthetic construct
QISLTEATLVVRKS

SEQ ID NO: 191 - protein, 14 aa, synthetic construct
DVLWEEPRYFVNNS

SEQ ID NO: 192 - protein, 14 aa, synthetic construct
HAHGQETYFLVKQS

SEQ ID NO: 193 - protein, 14 aa, synthetic construct
HFALQDPYQVFKKT

SEQ ID NO: 194 - protein, 14 aa, synthetic construct
QYPAERPTFDVTNA

SEQ ID NO: 195 - protein, 14 aa, synthetic construct
SLPMEERYYAVRTA

SEQ ID NO: 196 - protein, 14 aa, synthetic construct
DVEWEEPYYFLEKS

SEQ ID NO: 197 - protein, 14 aa, synthetic construct
DVTWEEPYYAVKNS

SEQ ID NO: 198 - protein, 14 aa, synthetic construct
EHPRVESVYLFPHT

SEQ ID NO: 199 - protein, 14 aa, synthetic construct
HDSLDLHYYAMHES

SEQ ID NO: 200 - protein, 14 aa, synthetic construct
QCPLEESCFDGQKS

SEQ ID NO: 201 - protein, 14 aa, synthetic construct
QLTWPKQNYTMTQS

SEQ ID NO: 202 - protein, 14 aa, synthetic construct
QLPWGAPYYVVKNS

SEQ ID NO: 203 - protein, 14 aa, synthetic construct
RLPSEEPYTVAKRP

SEQ ID NO: 204 - protein, 14 aa, synthetic construct
DFPWEQRSSTVKQY

SEQ ID NO: 205 - protein, 14 aa, synthetic construct
DHTRVHPYYDFEIP

SEQ ID NO: 206 - protein, 14 aa, synthetic construct
HATLQEPYFVVNHY

SEQ ID NO: 207 - protein, 14 aa, synthetic construct
HDTWKGPYYPANKS

SEQ ID NO: 208 - protein, 14 aa, synthetic construct
QLPSQEPYNVQRKS

SEQ ID NO: 209 - protein, 14 aa, synthetic construct
SVTRYDLSTDVTKS

SEQ ID NO: 210 - protein, 14 aa, synthetic construct
ESQWEQPSDVFKVV

SEQ ID NO: 211 - protein, 14 aa, synthetic construct
KLPLEEPFYIVTPS

SEQ ID NO: 212 - protein, 14 aa, synthetic construct
QIRWEDASYVEKTS

SEQ ID NO: 213 - protein, 14 aa, synthetic construct
QVPWKGQYYVVASS

SEQ ID NO: 214 - protein, 14 aa, synthetic construct
HGPTDGPYYVSKKS

SEQ ID NO: 215 - protein, 14 aa, synthetic construct
HILRQEPYYVLTNY

SEQ ID NO: 216 - protein, 14 aa, synthetic construct
HSHGQELFYVVTNT

SEQ ID NO: 217 - protein, 14 aa, synthetic construct
HVPWGEPEALVKKS

SEQ ID NO: 218 - protein, 14 aa, synthetic construct
QPPREQPYHIMKNS

SEQ ID NO: 219 - protein, 14 aa, synthetic construct
QTSWAGPHDRVTKA

SEQ ID NO: 220 - protein, 14 aa, synthetic construct
QVPCEEPWCLVKVN

SEQ ID NO: 221 - protein, 14 aa, synthetic construct
QVPWQDPVYDVNNA

SEQ ID NO: 222 - protein, 14 aa, synthetic construct
YVKTVNPDSVLKIS

SEQ ID NO: 223 - protein, 14 aa, synthetic construct
HVHFTDPHYVMKKS

SEQ ID NO: 224 - protein, 14 aa, synthetic construct
HVPLQGPYYVKQKP

SEQ ID NO: 225 - protein, 14 aa, synthetic construct
NVEWEIPYYFVKKF

SEQ ID NO: 226 - protein, 14 aa, synthetic construct
QFAWVQPYYFAKKS

SEQ ID NO: 227 - protein, 14 aa, synthetic construct
QHPVKERDEFWKES

SEQ ID NO: 228 - protein, 14 aa, synthetic construct
QIPWGETTAGIKKL

SEQ ID NO: 229 - protein, 14 aa, synthetic construct
EFSREQPNDFLNKS

SEQ ID NO: 230 - protein, 14 aa, synthetic construct
HIPFAEHDGVVPIS

SEQ ID NO: 231 - protein, 14 aa, synthetic construct
HVTSHEARYLAQNS

SEQ ID NO: 232 - protein, 14 aa, synthetic construct
EFTWEGLSEGVNEP

SEQ ID NO: 233 - protein, 14 aa, synthetic construct
HVSSEYPYYVVKHT

SEQ ID NO: 234 - protein, 14 aa, synthetic construct
HVPAEPLVYENQKS

SEQ ID NO: 235 - protein, 14 aa, synthetic construct
LIVSQPSYLAVQYS

SEQ ID NO: 236 - protein, 14 aa, synthetic construct
QLPLAKPNSVVATS

SEQ ID NO: 237 - protein, 14 aa, synthetic construct
RLAWEEPHYVVKKS

SEQ ID NO: 238 - protein, 14 aa, synthetic construct
KFTTQDSYYFGTQT

SEQ ID NO: 239 - protein, 14 aa, synthetic construct
NVTSREPYHLLEKS

SEQ ID NO: 240 - protein, 14 aa, synthetic construct
QLPFIGPYHVAKKS

SEQ ID NO: 241 - protein, 14 aa, synthetic construct
HDASGVSYYDRRES

SEQ ID NO: 242 - protein, 14 aa, synthetic construct
HVGLPEPHHVMIHS

SEQ ID NO: 243 - protein, 14 aa, synthetic construct
EVPSLQPYHGGKKS

SEQ ID NO: 244 - protein, 14 aa, synthetic construct
QIPSQEPYHRFKES

SEQ ID NO: 245 - protein, 14 aa, synthetic construct
QIPGEAPYSFGKKY

SEQ ID NO: 246 - protein, 14 aa, synthetic construct
QNPWHYPYSLVSTA

SEQ ID NO: 247 - protein, 14 aa, synthetic construct
DVPFRLWNFAVVDS

SEQ ID NO: 248 - protein, 14 aa, synthetic construct
HFAWVVPNDAVKAS

SEQ ID NO: 249 - protein, 14 aa, synthetic construct
HFSWEERYRVVHKS

SEQ ID NO: 250 - protein, 14 aa, synthetic construct
RVPSDEPYSFLRQS

SEQ ID NO: 251 - protein, 14 aa, synthetic construct
TQRWAEPYHVFHKS

SEQ ID NO: 252 - protein, 14 aa, synthetic construct
HVLRHEPYYVVAQA

SEQ ID NO: 253 - protein, 14 aa, synthetic construct
LLPSDEAHYIVKHS

SEQ ID NO: 254 - protein, 14 aa, synthetic construct
NVLSEQPGYVMEYL

SEQ ID NO: 255 - protein, 14 aa, synthetic construct
QLPWTNPFHVVNKY

SEQ ID NO: 256 - protein, 14 aa, synthetic construct
QYSLDESPYGAKKS

SEQ ID NO: 257 - protein, 14 aa, synthetic construct
YVPGAAPIGIVHKS

SEQ ID NO: 258 - protein, 14 aa, synthetic construct
HFAWEEVFHVKKNF

SEQ ID NO: 259 - protein, 14 aa, synthetic construct
HSPSKESHYVLENM

SEQ ID NO: 260 - protein, 14 aa, synthetic construct
HVPFGGTYSVEGES

SEQ ID NO: 261 - protein, 14 aa, synthetic construct
HYRCVGSCYVVAKS

SEQ ID NO: 262 - protein, 14 aa, synthetic construct
LFRSQAQNVLVISH

SEQ ID NO: 263 - protein, 14 aa, synthetic construct
QFPGSELTVSVIQV

SEQ ID NO: 264 - protein, 14 aa, synthetic construct
QFSSQEAHYELNTP

SEQ ID NO: 265 - protein, 14 aa, synthetic construct
QSPWDEPYYGVLMA

SEQ ID NO: 266 - protein, 14 aa, synthetic construct
QSTPEERYHRVIQA

SEQ ID NO: 267 - protein, 14 aa, synthetic construct
QVPLGAPFYAVRKS

SEQ ID NO: 268 - protein, 14 aa, synthetic construct
YERWEAPFYDVQKS

SEQ ID NO: 269 - protein, 14 aa, synthetic construct
HVAWNEPHHVLLQI

SEQ ID NO: 270 - protein, 14 aa, synthetic construct
HVLWEDPYYVVRMP

SEQ ID NO: 271 - protein, 14 aa, synthetic construct
HVQLREHYFVVYSS

SEQ ID NO: 272 - protein, 14 aa, synthetic construct
HVSSEAPYYVLNKS

SEQ ID NO: 273 - protein, 14 aa, synthetic construct
LFPLDETYYVAKVS

SEQ ID NO: 274 - protein, 14 aa, synthetic construct
QALLEEKYTALKKS

SEQ ID NO: 275 - protein, 14 aa, synthetic construct
QFPWEQRNYDVKKY

SEQ ID NO: 276 - protein, 14 aa, synthetic construct
QIPLDARSYLLKRP

SEQ ID NO: 277 - protein, 14 aa, synthetic construct
QIQREEPFYVLKLY

SEQ ID NO: 278 - protein, 14 aa, synthetic construct
QLPSKASNSVQQTF

SEQ ID NO: 279 - protein, 14 aa, synthetic construct
QLPWEKPYYDVEVT

SEQ ID NO: 280 - protein, 14 aa, synthetic construct
QNPGKSPYYVVHKS

SEQ ID NO: 281 - protein, 14 aa, synthetic construct
QVWGEVPLSGAKRF

SEQ ID NO: 282 - protein, 14 aa, synthetic construct
RVPSAAAYYGAKIS

SEQ ID NO: 283 - protein, 14 aa, synthetic construct
VFSSDSQYLFVPNF

SEQ ID NO: 284 - protein, 14 aa, synthetic construct
DRPVEVPSDFVNNS

SEQ ID NO: 285 - protein, 14 aa, synthetic construct
DVLGIERYYAVKDP

SEQ ID NO: 286 - protein, 14 aa, synthetic construct
HHPGVGPFHVVKRS

SEQ ID NO: 287 - protein, 14 aa, synthetic construct
HLSQYEPYDPVDKS

SEQ ID NO: 288 - protein, 14 aa, synthetic construct
HVAWDGAYNLVQTT

SEQ ID NO: 289 - protein, 14 aa, synthetic construct
HVPGQDPYDVQATY

SEQ ID NO: 290 - protein, 14 aa, synthetic construct
HVPLEGSYLDDINS

SEQ ID NO: 291 - protein, 14 aa, synthetic construct
HVTLAEPHLLLGKS

SEQ ID NO: 292 - protein, 14 aa, synthetic construct
KFSLVHPEYYGKET

SEQ ID NO: 293 - protein, 14 aa, synthetic construct
NLPGVEQYNLPNSS

SEQ ID NO: 294 - protein, 14 aa, synthetic construct
NVPWQVPYSVLKMP

SEQ ID NO: 295 - protein, 14 aa, synthetic construct
QFAGEVPYYFATDL

SEQ ID NO: 296 - protein, 14 aa, synthetic construct
QHPMDPTYHVVIKA

SEQ ID NO: 297 - protein, 14 aa, synthetic construct
QILSEEPYYRVDMP

SEQ ID NO: 298 - protein, 14 aa, synthetic construct
QIPLAYSNYVVNKY

SEQ ID NO: 299 - protein, 14 aa, synthetic construct
QITFEVPCCLVRKG

SEQ ID NO: 300 - protein, 14 aa, synthetic construct
QLGLDKLNHLVKKS

SEQ ID NO: 301 - protein, 14 aa, synthetic construct
QSSSVEPYYVLQNS

SEQ ID NO: 302 - protein, 14 aa, synthetic construct
QVACEQPYYNVNCS

SEQ ID NO: 303 - protein, 14 aa, synthetic construct
RLPEAEPYYLMTQP

SEQ ID NO: 304 - protein, 14 aa, synthetic construct
RVPGERQYDALNLS

SEQ ID NO: 305 - protein, 14 aa, synthetic construct
YVPLQEPYYVVKQS

SEQ ID NO: 306 - protein, 14 aa, synthetic construct
ESALKEPHQVLKHS

SEQ ID NO: 307 - protein, 14 aa, synthetic construct
HAPADQLYYPVWKP

SEQ ID NO: 308 - protein, 14 aa, synthetic construct
HFPWEEPYHVAKRS

SEQ ID NO: 309 - protein, 14 aa, synthetic construct
HFQSEEPYHIPHKH

SEQ ID NO: 310 - protein, 14 aa, synthetic construct
HHPLQEPYYVVEKS

SEQ ID NO: 311 - protein, 14 aa, synthetic construct
HMPGREPYYVVTTS

SEQ ID NO: 312 - protein, 14 aa, synthetic construct
HVAWKGPYYVLETH

SEQ ID NO: 313 - protein, 14 aa, synthetic construct
HVHGAPSYSVLKHS

SEQ ID NO: 314 - protein, 14 aa, synthetic construct
HVLWVEPYYVINKY

SEQ ID NO: 315 - protein, 14 aa, synthetic construct
HVPRVDHLYVETKS

SEQ ID NO: 316 - protein, 14 aa, synthetic construct
HVPSGERYYVVKTI

SEQ ID NO: 317 - protein, 14 aa, synthetic construct
HVPYVGAYNGVKKS

SEQ ID NO: 318 - protein, 14 aa, synthetic construct
HVQWEGPYYDVKKS

SEQ ID NO: 319 - protein, 14 aa, synthetic construct
HVRGAELSYVLTKS

SEQ ID NO: 320 - protein, 14 aa, synthetic construct
HVTWGEPYQVLNKS

SEQ ID NO: 321 - protein, 14 aa, synthetic construct
KLLTEAPYMFRTQA

SEQ ID NO: 322 - protein, 14 aa, synthetic construct
KLPWQESYDIVRKS

SEQ ID NO: 323 - protein, 14 aa, synthetic construct
KLQWQDPDYVVKNS

SEQ ID NO: 324 - protein, 14 aa, synthetic construct
KQLGVLPDYVRKNT

SEQ ID NO: 325 - protein, 14 aa, synthetic construct
LAPFEERSYALKSP

SEQ ID NO: 326 - protein, 14 aa, synthetic construct
LASWDEPSSVLRES

SEQ ID NO: 327 - protein, 14 aa, synthetic construct
LLPPDEPYYPMKKS

SEQ ID NO: 328 - protein, 14 aa, synthetic construct
NLQWEQPSYVAKDY

SEQ ID NO: 329 - protein, 14 aa, synthetic construct
NVPWSEQYNALKTS

SEQ ID NO: 330 - protein, 14 aa, synthetic construct
QFASSHTDYVADKS

SEQ ID NO: 331 - protein, 14 aa, synthetic construct
QIQWQGSYNDLNKS

SEQ ID NO: 332 - protein, 14 aa, synthetic construct
QISSEEPTSLLDRP

SEQ ID NO: 333 - protein, 14 aa, synthetic construct
QKPTGASYYGTKPS

SEQ ID NO: 334 - protein, 14 aa, synthetic construct
QLAFDGPSPILTTS

SEQ ID NO: 335 - protein, 14 aa, synthetic construct
QLHWGQPYSILNKS

SEQ ID NO: 336 - protein, 14 aa, synthetic construct
QLPCEVPCYLVQYS

SEQ ID NO: 337 - protein, 14 aa, synthetic construct
QLPLQYRYYFVDQS

SEQ ID NO: 338 - protein, 14 aa, synthetic construct
QLPWEGKYQLGKKR

SEQ ID NO: 339 - protein, 14 aa, synthetic construct
QLRWHEPYHVVNRY

SEQ ID NO: 340 - protein, 14 aa, synthetic construct
QSQCAGPGSAYKKC

SEQ ID NO: 341 - protein, 14 aa, synthetic construct
QTLAGETNSVVLKF

SEQ ID NO: 342 - protein, 14 aa, synthetic construct
QTQCEEPCYVVKFA

SEQ ID NO: 343 - protein, 14 aa, synthetic construct
QVPSEEQFYAAVKS

SEQ ID NO: 344 - protein, 14 aa, synthetic construct
QVPSENPFRVLKNL

SEQ ID NO: 345 - protein, 14 aa, synthetic construct
QVRYVASSDLVRIP

SEQ ID NO: 346 - protein, 14 aa, synthetic construct
QVSLQEPGDVLKEF

SEQ ID NO: 347 - protein, 14 aa, synthetic construct
QVSREEPSGVVNKP

SEQ ID NO: 348 - protein, 14 aa, synthetic construct
RLPSEALYYLVKRS

SEQ ID NO: 349 - protein, 14 aa, synthetic construct
YVPVEGIQHVVEKS

SEQ ID NO: 350 - protein, 14 aa, synthetic construct
HVRCEEPYCHVRKS

SEQ ID NO: 351 - protein, 14 aa, synthetic construct
ELPWQKRKDVMKYP

SEQ ID NO: 352 - protein, 14 aa, synthetic construct
QMPWEVPYYVVKRS

SEQ ID NO: 353 - protein, 14 aa, synthetic construct
DDIWEVPKYRPKNS

SEQ ID NO: 354 - protein, 14 aa, synthetic construct
QIASEERDNIVKKS

SEQ ID NO: 355 - protein, 14 aa, synthetic construct
RVPWQEPYYVVLKP

SEQ ID NO: 356 - protein, 14 aa, synthetic construct
QLPGYEPYYVGTVS

SEQ ID NO: 357 - protein, 14 aa, synthetic construct
RIPSVGPYGVVKTF

SEQ ID NO: 358 - protein, 14 aa, synthetic construct
QDVWAEPYYAGKQP

SEQ ID NO: 359 - protein, 14 aa, synthetic construct
HVAWQEPRYFVKKS

SEQ ID NO: 360 - protein, 14 aa, synthetic construct
YLFAEELAYNLTKS

SEQ ID NO: 361 - protein, 14 aa, synthetic construct
QRPWEQAYFVVRST

SEQ ID NO: 362 - protein, 14 aa, synthetic construct
FVSREEPYYVVKKS

SEQ ID NO: 363 - protein, 14 aa, synthetic construct
DVAREKPYYDMKKS

SEQ ID NO: 364 - protein, 14 aa, synthetic construct
QFPSVGSYYVVPQS

SEQ ID NO: 365 - protein, 14 aa, synthetic construct
HVVWESSHFVVIDS

SEQ ID NO: 366 - protein, 14 aa, synthetic construct
QLRLGDLYNVMTKE

SEQ ID NO: 367 - protein, 14 aa, synthetic construct
QFQWQELLNVVKDS

SEQ ID NO: 368 - protein, 14 aa, synthetic construct
HVPLQEADYVLKKF

SEQ ID NO: 369 - protein, 14 aa, synthetic construct
QFPLYDPYEVMPKS

SEQ ID NO: 370 - protein, 14 aa, synthetic construct
HFRWDEPYYIANKA

SEQ ID NO: 371 - protein, 14 aa, synthetic construct
RDRHEDSKFVMNHS

SEQ ID NO: 372 - protein, 14 aa, synthetic construct
QVPFAHPYYVVNMS

SEQ ID NO: 373 - protein, 14 aa, synthetic construct
HVRWGEPSYVLKNF

SEQ ID NO: 374 - protein, 14 aa, synthetic construct
QGQTETHSHDAKRY

SEQ ID NO: 375 - protein, 14 aa, synthetic construct
QLRFEGPDNPGVQS

SEQ ID NO: 376 - protein, 14 aa, synthetic construct
QLPWEVPYSVVRQS

SEQ ID NO: 377 - protein, 14 aa, synthetic construct
HVPLPGPYFVLKNY

SEQ ID NO: 378 - protein, 14 aa, synthetic construct
HVKRIETYYVFTKS

SEQ ID NO: 379 - protein, 14 aa, synthetic construct
RIHLYQPYSILKNY

SEQ ID NO: 380 - protein, 14 aa, synthetic construct
QFTSEETHYVVNKS

SEQ ID NO: 381 - protein, 14 aa, synthetic construct
QHPWEDPYKIFNKY

SEQ ID NO: 382 - protein, 14 aa, synthetic construct
DVPWPNAYNFGIKS

SEQ ID NO: 383 - protein, 14 aa, synthetic construct
DVPFEHSYYGEQQY

SEQ ID NO: 384 - protein, 14 aa, synthetic construct
EFLGEAPYYVVSRS

SEQ ID NO: 385 - protein, 14 aa, synthetic construct
HVSGEEPYTVGNTS

SEQ ID NO: 386 - protein, 14 aa, synthetic construct
QFQLDQSYYSFNTL

SEQ ID NO: 387 - protein, 14 aa, synthetic construct
QILRVHPDYVVVNP

SEQ ID NO: 388 - protein, 14 aa, synthetic construct
HFPWDEAYLVVKNP

SEQ ID NO: 389 - protein, 14 aa, synthetic construct
HFSSQESNSGGKKW

SEQ ID NO: 390 - protein, 14 aa, synthetic construct
HISGEDPYFILQNS

SEQ ID NO: 391 - protein, 14 aa, synthetic construct
TFQYEQPNLVVKNA

SEQ ID NO: 392 - protein, 14 aa, synthetic construct
LLAWDEQDYFLRKT

SEQ ID NO: 393 - protein, 14 aa, synthetic construct
HINWDKRYAVVITY

SEQ ID NO: 394 - protein, 14 aa, synthetic construct
HFPSEYPFDLVSKS

SEQ ID NO: 395 - protein, 14 aa, synthetic construct
QFLWQQPDHVRRKS

SEQ ID NO: 396 - protein, 14 aa, synthetic construct
QIVLNAPYYVVKTS

SEQ ID NO: 397 - protein, 14 aa, synthetic construct
TVPAHNPYYLVKQT

SEQ ID NO: 398 - protein, 14 aa, synthetic construct
QDPSVDPYYVDRSS

SEQ ID NO: 399 - protein, 14 aa, synthetic construct
QFHCHGACYIVKNS

SEQ ID NO: 400 - protein, 14 aa, synthetic construct
RLPQHESYIVGKIS

SEQ ID NO: 401 - protein, 14 aa, synthetic construct
RVYWIDPYYIVNES

SEQ ID NO: 402 - protein, 14 aa, synthetic construct
QIPLKTPYNVVKKF

SEQ ID NO: 403 - protein, 14 aa, synthetic construct
QFPQGEAGYIAKSF

SEQ ID NO: 404 - protein, 14 aa, synthetic construct
QFYWDQSNYGEQKP

SEQ ID NO: 405 - protein, 14 aa, synthetic construct
HVPWAGSSDVLKVT

SEQ ID NO: 406 - protein, 14 aa, synthetic construct
QVPWEKPVSFVQKS

SEQ ID NO: 407 - protein, 14 aa, synthetic construct
HVWWEDPYYVVRKT

SEQ ID NO: 408 - protein, 14 aa, synthetic construct
WLHWEQPYWTVKKS

SEQ ID NO: 409 - protein, 14 aa, synthetic construct
LFAGENGYFTVRQF

SEQ ID NO: 410 - protein, 14 aa, synthetic construct
QVSWVYPPNDVIVS

SEQ ID NO: 411 - protein, 14 aa, synthetic construct
NDPWPVPYYMVKNS

SEQ ID NO: 412 - protein, 14 aa, synthetic construct
QVQWQVPYYVVKKP

SEQ ID NO: 413 - protein, 14 aa, synthetic construct
HFPLQEPYYVVKNS

SEQ ID NO: 414 - protein, 14 aa, synthetic construct
QGSSVATSSVLTML

SEQ ID NO: 415 - protein, 14 aa, synthetic construct
QPCADKPYCVHTIT

SEQ ID NO: 416 - protein, 14 aa, synthetic construct
HVWWEEAHYSWKHS

SEQ ID NO: 417 - protein, 14 aa, synthetic construct
DLFVEEPYLGVIKF

SEQ ID NO: 418 - protein, 14 aa, synthetic construct
YSLWEDLIFNMQNS

SEQ ID NO: 419 - protein, 14 aa, synthetic construct
QIPWVEASNVLISF

SEQ ID NO: 420 - protein, 14 aa, synthetic construct
QFGQENHNHFVRKS

SEQ ID NO: 421 - protein, 14 aa, synthetic construct
QVSLVESRSVLKIL

SEQ ID NO: 422 - protein, 14 aa, synthetic construct
AVQRAKPYYAVQNS

SEQ ID NO: 423 - protein, 14 aa, synthetic construct
HLLGDYRFWAFTNA

SEQ ID NO: 424 - protein, 14 aa, synthetic construct
ESPWAPPYYVVKKS

SEQ ID NO: 425 - protein, 14 aa, synthetic construct
QIPTVEPYYEGKKS

SEQ ID NO: 426 - protein, 14 aa, synthetic construct
QVRGGQPDQVLRLS

SEQ ID NO: 427 - protein, 14 aa, synthetic construct
FVARNEPFYVVGTS

SEQ ID NO: 428 - protein, 14 aa, synthetic construct
QIPLEEMYYVVRTY

SEQ ID NO: 429 - protein, 14 aa, synthetic construct
QVSSVGPNHVVKKI

SEQ ID NO: 430 - protein, 14 aa, synthetic construct
QLPWGKPYNVVVYS

SEQ ID NO: 431 - protein, 14 aa, synthetic construct
QTPWQVPGYNMLNS

SEQ ID NO: 432 - protein, 14 aa, synthetic construct
AYLSEQLNYVMKNS

SEQ ID NO: 433 - protein, 14 aa, synthetic construct
QASSEAPSYPVVKS

SEQ ID NO: 434 - protein, 14 aa, synthetic construct
LDQWHTPNYVVRKS

SEQ ID NO: 435 - protein, 14 aa, synthetic construct
QLLWDPPSYFVNNS

SEQ ID NO: 436 - protein, 14 aa, synthetic construct
DVPWEQPYFVVRNS

SEQ ID NO: 437 - protein, 14 aa, synthetic construct
QVPYGQSFVVGTSS

SEQ ID NO: 438 - protein, 14 aa, synthetic construct
QLRWEDQYAHVEKS

SEQ ID NO: 439 - protein, 14 aa, synthetic construct
HVAWKAPYHFTKNS

SEQ ID NO: 440 - protein, 14 aa, synthetic construct
DGLYVRSFYVLQHS

SEQ ID NO: 441 - protein, 14 aa, synthetic construct
QFALKGLNGDFQKS

SEQ ID NO: 442 - protein, 14 aa, synthetic construct
QFPSAETYRIVKKF

SEQ ID NO: 443 - protein, 14 aa, synthetic construct
QLTWELGYHAFVKP

SEQ ID NO: 444 - protein, 14 aa, synthetic construct
QLLRNLSYDGAKYS

SEQ ID NO: 445 - protein, 14 aa, synthetic construct
QLRVKLQYDVIANN

SEQ ID NO: 446 - protein, 14 aa, synthetic construct
QVPAREPNHIVKKS

SEQ ID NO: 447 - protein, 14 aa, synthetic construct
SAREQGTFYFVNNS

SEQ ID NO: 448 - protein, 14 aa, synthetic construct
LFPKYAPHESVKMS

SEQ ID NO: 449 - protein, 14 aa, synthetic construct
QVPGNELYFVLKQY

SEQ ID NO: 450 - protein, 14 aa, synthetic construct
QFAWSQSYAYSSTT

SEQ ID NO: 451 - protein, 14 aa, synthetic construct
QGQWDKPYDAVQNS

SEQ ID NO: 452 - protein, 14 aa, synthetic construct
QIPGDAQYRLVKRS

SEQ ID NO: 453 - protein, 14 aa, synthetic construct
QIPWTQPQYDVTPP

SEQ ID NO: 454 - protein, 14 aa, synthetic construct
HVELESSYYFVNNS

SEQ ID NO: 455 - protein, 14 aa, synthetic construct
QFPLAEPYYHLTES

SEQ ID NO: 456 - protein, 14 aa, synthetic construct
HFDWEEPYYDASKA

SEQ ID NO: 457 - protein, 14 aa, synthetic construct
SYEWEDPYYDLKYS

SEQ ID NO: 458 - protein, 14 aa, synthetic construct
NVTGAEYYYRMNNY

SEQ ID NO: 459 - protein, 14 aa, synthetic construct
HVSCEVPCYGALAS

SEQ ID NO: 460 - protein, 14 aa, synthetic construct
HDASEGAYYVERNS

SEQ ID NO: 461 - protein, 14 aa, synthetic construct
QFGWEEPYYVLHTV

SEQ ID NO: 462 - protein, 14 aa, synthetic construct
HLSREGTYYAVKQP

SEQ ID NO: 463 - protein, 14 aa, synthetic construct
QLPLYDPYYTVRTS

SEQ ID NO: 464 - protein, 14 aa, synthetic construct
ELPYEEPGTFVEKA

SEQ ID NO: 465 - protein, 14 aa, synthetic construct
QFPWEEPYYVFTNS

SEQ ID NO: 466 - protein, 14 aa, synthetic construct
QIPGGERSYVLENS

SEQ ID NO: 467 - protein, 14 aa, synthetic construct
QITSDKPYYVVKNS

SEQ ID NO: 468 - protein, 14 aa, synthetic construct
YDPSGEPYYRVNNH

SEQ ID NO: 469 - protein, 14 aa, synthetic construct
QVPRPASSYIGKTS

SEQ ID NO: 470 - protein, 14 aa, synthetic construct
QIPLEGPTYVTNPS

SEQ ID NO: 471 - protein, 14 aa, synthetic construct
AIPSGAPYYKVNKY

SEQ ID NO: 472 - protein, 14 aa, synthetic construct
EFRSEQPYYFVNKY

SEQ ID NO: 473 - protein, 14 aa, synthetic construct
QIRWDETYYDIQKT

SEQ ID NO: 474 - protein, 14 aa, synthetic construct
QLALEQQSYDAKMS

SEQ ID NO: 475 - protein, 14 aa, synthetic construct
ALPWEHPWCVSKNC

SEQ ID NO: 476 - protein, 14 aa, synthetic construct
HVPVLEPYDLVKKS

SEQ ID NO: 477 - protein, 14 aa, synthetic construct
QIRSTEPYYVFKKT

SEQ ID NO: 478 - protein, 14 aa, synthetic construct
QSPGTNPQNVLIKS

SEQ ID NO: 479 - protein, 14 aa, synthetic construct
QITSVDPSSLAYRS

SEQ ID NO: 480 - protein, 14 aa, synthetic construct
QMPSGAPYDATLNS

SEQ ID NO: 481 - protein, 14 aa, synthetic construct
QNPWDEPSYVVKNV

SEQ ID NO: 482 - protein, 14 aa, synthetic construct
QSPFDETAYGLTHS

SEQ ID NO: 483 - protein, 14 aa, synthetic construct
QVLFAKSYYSVKDS

SEQ ID NO: 484 - protein, 14 aa, synthetic construct
RFPADEEHYVGKLS

SEQ ID NO: 485 - protein, 14 aa, synthetic construct
YVPYEEQKYVVTKS

SEQ ID NO: 486 - protein, 14 aa, synthetic construct
HSQGQTTEYVGKQS

SEQ ID NO: 487 - protein, 14 aa, synthetic construct
DFQSEETRNLAKMP

SEQ ID NO: 488 - protein, 14 aa, synthetic construct
DVPWPAPYYAVSKY

SEQ ID NO: 489 - protein, 14 aa, synthetic construct
QFPGEESHYVVRNS

SEQ ID NO: 490 - protein, 14 aa, synthetic construct
QFQWETPSHVINDS

SEQ ID NO: 491 - protein, 14 aa, synthetic construct
QTPRQTLYYSVKAY

SEQ ID NO: 492 - protein, 14 aa, synthetic construct
HVAYQEPFVYQKTS

SEQ ID NO: 493 - protein, 14 aa, synthetic construct
QISRGDTYYVMNKS

SEQ ID NO: 494 - protein, 14 aa, synthetic construct
HIPGEEPSDGVRSY

SEQ ID NO: 495 - protein, 14 aa, synthetic construct
HLSVAEPYYAVRNS

SEQ ID NO: 496 - protein, 14 aa, synthetic construct
QLQWQKSNWVGKIT

SEQ ID NO: 497 - protein, 14 aa, synthetic construct
EIPWEGTYRLVKHS

SEQ ID NO: 498 - protein, 14 aa, synthetic construct
HVPQKEPYYVVKTF

SEQ ID NO: 499 - protein, 14 aa, synthetic construct
QFPLFTTFYNVEES

SEQ ID NO: 500 - protein, 14 aa, synthetic construct
QFPWETSYYIVKNS

SEQ ID NO: 501 - protein, 14 aa, synthetic construct
QLAGGDPYVLPKLS

SEQ ID NO: 502 - protein, 14 aa, synthetic construct
DVASGEQSYIVVKY

SEQ ID NO: 503 - protein, 14 aa, synthetic construct
HVWLEDTISAEKKS

SEQ ID NO: 504 - protein, 14 aa, synthetic construct
QVQWDGASSIVQKT

SEQ ID NO: 505 - protein, 14 aa, synthetic construct
HFRGPEPYKFVENP

SEQ ID NO: 506 - protein, 14 aa, synthetic construct
HVPWEGPEYIVTQS

SEQ ID NO: 507 - protein, 14 aa, synthetic construct
QFPRHDPNEVVHKS

SEQ ID NO: 508 - protein, 14 aa, synthetic construct
QIPSEEPNYILKSS

SEQ ID NO: 509 - protein, 14 aa, synthetic construct
QRPGDEPSYFVKNS

SEQ ID NO: 510 - protein, 14 aa, synthetic construct
ELTMEQPSYVGNVS

SEQ ID NO: 511 - protein, 14 aa, synthetic construct
QLPWHEAFFGVNNG

SEQ ID NO: 512 - protein, 14 aa, synthetic construct
QVLEKEPSSNVHKS

SEQ ID NO: 513 - protein, 14 aa, synthetic construct
HVPSYAPNYEIKES

SEQ ID NO: 514 - protein, 14 aa, synthetic construct
QHLGEEPSYSGEYA

SEQ ID NO: 515 - protein, 14 aa, synthetic construct
SISSKDPYALVKYS

SEQ ID NO: 516 - protein, 14 aa, synthetic construct
QFPWDEPNYVVKNF

SEQ ID NO: 517 - protein, 14 aa, synthetic construct
QLPFNEPYYSQQVS

SEQ ID NO: 518 - protein, 14 aa, synthetic construct
HLSMEAPYYGVKEY

SEQ ID NO: 519 - protein, 14 aa, synthetic construct
HVLGEQPYSVVKTS

SEQ ID NO: 520 - protein, 14 aa, synthetic construct
HVSWEEPRYIVKDS

SEQ ID NO: 521 - protein, 14 aa, synthetic construct
QLPWEGPYYIVKKS

SEQ ID NO: 522 - protein, 14 aa, synthetic construct
QTPAEKPYYVVKKY

SEQ ID NO: 523 - protein, 14 aa, synthetic construct
HVPSQEWYYDLHKS

SEQ ID NO: 524 - protein, 14 aa, synthetic construct
QFPWHKQFYVGKSS

SEQ ID NO: 525 - protein, 14 aa, synthetic construct
DFQIVGPSYVGKNS

SEQ ID NO: 526 - protein, 14 aa, synthetic construct
HLPSEKPWYDLDGS

SEQ ID NO: 527 - protein, 14 aa, synthetic construct
HVSWSTSKYVVNHS

SEQ ID NO: 528 - protein, 14 aa, synthetic construct
QISWEDPDYGVRKS

SEQ ID NO: 529 - protein, 14 aa, synthetic construct
QVPCEQQSCVVKNS

SEQ ID NO: 530 - protein, 14 aa, synthetic construct
LFTGQQSYYEVQES

SEQ ID NO: 531 - protein, 14 aa, synthetic construct
LVSPTGPIYVMNKS

SEQ ID NO: 532 - protein, 14 aa, synthetic construct
QFASEEPYHVMKKP

SEQ ID NO: 533 - protein, 14 aa, synthetic construct
QFSRADSQYLVKNS

SEQ ID NO: 534 - protein, 14 aa, synthetic construct
QIPLQDPYHVVEKS

SEQ ID NO: 535 - protein, 14 aa, synthetic construct
QLLWGESYSIVKNY

SEQ ID NO: 536 - protein, 14 aa, synthetic construct
QLPSQTQNYYDTNA

SEQ ID NO: 537 - protein, 14 aa, synthetic construct
QLRSEDTYYTMQSY

SEQ ID NO: 538 - protein, 14 aa, synthetic construct
SVAGQQHYYVSTKP

SEQ ID NO: 539 - protein, 14 aa, synthetic construct
HLPCKKSFYACKKH

SEQ ID NO: 540 - protein, 14 aa, synthetic construct
HVRFAEPYYAGKTS

SEQ ID NO: 541 - protein, 14 aa, synthetic construct
LLTGAEPYYLGKRS

SEQ ID NO: 542 - protein, 14 aa, synthetic construct
QLPGAEAQNVVKYS

SEQ ID NO: 543 - protein, 14 aa, synthetic construct
QLRFDEHHSVVTHS

SEQ ID NO: 544 - protein, 14 aa, synthetic construct
QYPWEQRYYFERKS

SEQ ID NO: 545 - protein, 14 aa, synthetic construct
RVTWEGPYNSVKKS

SEQ ID NO: 546 - protein, 14 aa, synthetic construct
DVALGESHYNVKKY

SEQ ID NO: 547 - protein, 14 aa, synthetic construct
DVPNDLPSYLVKNS

SEQ ID NO: 548 - protein, 14 aa, synthetic construct
EFPYQESEYIVKQS

SEQ ID NO: 549 - protein, 14 aa, synthetic construct
GLPSKEPYYVVKPA

SEQ ID NO: 550 - protein, 14 aa, synthetic construct
HVPWDDQYYVWRTN

SEQ ID NO: 551 - protein, 14 aa, synthetic construct
HVQWDQTSYVVKNS

SEQ ID NO: 552 - protein, 14 aa, synthetic construct
HVTSDDSYYAVKNP

SEQ ID NO: 553 - protein, 14 aa, synthetic construct
QDPFGDLYYVTKMS

SEQ ID NO: 554 - protein, 14 aa, synthetic construct
QFPYDEPYNVVKKS

SEQ ID NO: 555 - protein, 14 aa, synthetic construct
QHPWKEQYYMATKP

SEQ ID NO: 556 - protein, 14 aa, synthetic construct
QLNSEEPYYHFKNY

SEQ ID NO: 557 - protein, 14 aa, synthetic construct
QLPTNDPYYVLMES

SEQ ID NO: 558 - protein, 14 aa, synthetic construct
QVPWEEPYYVVKKS

SEQ ID NO: 559 - protein, 17 aa, synthetic construct
  source 1..17: note = HNP-401
SGQVPWEEPYYVVKKSS

SEQ ID NO: 560 - protein, 20 aa, synthetic construct
  source 1..20: note = HNP-401 with GGC linker
SGQVPWEEPYYVVKKSSGGC

SEQ ID NO: 561 - protein, 15 aa, synthetic construct
  source 1..15: note = HNP401-N-2
QVPWEEPYYVVKKSS

SEQ ID NO: 562 - protein, 17 aa, synthetic construct
  source 1..17: note = HNP401-N-2 with GG linker
QVPWEEPYYVVKKSSGG

SEQ ID NO: 563 - protein, 18 aa, synthetic construct
  source 1..18: note = HNP401-N-2 with GGC linker
QVPWEEPYYVVKKSSGGC

SEQ ID NO: 564 - protein, 11 aa, synthetic construct
  source 1..11: note = JNK Inhibitor VI
RPKRPTTLNLF

SEQ ID NO: 565 - protein, 16 aa, synthetic construct
  source 1..16: note = SC 3036
KKHTDDGYMPMSPGVA

SEQ ID NO: 566 - protein, 28 aa, synthetic construct
  source 1..28: note = NEMO-Binding Domain Binding Peptide
DRQIKIWFQNRRMKWKKTALDWSWLQTE

SEQ ID NO: 567 - protein, 26 aa, synthetic construct
  source 1..26: note = NF-kB SN50
AAVALLPAVLLALLAPVQRKRQKLMP

SEQ ID NO: 568 - protein, 29 aa, synthetic construct
  source 1..29: note = TIRAP Inhibitor Peptide
RQIKIWFNRRMKWKKLQLRDAAPGGAIVS

SEQ ID NO: 569 - protein, 4 aa, synthetic construct
LEHD

SEQ ID NO: 570 - protein, 4 aa, synthetic construct
IETD

SEQ ID NO: 571 - protein, 4 aa, synthetic construct
LETD

SEQ ID NO: 572 - protein, 4 aa, synthetic construct
GGGC

SEQ ID NO: 573 - protein, 4 aa, synthetic construct
KKKK

SEQ ID NO: 574 - protein, 4 aa, synthetic construct
GGGG

SEQ ID NO: 575 - protein, 5 aa, synthetic construct
SGGGG

SEQ ID NO: 576 - protein, 18 aa, synthetic construct
QVPWEEPYYVVKKSSGGL